(19) United States
(12) Patent Application Publication
(10) Pub. No.: US 2024/0327850 A1
(43) Pub. Date: Oct. 3, 2024

(54) METHODS AND COMPOSITIONS FOR CONTROLLING RELEASE FACTOR ACTIVITY AND USES THEREOF

(71) Applicants: Opentrons Labworks Inc., Brooklyn, NY (US); New York University, New York, NY (US)

(72) Inventors: Joel S. BADER, Bronx, NY (US); Jef D. BOEKE, New York, NY (US); Leslie MITCHELL, New York, NY (US); Akil HAMZA, Long Island City, NY (US)

(21) Appl. No.: 18/558,656

(22) PCT Filed: May 4, 2022
(86) PCT No.: PCT/US2022/027706

§ 371 Date: Nov. 2, 2023

Related U.S. Application Data
(60) Provisional application No. 63/184,115, filed on May 4, 2021.

(51) Int. Cl. C12N 15/81

(52) U.S. Cl. CPC C12N 15/81

(57) ABSTRACT

Provided herein are systems and methods for stop codon rewriting and replacement. Also provided herein are systems and methods for producing a polypeptide comprising a non-canonical amino acid.

### CROSS REFERENCE

This application is a national phase entry of International Application No. PCT/US2022/027706, filed on May 4, 2022, which claims the benefit of U.S. Provisional Application No. 63/184,115, filed on May 4, 2021, each of which is incorporated herein by reference in its entirety.

### SEQUENCE LISTING

This instant application contains a Sequence Listing, which has been submitted electronically in ASCII format and is hereby incorporated by reference in its entirety. Said ASCII copy, created on Jun. 7, 2022, is named 59725-705_601_SL.txt and is 403,196 bytes in size.

### BACKGROUND

Codon rewriting and repurposing translational machinery may be important tools to expand the genetic code artificially. These may also be important tools to enable incorporation of non-canonical amino acids (ncAAs) into proteins. Many methods for ncAA incorporation use a stop codon together with a suppressor tRNA to convert the stop codon into a sense codon. These methods suffer, however, because the suppressor tRNA competes with the native release factor, resulting in early termination and poor readthrough. Methods that control release factor activity to avoid recognizing a defined subset of stop codons, especially in eukaryotic cells, would have great utility in improving the performance of methods for genetic code expansion and ncAA incorporation.

### SUMMARY

In some aspects, provided herein is a method comprising: rewriting a first stop codon to a second stop codon in a genome of a first organism; and introducing a release factor into the first organism, wherein the release factor is configured to recognize only the second stop codon as a stop codon, and wherein the release factor does not recognize the first stop codon as a stop codon.

In some aspects, provided herein, is a method of producing a polypeptide molecule comprising a non-canonical amino acid (ncAA) or a population of polypeptide molecules comprising the ncAA in a first organism, the method comprising: a. rewriting a first stop codon to a second stop codon; b. reassigning the first stop codon to encode the ncAA in the genome of the first organism; and c. introducing an aminoacyl-tRNA synthetase (aaRS)/tRNA pair into the first organism, wherein the aaRS/tRNA pair is configured to recognize the first stop codon and incorporate the ncAA into an amino acid sequence of the polypeptide or the population of the polypeptide molecules.

In some aspects, provided herein, is a cell or a population of cells comprising a first stop codon rewritten to a second stop codon and further comprising (a) a release factor that recognizes only the second stop codon as a stop codon, (b) a release factor that recognizes only the second stop codon as a stop codon, (c) a release factor that recognizes only the third stop codon as a stop codon, or (d) a combination thereof.

In some aspects, provided herein, is an organism comprising the cell or the population of cells described herein.

In some aspects, provided herein is a method of producing a polypeptide molecule comprising a non-canonical amino acid (ncAA) or a population of polypeptide molecules comprising the ncAA, the method comprising introducing into the cell or the population of cells described herein, a) a first nucleic acid sequence construct encoding the polypeptide wherein the first nucleic acid sequence construct comprises the first stop codon reassigned to encode the ncAA; and b) a second nucleic acid sequence construct encoding an aminoacyl-tRNA synthetase (aaRS)/tRNA pair engineered to recognize the first stop codon and incorporate the ncAA into an amino acid sequence of the polypeptide, thereby producing the polypeptide molecule comprising the ncAA or the population of polypeptide molecules comprising the ncAA.

In some aspects, provided herein, is a composition comprising: (a) a recombinant release factor configured to recognize only a second stop codon, (b) a recombinant release factor configured to recognize only a first stop codon as a stop codon, (c) a recombinant release factor configured to recognize only the third stop codon as a stop codon, or (d) a combination thereof.

In some aspects, provided herein, is a method comprising: a. rewriting UAA and UAG to UGA in a genome of a yeast; b. introducing a release factor into the yeast, wherein the release factor is configured to recognize only UGA as a stop codon, and wherein the release factor does not recognize UAA and UAG as a stop codon; and c. reassigning UAA or UAG to encode a natural amino acid or a non-canonical amino acid (ncAA).

In some aspects, provided herein, is a system for producing a polypeptide molecule comprising a non-canonical amino acid (ncAA) comprising the ncAA comprising: a. a gene encoding the polypeptide molecule, wherein the gene comprises a first stop codon rewritten to a second stop codon, and wherein the first stop codon is reassigned to encode the ncAA; b. a release factor, wherein (i) the release factor is configured to recognize only the second stop codon as a stop codon, and wherein the release factor does not recognize the first stop codon as a stop codon, (ii) the release factor is configured to recognize only the first stop codon as a stop codon, (iii) the release factor is configured to recognize only a third stop codon as a stop codon, or (iv) a combination thereof; and c. an aminoacyl-tRNA synthetase (aaRS)/tRNA pair, wherein the aaRS/tRNA pair is configured to recognize the first stop codon and incorporate the ncAA into an amino acid sequence of the polypeptide molecule.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows the recognition of the three stop codons UAG, UAA and UGA by prokaryotic (upper line) and eukaryotic (lower line) release factors. Prokaryotes contain two distinct single subunit release factors with the indicated specificities. Eukaryotes contain a single, release factor eRF1 which in conjunction with eRF3 recognizes all three stop codons. In certain species, such as the ciliate Tetrahymena and others, only the UGA stop codon is recognized by eRF1, while in others species such as the ciliate Euplotes only the UAG and UAA stop codons are recognized.

FIG. 2 shows an example embodiment of a shuffle episome system for the yeast S. cerevisiae. In some embodiments, the payload may comprise a SUP45 gene, encoding eRF1. In some embodiments, the payload may comprise a SUP35 gene, encoding eRF3. In some embodiments, the payload may comprise both a SUP45 gene and a SUP35 gene. In other embodiments, additional payload elements may be included such as homologs of the genes MTQ2, TRM112, and genes encoding tRNA^{Trp}. The diagram in the center illustrates the generic architecture of a plasmid system used to build yeast strains that can either assess the specificity of a given eRF system or survive solely on one or more ciliate eRF proteins in the absence of the cognate yeast eRF protein or proteins. The diagram indicates the position of the payload (a release factor gene or genes with optionally, additional payload genes) and vector components. The vector components include a selectable marker and may include other sequences such as a centromere and/or an origin of replication. Two types of vector may be used, a payload vector containing a positive selection marker such as LEU2, HIS3. ADE2 intended to host a non-S. cerevisiae payload. A second type of vector is a shuffle vector (shown in the diagram) that includes the S. cerevisiae payload eRF gene or genes and a counter-selectable marker such as one or more copies of URA3. The diagram on the left shows how plasmid shuffling can result in the replacement of the shuffle vector and its S. cerevisiae payload can be replaced by one or more payload plasmids, if and only if those payload plasmids produce one more eRF1 proteins that are able to substitute for the essential function of the S.cerevisiae eRF1 protein. Further details can be seen in FIGS. 4 and 5.

FIG. 3 shows phylogenetic trees for ciliates. FIG. 3A shows a phylogenetic tree for ciliate organisms. FIG. 3B shows a phylogenetic tree for ciliate organisms with examples of specific ciliates that only recognize the UGA stop codon

FIG. 4 shows examples of ciliate gene constructs that can be tested for function and stop codon specificity in yeast. A specific example embodiment of how these gene constructs can be deployed is given in FIG. 5.

FIG. 5 shows an example embodiment of a shuffle episome system. This system is specifically designed to evaluate function of ciliate-derived engineered RF sequences in yeast. In this embodiment, a yeast strain is constructed encoding its only copy of the yeast eRF1 gene on a shuffle plasmid, such as a Superloser plasmid, which is marked with a counterselectable marker such as URA3. Into this strain, two separate ciliate-derived engineered eRF constructs (or appropriately marked empty vectors) can be transformed. The first, marked with LEU2, is designed to exclusively recognize the UAA and UAG stop codons, and the second, marked with HIS3, is designed to exclusively recognize UGA. After removal of the shuffle plasmid by selection on 5-FOA, strains carrying vectors either the UGA-specific or the UAG/UAA-specific eRF gene alone will be unable to grow since not all stop codon types can be decoded. A strain carrying vectors expressing both types of ciliate-derived engineered eRF genes will be able to grow because all three stop codons can be decoded.

FIG. 6 shows stop-codon selectivity of ciliate domain/motif-swapped eRF1 proteins in yeast.

FIG. 7 shows stop-codon selectivity of whole-gene ciliate eRF1/eRF3 constructs in yeast.

FIG. 8 shows the assessment of plasmid dependency of erf14 strains carrying ciliate release factor constructs.

FIG. 9 shows an example embodiment of a computer system a program configured to implement methods provided herein. In some cases, the program comprises an algorithm. The computer system may be a machine learning-based or statistical learning-based computer system that uses observed patterns of codon usage to select replacement codons. In some cases, the computer system comprises a computer processing unit and a sequence processing unit, wherein the computer processing unit and the sequence processing unit are bilaterally communicatively coupled. In some embodiments, the sequence processing unit and the computer processing unit comprise a storage component. 901: Computer system. 905: Central processing unit (CPU). 910: Memory. 915: Electronic storage unit. 920: Central processing unit of computer system. 925: Peripheral devices. 930: Data storage with files containing the translation tables representing the genetic code of the organism whose genome is being rewritten. 935: electronic display. 940: Instructions describing which translation table to use, the codons to be eliminated, and the locations of input and output files. 950: Computer program implementing the methods to perform the codon rewriting.

### INCORPORATION BY REFERENCE

Each patent, publication, and non-patent literature cited in the application is hereby incorporated by reference in its entirety as if each was incorporated by reference individually.

### DETAILED DESCRIPTION

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

The nomenclature used to describe polypeptides or proteins follows the conventional practice wherein the amino group is presented to the left (the amino- or N-terminus) and the carboxyl group to the right (the carboxy- or C-terminus) of each amino acid residue. When amino acid residue positions are referred to in a polypeptide or a protein, they are numbered in an amino to carboxyl direction with position one being the residue located at the amino terminal end of the polypeptide or the protein of which it can be a part. The amino acid sequences of peptides set forth herein are generally designated using the standard single letter or three letter symbol. (A or Ala for Alanine; C or Cys for Cysteine; D or Asp for Aspartic Acid; E or Glu for Glutamic Acid; F or Phe for Phenylalanine; G or Gly for Glycine; H or His for Histidine; I or Ile for Isoleucine; K or Lys for Lysine; L or Leu for Leucine; M or Met for Methionine; N or Asn for Asparagine; P or Pro for Proline; Q or Gln for Glutamine; R or Arg for Arginine; S or Ser for Serine; T or Thr for Threonine; V or Val for Valine; W or Trp for Tryptophan; and Y or Tyr for Tyrosine).

The term “non-canonical amino acid” or “ncAA” refers to any amino acid other than the 20 standard amino acids (alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine). There are over 700 known ncAA any of which may be used in the methods described herein. In some embodiments, examples of ncAA include, but are not limited to, L-Tryptazan, 5-Fluoro-L-tryptophan, L-Ethionine, L-Selenomethionine, Trifluoro-L-methionine, L-Norleucine, L-Homopropargylglycine, (2S)-2-amino-5-(methylsulfanyl) pentanoic acid, (2S)-2-amino-6-(methylsulfanyl) hexanoic acid, Para-fluoro-L-phenylalanine, Para-iodo-L-phenylalanine, Para-azido-L-phenylalanine, Para-acetyl-L-phenylalanine, Para-benzoyl-L-phenylalanine, Meta-fluoro-L-tyrosine, O-methyl-L-tyrosine, Para-propargyloxy-L-phenylalanine, (2S)-2-aminooctanoic acid, (2S)-2-aminononanoic acid, (2S)-2-aminodecanoic acid, (2S)-2-aminohept-6-enoic acid, (2S)-2-aminooct-7-enoic acid, L-Homocysteine, (2S)-2-amino-5-sulfanylpentanoic acid, (2S)-2-amino-6-sulfanylhexanoic acid, L-S-(2-nitrobenzyl) cysteine, L-S-ferrocenyl-cysteine, L-O-crotylserine, L-O-(pent-4-en-1-yl) serine, L-O-(4,5-dimethoxy-2-nitrobenzyl) serine, (2S)-2-amino-3-({[5-(dimethylamino) naphthalen-1-yl]sulfonyl}amino) propanoic acid, (2S)-3-[(6-acetyl-naphthalen-1-yl)amino]-2-aminopropanoic acid, L-Pyrrolysine, N⁶-[(propargyloxy) carbonyl]-L-lysine, L-N⁶-acetyllysine, N⁶-trifluoroacetyl-L-lysine, N⁶-{[1-(6-nitro-1,3-benzodioxol-5-yl)ethoxy]carbonyl}-L-lysine, N⁶-{[2-(3-methyl-3H-diaziren-3-yl)ethoxy]carbonyl}-L-lysine, p-azidophenylalanine, and 2-aminoisobutyric acid. In some embodiments, examples of ncAA include, but are not limited to, AbK (unnatural amino acid for Photo-crosslinking probe), 3-Aminotyrosine (unnatural amino acid for inducing red shift in fluorescent proteins and fluorescent protein-based biosensors), L-Azidohomoalanine hydrochloride (unnatural amino acid for bio-orthogonal labeling of newly synthesized proteins), L-Azidonorleucine hydrochloride (unnatural amino acid for bio-orthogonal or fluorescent labeling of newly synthesized proteins), BzF (photoreactive unnatural amino acid; photo-crosslinker), DMNB-caged-Serine (caged serine; excited by visible blue light), HADA (blue fluorescent D-amino acid for labeling peptidoglycans in live bacteria), NADA-green (fluorescent D-amino acid for labeling peptidoglycans in live bacteria), NB-caged Tyrosine hydrochloride (ortho-nitrobenzyl caged L-tyrosine), RADA (orange-red TAMRA-based fluorescent D-amino acid for labeling peptidoglycans in live bacteria), Rf470DL (blue rotor-fluorogenic fluorescent D-amino acid for labeling peptidoglycans in live bacteria), sBADA (green fluorescent D-amino acid for labeling peptidoglycans in bacteria), and YADA (green-yellow lucifer yellow-based fluorescent D-amino acid for labeling peptidoglycans in live bacteria). In some embodiments, examples of ncAA include, but are not limited to, β-alanine, D-alanine, 4-hydroxyproline, desmosine, D-glutamic acid, γ-aminobutyric acid, β-cyanoalanine, norvaline, 4-(E)-butenyl-4 (R)-methyl-N-methyl-L-threonine, N-methyl-L-leucine, selenocysteine, and statine. In some embodiments, a ncAA comprises p-azidophenylalanine or 2-aminoisobutyric acid (also known as α-aminoisobutyric acid, AIB, α-methylalanine, or 2-methylalanine).

The terms “codon” and “anticodon” as used herein may refer to DNA or RNA. In some embodiments, DNA comprises nucleotide bases adenine (A), guanine (G), cytosine (C), or thymine (T). In some embodiments, RNA comprises nucleotide bases adenine (A), guanine (G), cytosine (C), or uracil (U). In some embodiments, DNA or RNA may comprise inosine (I). in some embodiments, inosine (I) may pair with adenine (A), cytosine (C), or uracil (U). In some embodiments, DNA or RNA may comprise queuosine (Q). In some embodiments, queuosine (Q) may pair with cytosine (C) or uracil (U).

Although methods and materials similar or equivalent to those described herein can be used in the practice or testing of the present disclosure, suitable methods, and materials are described below.

### Stop Codon Removal and Replacement

### Stop Codons

In standard translation tables, the codons UGA, UAA, and UAG are stop codons. In some embodiments, one or two of these codons may be selected to serve as sense codons. In some embodiments, the UAG codon may be selected to serve as a sense codon.

In some embodiments, the standard stop codons that are not used as sense codons are repeated in the 3′ UTR to improve the efficiency of translational termination. In some embodiments, UGA may remain as the stop codon, and stop signals in coding domains are rewritten from a single stop codon (either UGA, UAA, or UAG) to a double stop, UGAUGA.

In some embodiments, stop codons can not encode amino acids and can not bind tRNAs.

In some embodiments, singleton UGA (opal) can be next to UGG (Tryptophan).

In some embodiments, pair UAA (ochre) and UAG (amber) can be next to UAU/C (Tyrosine).

### Release Factors (RFs)

In some embodiments, release Factors (RFs) can comprise protein adaptors with two major activities. In some embodiments, the first major activity can comprise a Class 1 activity. In some embodiments, the Class 1 activity can comprise mRNA-binding and recognizing the stop codon. In some embodiments, the Class 1 activity may be provided by a release factor 1 (RF1) or an RF2. In some embodiments, the Class I activity may be provided by a eukaryotic release factor 1 (eRF1). In some embodiments, the second major activity can comprise a Class 2 activity. In some embodiments, the Class 2 activity may be provided by an RF3. In some embodiments, the Class 1 activity may be provided by an eRF3. In some embodiments, the Class 2 activity can comprise protein-binding and recognizing the ribosome to release the translated protein.

Wobble rules can be different for RFs than for tRNAs. Release factors can recognize NNA separately from NNG (anti-codon starts with U) and from NNA/C/U (anti-codon starts with A modified to I). For sense codons, NNA can be either recognized with NNU/A as a two-codon block or with NNT/C/A as a three-codon block, or as part of NNT/C/G/A as a four-codon block.

### Release Factors (RFs) in Prokaryotes and Eukaryotes

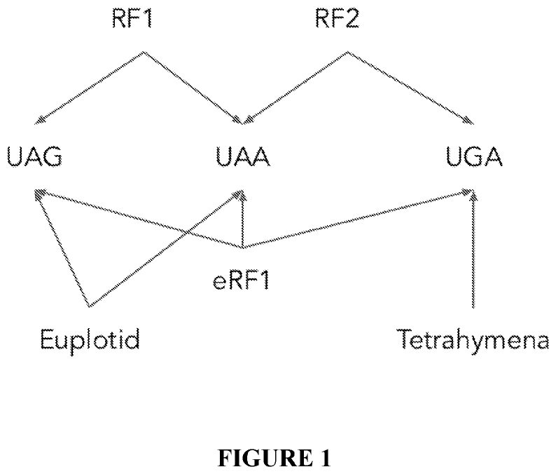

In some embodiments, the release factors can comprise release factors (RFs) from prokaryotes. In some embodiments, the prokaryotic release factors can comprise release factors from Eubacteria and/or mitochondria. In some embodiments, the prokaryotic release factors can comprise two classes (FIG. 1). In some embodiments, the prokaryotic Class 1 release factors can comprise RF1 and RF2. In some embodiments, RF1 can recognize the stop codons UAA and UAG. In some embodiments, RF2 can recognize the stop codons UAA and UGA. In some embodiments, the prokaryotic Class 2 release factors can comprise RF3. In some embodiments, release factors can comprise a recognition domain. In some embodiments, the recognition domain can recognize a stop codon.

In some embodiments, the release factors can comprise release factors from eukaryotes. In some embodiments, the eukaryotic release factors can comprise release factors from Eukaryotes and/or Archaebacteria. In some embodiments, the eukaryotic release factors can comprise two classes (FIG. 1). In some embodiments, the eukaryotic Class 1 release factors can comprise eRF1. In some embodiments, eRF1 can recognize the stop codons UAA, UAG, and UGA. Table 1 shows the activity of eRF1 in different eukaryotic organisms. In some embodiments, the eukaryotic Class 2 release factors can comprise eRF3.

### Evolution

RF1/2 and eRF1 may not be homologous. This lack of homology may suggest that RF activity was provided by RNA adapters prior to the Eubacteria-Archaebacteria split.

Most wild type (WT) eukaryotic RFs (eRFs), including but not limited to yeasts, may recognize all three stop codons, UAG, UAA and UGA. eRFs may form a heterodimer comprising eRF1 and eRF3. In yeast, and more specifically Saccharomyces cerevisiae, eRF1 and eRF3 can be referred to as SUP45 and SUP35, respectively. Some ciliates may have RFs that recognize a subset of the stop codons. For example, a ciliate may have RFs recognizing UAA and UAG. In another example, a ciliate may have RFs recognizing UGA. A yeast system can be engineered with all of the advantages of yeast, for example better suitability for producing certain proteins or other biologics that can be more difficult to produce in bacterial systems. For example, one or more specific domains in yeast eRF1 may be engineered to enable stop codon selectivity conferred in RF of ciliates by replacing one or more yeast amino acids with the corresponding ciliate amino acids. In some embodiments, the yeast eRF1 can be replaced with ciliate eRF1. In some embodiments, the eRF1/eRF3 heterodimer can be replaced with ciliate eRF1/eRF3.

TABLE 1
eRF1 activity in different organisms.

| | | Table 6: | | | | | | |
|---|---|---|---|---|---|---|---|---|
| | | Ciliate (Spirotrichea/ | | | | | | |
| | | Oxytricha/Stylonychia, | | | | | | |
| | | Paramecium, Tetrahymena, | Table 10: | | Table 28: | | | Table 31: |
| | | Heterotrichea/Blepharisma), | Ciliate | Table 27: | Ciliate | Table 29: | Table: 30 | Euglenozoa |
| | Table 1: | Green algae (Dasycladacean), | (Spirotrichea/ | Ciliate | (Heterotrichea/ | Ciliate | Ciliate | (Trypanosomatida/ |
| Codon | Standard | Flagellate (Hexamita) | Euplotid) | (Karyorelict) | Condylostoma) | (Mesodinium) | (Peritrich) | Blastocrithidia) |
| UAU/C | Tyr | | | | | Tyr | | |
| UAA | Stop | Gln | | Gln | Gln/Stop | Tyr | Glu | Glu/Stop |
| | (Ochre) | | | | | | | |
| UAG | Stop | Gln | | Gln | Gln/Stop | Tyr | Glu | Glu/Stop |
| | (Amber) | | | | | | | |
| UGU/C | Cys | | Cys | | | | | |
| UGA | Stop | | Cys | Trp/Stop | Trp/Stop | | | Trp |
| | (Opal) | | | | | | | |
| UGG | Trp | | | Trip | Trip | | | |
| CAA/G | Gln | | | | | | | |
| GAA/G | Glu | | | | | | | |
| Release | | UGA | UAA/G | UGA | Standard | UGA | UGA | UAA/G |
| factor | | only | only | with 3′ | with 3′ | only | only | with 3′ |
| recog- | | | | UTR | UTR | | | UTR |
| nition | | | | | | | | |

Tables here refer to NCBI Genetic Code Tables, which can be found here:
https://www.ncbi.nlm.nih.gov/Taxonomy/Utils/wprintgc.cgi.
The Standard scheme shown in Table 1 is used by most organisms.

Stop-codon assignment to sense codon may have happened as multiple independent events (ciliate, flagellate, green algae lineages). For example, ciliates can comprise a unicellular eukaryote that includes several lineages where stop codons in the standard genetic code have been reassigned to amino acids.

In some embodiments, eRF1 can comprise two main patterns of eRF1 activity. In some embodiments, the first pattern of eRF1 activity can comprise the recognition of the stop codon UGA only. In some embodiments, the stop codons UAA and UAG can be captured by wobble (e.g., UAC/U Tyr). In some embodiments, the stop codons UAA and UGA can be captured by a 1^{st} position neighbor (e.g., CAA/G Gln or GAA/G Glu).

In some embodiments, the second pattern of eRF1 activity can comprise the recognition of UAA/UAG only. In some embodiments, the stop codon UGA can be captured by wobble (e.g., UGU/C Cys, UGG Trp).

In some ciliates, the eRF1 recognition can be “clean” and can depend only on the codon. In other ciliates, stop-codon recognition can depend on 3′ UTR structure.

In some embodiments, UAG can be useful for recoding. In some embodiments, the anticodons for UAA and UGA may have too much wobble for recoding.

Unlike prokaryotes where recognition patterns are UAA/UAG and UAA/UGA, in eukaryotic species where stop codons have been captured as sense codons, evolution seems to favor UAA/UAG and UGA alone.

In some embodiments, UAG can be rewritten to UGA. In some embodiments, rewriting both UAG and UAA to UGA can be advantageous.

### Release Factor Engineering

### Embodiment 1. Amino Acid Swap

In some embodiments, an endogenous release factor can be mutated. In some embodiments, the endogenous release factor can comprise one or more mutations. In some embodiments, the endogenous release factor can comprise at least one, at least two, at least three, at least four, at least five, at least about six, at least about seven, at least about eight, at least about nine, at least about ten, at least about 20, at least about 30, at least about 40, at least about 50, at least about 60, at least about 70, at least about 80, at least about 90, at least about 100, or more mutations. In some embodiments, the mutations can result in the endogenous release factor not recognizing a stop codon. In some embodiments, the mutated endogenous release factor may not recognize UGA. In some embodiments, the mutated endogenous release factor may not recognize UAG. In some embodiments, the mutated endogenous release factor may not recognize UAA. In some embodiments, the mutated endogenous release factor may not recognize UGA and UAG. In some embodiments, the mutated endogenous release factor may not recognize UGA and UAA. In some embodiments, the mutated endogenous release factor may not recognize UAG and UAA. In some embodiments, a tRNA may incorporate an amino acid at a codon that in the native system is recognized as a stop codon rather than a sense codon.

In some embodiments, the mutations may modify a domain or a motif in the endogenous release factor to resemble a domain or motif of a release factor from another organism comprising, but not limited to a ciliate. In some embodiments, a ciliate can comprise any ciliate that uses UGA codons as a termination or stop codon. In some embodiments, a ciliate can comprise, but is not limited to, Blepharisma americanum, Paramecium tetraurelia, Tetrahymena thermophila, Blepharisma japonicum, Euplotes aediculatus, Euplotes octocarinatus, Stentor coeruleus, Nyctotherus ovalis, Stylonychia lemnae, Oxytricha trifallax, Stylonychia pustulata, Stylonychia Mytilus, Eschaneustyla sp. HL-2004, Gonostomum so. HL-2004, Holosticha sp. HL-2004, Urostyla sp. HL-2004, Uroleptus sp. WJC-2003, Paraurostyla weissei, Stichotrichida sp Misty, Stichotrichida sp Alaska, Spiromucleus salmonicida, or Loxodes striatus.

### Embodiment 2. Domain/Motif Swap

In some cases, a recognition domain from a release factor (e.g., a recognition domain of a ciliate (or some green algae or some flagellates) can be swapped into a host cell (e.g., a eukaryotic platform, such as a yeast). In some cases, one or more recognition domains of the host cell can be replaced with one or more recognition domain of an identified release factor (e.g., a ciliate, green algae, or flagellate), for example, via point mutation or via replacement of a continuous segment of the recognition domain. In some embodiments, the domain/motif swapping in the endogenous release factor can result in not recognizing a stop codon. In some embodiments, the domain/motif-swapped release factor may not recognize UGA. In some embodiments, the domain/motif-swapped release factor may not recognize UAG. In some embodiments, the domain/motif-swapped release factor may not recognize UAA. In some embodiments, the domain/motif-swapped release factor may not recognize UGA and UAG. In some embodiments, the domain/motif-swapped release factor may not recognize UGA and UAA. In some embodiments, the domain/motif-swapped release factor may not recognize UAG and UAA. In some embodiments, a tRNA may incorporate an amino acid at a codon that in the native system is recognized as a stop codon rather than a sense codon.

In some embodiments, a domain or motif in the endogenous release factor may be swapped with a domain or motif of a release factor from another organism comprising, but not limited to, a ciliate. In some embodiments, a ciliate can comprise any ciliate that uses UGA codons as a termination or stop codon. In some embodiments, a ciliate can comprise, but is not limited to, Blepharisma americanum, Paramecium tetraurelia, Tetrahymena thermophila, Blepharisma japonicum, Euplotes aediculatus, Euplotes octocarinatus, Stentor coeruleus, Nyctotherus ovalis, Stylonychia lemnae, Oxytricha trifallax, Stylonychia pustulata, Stylonychia Mytilus, Eschaneustyla sp. HL-2004, Gonostomum so. HL-2004, Holosticha sp. HL-2004, Urostyla sp. HL-2004, Uroleptus sp. WJC-2003, Paraurostyla weissei, Stichotrichida sp Misty, Stichotrichida sp Alaska, Spiromucleus salmonicida, or Loxodes striatus.

Domain or motif swapping and mutagenesis experiments in vivo can be allowed in part by temperature-sensitive mutants of the release factor, eRF1-ts. Known mutants can be permissive at lower temperature (30° C.) and restrictive at higher temperature (37° C.). RFs can be engineered to be introduced into a host cell. For example, eRF1-eng can be engineered to be introduced into a yeast cell that also has the eRF1-ts rather than the wild-type, eRF1-wt. After the engineered factor is introduced to the cell with eRF1-ts and lacking eRF1-wt at 30° C., viability can be checked at a higher temperature to see whether the engineered eRF1-eng can complement the reduced function of the ts-mutant eRF1-ts.

Domain/motif-swapped eRF1 can ignore UAA/G in vitro at 37° C., but can recognize UAA/G in vivo at 30° C.

Recognition of UAA/G could be reduced in the presence of competition from ncAA-tRNA (or with further optimization).

### Embodiment 3. Native Ciliate Machinery

Native ciliate machinery may outperform chimeras and mutants.

Native ciliate tRNA^{Trp} may perform better at avoiding UGA codons than endogenous (tRNA^{Trp}).

In some embodiments, the endogenous yeast release factors can be replaced with native ciliate machinery. In some embodiments, native ciliate machinery can comprise non-mutated release factors from a ciliate. In some embodiments, the non-mutated ciliate release factors can recognize one or more stop codons. In some embodiments, the non-mutated ciliate release factors can recognize UGA. In some embodiments, the non-mutated ciliate release factors can recognize UAG. In some embodiments, the non-mutated ciliate release factors can recognize UAA. In some embodiments, the non-mutated ciliate release factors can recognize UGA and UAG. In some embodiments, the non-mutated ciliate release factors can recognize UGA and UAA. In some embodiments, the non-mutated ciliate release factors can recognize UAG and UAA. In some embodiments, the non-mutated ciliate release factors can recognize UGA. In some embodiments, a ciliate can comprise any ciliate that uses UAA and UAG as a termination or stop codon. In some embodiments, a ciliate can comprise any ciliate that uses UGA codons as a termination or stop codon. In some embodiments, a ciliate can comprise, but is not limited to, Blepharisma americanum, Paramecium tetraurelia, Tetrahymena thermophila, Blepharisma japonicum, Euplotes aediculatus, Euplotes octocarinatus, Stentor coeruleus, Nyctotherus ovalis, Stylonychia lemnae, Oxytricha trifallax, Stylonychia pustulata, Stylonychia Mytilus, Eschaneustyla sp. HL-2004, Gonostomum so. HL-2004, Holosticha sp. HL-2004, Urostyla sp. HL-2004, Uroleptus sp. WJC-2003, Paraurostyla weissei, Stichotrichida sp Misty, Stichotrichida sp Alaska, Spiromucleus salmonicida, or Loxodes striatus.

### Methods for Testing Function of Engineered Release Factors

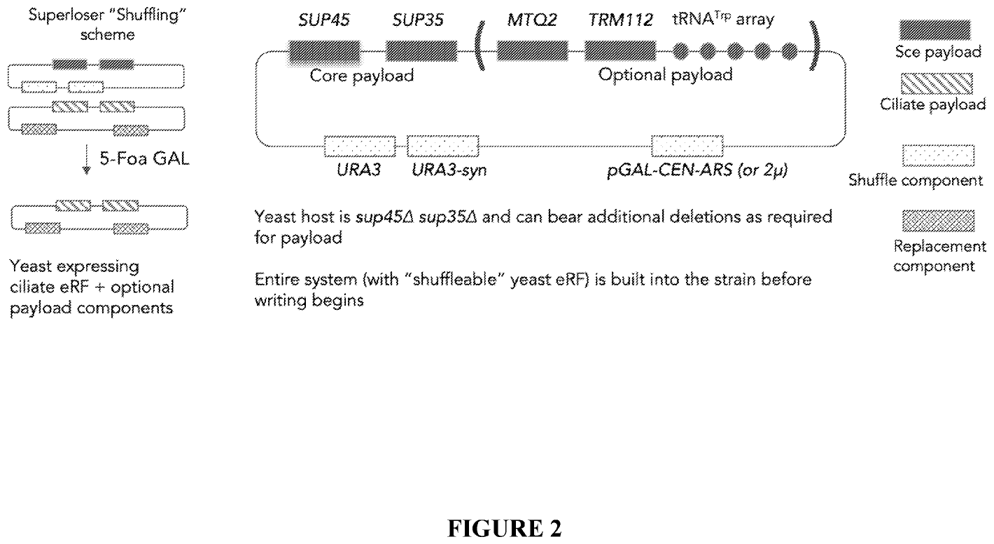

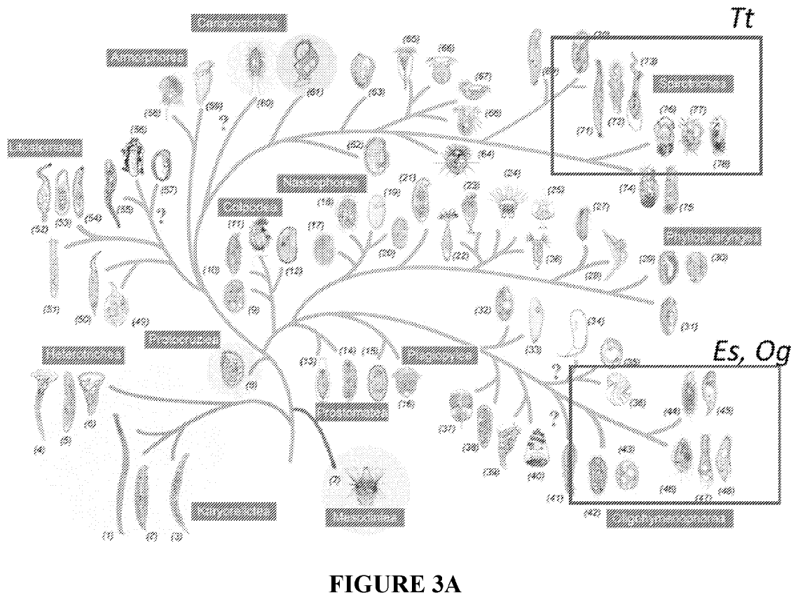

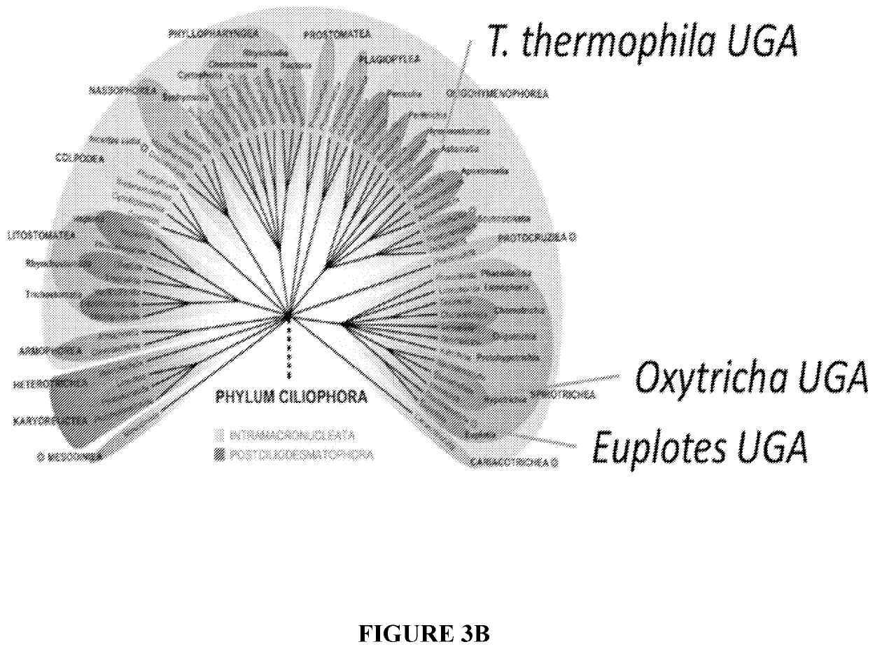

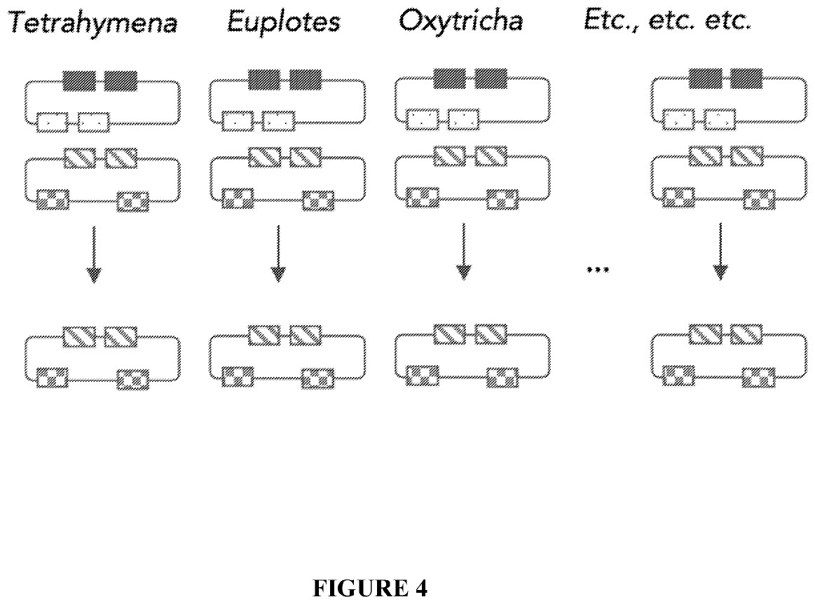

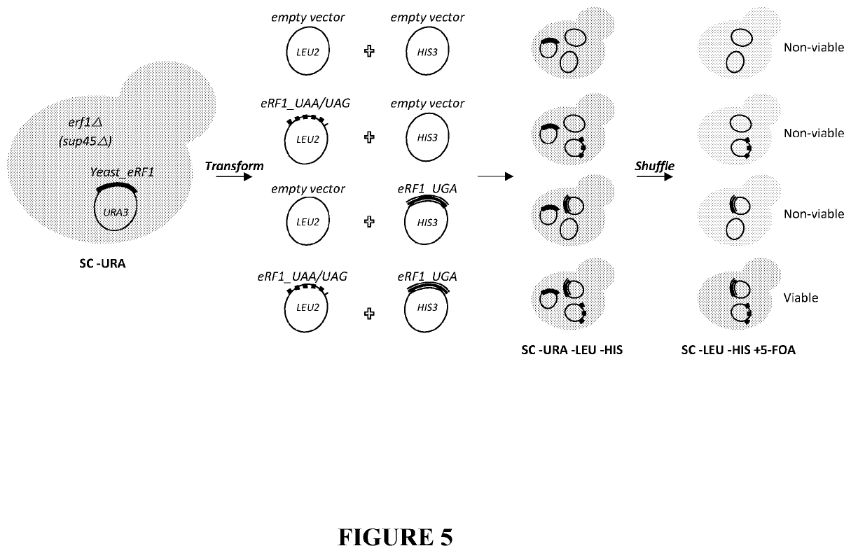

In some aspects, a “shuffle episome” or a “shuffle episome system,” refers to one or more plasmids encoding release factors that are subsequently transformed into yeast. In some embodiments, the shuffle episome or the shuffle episome system can be used in any methods, systems, or embodiments described herein. Ciliate release factors that exclusively recognize UAA/UAG may fail to replace omnipotent release factors because such a strain cannot decode UGA stop codons. Ciliate release factors that exclusively recognize UGA may fail to replace omnipotent yeast release factors because such a strain cannot decode UAA/UAG stop codons. In some embodiments, combining two distinct ciliate release factors, one release factor which can recognize UAA/UAG and the second release factor can recognize UGA in the same stain, can allow “replaceability.” In some embodiments, this “replaceability” can prove the stop codon specificity of the two release factors and simultaneously show that both release factors can function in yeast. In some embodiments, the experimental readout for testing replaceability of the yeast release factors can be cell viability. In some embodiments, the release factors tested can be eRF1/eRF3. In some embodiments, the plasmids can encode a mutated yeast release factor. In some embodiments, the plasmids can encode a native ciliate release factor. In some embodiments, the plasmids can encode a mutated ciliate release factor. In some embodiments, the plasmids can encode a mutated endogenous recognition domain for a release factor. In some embodiments, the plasmids can encode a recognition domain from a second organism. In some embodiments, the plasmids can encode a mutated recognition domain from a second organism. In some embodiments, the expression of the plasmids can be driven by a promoter. In some embodiments, the promoter can comprise an endogenous promoter (e.g., endogenous eRF1/eRF3 promoter). In some embodiments, the promoter can comprise an inducible promoter system (e.g., GAL1/10 system). In some embodiments, the plasmid can encode a selectable marker (e.g., URA3, LEU2, or HIS3). In some embodiments, the plasmid can encode a counter-selectable marker (e.g., URA3). In some example embodiments, the shuffle episome system can be built with all native proteins and/or tRNAs on a supernumerary designer chromosome. Example embodiments of a shuffle episome system are shown in FIG. 2, FIG. 4, and FIG. 5.

Engineered ciliate-derived eRF systems can be tested (FIG. 5). In some embodiments, yeast that only have the UAA/UAG-specific eRF1 constructs post-shuffle may be non-viable. In some embodiments, the UAA-UGA-specific eRF1 yeast strain may be non-viable because the strain cannot decode UGA stop codons. In some embodiments, yeast that only have the UGA-specific eRF1 constructs post-shuffle will be non-viable. In some embodiments, the UGA-specific eRF1 yeast strain may be non-viable because the strain cannot decode UAA/UAG stop codons. In some embodiments, yeast strains that have both the UAA/UAG-specific eRF1 and the UGA-specific eRF1 constructs post-shuffle can be viable. In some embodiments, yeast that have both the UAA/UAG-specific eRF1 and the UGA-specific eRF1 can be viable, which is consistent with stop codon specificity of the two eRF1 constructs and demonstrates that both eRF1 constructs are functional in yeast.

In some embodiments, the engineered eRF machinery can be integrated into the host genome.

### Stop Codon Capture

In some embodiments, the stop codons UAA and UAG can be rewritten to UGA. In some embodiments, rewriting UAA and UAG to UGA may not result in fitness defects.

In some embodiments, the stop codon UAG can be rewritten to UAA. In some embodiments, the stop codon UAG can be rewritten to UAA. In some embodiments, the stop codon UAA can be rewritten to UAG. In some embodiments, the stop codon UAA can be rewritten to UGA. In some embodiments, the stop codon UGA can be rewritten to UAA. In some embodiments, the stop codon UGA can be rewritten to UAG.

In some embodiments, the OAZ1 frameshift can use UGA. In some cases, the OAZ1 frameshift may not be affected by rewriting stop codons.

In some embodiments, a Stop+3 analysis of Saccharomyces and Tetrahymena can be performed to determine whether eRF1 can recognize more than 3 nucleotides.

In some embodiments, eRF1 can be replaced with a de-risked domain-swapped eRF1.

In some embodiments, a native strain can comprise a high-temperature growth defect.

In some embodiments, growth defects in yeast can decrease as UAA/UAG is rewritten to UGA.

In some embodiments, sequence variation, screens, directed evolution, machine learning of eRF1 and interacting proteins can be evaluated. In some embodiments, sequence variation, screens, directed evolution, and machine learning of eRF1 can improve performance of a system, including performance at 30° C.

### Methods for Genome Design

Provided herein are methods, systems, and compositions for designing a genome of an organism. In some embodiments, the organism may be a yeast. In some embodiments, the yeast may be Saccharomyces cerevisiae. In some embodiments, the yeast may be Saccharomyces pastorianus. In some embodiments, the yeast may be Schizosaccharomyces pombe. In some embodiments, the yeast may be Aureobasidium pullulans, Candida albicans, Candida blattae, Candida catenulate, Candida glabrata, Candida humilis, Candida intermedia, Candida melibiosica, Candida pararugosa, Debaryomyces hansenii, Debaryomyces prosopidis, Geotrichum silvicola, Hanseniaspora opuntiae, Hanseniaspora uvarum, Kluyveromyces marxianus, Kodamaea ohmeri, Lachancea thermotolerans, Lodderomyces elongisporus, Meyerozyma guilliermondii, Pichia barkeri, Pichia kudriavzevii, Pichia occidentalis, Rhoditorula mucilaginosa, Saccharomycopsis malanga, Torulaspora delbrueckii, or Yarrowia lipolytica. In some embodiments, native stop codons may be rewritten so that UAG no longer appears as a stop codon. In some embodiments, UAG can be changed to UAA or UGA. In some embodiments, UAG and UAA can be changed to UGA. In some embodiments, all occurrences of UAG and UAA are changed to UGA. In some embodiments, native stop codons may be rewritten so that UAA no longer appears as a stop codon. In some embodiments, UAA can be changed to UGA or UAG. In some embodiments, UGA and UAG can be changed to UAA. In some embodiments, all occurrences of UGA and UAG can be changed UAA. In some embodiments, native stop codons may be rewritten so that UGA no longer appears as a stop codon. In some embodiments, UGA can be changed to UAG or UAA. In some embodiments, UGA and UAA can be changed to UAG. In some embodiments, all occurrences of UGA and UAA can be changed to UAG.

In some embodiments, the first stop codon can comprise UGA, the second stop codon can comprise UAG, and third stop codon can comprise UAA. In some embodiments, the first stop codon can comprise UGA, the second stop codon can comprise UAA, and third stop codon can comprise UAG In some embodiments, the first stop codon can comprise UAG, the second stop codon can comprise UAA, and the third stop can comprise UGA. In some embodiments, the first stop codon can comprise UAG, the second stop codon can comprise UGA, and the third stop codon can comprise UAA. In some embodiments, the first stop codon can comprise UAA, the second stop codon can comprise UGA, and the third codon can comprise UAG. In some embodiments, the first stop codon can comprise UAA, the second stop codon can comprise UAG, and the third stop codon can comprise UGA.

Most wild-type eukaryotic release factors, generally named eRF1, can recognize all three stop codons (e.g., UAG/UAA/UGA). In some cases, a ciliate or other eukaryote, may have release factors that may not recognize all the stop codons. In some cases, a ciliate or a eukaryote may have release factors that may require additional sequence at the 3′ of a stop codon for recognition as a stop codon. For example, some release factors may recognize only UGA as a stop codon and UAA/UAG as sense codons. For example, other release factors may recognize UAA/UAG as stop codons and UGA as a sense codon. In a preferred embodiment, a release factor may recognize UGA as a stop codon.

In some embodiments, some release factors can recognize UGA as a stop codon. In some embodiments, some release factors can recognize UGA as a stop codon and UAG/UAA as sense codons. In some embodiments, some release factors can recognize UGA/UAG as stop codons. In some embodiments, some release factors can recognize UGA/UAG as stop codons and recognize UAA as a sense codon. In some embodiments, some release factors can recognize UGA/UAA as stop codons. In some embodiments, some release factors can recognize UGA/UAA as stop codons and recognize UAG as a sense codon. In some embodiments, some release factors can recognize UAG as a stop codon. In some embodiments, some release factors can recognize UAG as a stop codon and recognize UGA/UAA as sense codons. In some embodiments, some release factors can recognize UAG/UAA as stop codons. In some embodiments, some release factors can recognize UAG/UAA as stop codons and recognize UGA as a sense codon. In some embodiments, some release factors can recognize UAA as a stop codon. In some embodiments, some release factors can recognize UAA as a stop codon and recognize UGA/UAG as stop codons. In some embodiments, some release factors may recognize UGA/UAG/UAA as stop codons. In some embodiments, some release factors may recognize UGA/UAG/UAA as sense codons.

In some embodiments, the release factor can comprise a class 1 release factor. In some embodiments, the class 1 release factor can comprise a prokaryotic release factor 1 (RF1). In some cases, the RF1 can be a eukaryotic RF1 (eRF1). In some embodiments, the eRF1 can be from a ciliate. In some embodiments, the class 1 release factor can comprise a prokaryotic release factor 2 (RF2). In some embodiments, the class 1 release factor can comprise RF1 and RF2. In some embodiments, the release factor can comprise a class 2 release factor. In some embodiments, the class 2 release factor can comprise a release factor 3 (RF3). In some embodiments, the RF3 can be a eukaryotic RF3 (eRF3). In some embodiments, the release factor can be a class 1 release factor or a class 2 release factor. In some embodiments, the release factor can be a class 1 release factor and a class 2 release factor. In some embodiments, the release factor can be a chimeric release factor. In some embodiments, the release factor can be a release factor complex. In some cases, the release factor complex can comprise a release factor 1/release factor 3 (RF1/RF3) complex. In some cases, the release factor complex can comprise a eukaryotic release factor 1/eukaryotic release factor 3 (eRF1/eRF3) complex. In some cases, the release factor complex can comprise a eRF1/chimeric yeast-ciliate eRF3.

In some embodiments, a release factor can comprise one or more mutations. In some cases, the one or more mutations can allow the release factor to recognize only a subset of stop codons (e.g., recognize only one or two stop codons, but not all three stop codons).

In some embodiments, a release factor can comprise a first recognition domain. In some embodiments, a release factor can comprise a first recognition domain swapped with a second recognition domain. In some embodiments, the second recognition domain can be from a second organism. In some embodiments, the second organism can be from a different species of yeast. In some embodiments, the second organism can comprise a ciliate. In some embodiments, a ciliate can comprise any ciliate that uses UGA codons as a termination or stop codon. In some embodiments, a ciliate can comprise any ciliate that uses UAA and/or UAG codons as a termination or stop codon. In some cases, the ciliate can comprise, but is not limited to, Blepharisma americanum, Paramecium tetraurelia, Tetrahymena thermophila, Blepharisma japonicum, Euplotes aediculatus, Euplotes octocarinatus, Stentor coeruleus, Nyctotherus ovalis, Stylonychia lemnae, Oxytricha trifallax, Stylonychia pustulata, Stylonychia Mytilus, Eschaneustyla sp. HL-2004, Gonostomum so. HL-2004, Holosticha sp. HL-2004, Urostyla sp. HL-2004, Uroleptus sp. WJC-2003, Paraurostyla weissei, Stichotrichida sp Misty, Stichotrichida sp Alaska, Spiromucleus salmonicida, or Loxodes striatus. In some embodiments, the second recognition domain can be identified using phylogenetic screening, directed evolution, library screening, or machine learning.

In some embodiments, the second recognition domain can comprise an amino acid sequence comprising KSSNIKS (SEQ ID NO: 3). In some embodiments, the second recognition domain can comprise an amino acid sequence comprising YICDNKF (SEQ ID NO: 4). In some embodiments, the second recognition domain can comprise an amino acid sequence comprising KSSNIKS (SEQ ID NO: 3) and YICDNKF (SEQ ID NO: 4). In some embodiments, the second recognition domain can comprise an amino acid sequence comprising YFCDPQF (SEQ ID NO: 10). In some embodiments, the second recognition domain can comprise an amino acid sequence comprising EAASIKD (SEQ ID NO: 11). In some embodiments, the second recognition domain can comprise an amino acid sequence comprising KATNIKD (SEQ ID NO: 12). In some embodiments, the second recognition domain can comprise an amino acid sequence comprising YFCDSKF (SEQ ID NO: 13). In some embodiments, the second recognition domain can comprise an amino acid sequence comprising TAVNIKS (SEQ ID NO: 5). In some embodiments, the second recognition domain can comprise an amino acid sequence comprising KAANIKS (SEQ ID NO: 6). In some embodiments, the second recognition domain can comprise an amino acid sequence comprising KASNIKS (SEQ ID NO: 7). In some embodiments, the second recognition domain can comprise an amino acid sequence comprising YYCGERF (SEQ ID NO: 8). In some embodiments, the second recognition domain can comprise an amino acid sequence comprising TAESIKS (SEQ ID NO: 9). In some embodiments, the second recognition domain can comprise an amino acid sequence comprising FDFDAES (SEQ ID NO: 14). In some embodiments, the second recognition domain can comprise an amino acid sequence comprising TLIKPQF (SEQ ID NO: 15). In some embodiments, the second recognition domain can comprise an amino acid sequence comprising TGDKIKS (SEQ ID NO: 16). In some embodiments, the second recognition domain can comprise an amino acid sequence comprising TIIKNDF (SEQ ID NO: 17). In some embodiments, the second recognition domain can comprise an amino acid sequence comprising EAASIQD (SEQ ID NO: 18). In some embodiments, the second recognition domain can comprise an amino acid sequence comprising FFCDNYF (SEQ ID NO: 19). In some embodiments, the second recognition domain can comprise an amino acid sequence comprising FVIVNKF (SEQ ID NO: 20). In some embodiments, the second recognition domain can comprise an amino acid sequence comprising AAQNIKS (SEQ ID NO: 21). In some embodiments, the second recognition domain can comprise an amino acid sequence comprising YFCGGKF (SEQ ID NO: 22). In some embodiments, the second recognition domain can comprise an amino acid sequence comprising QANSIKD (SEQ ID NO: 23). In some embodiments, the second recognition domain can comprise an amino acid sequence comprising YRCDSKF (SEQ ID NO: 24). In some embodiments, the second recognition domain can comprise an amino acid sequence comprising GAASIKN (SEQ ID NO: 25). In some embodiments, the second recognition domain can comprise an amino acid sequence comprising YSCNTIF (SEQ ID NO: 26). In some embodiments, the second recognition domain can comprise an amino acid sequence comprising SAQNIKS (SEQ ID NO: 27). In some embodiments, the second recognition domain can comprise an amino acid sequence comprising YYCDNRF (SEQ ID NO: 28). In some embodiments, the second recognition domain can comprise an amino acid sequence comprising SAGNIKS (SEQ ID NO: 29). In some embodiments, the second recognition domain can comprise an amino acid sequence comprising YFCDNSF (SEQ ID NO: 30). In some embodiments, the second recognition domain can comprise an amino acid sequence comprising TAQNIKS (SEQ ID NO: 31). In some embodiments, the second recognition domain can comprise an amino acid sequence comprising SAQSIKS (SEQ ID NO: 32). In some embodiments, the second recognition domain can comprise an amino acid sequence comprising AANNIKS (SEQ ID NO: 33). In some embodiments, the second recognition domain can comprise an amino acid sequence comprising YNCSGKF (SEQ ID NO: 34). In some embodiments, the second recognition domain can comprise an amino acid sequence comprising QAQNIKS (SEQ ID NO: 35). In some embodiments, the second recognition domain can comprise an amino acid sequence comprising QADCIKS (SEQ ID NO: 36). In some embodiments, the second recognition domain can comprise an amino acid sequence comprising YSCDGVF (SEQ ID NO: 37). In some embodiments, the second recognition domain can comprise an amino acid sequence comprising RAQNIKS (SEQ ID NO: 38). In some embodiments, the second recognition domain can comprise an amino acid sequence comprising FLCENTF (SEQ ID NO: 39).

In some embodiments, the release factor may comprise a second recognition domain comprising an amino acid sequence listed in Table 3. In some embodiments, the release factor may comprise a second recognition domain comprising an amino acid sequence selected from the group consisting of SEQ ID NOs: 3-39. In some embodiments, the release factor comprising an amino acid sequence listed in Table 3 can be expressed from a nucleic acid sequence selected from the group consisting of SEQ ID NOs: 101-125. In some embodiments, the release factor comprising a second recognition domain comprising an amino acid sequence selected from the group consisting of SEQ ID NOs: 3-39 can be expressed from a nucleic acid sequence selected from the group consisting of SEQ ID NOS: 101-125. In some embodiments, the release factor described herein may comprise an amino acid sequence selected from the group consisting of SEQ ID NOs: 40-64. In some embodiments, the release factor described herein may be expressed from a nucleic acid sequence selected from the group consisting of SEQ ID NOs: 101-125. In some embodiments, the release factor described herein may comprise an amino acid sequence selected from the group consisting of 65-74. In some embodiments, the release factor described herein may be expressed from a nucleic acid sequence selected from the group consisting of 126-135. In some embodiments, the release factor described herein may comprise an amino acid sequence selected from the group consisting of 75-92. In some embodiments, the release factor described herein may be expressed from a nucleic acid sequence selected from the group consisting of 136-153. In some embodiments, the release factor described herein may comprise an amino acid sequence selected from the group consisting of 93-100. In some embodiments, the release factor described herein may be expressed from a nucleic acid sequence selected from the group consisting of 154-161.

In some embodiments, the release factor from the second organism can comprise an eRF1. In some embodiments, the eRF1 from the second organism can comprise an amino acid sequence that has between about at least 10% to about at least 50% sequence identity to an eRF1 of the first organism. In some embodiments, the eRF1 from the second organism can comprise an amino acid sequence that has at least 10% sequence identity to an eRF1 of the first organism. In some embodiments, the eRF1 from the second organism can comprise an amino acid sequence that has at least 15% sequence identity to an eRF1 of the first organism. In some embodiments, the eRF1 from the second organism can comprise an amino acid sequence that has at least 20% sequence identity to an eRF1 of the first organism. In some embodiments, the eRF1 from the second organism can comprise an amino acid sequence that has at least 25% sequence identity to an eRF1 of the first organism. In some embodiments, the eRF1 from the second organism can comprise an amino acid sequence that has at least 30% sequence identity to an eRF1 of the first organism. In some embodiments, the eRF1 from the second organism can comprise an amino acid sequence that has at least 35% sequence identity to an eRF1 of the first organism. In some embodiments, the eRF1 from the second organism can comprise an amino acid sequence that has at least 40% sequence identity to an eRF1 of the first organism. In some embodiments, the eRF1 from the second organism can comprise an amino acid sequence that has at least 45% sequence identity to an eRF1 of the first organism. In some embodiments, the eRF1 from the second organism can comprise an amino acid sequence that has at least 50% sequence identity to an eRF1 of the first organism.

In some embodiments, the release factor from the second organism can comprise an eRF1/eRF3 complex. In some embodiments, the eRF1 of the eRF1/eRF3 complex from the second organism can comprise an amino acid sequence that has between about at least 10% to about at least 50% sequence identity to an eRF1 of the first organism. In some embodiments, the eRF1 of the eRF1/eRF3 complex from the second organism can comprise an amino acid sequence that has at least 10% sequence identity to an eRF1 of the first organism. In some embodiments, the eRF1 of the eRF1/eRF3 complex from the second organism can comprise an amino acid sequence that has at least 15% sequence identity to an eRF1 of the first organism. In some embodiments, the eRF1 of the eRF1/eRF3 complex from the second organism can comprise an amino acid sequence that has at least 20% sequence identity to an eRF1 of the first organism. In some embodiments, the eRF1 of the eRF1/eRF3 complex from the second organism can comprise an amino acid sequence that has at least 25% sequence identity to an eRF1 of the first organism. In some embodiments, the eRF1 of the eRF1/eRF3 complex from the second organism can comprise an amino acid sequence that has at least 30% sequence identity to an eRF1 of the first organism. In some embodiments, the eRF1 of the eRF1/eRF3 complex from the second organism can comprise an amino acid sequence that has at least 35% sequence identity to an eRF1 of the first organism. In some embodiments, the eRF1 of the eRF1/eRF3 complex from the second organism can comprise an amino acid sequence that has at least 40% sequence identity to an eRF1 of the first organism. In some embodiments, the eRF1 of the eRF1/eRF3 complex from the second organism can comprise an amino acid sequence that has at least 45% sequence identity to an eRF1 of the first organism. In some embodiments, the eRF1 of the eRF1/eRF3 complex from the second organism can comprise an amino acid sequence that has at least 50% sequence identity to an eRF1 of the first organism.

In some embodiments, the eRF3 of the eRF1/eRF3 complex from the second organism can comprise an amino acid sequence that has between about at least 10% to about at least 50% sequence identity to an eRF3 of the first organism. In some embodiments, the eRF3 of the eRF1/eRF3 complex from the second organism can comprise an amino acid sequence that has at least 10% sequence identity to an eRF3 of the first organism. In some embodiments, the eRF3 of the eRF1/eRF3 complex from the second organism can comprise an amino acid sequence that has at least 15% sequence identity to an eRF3 of the first organism. In some embodiments, the eRF3 of the eRF1/eRF3 complex from the second organism can comprise an amino acid sequence that has at least 20% sequence identity to an eRF3 of the first organism. In some embodiments, the eRF3 of the eRF1/eRF3 complex from the second organism can comprise an amino acid sequence that has at least 25% sequence identity to an eRF3 of the first organism. In some embodiments, the eRF3 of the eRF1/eRF3 complex from the second organism can comprise an amino acid sequence that has at least 30% sequence identity to an eRF3 of the first organism. In some embodiments, the eRF3 of the eRF1/eRF3 complex from the second organism can comprise an amino acid sequence that has at least 35% sequence identity to an eRF3 of the first organism. In some embodiments, the eRF3 of the eRF1/eRF3 complex from the second organism can comprise an amino acid sequence that has at least 40% sequence identity to an eRF3 of the first organism. In some embodiments, the eRF3 of the eRF1/eRF3 complex from the second organism can comprise an amino acid sequence that has at least 45% sequence identity to an eRF3 of the first organism. In some embodiments, the eRF3 of the eRF1/eRF3 complex from the second organism can comprise an amino acid sequence that has at least 50% sequence identity to an eRF3 of the first organism.

In some embodiments, the release factor from the second organism can comprise an eRF1. In some embodiments, the eRF1 from the second organism can form a complex with an eRF3 from the first organism. In some embodiments, the eRF1 from the second organism can form a complex with an eRF3 from the second organism. In some embodiments, the eRF1 from the second organism can form a complex with a chimeric eRF3. In some embodiments, the chimeric eRF3 can comprise an eRF3 from the first organism or a fragment thereof and an eRF3 from a second organism or a fragment thereof. In some embodiments, the second organism can comprise, but is not limited to, Euplotes octocarinatus or Paramecium tetraurelia. In some embodiments, the chimeric eRF3 can comprise an eRF3 from Euplotes octocarinatus. In some embodiments, the chimeric Euplotes octocarinatus eRF3 can comprise amino acids 7-298 of the eRF3 from Euplotes octocarinatus can be replaced with amino acids 6-253 of the eRF3 from the first organism. In some embodiments, the chimeric Euplotes octocarinatus eRF3 can comprise an amino acid sequence comprising SEQ ID NO: 93 or SEQ ID NO: 94. In some embodiments, the chimeric Euplotes octocarinatus eRF3 can comprise a nucleic acid sequence comprising SEQ ID NO: 154 or SEQ ID NO: 155. In some embodiments, the chimeric Euplotes octocarinatus eRF3 can comprise amino acids 1-298 of the eRF3 from Euplotes octocarinatus can be replaced with amino acids 1-253 of the eRF3 from the first organism. In some embodiments, the chimeric Euplotes octocarinatus eRF3 can comprise an amino acid sequence comprising SEQ ID NO: 95 or SEQ ID NO: 96. In some embodiments, the chimeric Euplotes octocarinatus eRF3 can comprise a nucleic acid sequence comprising SEQ ID NO: 156 or SEQ ID NP: 157. In some embodiments, the chimeric eRF3 can comprise an eRF3 from Paramecium tetraurelia. In some embodiments, the chimeric Paramecium tetraurelia eRF3 can comprise amino acid 1-321 of the eRF3 from Paramecium tetraurelia can be replaced with amino acids 1-253 of the eRF3 from the first organism. In some embodiments, the chimeric Paramecium tetraurelia eRF3 can comprise an amino acid sequence comprising SEQ ID NO: 97, SEQ ID NO: 98, SEQ ID NO: 99, or SEQ ID NO: 100. In some embodiments, the chimeric Paramecium tetraurelia eRF3 can comprise a nucleic acid sequence comprising SEQ ID NO: 158, SEQ ID NO: 159, SEQ ID NO: 160, or SEQ ID NO: 161.

In some embodiments, the first organism can comprise a eukaryotic cell. In some embodiments, the first organism can comprise a prokaryotic cell. In some embodiments, the prokaryotic cells can comprise an archaebacteria cell. In some embodiments, the prokaryotic cell can comprise a bacterial cell. In some embodiments, the prokaryotic cell can comprise a bacterial cell and an archaebacteria cell. In some embodiments, the eukaryotic cell can comprise a yeast cell, a fungal cell, a plant cell, an animal cell, an insect cell, a mammalian cell, or any combination thereof. In some embodiments, the yeast cell can comprise Saccharomyces cerevisiae.

In some embodiments, a stop codon can be reassigned to encode a natural amino acid. In some cases, the natural amino acid can be alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, a stop codon can be reassigned to encode a non-canonical amino acid (ncAA).

In some embodiments, one or more tRNA molecules configured to recognize a reassigned stop codon are provided. In some embodiments, one or more aminoacyl-tRNA synthetases (aaRSs) for charging the one or more tRNA molecules are provided. In some cases, the aminoacyl-tRNA can charge the one or more tRNA molecules that recognize a reassigned stop codon with a natural amino acid. In some cases, the aminoacyl-tRNA can charge the one or more tRNA molecules that recognize a reassigned stop codon with a ncAA. In some cases, the natural amino acid can comprise alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, a stop codon can be reassigned to encode a non-canonical amino acid (ncAA).

Non-Canonical Amino Acid (ncAA)

As used herein, a non-canonical amino acid (ncAA) can refer to any amino acid other than the 20 genetically encoded alpha-amino acids comprising alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some aspects, described herein are non-canonical amino acids (ncAAs) that may comprise side chain chemistries and/or structures that are not available from canonical amino acids (cAAs). In some embodiments, ncAAs may comprise fluorinated amino acids or amino acids comprising a reactive group (e.g., carbonyl, alkene, or alkyne moieties), or photoactivatable group (e.g., azide, benzophenone, or fluorophores). Translation of ncAAs into proteins may allow chemical modification and accordingly, ncAAs may be useful for in vivo structure-function studies, protein-protein interaction studies, protein localization studies, protein activity regulation studies or studies to generate new protein function. ncAA can be incorporated in different cells, including, but not limited to bacterial cells (e.g., Escherichia coli), yeast cells (e.g., Saccharomyces cerevisiae, Pichia pastoris, or Candida albicans), mammalian cells and plant cells or in organisms, including, but not limited to Drosophila melanogaster, Caenorhabditis elegans, Bombyx mori, rabbit and cow.

In some embodiments, a ncAA may comprise Para-fluoro-L-phenylalanine, Para-iodo-L-phenylalanine, Para-azido-L-phenylalanine, Para-acetyl-L-phenylalanine, Para-benzoyl-L-phenylalanine, Meta-fluoro-L-tyrosine, O-methyl-L-tyrosine, Para-propargyloxy-L-phenylalanine, (2S)-2-aminooctanoic acid, (2S)-2-aminononanoic acid, (2S)-2-aminodecanoic acid, (2S)-2-aminohept-6-enoic acid, (2S)-2-aminooct-7-enoic acid, L-Homocysteine, (2S)-2-amino-5-sulfanylpentanoic acid, (2S)-2-amino-6-sulfany lhexanoic acid, L-S-(2-nitrobenzyl) cysteine, L-S-ferrocenyl-cysteine, L-O-crotylserine, L-O-(pent-4-en-1-yl) serine, L-O-(4,5-dimethoxy-2-nitrobenzyl) serine, (2S)-2-amino-3-({[5-(dimethylamino) naphthalen-1-yl]sulfonyl}amino) propanoic acid, (2S)-3-[(6-acetyl-naphthalen-1-yl)amino]-2-aminopropanoic acid, L-Pyrrolysine, N6-[(propargyloxy) carbonyl]-L-lysine, L-N6-acetyllysine, N6-trifluoroacetyl-L-lysine, N6-{[1-(6-nitro-1,3-benzodioxol-5-yl)ethoxy]carbonyl}-L-lysine, N6-{[2-(3-methyl-3H-diaziren-3-yl)ethoxy]carbonyl}-L-lysine, p-azidophenylalanine or 2-aminoisobutyric acid (also known as α-aminoisobutyric acid, AIB, α-methylalanine, or 2-methylalanine).

In some embodiments, a ncAA may comprise AbK (unnatural amino acid for Photo-crosslinking probe), 3-Aminotyrosine (unnatural amino acid for inducing red shift in fluorescent proteins and fluorescent protein-based biosensors), L-Azidohomoalanine hydrochloride (unnatural amino acid for bio-orthogonal labeling of newly synthesized proteins), L-Azidonorleucine hydrochloride (unnatural amino acid for bio-orthogonal or fluorescent labeling of newly synthesized proteins), BzF (photoreactive unnatural amino acid; photo-crosslinker), DMNB-caged-Serine (caged serine; excited by visible blue light), HADA (blue fluorescent D-amino acid for labeling peptidoglycans in live bacteria), NADA-green (fluorescent D-amino acid for labeling peptidoglycans in live bacteria), NB-caged Tyrosine hydrochloride (ortho-nitrobenzyl caged L-tyrosine), RADA (orange-red TAMRA-based fluorescent D-amino acid for labeling peptidoglycans in live bacteria), Rf470DL (blue rotor-fluorogenic fluorescent D-amino acid for labeling peptidoglycans in live bacteria), sBADA (green fluorescent D-amino acid for labeling peptidoglycans in bacteria), or YADA (green-yellow lucifer yellow-based fluorescent D-amino acid for labeling peptidoglycans in live bacteria).

In some embodiments, a ncAA may comprise an O-methyl-L-tyrosine, an L-3-(2-naphthyl) alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, or an isopropyl-L-phenylalanine.

In some embodiments, a ncAA may comprise an unnatural analogue of a canonical amino acid. For example, a ncAA may comprise an unnatural analogue of a tyrosine amino acid, an unnatural analogue of a glutamine amino acid, an unnatural analogue of a phenylalanine amino acid, an unnatural analogue of a serine amino acid, an unnatural analogue of a threonine amino acid. In some embodiments, a ncAA may comprise an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof.

In some embodiments, a ncAA may comprise an amino acid with a photoactivatable cross-linker, a spin-labeled amino acid, a fluorescent amino acid, an amino acid with a novel functional group, an amino acid that covalently or noncovalently interacts with another molecule, a metal binding amino acid, a metal-containing amino acid, a radioactive amino acid, a photocaged amino acid, a photoisomerizable amino acid, a biotin or biotin-analogue containing amino acid, a glycosylated or carbohydrate modified amino acid, a keto containing amino acid, an amino acid comprising polyethylene glycol, an amino acid comprising polyether, a heavy atom substituted amino acid, a chemically cleavable or photocleavable amino acid, an amino acid with an elongated side chain, an amino acid containing a toxic group, or a sugar substituted amino acid. In some embodiments, a sugar substituted amino acid may comprise a sugar substituted serine. In some embodiments, a ncAA may comprise a carbon-linked sugar-containing amino acid, a redox-active amino acid, an α-hydroxy containing amino acid, an amino thio acid containing amino acid, an α,α-disubstituted amino acid, a β-amino acid, or a cyclic amino acid other than proline.

In some embodiments, a ncAA may comprise p-azidophenylalanine or 2-aminoisobutyric acid (also known as α-aminoisobutyric acid, AIB, α-methylalanine, or 2-methylalanine).

Alternatively, the one or more tRNA molecules configured to recognize the reassigned stop codon can be pre-charged. In some cases, the pre-charged tRNA can be charged with a natural amino acid. In some cases, the pre-charged tRNA can be charged with a ncAA. In some cases, the natural amino acid can comprise alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, a stop codon can be reassigned to encode a non-canonical amino acid (ncAA).

In some embodiments, a release factor can be expressed from a gene integrated into a genome. In some cases, the gene can be integrated into the genome of a yeast. In some embodiments, the gene can be integrated into the genome via transformation. In some cases, the transformation can comprise heat-shock transformation. In some cases, the transformation can comprise electroporation. In some cases, the transformation can comprise cell-cell fusion. In some embodiments, the gene can be integrated into the genome via transfection. In some cases, the transfection can comprise a physical transfection. In some non-limiting example embodiments, physical transfection includes: electroporation, sonoporation, optical transfection, or hydrodynamic delivery. In some cases, the transfection can use a chemical transfection method. In some non-limiting example embodiments, a chemical transfection method can include: calcium phosphate, cationic polymers, lipofection, fugene, or dendrimers. In some embodiments, the gene can be integrated into the genome via transduction (e.g., foreign nucleic DNA introduced into a cell by a virus or viral vector). In some non-limiting example embodiments, viral vectors or viruses that can be used for transduction include: adenoviruses, adeno-associated viral vectors, lentiviruses, retroviruses, herpes simplex viruses, chimeric viral vectors, viral-like particles, pox viruses, or pseudotyped viruses. In some embodiments, the gene can be integrated into the genome via gene editing methods. In some non-limiting example embodiments, gene editing methods include: homologous recombination, site specific recombinases, meganucleases, zinc finger nucleases (ZFNs), transcription activator-like effector nucleases (TALEN), and clustered regularly interspaced short palindromic repeat/CRISPR-associated protein (e.g., CRISPR/Cas). In some non-limiting example embodiments, Cas proteins include: Cas9, Cas12, or Cas13.

In some embodiments, the release factor can be expressed from an episomal element. In some cases, the episomal element comprises a plasmid. In some cases, the plasmid can be a Superloser plasmid, a YIp plasmid, a YRp plasmid, a YCp plasmid, YEp plasmid, or a YLp plasmid. In some cases, the episomal element can exist autonomously in the cell (e.g., in the cytoplasm). In some cases, the episomal element can integrate into the genome. In some embodiments, the episomal element comprises regulatory sequences. In some embodiments, the regulatory sequences include: promoters, enhancers, silencers, or operators. In some embodiments, the promoter includes: endogenous RF1 promoter, endogenous RF3 promoter, endogenous eRF1 promoter, endogenous eRF3 promoter, Gal1/10 inducible promoter, In some embodiments, the episomal element further comprise one or more genes encoding a counter-selectable marker. In some embodiments, the counter-selectable gene can be a URA3 gene. In some embodiments, the counter-selectable gene can be a TRP1 gene. In some embodiments, the episomal element may further comprise one or more genes encoding a selectable marker. In some embodiments, the selectable marker gene can be a LEU2 gene. In some embodiments, the selectable gene can be a HIS3 gene.

In some embodiments, rewriting a stop codon can modulate protein translation. In some embodiments, protein translation can be modulated by terminating protein translation. In some cases, protein translation can be terminated early (e.g., a protein can be shorter than the wild-type protein). In some cases, protein translation can be terminated late (e.g., a protein can be longer than the wild-type protein).

One aspect of the present disclosure provides a method of producing a polypeptide molecule comprising a non-canonical amino acid (ncAA) or a population of polypeptide molecules comprising the ncAA in an organism. In some embodiments, the method can comprise rewriting a first stop codon to a second stop codon; reassigning the first stop codon to encode the ncAA in the genome of the organism; and introducing an aminoacyl-tRNA synthetase (aaRS)/tRNA part into the organism, wherein the aaRS/tRNA pair is configured to recognize the first stop codon and incorporate the ncAA into an amino acid sequence of the polypeptide or the population of the polypeptide molecules.

One aspect of the present disclosure provides a cell or population of cells or organism comprising a first stop codon rewritten to a second stop codon. In some embodiment, the cell or the population of cells can further comprise a release factor that recognizes only the second stop codon as a stop codon.

In some embodiments, the release factor recognition domain of the host cell can be changed by replacing its native eRF1 domain with a non-native recognition domain. In one embodiment, amino acid residues of the native eRF1 can be mutated. The mutated eRF1 can be configured to not recognize UGA or both UAG and UAA. In another embodiment, a recognition domain of a native eRF1 is swapped with a recognition domain of a ciliate eRF1 that recognizes only UGA as a stop codon. In some embodiments, a recognition domain of a native eRF1 is swapped with a recognition domain of a native eRF1 from a different organism that is known to work in the host organism. In some embodiments, the entire host eRF1 can be replaced with a foreign eRF1 that recognizes only UGA as a stop codon.

These embodiments may include the foreign eRF3, which works with eRF1 to provide release activity, and foreign enzymes that provide post-translational modifications for release factor proteins. For example, a post-translational modification can include, but is not limited to, a methyl-transferase activity. Embodiments described herein may include the foreign tRNA providing UGG recognition, together with its post-transcriptional modification machinery, to provide possible reduced cross-talk between the UGA stop codon and the UGG tryptophan codon. Embodiments disclosed herein may further comprise methods for protein engineering. In some embodiments, methods for protein engineering comprise directed evolution, library screens, machine learning, or a combination thereof. In some embodiments, library screens may be enhanced by phylogenetic data mining to identify organisms whose release factor machinery recognizes only UGA as a stop codon. Release factor machinery from the identified organisms are then tested systematically to identify the organism comprising release factors with a high level of fitness in the host organism. Testing the release factor machinery is accomplished by providing the sequences encoding the foreign release factor proteins, release factor modifying proteins, and tRNAs either integrated into the host genome or supplied on an episomal element, e.g., a Superloser plasmid. Haase, M., et al. “Superloser: A Plamid Shuffling Vector for Saccharomyces cerevisiae with Exceedingly Low Background.” G3 (Bethesda). 2019 Aug.: 9 (8): 2699-2707. In some embodiments, an episomal element comprising a native gene or a gene of the host organism may further comprise a counter-selectable gene (e.g., URA3). In some embodiments, one or more episomal elements comprising a foreign gene(s) may further comprise a selectable gene (e.g., HIS3, LEU2). The loss of the episomal element comprising the native gene or the gene of the host organism may be selected on 5-FOA. In some embodiments, the superloser plasmid may allow highly efficient counterselection.

Embodiments described herein may also comprise providing additional context after the UGA stop codon for enhanced recognition by the foreign release factor. In some embodiments, this may be accomplished via sequence analysis of the foreign genome to identify and determine nucleotide preference following stop codons. In some embodiments, a stop codon may comprise A or G at the +4 position, so that the in-frame sequence is UGA-A or UGA-G. An additional improvement may be made to reduce the recognition of sense codons by the release factor. For example, UAU can be recognized by release factors to introduce an early stop. This recognition may also occur with an A or G in the +4 position. In some example embodiments, synonymous codons for Arg may permit a choice between C and A in the first position, and synonymous codons for Ser may permit a choice between U and A in the first position. In some embodiments, following a sense codon whose first two positions match a stop codon (e.g., UG or UA), use of synonymous recoding avoids having an A codon in the +4 position. In some embodiments, recoding may result in a cell lacking UAG as a stop codon, and further lacking any release factor recognition of UAG as a stop codon. Thus, in this embodiment, the UAG codon can be available for encoding a non-canonical amino acid as part of an orthogonal translation system. The corresponding anti-codon may comprise CUA. Anticodons starting with C generally have no wobble, and the CUA IRNA can recognize UAG and no other codon.

In some embodiments, enhanced recognition by the foreign release factor may be provided by providing additional stop codon sequences after the first stop codon that is rewritten to a second stop codon. In some embodiments, these additional stop codons occur in the same reading frame as the first stop codon that is rewritten to second stop codon to enhance termination after readthrough of the first stop codon that is rewritten to the second stop codon. In some embodiments, the additional stop codon may be inserted immediately after the first stop codon that is rewritten to a second stop codon, or 3 nucleotides after the first stop codon that is rewritten to a second stop codon, or 6 nucleotides after the first stop codon that is rewritten to a second stop codon. In some embodiments, the second stop codon may comprise UGA. In some embodiments, the additional stop codon comprises UGA. In some embodiments, the additional stop codon may be inserted immediately after the first stop codon that is rewritten to a second stop codon. In some embodiments, the rewritten stop codon may comprise UGAUGA.

The method herein describes experimental procedures for testing the ability of ciliate release factors (RFs) that exclusively recognize either UAA/UAG or UGA to function in Saccharomyces cerevisiae (hereafter referred to as “yeast”). The methods of the present disclosure can test the ability of ciliate release factors, either individually or in combination, to replace the yeast native omnipotent RF, which recognizes all three stop codons. In some embodiments, replacement of a native RF comprises targeted engineering of specific motifs in the yeast RF to resemble motifs that can confer stop codon selectivity in ciliates (e.g. Amino Acid swap, Domain/Motif swap). In other embodiments, the targeted engineering can involve the complete gene replacement of yeast RFs with ciliate RFs (e.g. Native Ciliate Machinery). In the case of gene replacements, the ciliate RFs may be introduced as whole gene ciliate constructs or as chimeric yeast-ciliate constructs. In less preferred embodiments, addition of other ciliate genes that have regulatory functions that act on ciliate RFs may be required. Ciliate RFs that exclusively recognize UAA/UAG may fail to replace omnipotent yeast RFs because such a ciliate strain cannot decode UGA stop codons. Ciliate RFs that exclusively recognize UGA may fail to replace yeast RFs because such a strain cannot decode UAA/UAG stop codons. Combining two distinct ciliate RFs, one of which recognizes UAA/UAG, and the second that recognizes UGA, in the same strain, can allow “replaceability” of the native yeast RF that recognizes all three standard stop codons, demonstrating the stop codon specificity of the two RFs and simultaneously showing that both can function in yeast. In some embodiments, the experimental readout for testing replaceability of the yeast native RFs can be cell viability.

Class 1 and 2 S. cerevisiae RFs can be encoded by the essential genes SUP45 (eRF1) and SUP35 (eRF3), respectively. Replaceability of the yeast RFs by ciliate RFs can be tested in sup45Δ or sup45Δ sup35Δ mutants.

In some embodiments, the episomal-based shuffle system can be employed to test replaceability of wild-type yeast eRF1 by a motif-swapped yeast eRF1. In some cases, amino acid mutations are introduced into the yeast eRF1 protein's TASNIKS/YLCDNKF motifs (SEQ ID NOs: 1 and 2, respectively, in order of appearance), such that these motifs can resemble the TASNIKS/YLCDNKF motifs (SEQ ID NOs: 1 and 2, respectively, in order of appearance) of the ciliate eRF1 proteins. In these cases, replaceability is tested in a sup45Δ mutant which lacks yeast eRF1.

In some embodiments, the episomal-based shuffle system can be employed to test replaceability of wild-type yeast eRF1 by the entire ciliate eRF1 protein. In these cases, the ciliate eRF1 protein can be expressed from the yeast endogenous eRF1 promoter. In this embodiment, replaceability can be tested in a sup45Δ mutant. In other embodiments, the corresponding ciliate eRF3 may be required for ciliate eRF1 function in yeast. In these cases, the ciliate eRF1/eRF3 proteins can be expressed from the same vector using the GAL1/10 bi-directional promoter. In other embodiments, the ciliate eRF3 can be modified to create a chimeric yeast-ciliate eRF3 protein. In some cases, the yeast N-terminal domain (residues 1-253), which contains the poly(A)-binding site, can replace the more divergent ciliate N-terminal domain. When testing eRF1 in conjunction with eRF3, replaceability can be tested in a sup45Δ or sup45Δ sup35Δ mutant.

The sup45Δ or sup45Δ sup35Δ deletion mutants can be constructed by replacing the genomic copies of each gene in a diploid strain with selectable markers that confer drug resistance (such as kanMX, natMX or hphMX). Viability of the strain can be maintained by pre-transformation of the counter-selectable vector containing the corresponding yeast gene(s). In the case where expression of the vector-based yeast gene(s) is being driven by their endogenous promoter(s), the strains can be grown in medium with any sugar source (e.g., dextrose, galactose). In the case where expression of the vector-based yeast gene(s) is being driven by the inducible GAL1-10 promoter, the strains can be grown in a medium containing galactose as the sugar source. Following sporulation of the heterozygous diploid sup45Δ/SUP45 or homozygous diploid sup45Δ/sup45Δ strains, haploids containing the appropriate drug cassettes, as well as the counter-selectable vector, can be isolated by tetrad analysis. Yeast haploid strains bearing genomic deletions of sup45Δ or sup45Δ sup35Δ can be tested for plasmid-dependence by growing on a medium that counter-selects against the vector containing the wild-type yeast genes. In the case that this vector is marked by URA3, this medium can contain 5-FOA. In some embodiments, this vector can comprise a supernumerary designer chromosome. In some embodiments, this vector can comprise a supernumerary designed scaffold or a supernumerary designer chromosome.

In an embodiment, UAA may encode a non-canonical amino acid. In some embodiments, an anticodon for UAA starts with U, and anticodons starting with U usually have at least 2-codon wobble, recognizing UAA and UAG, or possible 4-codon wobble, recognizing the entire 4-codon block. This may introduce a single non-canonical amino acid encoded by the two codons UAA/UAG, or it could give cross-talk with the UAC/UAU codons encoding Tyrosine.

In another embodiment, a release factor that recognizes UAA/UAG as stop codons, but not UGA, may be used. In this embodiment, the anti-codon for UGA is UCA, and the U in the first position of the anti-codon could give wobble recognition with UGG, the tryptophan sense codon.

In some embodiments, the resulting cells could be viable with a reduced number of stop codons, but the cells may not improve on the ability to encode a non-canonical amino acid with the UAG codon, and they could introduce cross-talk absent from the preferred embodiment. Table 2 shows a risk analysis on rewriting/recoding stop codons in yeast.

TABLE 2
Risk analysis on rewriting/recoding

| Rewritten | # codons | # ncAA | | |
|---|---|---|---|---|
| genome | to rewrite | for recode | Rewrite risk | Recode risk |
| Sense | Up to 6 | Up to 3 | Very low: | Low: |
| codons | | | Predict ~0-5 | Clean codon |
| | | | bugs per | reassignment |
| | | | genome per | risk |
| | | | pair of sense | minimized by |
| | | | codons based | rewriting up |
| | | | on Sc2.0 | to 6 sense |
| | | | Derisk in | codons |
| | | | pilot | Efficiency and |
| | | | Bugs will be | fidelity of Aib |
| | | | rapidly fixed | recoding system |
| | | | | already derisked |
| | | | | in E. coli |
| Stop | 2 | 1 | Near zero: | Low: |
| codons | | | Derisked by | Release factor |
| | | | Sc2.0, zero | engineering |
| | | | instances of | has been done |
| | | | bugs in entire | in vitro and in |
| | | | synthetic | E. coli |
| | | | genome | |
| Sense + | Up to 8 | Up to 4 | Very low | Low: |
| stop | | | | Multiple |
| codons | | | | routes to |
| | | | | success for 2- |
| | | | | 3 ncAAs |

Provided herein are methods for designing a genome of an organism comprising rewriting a codon from the genome. In some aspects, rewriting a codon may comprise removing or replacing a codon such as a stop codon. In some embodiments, the stop codon may comprise UAG or UAA. In some embodiments, rewriting a codon may comprise removing or replacing UAG and UAA. In some embodiments, rewriting a codon may comprise replacing one or more of UAG and UAA with UGA. In some embodiments, all stop codons may be rewritten as UGAUGA. In some embodiments, the genome may be a yeast genome. In some embodiments, release factors may be modified by mutagenesis or domain/motif swapping.

In some aspects, methods provided herein may further comprise engineering a release factor (RF), for example, such that the RF is engineered to recognize at most two or at most one stop codon. In some embodiments, engineered RFs described herein may recognize UAG. In some embodiments, engineered RFs described herein may recognize UAA. In some embodiments, engineered RFs described herein may recognize UAG and UAA. In some embodiments, engineered RFs described herein recognize only UGA. In some embodiments, RFs may have evolved naturally to recognize at most one stop codon. In some embodiments, a recognition domain of RFs may be swapped. For example, a recognition domain of RFs from the ciliate may be swapped for a native yeast recognition domain to engineer a domain/motif-swapped RF. In some embodiments, a recognition domain of RFs may be swapped as a contiguous segment or as one or more non-contiguous amino acid changes.

In some aspects, methods provided herein may further comprise incorporating one or more non-canonical amino acids (ncAA). In some embodiments, incorporating one or more ncAA may utilize an orthogonal translation system. In some embodiments, the orthogonal translation system may decode a stop codon (e.g., UAG and/or UAA) as a sense codon.

### New Assignment of Rewritten/Replaced Codons

In some aspects, methods provided herein comprise stop codon rewriting and replacement. In some embodiments, stop codons rewritten or replaced are used to encode a new amino acid. In some embodiments, the new amino acid comprises a canonical amino acid. In some embodiments, the canonical amino acid comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, the new amino acid can be a non-canonical amino acid (ncAA).

In some aspects, methods provided herein comprise genetic code expansion using stop codon rewriting and replacement. In some embodiments, methods described herein comprise site-specific incorporation of one or more ncAAs into a polypeptide or a protein at a rewritten stop codon. In some embodiments, methods described herein can provide transformational approaches to understand and control one or more biological functions. For example, stop codon rewriting/replacement can allow genetically encoding amino acids corresponding to post-translationally modified versions of natural amino acids. For example, stop codon rewriting/replacement to allow genetically encoding photocaged amino acids can enable the rapid activation of protein function with light to dissect dynamic processes in cells. For example, stop codon rewriting/replacement to allow genetically encoding crosslinkers can provide a way to map protein interactions. For example, ncAAs containing fluorophores or other biophysical probes can be used to follow changes in protein structure and/or activity. In some embodiments, ncAAs may be used to alter enzyme function. In some embodiments, ncAAs may be used to trap labile enzyme-substrate intermediates for structural studies and substrate identification. In some embodiments, ncAAs bearing bio-orthogonal and chemically reactive groups may provide strategies for rapidly attaching a wide range of functionalities to proteins to precisely control and image protein function in cells and to create protein conjugates, including defined therapeutic conjugates. In some embodiments, genetic code expansion using stop codon rewriting and replacement methods described herein may form the basis of strategies for the reversible control of gene expression in animals and strategies for determining cell type-specific proteomes in animals. In some embodiments, genetic code expansion using stop codon rewriting and replacement methods described herein may allow incorporating multiple distinct ncAAs into polypeptides or proteins.

### Orthogonal Translation System

In some embodiments, a ribosome uses tRNA adaptors, aminoacylated with their cognate amino acids by specific aminoacyl-tRNA synthetases (aaRSs), to progressively decode the triplet codons in a coding sequence and polymerize the corresponding sequence of amino acids into a protein. 64 triplet codons are used to encode the 20 canonical amino acids, and the initiation and termination of protein synthesis. In some aspects, stop codon rewriting and replacement methods described herein may allow reassigning those rewritten stop codons to encode a new amino acid (referred to as orthogonal codons). In some embodiments, orthogonal codons can be assigned to ncAAs. In some embodiments, each new orthogonal codon must be decoded by an additional aminoacyl-tRNA synthetase (aaRS)/tRNA pair. In some embodiments, these aaRS/tRNA pairs may uniquely decode distinct codons and recognize distinct ncAAs. In some embodiments, orthogonal codons can be assigned to canonical amino acids. In some embodiments, these aaRS/tRNA pairs may uniquely decode distinct codons and recognize distinct canonical amino acids.

In some aspects, methods described herein may comprise orthogonal aaRS/tRNA pairs. In some embodiments, each orthogonal aaRS may aminoacylate its cognate orthogonal tRNA, and/or minimally aminoacy late the other tRNAs in an organism. In some embodiments, the orthogonal tRNA may be aminoacylated by its cognate synthetase and/or minimally be aminoacylated by the aaRSs of the organism. In some embodiments, the orthogonal tRNA may be engineered to recognize an orthogonal codon that is not assigned to a canonical amino acid (i.e., rewritten/replaced codons), while maintaining selective aminoacylation by the orthogonal synthetase. In some embodiments, an active site of the orthogonal synthetase may be engineered.

In some aspects, provided herein are methods for reassigning a stop codon to encode an amino acid that the codon does not naturally encode. For example, a codon may be reassigned to a ncAA, i.e., the codon encodes a ncAA instead of an amino acid naturally encoded by the codon. Over 100 ncAAs with diverse chemistries may be synthesized and co-translationally incorporated into polypeptides and proteins using evolved orthogonal aminoacyl-tRNA synthetase (aaRSs)/tRNA pairs. Various aaRS/tRNA pairs can be used for methods described herein. In some embodiments, an ncAA may be designed based on tyrosine or pyrrolysine. In some embodiments, an aaRS/tRNA pair may be provided on a plasmid or into the genome of a cell or an organism comprising one or more reassigned codons. In some embodiments, an orthogonal aaRS/tRNA pair can be used to bioorthogonally incorporate ncAAs into polypeptides or proteins.

In some embodiments, vector-based over-expression systems may be used. In some embodiments, vector-based over-expression systems may outcompete natural stop codon function via a reassigned function. In some embodiments where natural aaRS and/or tRNAs for the rewritten stop codon are completely abolished or removed, lower amount of aaRS/tRNA for the newly assigned ncAA may be sufficient to achieve efficient ncAA incorporation. In some embodiments, genome-based aaRS/tRNA pairs (i.e., aaRS/tRNA pairs incorporated into the genome of the cell or organism) may be used to reduce the mis-incorporation of canonical amino acids in the absence of available ncAAs. In some embodiments, ncAA incorporation into polypeptides or proteins may involve supplementing the growth media with the ncAA described herein and an inducer for the aaRS expression. Alternatively, the aaRS may be expressed constitutively.

In some embodiments, aaRS/tRNA pairs may be imported from evolutionarily divergent organisms, wherein the sequence has diverged from that of the aaRS/tRNA pairs in the host organism or cell of interest (e.g., archaeal and eukaryotic pairs in an E. coli host). In some embodiments, derivatives of the Methanocaldococcus janaschii tyrosyl-tRNA synthetase (MjTyrRS)/MjtRNA^{Tyr} pair may be used to incorporate a wide variety of ncAAs into polypeptides or proteins. In some embodiments, derivatives of the E. coli leucyl-tRNA synthetase (EcLeuRS)/EctRNA^{Leu}, E. coli tryptophanyl-tRNA synthetase (EcTrpRS)/EctRNA^{Trp}, or EcTyrRS/EetRNA^{Tyr} pairs may be used to incorporate one or more ncAAs into polypeptides or proteins. In some embodiments, EcTyrRS/EctRNA^{Tyr} pair or EcTrpRS/EctRNA^{Trp} pair may be directly evolved for a new ncAA specificity. In some embodiments, endogenous copies of aaRS/tRNA pairs maybe replaced with pairs that are orthogonal in another host organism.

In some embodiments, evolved derivatives of a Methanococcus maripaludis phosphoseryl-tRNA synthetase (MmpSepRS)/MjtRNA^{Sep} pair may be used to incorporate phosphoserine, its non-hydrolysable analogue, or phosphothreonine. In some embodiments, Methanosarcina mazei pyrrolysyl-tRNA synthetase (MmPylRS)/MmtRNA^{Pyl}_{CUA} pair, Methanosarcina barkeri PylRS (MbPylRS)/MbtRNA^{Pyl}_{CUA} pair, or derivatives thereof, may be used to incorporate one or more ncAAs. In some embodiments, Archaeoglobus fulgidus (Af) TyrRS/AftRNA^{Tyr}_{CUA} may be used to incorporate one or more ncAAs. In some embodiments, engineered aaRS/tRNA pairs may be used to incorporate one or more ncAAs.

In some embodiments, an organism or a host organism described herein can comprise an animal. In some embodiments, the animal may comprise a mammal. In some embodiments, the mammal comprises a human, non-human primate, rodent, caprine, bovine, ovine, equine, canine, feline, mouse, rat, rabbit, horse or goat. In some embodiments, an organism or a host organism may comprise E. coli, Salmonella enterica subsp. enterica serovar Typhimurium, Saccharomyces cerevisiae, cultured mammalian cells, Arabidopsis thaliana, Caenorhabditis elegans, Drosophila melanogaster or Mus musculus.

A cell or a host cell described herein can be a bacterial cell, a yeast cell, a fungal cell, an insect cell, or a mammalian cell. In some embodiments, a cell may comprise a mammalian cell. Mammalian cells can be derived or isolated from a tissue of a mammal. In some embodiments, mammalian cells may comprise COS cells, BHK cells, 293 cells, 3T3 cells, NS0 hybridoma cells, baby hamster kidney (BHK) cells, PER.C6™ human cells, HEK293 cells or Cricetulus griseus (CHO) cells. In some embodiments, a mammalian cell may comprise a human cell, a rodent cell, or a mouse cell. Examples of mammalian cells can also include but are not limited to cells from humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some embodiments, a mammalian cell is a human cell. In some embodiments, a mammalian cell is a mouse cell. In some embodiments, a mammalian cell comprises an embryonic stem cell (ESC), a pluripotent stem cell (PSC), or an induced pluripotent stem cell (iPSC). In some embodiments, a cell or a host cell may comprise a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the mammalian cell comprises a rodent cell, a mouse cell, or a human cell, or a combination thereof.

Methods for incorporating non-canonical amino acids in yeast are described in, for example, Stieglitz J. T., Van Deventer J. A. (2022) Incorporating, Quantifying, and Leveraging Noncanonical Amino Acids in Yeast. In: Rasooly A., Baker H., Ossandon M. R. (eds) Biomedical Engineering Technologies. Methods in Molecular Biology, vol 2394. Humana, New York, NY (doi.org/10.1007/978-1-0716-1811-0_21), which is incorporated by reference herein in its entirety.

Applications of proteins with non-canonical amino acids are described in, for example, Jeremiah A Johnson, Ying Y Lu, James A Van Deventer, David A Tirrell, Residue-specific incorporation of non-canonical amino acids into proteins: recent developments and applications,

Current Opinion in Chemical Biology, Volume 14, Issue 6, 2010, Pages 774-780, ISSN 1367-5931, doi.org/10.1016/j.cbpa.2010.09.013 (www.sciencedirect.com/science/article/pii/S1367593110001390), which is incorporated by reference herein in its entirety.

Examples of orthogonal translation in E. coli with a genome rewritten to exclude a subset of sense codons are described in, for example, Robertson W E, Funke L F H, de la Torre D, Fredens J, Elliott T S, Spinck M, Christova Y, Cervettini D, Böge F L, Liu K C, Buse S, Maslen S, Salmond G P C, Chin J W. Sense codon reassignment enables viral resistance and encoded polymer synthesis. Science. 2021 Jun. 4; 372 (6546): 1057-1062. doi: 10.1126/science.abg3029. PMID: 34083482; PMCID: PMC7611380, which is incorporated by reference herein in its entirety.

Additional examples of orthogonal translation are described in, for example, de la Torre, D., Chin, J. W. Reprogramming the genetic code. Nat Rev Genet 22, 169-184 (2021) (doi.org/10.1038/s41576-020-00307-7), which is incorporated by reference herein in its entirety.

In some embodiments, a host genome may be divided into multiple regions for stop codon replacement design. In some embodiments, a host genome may be divided into at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 regions for stop codon replacement design. In some embodiments, a host genome may be divided into approximately 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 regions for stop codon replacement design. In some embodiments, a host genome may be divided into 5 regions for stop codon replacement design.

In some embodiments, each region may be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least about 50 kilobases (kb). In some embodiments, each region may be approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 kb. In some embodiments, each region may have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 designs. In some embodiments, each region may have approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 designs.

In some embodiments, the total number of stop codons rewritten or replaced may comprise at least 1, 10, 50, 100, 150, 200, 250, 300, 350, 400, 450, 500, 550, 600, 650, 700, 750, 800, 850, 900, 950, or at least 1000 stop codons. In some embodiments, the total number of stop codons rewritten or replaced may comprise approximately 1, 10, 50, 100, 150, 200, 250, 300, 350, 400, 450, 500, 550, 600, 650, 700, 750, 800, 850, 900, 950, or approximately 1000 stop codons. In some embodiments, the total number of stop codons rewritten or replaced may comprise at least 1K, 2K, 3K, 4K, 5K, 6K, 7K, 8K, 9K, 10K, 20K, 30K, 40K, 50K, 60K, 70K, 80K, 90K, 100K, 110K, 120K, 130K, 140K, 150K, 160K, 170K, 180K, 190K, 200K, 250K, 300K, 350K, 400K, 450K, 500K, 550K, 600K, 650K, 700K, 750 K, 800 K, 850 K, 900 K, 950 K, or at least 1000K stop codons. In some embodiments, the total number of stop codons rewritten or replaced may comprise approximately 1K, 2K, 3K, 4K, 5K, 6K, 7K, 8K, 9K, 10K, 20K, 30K, 40K, 50K, 60K, 70K, 80K, 90K, 100K, 110K, 120K, 130K, 140K, 150K, 160K, 170K, 180K, 190K, 200K, 250K, 300K, 350K, 400K, 450K, 500K, 550K, 600K, 650K, 700K, 750 K, 800 K, 850 K, 900 K, 950 K, or approximately 1000K stop codons.

### Computer Systems

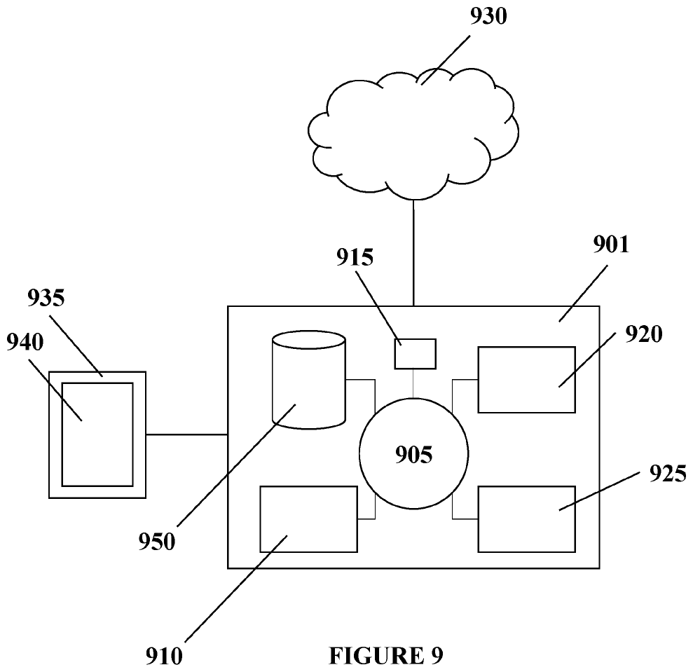

The present disclosure provides computer systems that are programmed to implement methods of the disclosure. FIG. 9 shows a computer system 901.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

The network 930 in some cases is a telecommunication and/or data network. The network 930 can include one or more computer servers, which can enable distributed computing, such as cloud computing. For example, one or more computer servers may enable cloud computing over the network 930 (“the cloud”) to perform various aspects of analysis, calculation, and generation of the present disclosure. Such cloud computing may be provided by cloud computing platforms such as, for example, Amazon Web Services (AWS), Microsoft Azure, Google Cloud Platform, and IBM cloud. The network 930, in some cases with the aid of the computer system 901, can implement a peer-to-peer network, which may enable devices coupled to the computer system 901 to behave as a client or a server.

The CPU 905 may comprise one or more computer processors and/or one or more graphics processing units (GPUs). The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung®; Galaxy Tab), telephones, Smart phones (e.g., Apple® iphone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine-executable or machine-readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 901 can include or be in communication with an electronic display 935 that comprises a user interface (UI) 940.

### IRNA Supplementation

In some embodiments, additional tRNAs with anticodons recognizing the newly assigned codons (i.e., stop codons encoding a newly assigned canonical amino acid or an ncAA) may be provided. In some embodiments, the total number of tRNA genes deleted can be determined, and the copy number of the remaining tRNA genes for an amino acid can be increased by the same amount. In some embodiments, wobble rules can be used to identify the tRNA genes responsible for decoding the replacement codons, and copy number increases can be allocated proportionally. In some embodiments, one or more non-native tRNA genes may be introduced. For example, for leucine, tL(AAG) from Candida species may be introduced.

### Nucleic Acid Construction and Replacing Genome

In some aspects, methods described herein may comprise synthesizing a nucleic acid construct comprising one or more stop codons rewritten based on codon rewriting/replacement methods described herein. Any known method in the art can be used to synthesize the nucleic acid construct comprising one or more stop codons rewritten based on codon rewriting/replacement methods described herein. In some embodiments, a chromosome can be computationally divided into 30-60 kilobase long constructs, each comprising a set of segments that is less than about 10 kilobase in length. Each segment can be synthesized using any known methods in the art, e.g., a polymerase chain reaction (PCR), and/or restriction enzyme digestion/ligation. In some embodiments, these segments can be assembled into a construct by restriction enzyme cutting and ligation in vitro, or any other methods known in the art. In some embodiments, the construct can be sequenced to confirm the sequence of the nucleic acid construct and subsequently integrated into the host genome, e.g., a yeast genome, using any known methods in the art to replace the corresponding portion, region, or segment of the wile-type.

In some aspects, methods described herein may further comprise replacing a portion of a genome with a nucleic acid construct comprising one or more codons rewritten based on codon rewriting/replacement methods described herein. In some embodiments, site-specific nucleases (SSNs) or homology-directed recombination (HR) can be used to replace a portion of a genome. In some embodiments, HR can be used utilizing an endogenous homologous recombination machinery.

In some embodiments, SSN may comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques, namely, meganucleases, ZFNs, TALENs, CRISPR/Cas systems share a common mode of action in binding a user-defined sequence of DNA and mediating a double-stranded DNA break (DSB). DSB may then be repaired by HR, an event that introduces the homologous sequence from a donor DNA fragment, or by non-homologous end joining (NHEJ), when there is no donor DNA present.

In some embodiments, a CRISPR-Cas system may be used with a guide target sequence for genetic screening, targeted transcriptional regulation, targeted knock-in, and targeted genome editing, including base editing, epigenetic editing, and introducing double strand breaks (DSBs) for homologous recombination-mediated insertion of a nucleotide sequence. CRISPR-Cas system comprises an endonuclease protein whose DNA-targeting specificity and cutting activity can be programmed by a short guide RNA or a duplex crRNA/TracrRNA. A CRISPR endonuclease comprises a caspase effector nuclease, typically microbial Cas9 and a short guide RNA (gRNA) or a RNA duplex comprising a 18 to 20 nucleotide targeting sequence that directs the nuclease to a location of interest in the genome. Genome editing can refer to the targeted modification of a DNA sequence, including but not limited to, adding, removing, replacing, or modifying existing DNA sequences, and inducing chromosomal rearrangements or modifying transcription regulation elements (e.g., methylation/demethylation of a promoter sequence of a gene) to alter gene expression. As described above CRISPR-Cas system requires a guide system that can locate Cas protein to the target DNA site in the genome. In some instances, the guide system comprises a crispr RNA (crRNA) with a 17-20 nucleotide sequence that is complementary to a target DNA site and a trans-activating crRNA (tracrRNA) scaffold recognized by the Cas protein (e.g., Cas9). The 17-20 nucleotide sequence complementary to a target DNA site is referred to as a spacer while the 17-20 nucleotide target DNA sequence is referred to a protospacer. While crRNAs and tracrRNAs exist as two separate RNA molecules in nature, single guide RNA (sgRNA or gRNA) can be engineered to combine and fuse crRNA and tracrRNA elements into one single RNA molecule. Thus, in one embodiment, the gRNA comprises two or more RNAs, e.g., crRNA and tracrRNA. In another embodiment, the gRNA comprises a sgRNA comprising a spacer sequence for genomic targeting and a scaffold sequence for Cas protein binding. In some instances, the guide system naturally comprises a sgRNA. For example, Cas12a/Cpf1 utilizes a guide system lacking tracrRNA and comprising only a crRNA containing a spacer sequence and a scaffold for Cas12a/Cpf1 binding. While the spacer sequence can be varied depending on a target site in the genome, the scaffold sequence for Cas protein binding can be identical for all gRNAs.

CRISPR-Cas systems described herein can comprise different CRISPR enzymes. For example, the CRISPR-Cas system can comprise Cas9, Cas12a/Cpf1, Cas12b/C2cl, Cas12c/C2c3, Cas12d/CasY, Cas12e/CasX, Cas12g, Cas12h, or Cas12i. In some non-limiting example embodiments, Cas enzymes include, but are not limited to, Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas) (also known as Csn1 or Csx12), Cas10, Cas10d, Cas12a/Cpf1, Cas12b/C2cl, Cas12c/C2c3, Cas12d/CasY, Cas12e/CasX, Cas12f/Cas14/C2c10, Cas12g, Cas12h, Cas12i, Cas12k/C2c5, Cas13a/C2c2, Cas13b, Cas13c, Cas13d, C2c4, C2c8, C2c9, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, GSU0054, Type II Cas effector proteins, Type V Cas effector proteins, Type VI Cas effector proteins, CARF, DinG, homologues thereof, or modified or engineered versions thereof such as dCas9 (endonuclease-dead Cas9) and nCas9 (Cas9 nickase that has inactive DNA cleavage domain). In some cases, the compositions, methods, devices, and systems, described herein, may use the Cas9 nuclease from Streptococcus pyogenes, of which amino acid sequences and structures are well known to those skilled in the art.

In some aspects, described herein, are methods for contacting a genome from a sample with one or more agents configured to cleave the genome at a locus. In some embodiments, the contacting may occur in vitro. In some embodiments, the contacting may occur in vivo, e.g., in a cell. In some embodiments, the one or more agents comprise a polypeptide, a polynucleotide, or a combination thereof. In some embodiments, the polypeptide comprises an enzyme, e.g., a site-specific nuclease. Examples of a site-specific nuclease are shown above. In some embodiments, a site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas), or a combination thereof. In some embodiments, the polynucleotide comprises a guide RNA (gRNA). In some embodiments, the one or more agents comprise a site-specific nuclease and a gRNA (e.g., CRISPR/Cas system).

Agents described herein can be delivered into cells in vitro or in vivo by art-known methods or as described herein. Delivery methods such as physical, chemical, and viral methods are also known in the art. In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus. In some embodiments, agents described herein can be delivered via a carrier. In some embodiments, agents described herein can be delivered by, e.g., vectors (e.g., viral or non-viral vectors), non-vector-based methods (e.g., using naked DNA, DNA complexes, lipid nanoparticles, RNA such as mRNA), or a combination thereof. In some embodiments, a carrier can comprise comprises a vector, a messenger RNA (mRNA), double stranded DNA (dsDNA), single stranded DNA (ssDNA), or a plasmid. In some embodiments, agents can be delivered directly to cells as naked DNA or RNA. Direct delivery, in some cases, is facilitated by, for instance by means of transfection or electroporation. In some cases, the agents are, or can be conjugated to molecules (e.g., N-acetylgalactosamine) promoting uptake by cells.

In some embodiments, vectors can comprise one or more sequences encoding one or more agents described herein. Vectors can also comprise a sequence encoding a signal peptide (e.g., for nuclear localization, nucleolar localization, or mitochondrial localization), associated with (e.g., inserted into or fused to) a sequence coding for a protein. As one example, vectors can include a Cas9 coding sequence that includes one or more nuclear localization sequences (e.g., a nuclear localization sequence from SV40). Vectors described herein can also include any suitable number of regulatory/control elements, e.g., promoters, enhancers, introns, polyadenylation signals, Kozak consensus sequences, or internal ribosome entry sites (IRES). These elements are well known in the art. Vectors described herein may include recombinant viral vectors. Any viral vectors known in the art can be used. Examples of viral vectors include, but are not limited to lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types. In some embodiments, agents described herein may be delivered in one carrier (e.g., one vector). In some embodiments, agents described herein may be delivered in in multiple carriers (e.g., multiple vectors).

In addition, viral particles can be used to deliver agents in nucleic acid and/or peptide form. For example, “empty” viral particles can be assembled to contain any suitable cargo. Viral vectors and viral particles can also be engineered to incorporate targeting ligands to alter target tissue specificity. Non-viral vectors can be also used to deliver agents according to the present disclosure. One example of non-viral nucleic acid vectors is an nanoparticle, which can be organic or inorganic. Nanoparticles are well known in the art. Any suitable nanoparticle design can be used to deliver agents described herein (e.g., nucleic acids encoding such agents).

In some embodiments, agents described herein can be delivered as a ribonucleoprotein (RNP) to cells. An RNP may comprise a nucleic acid binding protein, e.g., Cas9, in a complex with a gRNA targeting a genome/locus/sequence of interest. RNPs can be delivered to cells using known methods in the art, including, but not limited to electroporation, nucleofection, or cationic lipid-mediated methods, for example, as reported by Zuris, J. A. et al., 2015, Nat. Biotechnology, 33 (1): 73-80.

### Machine Learning-Based Computer Systems

In some aspects, methods described herein may comprise utilizing a machine learning-based computer system. In some embodiments, machine learning-based computer systems described herein may comprise one or more storage units comprising, respectively, one or more storage devices included within respective storage arrays controlled by a respective one or more storage controllers; and one or more computer processing units, wherein the one or more computer processing units are configured to communicate with the one or more storage units over a communication interface.

In some non-limiting example embodiments, machine learning can include: supervised machine learning, Random Forest, support vector machine, neural network, regression tree, or unsupervised machine learning.

In some embodiments, the machine learning-based computer system provides the plurality of intermediate scores to a machine learning algorithm that processes the plurality of intermediate scores to generate the rewritten stop codons (e.g., the first plurality of stop codons that are selected to be rewritten into a second stop codon). The machine learning algorithm may comprise a function that determines how intermediate scores are combined and weighted. The machine learning algorithm may comprise a supervised machine learning algorithm. The supervised machine learning algorithm may be trained on prior data from a reference genome, or on prior data from multiple genomes. The prior data may include observed fitness values for genomes, including growth rates on different media. The machine learning-based computer system can train the supervised machine learning algorithm by providing examples of fitness values to an untrained or partially trained version of the algorithm to generate replacement codons for one or more of the input genomes or of a different genome. The system can compare the predicted fitness to the measured fitness (i.e., whether the cell growth rate was maintained), and if there is a difference, the system can perform training at least in part by updating the parameters of the supervised machine learning algorithm. The supervised machine learning algorithm may comprise a regression algorithm, a support vector machine, a decision tree, a neural network, or the like. In cases in which the machine learning algorithm comprises a regression algorithm, the weights may be regression parameters. The supervised machine learning algorithm may comprise a classifier or a predictor that determines a prediction of which replacement codons (e.g., selected from among a plurality of possible replacement codons) are least likely to result in a fitness deficit. The predictor may generate a fitness risk score that is indicative of a likelihood of being indicative of a fitness risk (e.g., probabilistic fitness risk score between 0 and 1). In some cases, the machine learning-based computer system may map the probabilistic risk score to a qualitative risk category (e.g., selected from among a plurality of risk categories). For example, a fitness risk score that is at least 0.5 may be considered a high risk, while a fitness risk score that is less than 0.5 may be considered a low risk. Alternatively, the supervised machine learning algorithm may be a multi-class classifier (e.g., binary classifier) that predicts a qualitative risk category directly.

The machine learning algorithm may comprise unsupervised machine learning algorithm. The unsupervised machine learning algorithm may identify patterns in a genome or multiple genomes of interest. For example, it may identify a set of codon usage contexts that are an outlier as compared to other sets of codon usage for the same amino acid. If the unsupervised machine learning algorithm determines that a particular context-dependent codon usage is an outlier, the machine learning-based computer system may determine that relying on genome-wide codon usage for codon selection may lead to a fitness deficit. On the other hand, a set of codon usage scores that is consistent with overall codon usage for the genome may indicate that codon replacement has lower risk of generating a fitness defect. The unsupervised machine learning algorithm may comprise a clustering algorithm, an isolation forest, an autoencoder, or the like.

### Trained Algorithms

In some aspects, methods and systems described herein may employ one or more trained algorithms. The trained algorithm(s) may process or operate on one or more datasets comprising information about a codon-of-interest, a codon upstream of (or 5′ to) the stop codon-of-interest, a codon downstream of (or 3′ to) the stop codon-of-interest, or any combination thereof. The trained algorithm(s) may process or operate on one or more datasets comprising information about a stop codon-of-interest. In some embodiments, the datasets comprise structural or sequence information about codons. In some embodiments, the datasets comprise one or more datasets of codons. The one or more datasets may be observed empirically, derived from computational studies, be derived or retrieved from one or more databases, be artificially generated (e.g., as in silico variants of empirically observed datasets), or any combination thereof.

The trained algorithm may comprise an unsupervised machine learning algorithm. The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm. The trained algorithm may comprise a statistical model, statistical analysis, or statistical learning.

In some embodiments, a machine learning algorithm (or software module) of a platform as described herein utilizes one or more neural networks. In some embodiments, a neural network is a type of computational system that can learn the relationships between an input dataset and a target dataset. A neural network may be a software representation of a human neural system (e.g., cognitive system), intended to capture “learning” and “generalization” abilities as used by a human. In some embodiments, the machine learning algorithm (or software module) comprises a neural network comprising a convolutional neural network (CNN). In some non-limiting example embodiments, structural components of embodiments of the machine learning software described herein include: CNNs, recurrent neural networks, dilated CNNs, fully-connected neural networks, deep generative models, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from a set of the previous layers into more complex relationships. In addition, whereas some software programs require writing specific instructions to perform a task, neural networks are programmed by training them with a known sample set and allowing them to modify themselves during (and after) training so as to provide a desired output such as an output value (e.g., predicted value). After training, when a neural network is presented with new input data, it generalizes what was “learned” during training and applies what was learned from training to the new, previously unseen, input data in order to generate an output associated with that input (e.g., a predicted value). The output may be generated in order to minimize an expected error or loss function between the output value and an expected value.

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network, or DNN) is an ANN comprising a plurality of hidden layers, e.g., two or more hidden layers. Each layer of the neural network may comprise a number of nodes (or “neurons”). A node receives a set of inputs that are retrieved from either directly from the input data or the output of nodes in previous layers, and performs a specific operation, e.g., a summation operation, on the set of inputs. A connection from an input to a node is associated with a weight (or weighting factor). The node may determine a sum of the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN determines are consistent with the examples included in the training dataset.

The number of nodes used in the input layer of the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In other instances, the number of node used in the input layer may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or fewer. In some instances, the total number of layers used in the ANN or DNN (including input and output layers) may be at least about 3, 4, 5, 10, 15, 20, or greater. In other instances, the total number of layers may be at most about 20, 15, 10, 5, 4, 3, or fewer.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In other instances, the number of learnable parameters may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or fewer.

In some embodiments described herein, a machine learning software module comprises a neural network such as a deep CNN. In some embodiments in which a CNN is used, the network is constructed with any number of convolutional layers, dilated layers, or fully connected layers. In some embodiments, the number of convolutional layers is between 1-10, and the number of dilated layers is between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or fewer, and the total number of dilated layers may be at most about 20, 15, 10, 5, 4, 3, or fewer. In some embodiments, the number of convolutional layers is between 1-10 and the fully connected layers between 0-10. The total number of convolutional layers (including input and output layers) may be at least about 1, 2, 3, 4, 5, 10, 15, 20, or greater, and the total number of fully connected layers may be at least about 1, 2, 3, 4, 5, 10, 15, 20, or greater. The total number of convolutional layers may be at most about 20, 15, 10, 5, 4, 3, 2, 1, or less, and the total number of fully connected layers may be at most about 20, 15, 10, 5, 4, 3, 2, 1, or fewer.

In some embodiments, the input data for training of the ANN may comprise a variety of input values depending whether the machine learning algorithm is used for processing sequence or structural data. In some embodiments, the ANN or deep learning algorithm may be trained using one or more training datasets comprising the same or different sets of input and paired output data.

In some embodiments, a machine learning software module comprises a neural network comprising a CNN, recurrent neural network (RNN), dilated CNN, fully connected neural networks, deep generative models, and deep restricted Boltzmann machines.

In some embodiments, a machine learning algorithm comprises CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully connected layers, and normalization layers. The layers may be organized in 3 dimensions: width, height, and depth.

The convolutional layers may apply a convolution operation to the input and pass results of the convolution operation to the next layer. For processing sequence data, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the length of the input sequence, determine the dot product between the entries of the filter and the input, and produce a two-dimensional activation map of that filter. As a result, the network may learn filters that activate when it detects some specific type of feature at some spatial position in the input.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully connected layers connect every neuron in one layer to every neuron in another layer. In neural networks, each neuron may receive input from some number locations in the previous layer. In a fully connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

In some embodiments, a machine learning software module comprises a RNN software module. A RNN software module may receive sequential data as an input, such as consecutive data inputs, and the RNN software module updates an internal state at every time step. A RNN can use internal state (memory) to process sequences of inputs. The RNN may be applicable to tasks such as codon selection. The RNN may also be applicable to next codon prediction, and codon usage anomaly detection. In some embodiments, a RNN may comprise a fully recurrent neural network, an independently recurrent neural network, Elman networks, Jordan networks, an Echo state, a neural history compressor, a long short-term memory, a gated a recurrent unit, a multiple timescales model, neural Turing machines, a differentiable neural computer, and a neural network pushdown automata.

In some embodiments, a machine learning software module comprises a supervised or unsupervised learning method such as, for example, support vector machines (“SVMs”), random forests, clustering algorithm (or software module), gradient boosting, linear regression, logistic regression, and/or decision trees. The supervised learning algorithms may be algorithms that rely on the use of a set of labeled, paired training data examples to infer the relationship between an input data and output data. The unsupervised learning algorithms may be algorithms used to draw inferences from training datasets to the output data. The unsupervised learning algorithm may comprise cluster analysis, which may be used for exploratory data analysis to find hidden patterns or groupings in process data. One example of unsupervised learning method may comprise principal component analysis. The principal component analysis may comprise reducing the dimensionality of one or more variables. The dimensionality of a given variable may be at least 1, 5, 10, 50, 100, 200, 300, 400, 500, 600, 700, 800, 900, 1,000, 1,100, 1,200 1,300, 1,400, 1,500, 1,600, 1,700, 1,800, or greater. The dimensionality of a given variables may be at most 1,800, 1,700, 1,600, 1,500, 1,400, 1,300, 1,200, 1,100, 1,000, 900, 800, 700, 600, 500, 400, 300, 200, 100, 50, 10, or fewer.

In some embodiments, the machine learning algorithm may comprise reinforcement learning algorithms. The reinforcement learning algorithm may be used for optimizing Markov decision processes (i.e., mathematical models used for studying a wide range of optimization problems where future behavior cannot be accurately predicted from past behavior alone, but rather also depends on random chance or probability). One example of reinforcement learning may be Q-learning. Reinforcement learning algorithms may differ from supervised learning algorithms in that correct training data input/output pairs are not presented, nor are sub-optimal actions explicitly corrected. The reinforcement learning algorithms may be implemented with a focus on real-time performance through finding a balance between exploration of possible outcomes (e.g., correct compound identification) based on updated input data and exploitation of past training.

In some embodiments, training data resides in a cloud-based database that is accessible from local and/or remote computer systems on which the machine learning-based sensor signal processing algorithms are running. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site.

In some embodiments, the trained algorithm may accept a plurality of input variables and produce one or more output variables based on the plurality of input variables. The input variables may comprise one or more datasets of codons. For example, the input variables may comprise information about a codon-of-interest, a codon upstream of (or 5′ to) the codon-of-interest, a codon downstream of (or 3′ to) the codon-of-interest, or any combination thereof. For example, the input variables may comprise a stop codon.

In some embodiments, the trained algorithm may be trained with a plurality of independent training samples. Each of the independent training samples may comprise information about a codon-of-interest, a codon upstream of (or 5′ to) the codon-of-interest, a codon downstream of (or 3′ to) the codon-of-interest, or a combination thereof. Each of the independent training samples may comprise information about a stop codon. The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, at least about 500, at least about 1,000, at least about 1,500, at least about 2,000, at least about 2,500, at least about 3,000, at least about 3,500, at least about 4,000, at least about 4,500, at least about 5,000, at least about, 5,500, at least about 6,000, at least about 6,500, at least about 7,000, at least about 7,500, at least about 8,000, at least about 8,500, at least about 9,000, at least about 9,500, at least about 10,000, or more independent training samples.

In some embodiments, the trained algorithm may associate information about a codon-of-interest, a codon upstream of (or 5′ to) the codon-of-interest, a codon downstream of (or 3′ to) the codon-of-interest, or a combination thereof for the best selection of codons for rewriting/replacement at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The trained algorithm may associate information about a stop codon for the best selection of codons for rewriting/replacement at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The trained algorithm may be adjusted or tuned to improve a performance or accuracy of determining the prediction or classification. The trained algorithm may be adjusted or tuned by adjusting parameters of the trained algorithm. The trained algorithm may be adjusted or tuned continuously during the training process or after the training process has completed.

In some embodiments, after the trained algorithm is initially trained, a subset of the inputs may be identified as most influential or most important to be included for making high-quality predictions. For example, a subset of the data may be identified as most influential or most important to be included for making high-quality choice for selecting codons for rewriting and/or replacement. The data or a subset thereof may be ranked based on classification metrics indicative of each parameter's influence or importance toward making high-quality selection of codons for rewriting and/or replacement. Such metrics may be used to reduce, in some embodiments significantly, the number of input variables (e.g., predictor variables) that may be used to train the trained algorithm to a desired performance level (e.g., based on a desired minimum accuracy). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best association metrics.

Systems and methods as described herein may use more than one trained algorithm to determine an output. Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., sequence data, structural data). Alternatively, a trained algorithm may be trained on more than one type of data. The inputs of one trained algorithm may comprise the outputs of one or more other trained algorithms. Additionally, a trained algorithm may receive as its input the output of one or more trained algorithms. A set of outputs generated using one or more trained algorithms may be combined into a single output (e.g., by determining a sum, an average, a minimum, a maximum, or any other function applied to the set of outputs).

### Other Embodiments

In some aspects, provided herein is a method of modulating protein translation, the method comprising editing a genome of an organism, wherein the editing comprises: a. replacing a first stop codon with a second stop codon; and b. causing the organism to express one or more peptides capable of recognizing only the second stop codon as a stop codon, wherein the one or more peptides do not recognize the first stop codon as a stop codon.

In some embodiments, the editing the genome further comprises replacing a third stop codon with the second stop codon, wherein the one or more peptides recognize the second stop codon as a stop codon, wherein the one or more peptides do not recognize the first stop codon or the third stop codon as a stop codon. In some embodiments, the second stop codon is UGA. In some embodiments, the first stop codon is UAA or UAG. In some embodiments, the third stop codon is UAA or UAG and wherein the third stop codon is different than the first stop codon.

In some embodiments, the genome encodes a release factor comprising the one or more peptides, wherein the one or more peptides provide release factor activity. In some embodiments, the one or more peptides are eRF1, eRF3, a methylase, an enzyme, or a tRNA.

In some embodiments, the release factor is capable of modulating protein translation upon recognizing the second stop codon as a stop codon. In some embodiments, the modulating protein translation is terminating protein translation.

In some embodiments, the organism is further engineered to recognize the first stop codon as a sense codon. In some embodiments, the organism is further engineered to recognize the third stop codon as a sense codon.

In some embodiments, the release factor and associated protein-coding and tRNA-coding genes are integrated into the host genome. In some embodiments, the release factor and associated protein-coding and tRNA-coding genes are provided on an episomal element bearing one or more counter-selectable genes. In some embodiments, the episomal element is a Superloser plasmid.

In some embodiments, phylogenetic screening is used to identify the best eRF and additional genes. In some embodiments, fitness is optimized and cross-talk is minimized by additional methods including directed evolution, library screens, and machine learning.

In some aspects, provided herein is a method comprising: rewriting a first stop codon to a second stop codon in a genome of a first organism; and introducing a release factor into the first organism, wherein the release factor is configured to recognize only the second stop codon as a stop codon, and wherein the release factor does not recognize the first stop codon as a stop codon.

In some embodiments, the method further comprises rewriting a third stop codon to the second stop codon, wherein the release factor does not recognize the first stop codon or the third stop codon as a stop codon. In some embodiments, the release factor does not recognize the first stop codon and the third stop codon as stop codons.

In some embodiments, the second stop codon is UGA. In some embodiments, the first stop codon is UAA or UAG. In some embodiments, the third stop codon is UAA or UAG, and wherein the third stop codon is different from the first stop codon.

In some embodiments, the release factor comprises a class 1 release factor or a class 2 release factor. In some embodiments, the class 1 release factor comprises a release factor 1 (RF1) or a release factor 2 (RF2). In some embodiments, the RF1 is a eukaryotic RF1 (eRF1). In some embodiments, the class 2 release factor comprises a release factor 3 (RF3). In some embodiments, the RF3 is a eukaryotic RF3 (eRF3). In some embodiments, the release factor is a release factor 1/release factor 3 (RF1/RF3) complex. In some embodiments, the RF1/RF3 complex is a eukaryotic RF1/RF3 (eRF1/eRF3) complex.

In some embodiments, the release factor modulates protein translation upon recognizing the second stop codon as a stop codon. In some embodiments, the modulating protein translation comprises terminating protein translation.

In some embodiments, the release factor comprises a recognition domain comprising one or more mutations that allow the release factor to recognize only the second stop codon as a stop codon. In some embodiments, the release factor comprises a first recognition domain swapped with a second recognition domain. In some embodiments, the second recognition domain is from a release factor of a second organism. In some embodiments, the second recognition domain is identified using a phylogenetic screening, directed evolution, library screening, machine learning, or a combination thereof. In some embodiments, the release factor is from a second organism.

In some embodiments, the second organism comprises a ciliate. In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Urostyla sp. HL-2004, Uroleptus sp. WJC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof. In some embodiments, the release factor comprises an amino acid sequence selected from the group consisting of SEQ ID NOs: 40-64.

In some embodiments, the release factor from the second organism comprises an eRF1. In some embodiments, the eRF1 from the second organism comprises an amino acid sequence that has at least 20% sequence identity to an eRF1 of the first organism. In some embodiments, the release factor comprises an amino acid sequence selected from the group consisting of SEQ ID NOs: 65-74. In some embodiments, the release factor from the second organism comprises an eRF1/eRF3 complex. In some embodiments, the eRF1 of the eRF1/eRF3 complex from the second organism comprises an amino acid sequence that has at least 20% sequence identity to an eRF1 of the first organism. In some embodiments, the eRF1 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of the first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the release factor from the second organism comprises an eRF1 and forms a complex with a chimeric eRF3. In some embodiments, the eRF1 of the second organism comprises an amino acid sequence that has at least 40% sequence identity to an eRF1 of the first organism. In some embodiments, the chimeric eRF3 comprises (i) an eRF3 from the first organism or a fragment thereof and (ii) an eRF3 from a second organism or a fragment thereof. In some embodiments, the second organism comprises Euplotes octocarinatus or Paramecium tetraurelia.

In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some embodiments, the first organism comprises a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae.

In some embodiments, the method further comprises inserting an additional stop codon next to the second stop codon. In some embodiments, the additional stop codon is UGA. In some embodiments, the inserting the additional stop codon enhances translation termination.

In some embodiments, the first organism does not comprise a gene encoding an endogenous RF1, RF2, or a combination thereof in the genome. In some embodiments, the gene comprises SUP35, SUP45, or a combination thereof.

In some embodiments, the method further comprises reassigning the first stop codon to encode a natural amino acid or a non-canonical amino acid (ncAA). In some embodiments, the method further comprises reassigning the third stop codon to encode a natural amino acid or a non-canonical amino acid (ncAA). In some embodiments, the natural amino acid comprises alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, the ncAA comprises p-azidophenylalanine, 2-aminoisobutyric acid (Aib), or a combination thereof.

In some embodiments, the method further comprises providing one or more tRNA molecules that recognize the first stop codon and one or more aminoacyl-tRNA synthetases (aaRSs) for charging the one or more tRNA molecules with the natural amino acid or the ncAA. In some embodiments, the method further comprises providing a tRNA pre-charged with the natural amino acid or the ncAA.

In some embodiments, the release factor is expressed from a gene integrated into the genome. In some embodiments, the release factor is expressed from an episomal element.

In some aspects, provided herein, is a method of producing a polypeptide molecule comprising a non-canonical amino acid (ncAA) or a population of polypeptide molecules comprising the ncAA in a first organism, the method comprising: a. rewriting a first stop codon to a second stop codon; b. reassigning the first stop codon to encode the ncAA in the genome of the first organism; and c. introducing an aminoacyl-tRNA synthetase (aaRS)/tRNA pair into the first organism, wherein the aaRS/tRNA pair is configured to recognize the first stop codon and incorporate the ncAA into an amino acid sequence of the polypeptide or the population of the polypeptide molecules.

In some embodiments, the introducing further comprises providing a tRNA pre-charged with the ncAA. In some embodiments, the ncAA comprises p-azidophenylalanine, 2-aminoisobutyric acid (Aib), or a combination thereof.

In some embodiments, the method further comprises rewriting a third stop codon to the second stop codon. In some embodiments, the second stop codon is UGA. In some embodiments, the first stop codon is UAA or UAG. In some embodiments, the third stop codon is UAA or UAG, wherein the third stop codon is different from the first stop codon.

In some embodiments, the release factor comprises a recognition domain comprising one or more mutations that allow the release factor to recognize only the second stop codon as a stop codon.

In some embodiments, the method further comprises introducing a release factor to the organism. In some embodiments, the release factor comprises a first recognition domain swapped with a second recognition domain. In some embodiments, the second recognition domain is from a release factor of a second organism. In some embodiments, the release factor is from a second organism. In some embodiments, the second organism comprises a ciliate.

In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Urostyla sp. HL-2004, Uroleptus sp. WJC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof. In some embodiments, the release factor comprises an amino acid sequence selected from the group consisting of SEQ ID NOs: 40-64.

In some embodiments, the release factor from the second organism comprises an eRF1. In some embodiments, the eRF1 from the second organism comprises an amino acid sequence that has at least 20% sequence identity to an eRF1 of the first organism. In some embodiments, the release factor comprises an amino acid sequence selected from the group consisting of SEQ ID NOs: 65-74. In some embodiments, the release factor from the second organism comprises an eRF1/eRF3 complex. In some embodiments, the eRF1 of the eRF1/eRF3 complex from the second organism comprises an amino acid sequence that has at least 20% sequence identity to an eRF1 of the first organism. In some embodiments, the eRF1 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of the first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the release factor from the second organism comprises an eRF1 and forms a complex with a chimeric eRF3. In some embodiments, the eRF1 of the second organism comprises an amino acid sequence that has at least 40% sequence identity to an eRF1 of the first organism. In some embodiments, the chimeric eRF3 comprises (i) an eRF3 from the first organism or a fragment thereof and (ii) an eRF3 from a second organism or a fragment thereof.

In some embodiments, the second organism comprises Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some embodiments, the first organism comprises a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae.

In some embodiments, the method further comprises inserting an additional stop codon next to the second stop codon. In some embodiments, the additional stop codon is UGA. In some embodiments, the inserting the additional stop codon enhances translation termination.

In some embodiments, the first organism does not comprise a gene encoding an endogenous RF1, RF2, or a combination thereof in the genome. In some embodiments, the gene comprises SUP35, SUP45, or a combination thereof.

In some aspects, provided herein, is a cell or a population of cells comprising a first stop codon rewritten to a second stop codon and further comprising (a) a release factor that recognizes only the second stop codon as a stop codon, (b) a release factor that recognizes only the second stop codon as a stop codon, (c) a release factor that recognizes only the third stop codon as a stop codon, or (d) a combination thereof. In some embodiments, the second stop codon is UGA. In some embodiments, the first stop codon is UAA or UAG. In some embodiments, the third stop codon is UAA or UAG, wherein the third stop codon is different from the first stop codon.

In some embodiments, the release factor comprises a recognition domain comprising one or more mutations that allow the release factor to recognize only the second stop codon as a stop codon. In some embodiments, the release factor comprises a recognition domain comprising one or more mutations that allow the release factor to recognize the first stop codon, the third stop codon, or a combination thereof, as a stop codon. In some embodiments, the release factor comprises a first recognition domain swapped with a second recognition domain. In some embodiments, the recognition domain is from a release factor of a first organism and the second recognition domain is from a release factor of a second organism. In some embodiments, the release factor is from a second organism. In some embodiments, the second organism comprises a ciliate.

In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof. In some embodiments, the release factor comprises an amino acid sequence selected from the group consisting of SEQ ID NOs: 40-64.

In some embodiments, the release factor from the second organism comprises an eRF1. In some embodiments, the eRF1 from the second organism comprises an amino acid sequence that has at least 20% sequence identity to an eRF1 of a first organism. In some embodiments, the release factor comprises an amino acid sequence selected from the group consisting of SEQ ID NOs: 65-74. In some embodiments, the release factor from the second organism comprises an eRF1/eRF3 complex. In some embodiments, the eRF1 of the eRF1/eRF3 complex from the second organism comprises an amino acid sequence that has at least 20% sequence identity to an eRF1 of a first organism. In some embodiments, the eRF1 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of a first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the release factor from the second organism comprises an eRF1 and forms a complex with a chimeric eRF3. In some embodiments, the eRF1 of the second organism comprises an amino acid sequence that has at least 40% sequence identity to an eRF1 of a first organism. In some embodiments, the chimeric eRF3 comprises (i) an eRF3 from a first organism or a fragment thereof and (ii) an eRF3 from a second organism or a fragment thereof. In some embodiments, the second organism comprises Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some embodiments, the first organism comprises a eukaryotic cell or a prokaryotic cell. In some embodiments, the cell or the population of cells comprises a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae. In some embodiments, the mammalian cell comprises a rodent cell, a mouse cell, or a human cell, or a combination thereof.

In some embodiments, the cell or the population of cells further comprises additional stop codon next to the second stop codon. In some embodiments, the additional stop codon is UGA. In some embodiments, the additional stop codon enhances translation termination.

In some embodiments, the cell or the population of cells does not comprise a gene encoding an endogenous RF1, RF2, or a combination thereof in the genome. In some embodiments, the gene comprises SUP35, SUP45, or a combination thereof.

In some aspects, provided herein, is an organism comprising the cell or the population of cells described herein.

In some aspects, provided herein is a method of producing a polypeptide molecule comprising a non-canonical amino acid (ncAA) or a population of polypeptide molecules comprising the ncAA, the method comprising introducing into the cell or the population of cells described herein, a) a first nucleic acid sequence construct encoding the polypeptide wherein the first nucleic acid sequence construct comprises the first stop codon reassigned to encode the ncAA; and b) a second nucleic acid sequence construct encoding an aminoacyl-IRNA synthetase (aaRS)/tRNA pair engineered to recognize the first stop codon and incorporate the ncAA into an amino acid sequence of the polypeptide, thereby producing the polypeptide molecule comprising the ncAA or the population of polypeptide molecules comprising the ncAA.

In some embodiments, the introducing further comprises providing a tRNA pre-charged with the ncAA. In some embodiments, the ncAA comprises p-azidophenylalanine, 2-aminoisobutyric acid (Aib), or a combination thereof.

In some aspects, provided herein, is a composition comprising: (a) a recombinant release factor configured to recognize only a second stop codon, (b) a recombinant release factor configured to recognize only a first stop codon as a stop codon, (c) a recombinant release factor configured to recognize only the third stop codon as a stop codon, or (d) a combination thereof.

In some embodiments, the composition comprises the recombinant release factor configured to recognize only a second stop codon, wherein the release factor does not recognize a first stop codon as a stop codon. In some embodiments, the release factor further does not recognize a third stop codon as a stop codon. In some embodiments, the second stop codon is UGA. In some embodiments, the first stop codon is UAA or UAG. In some embodiments, the third stop codon is UAA or UAG, and wherein the third stop codon is different from the first stop codon.

In some embodiments, the release factor comprises a class 1 release factor or a class 2 release factor. In some embodiments, the class 1 release factor comprises a release factor 1 (RF1) or a release factor 2 (RF2). In some embodiments, the RF1 is a eukaryotic RF1 (eRF1). In some embodiments, the class 2 release factor comprises a release factor 3 (RF3). In some embodiments, the RF3 is a eukaryotic RF3 (eRF3). In some embodiments, the release factor is a release factor 1/release factor 3 (RF1/RF3) complex. In some embodiments, the RF1/RF3 complex is a eukaryotic RF1/RF3 (eRF1/eRF3) complex.

In some embodiments, the release factor modulates protein translation upon recognizing the second stop codon as a stop codon. In some embodiments, the modulating protein translation comprises terminating protein translation.

In some embodiments, the release factor comprises a recognition domain comprising one or more mutations that allow the release factor to recognize only the second stop codon as a stop codon. In some embodiments, the release factor comprises a recognition domain comprising one or more mutations that allow the release factor to recognize the first stop codon, the third stop codon, or a combination thereof, as a stop codon. In some embodiments, the release factor comprises a first recognition domain swapped with a second recognition domain. In some embodiments, the second recognition domain is from a release factor of a second organism. In some embodiments, the second recognition domain is identified using a phylogenetic screening, directed evolution, library screening, machine learning, or a combination thereof.

In some embodiments, the release factor is from a first organism. In some embodiments, the first organism comprises a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae.

In some embodiments, the release factor is from a second organism. In some embodiments, the second organism comprises a ciliate. In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Urostyla sp. HL-2004, Uroleptus sp. WJC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof. In some embodiments, the release factor comprises an amino acid sequence selected from the group consisting of SEQ ID NOs: 40-64.

In some embodiments, the release factor from the second organism comprises an eRF1. In some embodiments, the eRF1 from the second organism comprises an amino acid sequence that has at least 20% sequence identity to an eRF1 of the first organism. In some embodiments, the release factor comprises an amino acid sequence selected from the group consisting of SEQ ID NOs: 65-74. In some embodiments, the release factor from the second organism comprises an eRF1/eRF3 complex. In some embodiments, the eRF1 of the eRF1/eRF3 complex from the second organism comprises an amino acid sequence that has at least 20% sequence identity to an eRF1 of the first organism. In some embodiments, the eRF1 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of the first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the release factor from the second organism comprises an eRF1 and forms a complex with a chimeric eRF3. In some embodiments, the eRF1 of the second organism comprises an amino acid sequence that has at least 40% sequence identity to an eRF1 of the first organism. In some embodiments, the chimeric eRF3 comprises (i) an eRF3 from the first organism or a fragment thereof and (ii) an eRF3 from a second organism or a fragment thereof. In some embodiments, the second organism comprises Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some aspects, provided herein, is a method comprising: a. rewriting UAA and UAG to UGA in a genome of a yeast; b. introducing a release factor into the yeast, wherein the release factor is configured to recognize only UGA as a stop codon, and wherein the release factor does not recognize UAA and UAG as a stop codon; and c. reassigning UAA or UAG to encode a natural amino acid or a non-canonical amino acid (ncAA).

In some embodiments, the release factor comprises eukaryotic release factor 1 (eRF1), eRF2, eRF3, or a combination thereof. In some embodiments, the release factor comprises a eukaryotic RF1/RF3 (eRF1/eRF3) complex. In some embodiments, the release factor terminates protein translation upon recognizing UGA as a stop codon. In some embodiments, the release factor comprises a first recognition domain swapped with a second recognition domain from a ciliate. In some embodiments, the release factor is from a ciliate.

In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof. In some embodiments, the release factor comprises an amino acid sequence selected from the group consisting of SEQ ID NOs: 40-64.

In some embodiments, the release factor from the ciliate comprises an eRF1 comprising an amino acid sequence that has at least 20% sequence identity to a yeast eRF1. In some embodiments, the release factor comprises an amino acid sequence selected from the group consisting of SEQ ID NOs: 65-74. In some embodiments, the release factor from the ciliate comprises an eRF1/eRF3 complex, wherein the eRF1 comprises an amino acid sequence that has at least 20% sequence identity to a yeast eRF1, and wherein the eRF3 comprises an amino acid sequence that has at least 25% sequence identity to a yeast eRF3. In some embodiments, the eRF1 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the release factor from the ciliate comprises an eRF1 and forms a complex with a chimeric eRF3, wherein the eRF1 comprises an amino acid sequence that has at least 40% sequence identity to a yeast eRF1. In some embodiments, the chimeric eRF3 comprises (i) a yeast eRF3 or a fragment thereof and (ii) an eRF3 or a fragment thereof from Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the yeast eRF3. In some embodiments, the chimeric eRF3 comprises an eRF3 of Euplotes octocarinatus, wherein amino acids 1-298 of the eRF3 of Euplotes octocarinatus is replaced with amino acids 1-253 of the yeast eRF3. The method of 183, wherein the chimeric eRF3 comprises an eRF3 of Paramecium tetraurelia, wherein amino acids 1-321 of the eRF3 of Paramecium tetraurelia is replaced with amino acids 1-253 of the yeast eRF3. In some embodiments, the yeast comprises Saccharomyces cerevisiae.

In some embodiments, the ncAA comprises p-azidophenylalanine, 2-aminoisobutyric acid (Aib), or a combination thereof. In some embodiments, the release factor is expressed from a gene integrated into the genome or an episomal element.

In some embodiments, the method further comprises inserting an additional stop codon next to the second stop codon. In some embodiments, the additional stop codon is UGA. In some embodiments, the inserting the additional stop codon enhances translation termination.

In some embodiments, the yeast does not comprise a gene encoding an endogenous eRF1, eRF2, or a combination thereof in the genome. In some embodiments, the gene comprises SUP35, SUP45, or a combination thereof.

In some aspects, provided herein, is a system for producing a polypeptide molecule comprising a non-canonical amino acid (ncAA) comprising the ncAA comprising: a. a gene encoding the polypeptide molecule, wherein the gene comprises a first stop codon rewritten to a second stop codon, and wherein the first stop codon is reassigned to encode the ncAA; b. a release factor, wherein (i) the release factor is configured to recognize only the second stop codon as a stop codon, and wherein the release factor does not recognize the first stop codon as a stop codon, (ii) the release factor is configured to recognize only the first stop codon as a stop codon, (iii) the release factor is configured to recognize only a third stop codon as a stop codon, or (iv) a combination thereof; and c. an aminoacyl-tRNA synthetase (aaRS)/tRNA pair, wherein the aaRS/tRNA pair is configured to recognize the first stop codon and incorporate the ncAA into an amino acid sequence of the polypeptide molecule.

In some embodiments, the system further comprises a tRNA pre-charged with the ncAA. In some embodiments, the ncAA comprises p-azidophenylalanine, 2-aminoisobutyric acid (Aib), or a combination thereof.

In some embodiments, the second stop codon is UGA. In some embodiments, the first stop codon is UAA or UAG.

In some embodiments, the release factor comprises a recognition domain comprising one or more mutations that allow the release factor to recognize only the second stop codon as a stop codon. In some embodiments, the release factor comprises a recognition domain comprising one or more mutations that allow the release factor to recognize the first stop codon, the third stop codon, or a combination thereof, as a stop codon. In some embodiments, the release factor comprises a first recognition domain from a first organism swapped with a second recognition domain from a second organism. In some embodiments, the release factor is from a second organism. In some embodiments, the second organism comprises a ciliate.

In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Urostyla sp. HL-2004, Uroleptus sp. WJC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof. In some embodiments, the release factor comprises an amino acid sequence selected from the group consisting of SEQ ID NOs: 40-64.

In some embodiments, the release factor from the second organism comprises an eRF1. In some embodiments, the eRF1 from the second organism comprises an amino acid sequence that has at least 20% sequence identity to an eRF1 of the first organism. In some embodiments, the release factor comprises an amino acid sequence selected from the group consisting of SEQ ID NOs: 65-74. In some embodiments, the release factor from the second organism comprises an eRF1/eRF3 complex. In some embodiments, the eRF1 of the eRF1/eRF3 complex from the second organism comprises an amino acid sequence that has at least 20% sequence identity to an eRF1 of the first organism. In some embodiments, the eRF1 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of the first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the release factor from the second organism comprises an eRF1 and forms a complex with a chimeric eRF3. In some embodiments, the eRF1 of the second organism comprises an amino acid sequence that has at least 40% sequence identity to an eRF1 of the first organism. In some embodiments, the chimeric eRF3 comprises (i) an eRF3 from the first organism or a fragment thereof and (ii) an eRF3 from a second organism or a fragment thereof. In some embodiments, the second organism comprises Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some embodiments, the first organism comprises a eukaryotic cell or a prokaryotic cell. In some embodiments, the prokaryote comprises an archaebacterial cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae.

In some embodiments, the gene further comprises an additional stop codon next to the second stop codon. In some embodiments, the additional stop codon is UGA. In some embodiments, the additional stop codon enhances translation termination.

### Examples

These examples are provided for illustrative purposes only and not to limit the scope of the claims provided herein.

### Example 1: Release Factor (RF) Engineering-Mutagenesis

A release factor (RF) that recognizes all three stop codons (e.g., UAA, UAG, and UGA) can be mutated to recognize only one or two stop codons. Such mutation(s) can be made in a recognition domain of an RF.

First, a three-dimensional structure of one or more RFs of interest or a domain of one or more RFs of interest can be obtained. A domain with semi-conserved and invariant amino acid residues located near known amino acid residues important for functional role (e.g., NIKS (SEQ ID NO: 162) or YCF mini domain) can be identified. One or more semi-conserved and invariant amino acids in the aforementioned domain can be selected for mutagenesis.

The mutagenesis of selected amino acids can be performed according to any known methods in the art, including PCR-based megaprimer methods or site-directed mutagenesis. The PCR primers can be designed to contain relevant amino acid substitutions and restriction enzyme digestion sites for cloning. DNA amplifications can be carried out according to any methods in the art. The amplified DNA fragments can be digested by restriction enzymes selected for cloning and ligated into the same restriction sites of the host system (e.g., a plasmid containing a host RF gene). The ligated mixture can be transformed into Escherichia coli. The cloned DNAs can be sequenced to confirm that the cloned DNAs have the desired mutations.

The RF can be expressed and purified in vitro and the RF activity can be measured in vitro.

### Example 2: Release Factor (RF) Engineering-Domain/Motif Swapping I

A recognition domain of a release factor (RF) from an organism (e.g., a ciliate) can be swapped into an RF of a host (e.g., a eukaryotic platform, such as a yeast).

First, a three-dimensional structure of one or more RFs of interest can be obtained. Hinge regions (e.g., hinge 1 and hinge 2) and recognition domains (e.g., domain 1, domain 2, and domain 3) can be identified. Conserved amino acid sequences at the junctions of domain 1 and domain 2 (e.g., hinge 1), and at the junctions of domain 2 and domain 3 (e.g., hinge 2) of the RFs can be identified. Each domain can be swapped at the hinge.

Restriction enzyme sites at the conserved amino acid sequences at the junctions can be analyzed to identify a restriction enzyme site for domain swapping. PCR primers for amplifying one or more recognition domains can be designed to include the restriction enzyme site of choice. DNA amplifications can be carried out according to any methods in the art. The amplified recognition domain fragments can be digested with restriction enzymes and ligated into the same restriction sites of the host system (e.g., a plasmid comprising a host RF gene) to give rise to a hybrid RF gene.

The RF can be expressed and purified in vitro and the RF activity can be measured in vitro.

### Example 3: Release Factor (RF) Engineering-Domain Swapping II

Recognition domains in yeast eRF1 (encoded by SUP45 gene) were engineered to introduce the corresponding recognition domains of ciliate eRF1s. The resulting domain-swapped yeast eRF1 was tested in yeast for the ability to confer the stop codon selectivity of ciliate eRF1s. An episomal-based shuffle system was employed (FIG. 2). A yeast strain which lacks SUP45 gene (sup45/1) was generated. As the SUP45 gene is essential, the strain was introduced with the wild-type (WT) SUP45 gene on a counter-selectable plasmid. In this case, this counter-selectable marker is URA3, which can be selected against in media containing 5-FOA. Next, a set of “domain-swapped” sup45 constructs (see Table 3), which were under the control of the SUP45 promoter (SUP45pr), were generated with LEU2 or HIS3 markers. In an example of such a system, the candidate UAA/UAG-specific domain-swapped yeast eRF1 was cloned on a vector marked with LEU2, while the candidate UGA-specific eRF1 was cloned on a vector marked with HIS3. Once vectors were transformed into the yeast sup45/1 mutant. strains were maintained on media that selected for all three vectors (e.g. . . . Synthetic complete medium which lacked uracil, leucine, and histidine, aka SC-URA-LEU-HIS). Viability of the sup45/1 strain without the WT URA3-marked SUP45 was assessed post-shuffle on media containing 5-FOA.

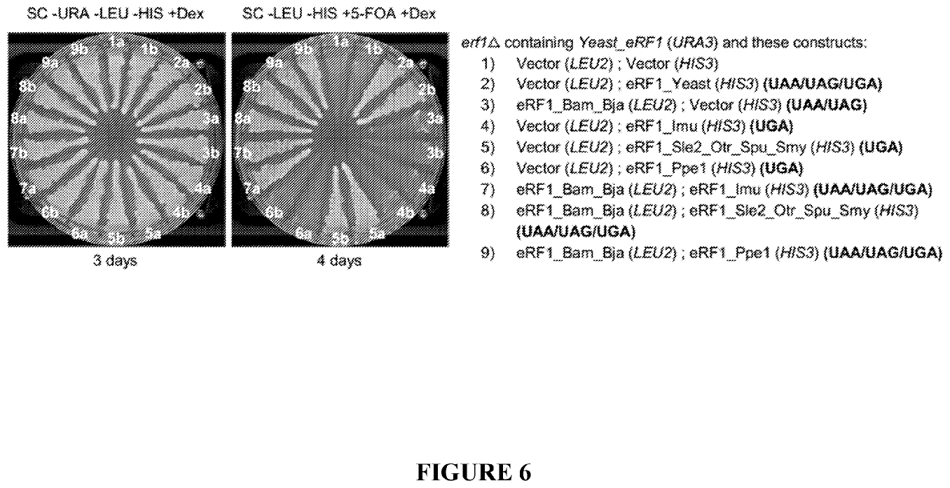

FIG. 6 illustrates an example of testing for stop-codon selectivity and functionality of a domain/motif-swapped yeast eRF1. A yeast erf1Δ strain pre-transformed with the endogenously regulated yeast eRF1 (URA3-marked plasmid), was subsequently transformed with the endogenously regulated, domain-swapped UAA/UAG-specific construct “eRF1_Bam_Bja” (LEU2-marked plasmid) or an empty LEU2 vector, and the endogenously regulated candidate UGA-specific motif-swapped ciliate eRF1 constructs (HIS3-marked plasmid) or an empty HIS3 vector. Yeast strains, post transformation, were maintained on dextrose media that selects for all three plasmid constructs (SC-URA-LEU-HIS+Dex). The same strains were also streaked on dextrose medium supplemented with 5-FOA, selecting for only the motif-swapped ciliate constructs (SC-LEU-HIS+5-FOA+Dex). Three different candidate UGA-specific constructs (different only in their yeast TASNIKS/YLCDNKF motifs (SEQ ID NOs: 1 and 2, respectively, in order of appearance)) were tested for their ability to complement erf1 deletion on 5-FOA media. The eRF1 Sle2_Otr_Spu_Smy construct (isolates #5a, #5b) supported viability of an erf1A strain in the absence of eRF1_Bam_Bja, suggesting that this construct was not specific to UGA in vivo. The other two UGA-specific constructs, eRF1_Imu and eRF1_Ppe1, suppressed the lethality of an erf14 mutant on 5-FOA media, only when combined with eRF1_Bam_Bja. Two independent transformants were tested for each strain (labelled a and b). Isolate #2 provided a positive control sample where the native yeast eRF1 gene was expressed from the HIS3-marked plasmid alongside a LEU2 empty vector (FIG. 6).

### Example 4: Release Factor (RF) Engineering-Whole-Gene Swap

The native whole-gene release factor (RF) from an organism (e.g., a ciliate) can replace the RF of a host (e.g., a eukaryotic platform, such as a yeast).

The wild-type yeast eRF1 can be replaced by the entire ciliate eRF1 protein. In this case, replaceability is tested in a sup45Δ mutant. In some cases, the corresponding ciliate eRF3 may be required for ciliate eRF1 function in yeast. In this case, replaceability can be tested in a sup45Δ or sup45Δ sup35Δ mutant.

An episomal-based shuffle system was employed (FIG. 2). The yeast genes, SUP45 and SUP35, (separate or together) were cloned on a vector that carries a counter-selectable marker (such as URA3), and their expression was driven using either the native endogenous promoters or an inducible promoter system (such as the bi-directional GAL1/10 system). Codon-optimized ciliate UAA/UAG- and UGA-specific RFs (eRF1 or eRF1/eRF3) were cloned on two separate vectors that carry different auxotrophic markers (such as LEU2 and HIS3), and their expression was driven using either the corresponding yeast endogenous promoters or an inducible promoter system (such as the bi-directional GAL1/10 system). In an example of such a system, the UAA/UAG-specific ciliate RFs were cloned on a vector marked with LEU2, while the UGA-specific ciliate RFs were cloned on a vector marked with HIS3. In the cases where ciliate eRF3 was not included, endogenous yeast eRF3 (SUP35) must be included in the host strain, and the yeast eRF3 protein may function with the ciliate eRF1. In cases where ciliate eRF3 was included, the experiments could be done with or without yeast eRF3. The episomal shuffle strains were derived by transformation of vectors (such as those marked by LEU2 or HIS3) containing ciliate RFs into the yeast haploid deletion mutants that already contain the counter-selectable vector. Examples of these episomal shuffle strains included, but were not limited to, the sup45Δ or sup45Δ sup35Δ haploids containing 3 vectors: the counter-selectable URA3-marked vector that contained the corresponding wildtype yeast RFs, the LEU2-marked vector contained the UAA/UAG-specific ciliate RFs, and the HIS3-marked vector contained the UGA-specific ciliate RFs. Once vectors were transformed, strains were maintained on media that selected for all three vectors (e.g. . . . Synthetic complete medium which lacked uracil, leucine, and histidine, aka SC-URA-LEU-HIS).

The episomal shuffle strategy tested viability of strains on media supplemented with 5-FOA. In the case where expression of the vector-based ciliate gene(s) was driven by the corresponding yeast endogenous promoter(s), the 5-FOA medium contained any sugar source (preferably dextrose). In the case where expression of the vector-based ciliate gene(s) was driven by the inducible GAL/10 promoter, the 5-FOA medium contained galactose as the sugar source and constructs were induced on galactose media before plating on 5-FOA.

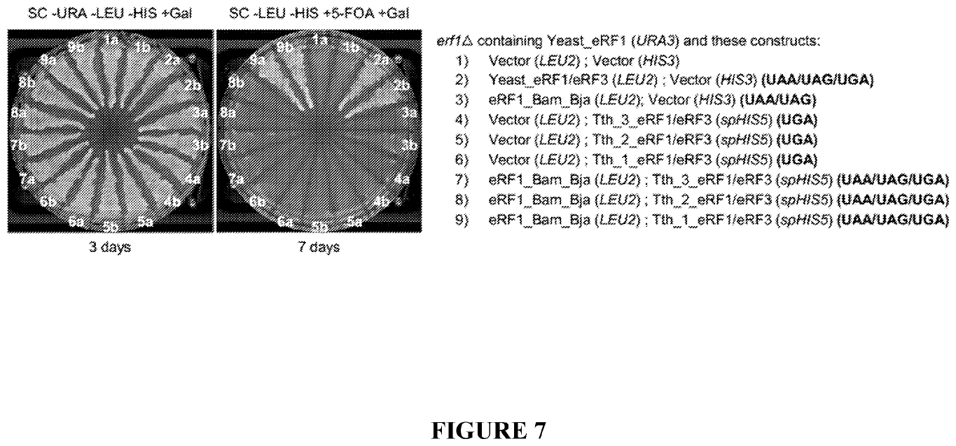

FIG. 7 illustrates an example of testing for stop-codon selectivity and functionality of whole-gene ciliate eRF1/eRF3 in yeast. A yeast erf1Δ strain pre-transformed with the endogenously regulated yeast eRF1 (URA3-marked plasmid), was subsequently transformed with the endogenously regulated, motif-swapped UAA/UAG-specific construct “eRF1_Bam_Bja” (LEU2-marked plasmid, or an empty vector) and/or the galactose-inducible candidate UGA-specific whole-gene ciliate eRF1/eRF3 constructs (spHIS5- or HIS3-marked plasmid, or an empty vector). Yeast strains, post transformation, are maintained on dextrose media that selects for all three plasmid constructs (SC-URA-LEU-HIS+Dex; not pictured). Galactose-regulated ciliate ORFs were induced on the same selective media containing galactose for 3 days (SC-URA-LEU-HIS+Gal), before re-streaking on galactose media containing 5-FOA, while selecting for only the whole-gene ciliate constructs (SC-LEU-HIS+5-FOA+Gal). Three different galactose-inducible Tth_eRF1/eRF3 constructs (different only in their eRF1 ORFs) were tested for their ability to complement deletion of erf1Δ deletion on 5-FOA media. Only Tth_1_eRF1/eRF3 (Tth_eRF1_XP_001018735.1/Tth_eRF3_XP_001011280.3), in combination with the UAA/UAG-specific construct, suppressed the lethality of an erf1Δ mutant on 5-FOA media. The results suggested that the whole-gene ciliate Tth_1_eRF1 construct was functional and UGA-specific, while the other two Tth_eRF1 constructs were non-functional in yeast. Two independent transformants were tested for each strain (labeled a and b). Isolate #2 provided a positive control sample where the native yeast eRF1leRF3 gene was expressed from the LEU2-marked plasmid (FIG. 7).

The 5-FOA media selects for two of the vector constructs (ex. LEU2-marked UAA/UAG-specific construct and HIS3-marked UGA-specific constructs) (FIGS. 6 and 7). Given that both eRF1 and eRF3 of yeast are essential genes, upon counter-selection on 5-FOA in the episomal shuffle system, if an expression of a single ciliate-derived engineered RF results in viability, this indicates that this RF recognizes all three stop codons in vivo in yeast (FIGS. 6, 5a and 5b). In this case, stop codon selectivity is not achieved (Table 3, “wild-type” result).

### Example 5: Plasmid-Dependency of Erf14 Strains

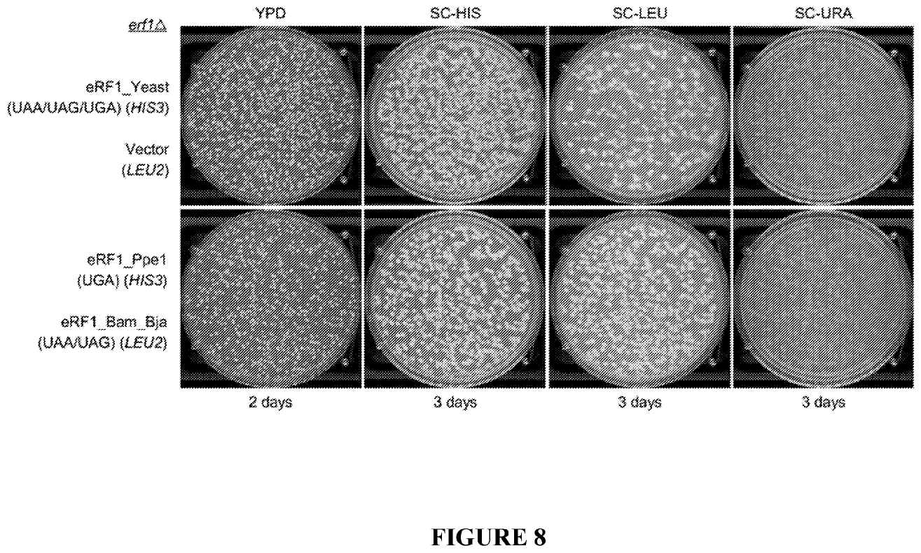

To test whether strains that are viable on 5-FOA are dependent on both the UAA/UAG- and UGA-specific constructs, colonies were isolated from the selective media (SC-LEU-HIS+5-FOA) and grown in non-selective YPD media. Only strains that required both plasmid constructs to decode all three stop codons formed viable LEU′ and HIS colonies after growth in YPD. As a control, these strains should not grow on-URA plates, given that they were isolated from media containing 5-FOA (FIG. 8).

FIG. 8 illustrates an example for assessing the plasmid-dependency of erf1/1 strains carrying ciliate release factor constructs. Yeast erf1Δ strains containing different combinations of plasmid constructs were isolated from SC-LEU-HIS+5-FOA plates. Strains were grown to saturation in non-selective liquid YPD medium at 30° C. for 1 day, and then re-inoculated in the same medium and grown to saturation for a second day. Cells were plated for single colonies on YPD and incubated for 2 days at 30° C., and then replica-plated to SC-HIS, SC-LEU, or SC-URA agar plates (all dextrose). Viability was assessed after 3 days. In the first example, the HIS3-marked plasmid encoding the endogenously regulated (SUP45pro) yeast eRF1 gene construct (UAA/UAG/UGA) was required for viability of an erf14 mutant. The LEU2-marked empty vector control was not required for viability and thus could be lost, resulting in colonies unable to grow on medium lacking leucine (SC-LEU). No growth was observed on SC-URA plates given that the strains were isolated from media supplemented with 5-FOA. In the second example, both the HIS3- and LEU2-marked plasmids encoding the endogenously regulated (SUP45pro) eRF1_Ppe1 (UGA) and the eRF1_Bam_Bja (UAA/UAG) gene constructs, respectively, were required for viability of an erf14 mutant. No growth was observed on SC-URA plates given that the strains were isolated from media supplemented with 5-FOA (FIG. 8).

### Example 6: Phylogenetic Screening for eRF1 Domain/Motif Swapping

This example described below was performed for eRF1 domain/motif swapping experiments, specifically the TASNIKS (SEQ ID NO: 1) and YCF domains.

To identify additional ciliate eRF1s for domain/motif swapping and functional testing in yeast, we extracted all proteins annotated in Gene Ontology as codon-specific release factors plus all proteins annotated as eRF1 by Uniprot's annotation system. We then narrowed down the list to organisms that use a subset of the 3 stop codons. And then we looked for the overlap with NCBI translation tables 4, 6, and 10. NCBI translation tables 4, 6, and 10 can be found: https://www.ncbi.nlm.nih.gov/Taxonomy/Utils/wprintgc.cgi #SG4.

NCBI Translation Table 4. The Mold, Protozoan, and Coelenterate Mitochondrial Code and the Mycoplasma/Spiroplasma Code (transl_table=4)

NCBI Translation Table 6. The Ciliate, Dasycladacean and Hexamita Nuclear Code (transl_table=6)

NCBI Translation Table 10. The Euplotid Nuclear Code (transl_table=10) This analysis uncovered:
1 example of NCBI translation table 4: Blepharisma; Mold Mitochondrial; Protozoan Mitochondrial; Coelenterate Mitochondrial; Mycoplasma; Spiroplasma
24 examples of NCBI translation table 6: Ciliate Nuclear; Dasycladacean Nuclear; Hexamita Nuclear
9 examples of NCBI translation table 10: Euplotid Nuclear

Within the 34 uncovered examples, there were 24 unique TASNIKS/YCF motifs (“TASNIKS” disclosed as SEQ ID NO: 1), which were tested using the episome-shuffle system (Table 3).

### Example 7: Stop Codon Capture

A Saccharomyces cerevisiae strain with the following genotype is built:
1. Inducibly expressed dual fluorescent reporter construct
2. p-azidophenylalanine (pAzF) orthogonal translation system (tRNA and synthetase)
3. deleted for yeast eRF1
4. a downregulatable yeast eRF1 UAA/UAG specific-construct
5. a constitutively expressed yeast eRF1 UGA specific-construct

Readthrough signals of the dual fluorescent reporter under all combination of the following conditions are evaluated:
1. Presence of the ncAA pAzF
2. Absence of the ncAA pAzF
3. Presence of the downregulatable yeast eRF1 UAA/UAG specific-construct
4. Absence of the downregulatable yeast eRF1 UAA/UAG specific-construct

Expected result: Increased readthrough signal in the presence of pAzF and in the absence of downregulatable yeast eRF1 UAA/UAG specific-construct as a function of eliminating competition between the pAzF orthogonal translation system and the release factor.

### Example 8: UAA/UAG-Specific Constructs

### Domain/Motif-Swap

Table 3 highlights all the UAA/UAG-specific domain-swapped yeast eRF1 constructs tested in yeast. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1Bam_Bja) (LEU2) and the indicated HIS3-marked candidate UGA-specific constructs, or with the endogenously-regulated (SUP45pro) motif-swap UGA-specific construct (eRF1Pte1_(m1)) (HIS3) and the indicated LEU2-marked candidate UAA/UAG-specific constructs. Yeast strains were maintained on SC-URA-LEU-HIS+Dex media, before testing for replaceability on SC-LEU-HIS+5-FOA+Dex media (Table 3).

The eRF1 protein has two “motifs” or highly conserved amino acid sequences important for specifying what stop codons are recognized. In yeast, the omnipotent eRF1 recognizes all three stop codons, and the motifs in question are TASNIKS (SEQ ID NO: 1) and YLCDNKF (SEQ ID NO: 2). Prior work has suggested that specific changes to these motifs underlie the exclusive recognition of either UGA or UAA/UAG found in ciliates. In these examples, the impact of introducing these motifs into the yeast protein is tested in the yeast cell. Two parameters are measured: the stop codon specificity of the construct in the context of the yeast cell, and the ability of the construct to function in yeast.

The eRF1 Bam_Bja construct was UAA/UAG-specific and could function in yeast. The eRF1_Bam_Bja construct was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of both organisms Blepharisma americanum and Blepharisma japonicum). The episomal-based shuffle system, which utilized 5-FOA to counter-select against the URA3-marked omnipotent (e.g., recognizing UGA, UAA and UAG) wild-type yeast eRF1, was employed to test the stop codon specificity and functionality of the construct when expressed in yeast. To provide UGA-specificity, another construct (eRF1_Pte1_(m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When individually expressed, the eRF1_Bam_Bja and eRF1_Pte1_(m1) eRF1 constructs did not support viability of a sup45Δ mutant on 5-FOA media, suggesting that these strains post-shuffle cannot decode UGA or UAA/UAG, respectively. When expressed in combination, the eRF1_Bam_Bja and eRF1_Pte1_(m1) constructs together supported viability of a sup45Δ mutant on 5-FOA media, consistent with the predicted exclusive stop codon specificity of the two eRF1 constructs and simultaneously demonstrating that each was functional in yeast (Table 3).

The eRF1_Eae1_Eoc1 construct was UAA/UAG-specific and could function in yeast. The eRF1_Eae1_Eoc1 construct was derived by swapping the TASNIKS/YLCDNKF motifs (SEQ ID NOs: 1 and 2, respectively, in order of appearance) in yeast eRF1 to TAVNIKS/YICDNKF (SEQ ID NOs: 5 and 4, respectively, in order of appearance; as found in the eRF1 protein sequences of the organisms Euplotes aediculatus and Euplotes octocarinatus). The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1 (m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When expressed individually, the eRF1_Eae1_Eoc1 and eRF1_Pte1_(m1) eRF1 constructs did not support viability of a sup454 mutant on 5-FOA media, suggesting that these strains post-shuffle cannot decode UGA or UAA/UAG, respectively. When expressed in combination, the eRF1_Eae1_Eoc1 and eRF1_Pte1_(m1) constructs together supported viability of a sup454 mutant on 5-FOA media, consistent with the predicted stop codon specificity of the two eRF1 constructs and simultaneously demonstrating that each was functional in yeast (Table 3).

TABLE 3
Summary of motif-swapped construct replacements.

| Stop | | Motif (underlined amino acids: mutations | | |
|---|---|---|---|---|
| Codon | Construct Name | introduced for each construct) | | |
| UAA/UAG/ | eRF1_ | TASNIKS | YLCDNKF | notes |
| UGA | Yeast | (SEQ ID NO: 1) | (SEQ ID NO: 2) | Status *** |
| UAA/UAG | eRF1_Bam_Bja * | KSSNIKS | YICDNKF | Replaceable |
| | | (SEQ ID NO: 3) | (SEQ ID NO: 4) | |
| UAA/UAG | eRF1_Eael_Eoc1 * | TAVNIKS | YICDNKF | Replaceable |
| | | (SEQ ID NO: 5) | (SEQ ID NO: 4) | |
| UAA/UAG | eRF1_Sco * | KAANIKS | YLCDNKF | WT |
| | | (SEQ ID NO: 6) | (SEQ ID NO: 2) | |
| UAA/UAG | eRF1_Nov * | KASNIKS | YYCGERF | WT |
| | | (SEQ ID NO: 7) | (SEQ ID NO: 8) | |
| UAA/UAG | eRF1_Eae2_Eoc2 * | TAESIKS | YICDNKF | Non-replaceable |
| | | (SEQ ID NO: 9) | (SEQ ID NO: 4) | |
| UGA | eRF1_Pte1_(m1) ** | TASNIKS | YFCDPQF | Replaceable |
| | | (SEQ ID NO: 1) | (SEQ ID NO: 10) | |
| UGA | eRF1_Pte1_(m2) ** | EAASIKD | YFCDPQF | Replaceable |
| | | (SEQ ID NO: 11) | (SEQ ID NO: 10) | |
| UGA | eRF1_Tth1 ** | KATNIKD | YFCDSKF | WT |
| | | (SEQ ID NO: 12) | (SEQ ID NO: 13) | |
| UGA | eRF1_Sle1 ** | FDFDAES | TLIKPQF | Non-replaceable |
| | | (SEQ ID NO: 14) | (SEQ ID NO: 15) | |
| UGA | eRF1_Ppe2 ** | TGDKIKS | TIIKNDF | Non-replaceable |
| | | (SEQ ID NO: 16) | (SEQ ID NO: 17) | |
| UGA | eRF1_Pte2 ** | EAASIQD | FFCDNYF | Non-replaceable |
| | | (SEQ ID NO: 18) | (SEQ ID NO: 19) | |
| UGA | eRFI_Imu ** | KATNIKD | FVIVNKF | Replaceable |
| | | (SEQ ID NO: 12) | (SEQ ID NO: 20) | |
| UGA | eRF1_S1e2_Otr_Spu_Smy ** | AAQNIKS | YFCGGKF | WT |
| | | (SEQ ID NO: 21) | (SEQ ID NO: 22) | |
| UGA | eRF1_Ppe1 ** | QANSIKD | YRCDSKF | Replaceable |
| | | (SEQ ID NO: 23) | (SEQ ID NO: 24) | |
| UGA | eRF1_Tth2 ** | GAASIKN | YSCNTIF | Replaceable |
| | | (SEQ ID NO: 25) | (SEQ ID NO: 26) | |
| UGA | eRF1_Eh1 ** | SAQNIKS | YYCDNRF | WT |
| | | (SEQ ID NO: 27) | (SEQ ID NO: 28) | |
| UGA | eRF1_Gh1 ** | SAGNIKS | YFCDNSF | WT |
| | | (SEQ ID NO: 29) | (SEQ ID NO: 30) | |
| UGA | eRF1_Hh1 ** | TAQNIKS | YFCGGKF | WT |
| | | (SEQ ID NO: 31) | (SEQ ID NO: 22) | |
| UGA | eRF1_Uh1 ** | SAQSIKS | YFCDNSF | Replaceable |
| | | (SEQ ID NO: 32) | (SEQ ID NO: 30) | |
| UGA | eRF1_Uwj_Pwe ** | AANNIKS | YFCGGKF | WT |
| | | (SEQ ID NO: 33) | (SEQ ID NO: 22) | |
| UGA | eRF1_Smi ** | TASNIKS | YNCSGKF | WT |
| | | (SEQ ID NO: 1) | (SEQ ID NO: 34) | |
| UGA | eRF1_Sa1 ** | QAQNIKS | YFCGGKF | WT |
| | | (SEQ ID NO: 35) | (SEQ ID NO: 22) | |
| UGA | eRF1_Ssa ** | QADCIKS | YSCDGVF | Replaceable |
| | | (SEQ ID NO: 36) | (SEQ ID NO: 37) | |
| UGA | eRF1_Lst ** | RAQNIKS | FLCENTF | Replaceable |
| | | (SEQ ID NO: 38) | (SEQ ID NO: 39) | |

* Candidate UAA/UAG-specific constructs tested against the UGA-specific eRF1_Ptel_(ml); all constructs regulated by a SUP45pro
** Candidate UGA-specific constructs tested against the UAA/UAG-specific eRF1_Bam_Bja; all constructs regulated by a SUP45pro
*** Status of construct when tested in an erf1Δ mutant:
Replaceable: Functional in yeast and confers stop codon selectivity, supports growth on 5-FOA only when expressed with the opposite construct
Non-replaceable: Not functional in yeast, unknown status on stop codon selectivity, does notsupport growth on 5-FOA when expressed with the opposite construct
WT: Functional in yeast but does not confer stop codon selectivity, supports growth on 5-FOA when expressed either individually or with the opposite construct

### Whole Gene Swaps

Table 4 highlights the UAA/UAG whole-gene ciliate eRF1 constructs tested in yeast. Ciliate eRF1 constructs, under the transcriptional control of the yeast eRF1 endogenous promoter (SUP45pro), were tested against the motif-swap constructs. A yeast erf1Δ strain pre-transformed with the endogenously regulated yeast eRF1 (URA3-marked plasmid), was subsequently transformed with the endogenously regulated (SUP45pro) motif-swap UAA/UAG-specific construct (eRF1_Bam_Bja) (LEU2) and the indicated HIS3-marked UGA-specific whole-gene constructs, or with the endogenously regulated (SUP45pro) motif-swap UGA-specific construct (eRF1_Pte1_(m1)) (HIS3) and the indicated LEU2-marked UAA/UAG-specific whole-gene constructs. Yeast strains were maintained on SC-URA-LEU-HIS+Dex media, before testing for replaceability on SC-LEU-HIS+5-FOA+Dex media.

The Eoc_eRF1_CAC14170.1 construct coded for a UAA/UAG-specific eRF1 protein that could function in yeast. The whole gene eRF1 construct was derived from the organism Euplotes octocarinatus. The episomal-based shuffle system, which utilized 5-FOA to counter-select against the URA3-marked omnipotent wild-type yeast eRF1, was employed to test the stop codon specificity and functionality of the ciliate construct when expressed in yeast. To provide UGA-specificity, another construct (eRF1_Pte1_(m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When expressed individually, the Eoc_eRF1_CAC14170.1 and eRF1_Pte1_(m1) eRF1 constructs did not support viability of a sup45Δ mutant on 5-FOA media, suggesting that these strains post-shuffle cannot decode either UGA or UAA/UAG, respectively. When expressed in combination, the Eoc_eRF1_CAC14170.1 and eRF1_Pte1_(m1) constructs supported viability of a sup45Δ mutant on 5-FOA media, consistent with the stop codon specificity of the two eRF1 constructs and simultaneously demonstrating that both could function in yeast (Table 4).

The Eoc_eRF1_AAG25924.1 construct coded for a UAA/UAG-specific eRF1 protein that could function in yeast. The whole gene-RF1 construct was derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1_(m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When expressed separately, the Eoc_eRF1_AAG25924.1 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_AAG25924.1 and eRF1_Pte1_(m1) constructs supported viability of a sup45Δ mutant on 5-FOA media, consistent with the stop codon specificity of the two eRF1 constructs and simultaneously demonstrating that both could function in yeast (Table 4).

TABLE 4
Summary of ciliate eRF1 whole-gene replacements.

| | | % Sequence identity to | |
|---|---|---|---|
| UAA/UAG/UGA | Yeast_eRF1_NP_009701.3 | Yeast_eRF1 | Status |
| UAA/UAG | Eoc_eRF1_CAC14170.1 * | 57 | Replaceable |
| UAA/UAG | Eoc_eRF1_AAG25924.1 * | 56 | Replaceable |
| UAA/UAG | Bja_eRF1_CAC16186.2 * | 59 | Non-replaceable |
| UGA | Tth_eRF1_XP_001018735.1 ** | 55 | Non-replaceable |
| UGA | Tth_eRF1_XP_001018211.4 | 35 | Non-replaceable |
| UGA | Tth_eRF1_XP_001008252.2 | 20 | Non-replaceable |
| UGA | Pte_eRF1_XP_001425245.1 * | 45 | Non-replaceable |
| UGA | Pte_eRF1_XP_001448143.1 | 42 | Non-replaceable |
| UGA | Smy_eRF1_Q9BMM1.1 | 56 | Non-replaceable |
| UGA | Ssa_eRF1_EST45466.1 * | 41 | Non-replaceable |

* UAA/UAG-specific constructs tested against the UGA-specific eRF1_Pte1_(m1); all constructs regulated by a SUP45pro
** UGA-specific constructs tested against the UAA/UAG-specific eRF1_Bam_Bja; all constructs regulated by a SUP45pro
*** Status of construct when tested in an erf1Δ mutant:
Replaceable: Functional in yeast and confers stop codon selectivity, supports growth on 5-FQA only when expressed with the opposite construct
Non-replaceable: Not functional in yeast, unknown status on stop codon selectivity, does not support growth on 5-FQA when expressed with the opposite construct

Table 5 highlights the UAA/UAG whole-gene ciliate eRF1 constructs that were tested in conjunction with ciliate eRF3 in yeast. Ciliate eRF1 and eRF3 constructs, under the transcriptional control of the yeast bi-directional GAL1/10 promoter, were tested against the motif-swap constructs. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_Bja) (LEU2) and the indicated spHIS5-marked UGA-specific whole-gene eRF1/eRF3 constructs, or with the endogenously-regulated (SUP45pro) motif-swap UGA-specific construct (eRF1_Pte1_(m1)) (HIS3) and the indicated LEU2-marked UAA/UAG-specific whole-gene eRF1/eRF3 constructs. Yeast strains were maintained on SC-URA-LEU-HIS+Dex media. Ciliate ORFs were induced on the same selective media containing galactose for 3 days, before re-streaking on media supplemented with 5-FOA, while selecting for only two of the plasmid constructs (LEU2- and spHIS5/HIS3-marked).

The Eoc_eRF1_CAC14170 construct coded for a UAA/UAG-specific eRF1 protein that could function in yeast. The Eoc_eRF3_AAL33628.1 construct coded for the corresponding eRF3 protein. The whole gene eRF1/eRF3 constructs were derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1_(m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When expressed separately, the Eoc_eRF1_CAC14170.1/Eoc_eRF3_AAL33628.1 eRF1/eRF3 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_CAC14170.1/Eoc_eRF3 AAL33628.1 eRF1/eRF3 and eRF1_Pte1_(m1) constructs supported viability of a sup45Δ mutant on 5-FOA media, consistent with the stop codon specificity of the two eRF1 constructs and simultaneously demonstrating that both could function in yeast (Table 5).

The Eoc_eRF1_AAG25924.1 construct coded for a UAA/UAG-specific eRF1 protein that could function in yeast. The Eoc_eRF3_AAL33628.1 construct coded for the corresponding eRF3 protein. The whole gene eRF1/eRF3 constructs were derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1 (m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When expressed separately, the Eoc_eRF1_AAG25924.1: Eoc_eRF3_AAL33628.1 eRF1/eRF3 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_AAG25924.1/Eoc_eRF3 AAL33628.1 eRF1/eRF3 and eRF1_Pte1_(m1) constructs supported viability of a sup45Δ mutant on 5-FOA media, consistent with the stop codon specificity of the two eRF1 constructs and simultaneously demonstrating that both could function in yeast (Table 5).

TABLE 5
Summary of ciliate eRF1 whole-gene replacements expressed in conjugation with
ciliate eRF3.

| | Yeast_eRF1_NP_009701.3; | % Sequence identity to | |
|---|---|---|---|
| UAA/UAG/UGA | Yeast_eRF3_NP_010457.3 | Yeast_eRF1; eRF3 | Status *** |
| UAA/UAG | Eoc_eRF1_CAC14170.1; | 57; | Replaceable |
| | Eoc_eRF3_AAL33628.1 * | 25 | |
| UAA/UAG | Eoc_eRF1_AAG25924.1; | 56; | Replaceable |
| | Eoc_eRF3_AAL33628.1 * | 25 | |
| UAA/UAG | Bja_eRF1_CAC16186.2; | 59; | Non-replaceable |
| | Bja_eRF3_AAD03251.1 * | 25 | |
| UGA | Tth_eRF1_XP_001018735.1; | 55; | Replaceable |
| | Tth_eRF3 XP_001011280.3 ** | 33 | |
| UGA | Tth_eRF1_XP_001018211.4; | 35; | Non-replaceable |
| | Tth_eRF3_XP_001011280.3 ** | 33 | |
| UGA | Tth_eRF1_XP_001008252.2; | 20; | Non-replaceable |
| | Tth_eRF3_XP_001011280.3 ** | 33 | |
| UGA | Pte_eRF1_XP_001425245.1; | 45; | Non-replaceable |
| | Pte_eRF3_XP_001459190.1 ** | 36 | |
| UGA | Pte_eRF1_XP_001448143.1; | 42; | Non-replaceable |
| | Pte_eRF3 XP_001459190.1 ** | 36 | |

* UAA/UAG-specific constructs tested against the UGA-specific eRF1_Pte1_(m1); UAA/UAG constructs regulated by a GAL1/10pro, UGA-specific construct regulated by a SUP45pro
** UGA-specific constructs tested against the UAA/UAG-specific eRF1_Bam_Bja; UGA constructs regulated by a GAL1/10pro, UAA/UAG-specific construct regulated by a SUP45pro
*** Status of construct when tested in an erf1Δ mutant:
Replaceable: Functional in yeast and confers stop codon selectivity, supports growth on 5-FQA only when expressed with the opposite construct
Non-replaceable: Not functional in yeast, unknown status on stop codon selectivity, does not support growth on 5-FQA when expressed with the opposite construct

Table 6 highlights the UAA/UAG whole-gene ciliate eRF1 constructs that were tested in conjunction with N-terminally-modified ciliate eRF3 in yeast. Ciliate eRF1 and eRF3 constructs, under the transcriptional control of the yeast bi-directional GAL1/10 promoter, were tested against the motif-swap constructs. Ciliate eRF3 ORFs were modified by replacing their N-terminal domain with the N-terminal domain of yeast eRF3, thereby creating a chimeric yeast_ciliate eRF3 gene construct. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_Bja) (LEU2) and the indicated spHIS5-marked UGA-specific whole-gene eRF1/eRF3 constructs, or with the endogenously-regulated (SUP45pro) motif-swap UGA-specific construct (eRF1_Pte1 (m1)) (HIS3) and the indicated LEU2-marked UAA/UAG-specific whole-gene eRF1/eRF3 constructs. Yeast strains were maintained on SC-URA-LEU-HIS+Dex media. Ciliate ORFs were induced on the same selective media containing galactose for 3 days, before re-streaking on media supplemented with 5-FOA, while selecting for only two of the plasmid constructs (LEU2- and spHIS5/HIS3-marked).

The Eoc_eRF1 CAC14170.1 construct coded for a UAA/UAG-specific eRF1 protein that could function in yeast. The N Yeast eRF3 Eoc_eRF3 AAL33628.1 construct coded for the corresponding eRF3 protein that was modified by swapping the divergent N-terminal domain of the ciliate eRF3 with the N-terminal domain of yeast eRF3. This chimeric yeast-ciliate eRF3 protein was a fusion of amino acid residues (6-253) from yeast eRF3 with amino acid residues (1-6 and 299-799) of ciliate eRF3. The whole gene eRF1 and C-terminal domain of the chimeric eRF3 constructs were derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1 (m1)) was derived by swapping the YLCDNKF motif (SEQ ID NO: 2) in yeast eRF1 to YECDPQF (SEQ ID NO: 10; as found in the eRF1 protein sequence of the organism Paramecium tetraurelia). When expressed separately, the Eoc_eRF1_CAC14170.1IN_Yeast_eRF3_Eoc_eRF3 AAL33628.1 eRF1/eRF3 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_CAC14170.1/N_Yeast_eRF3_Eoc_eRF3_AAL33628.1 eRF1/eRF3 and eRF1_Pte1_(m1) constructs supported viability of a sup45Δ mutant on 5-FOA media, consistent with the stop codon specificity of the two eRF1 constructs and simultaneously demonstrating that both could function in yeast (Table 6).

TABLE 6
Summary of ciliate eRF1 whole-gene replacements expressed in conjunction with
N-terminally modified ciliate eRF3.

| | Yeast_eRF1_NP_009701.3; | % Sequence identity | |
|---|---|---|---|
| UAA/UAG/UGA | Yeast_eRF3_NP_010457.3 | to Yeast_eRF1; eRF3 | Status *** |
| UAA/UAG | Eoc_eRF1_CAC14170.1; | 57; | Replaceable |
| | N_Yeast_eRF3_Eoc_eRF3_AAL33628.1 * | 67 | |
| UAA/UAG | Eoc_eRF1_AAG25924.1; | 56; | Non-replaceable |
| | N_Yeast_eRF3_Eoc_eRF3_AAL33628.1 * | 67 | |
| UGA | Pte_eRF1_XP_001425245.1; | 45; | Non-replaceable |
| | N_Yeast_eRF3_Pte_eRF3_XP_001459190.1 ** | 63 | |
| UGA | Pte_eRF1_XP_001448143.1; | 42; | Non-replaceable |
| | N_Yeast_eRF3_Pte_eRF3_XP_001459190.1 ** | 63 | |

* UAA/UAG-specific constructs tested against the UGA-specific eRF1_Pte1_(m1); UAA/UAG constructs regulated by a GAL1/10pro, UGA-specific construct regulated by a SUP45pro
** UGA-specific constructs tested against the UAA/UAG-specific eRF1_Bam_Bja; UGA constructs regulated by a GAL1/10pro, UAA/UAG-specific construct regulated by a SUP45pro
*** Status of construct when tested in an erf1Δ mutant:
Replaceable: Functional in yeast and confers stop codon selectivity, supports growth on 5-FQA only when expressed with the opposite construct
Non-replaceable: Not functional in yeast, unknown status on stop codon selectivity, does not support growth on 5-FQA when expressed with the opposite construct

### Example 9: UGA-Specific Constructs

### Domain/Motif-Swap

Table 3 highlights the UGA-specific domain-swapped yeast eRF1 constructs tested in yeast. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_Bja) (LEU2) and the indicated HIS3-marked candidate UGA-specific constructs, or with the endogenously-regulated (SUP45pro) motif-swap UGA-specific construct (eRF1_Pte1_(m1)) (HIS3) and the indicated LEU2-marked candidate UAA/UAG-specific constructs. Yeast strains were maintained on SC-URA-LEU-HIS+Dex media, before testing for replaceability on SC-LEU-HIS+5-FOA+Dex media (Table 3).

The eRF1_Pte1_(m1) construct was UGA-specific and could function in yeast. This construct was derived by swapping the YLCDNKF motif (SEQ ID NO: 2) in yeast eRF1 to YECDPQF (SEQ ID NO: 10; as found in the eRF1 protein sequence of the organism Paramecium tetraurelia). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1_Pte1_(m1) and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Pte1_(m1) and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Pte1 (m2) construct was UGA-specific and could function in yeast. This construct was derived by swapping the TASNIKS/YLCDNKF motifs (SEQ ID NOs: 1 and 2, respectively, in order of appearance) in yeast eRF1 to EAASIKD/YFCDPQF (SEQ ID NOS: 11 and 10, respectively, in order of appearance; as found in the eRF1 protein sequence of the organism Paramecium tetraurelia). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1_Pte1 (m2) and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Pte1 (m2) and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Imu construct was UGA-specific and could function in yeast. This construct was derived by swapping the TASNIKS/YLCDNKF motifs (SEQ ID NOs: 1 and 2, respectively, in order of appearance) in yeast eRF1 to KATNIKD/FVIVNKF (SEQ ID NOS: 12 and 20, respectively, in order of appearance; as found in the eRF1 protein sequence of the organism Ichthyophthirius multifiliis). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1_Imu and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Imu and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Ppe1 construct was UGA-specific and could function in yeast. This construct was derived by swapping the TASNIKS/YLCDNKF motifs (SEQ ID NOs: 1 and 2, respectively, in order of appearance) in yeast eRF1 to QANSIKD/YRCDSKF (SEQ ID NOS: 23 and 24, respectively, in order of appearance; as found in the eRF1 protein sequence of the organism Pseudocohnilembus persalinus). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1 Ppe1 and eRF1_Bam_BjaeRF1 constructs did not support viability of a sup45Δ mutant on 5-FOA media, suggesting that these strains post-shuffle cannot decode either UAA/UAG or UGA, respectively. When expressed together, the eRF1 Ppe1 and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Tth2 construct was UGA-specific and could function in yeast. This construct was derived by swapping the TASNIKS/YLCDNKF motifs (SEQ ID NOs: 1 and 2, respectively, in order of appearance) in yeast eRF1 to GAASIKN/YSCNTIF (SEQ ID NOS: 25 and 26, respectively, in order of appearance; as found in the eRF1 protein sequence of the organism Tetrahymena thermophila). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1_Tth2 and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Tth2 and eRF1_Bam_Bjaconstructs supported viability of a sup45Δ mutant on 5-FOA media, consistent with the predicted stop codon specificity of the two eRF1 constructs and simultaneously demonstrating that both could function in yeast (Table 3).

The eRF1_Uhl construct was UGA-specific and could function in yeast. This construct was derived by swapping the TASNIKS/YLCDNKF motifs (SEQ ID NOs: 1 and 2, respectively, in order of appearance) in yeast eRF1 to SAQSIKS/YECDNSF (SEQ ID NOS: 32 and 30, respectively, in order of appearance; as found in the eRF1 protein sequence of the organism Urostyla sp. HL-2004). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1_Uhl1 and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Uhl1 and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1 Ssa construct was UGA-specific and could function in yeast. This construct was derived by swapping the TASNIKS/YLCDNKF motifs (SEQ ID NOs: 1 and 2, respectively, in order of appearance) in yeast eRF1 to QADCIKS/YSCDGVF (SEQ ID NOS: 36 and 37, respectively, in order of appearance; as found in the eRF1 protein sequence of the organism Spironucleus salmonicida). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1 Ssa and eRF1_Bam_BjaeRF1 constructs did not support viability of a sup45Δ mutant on 5-FOA media, suggesting that these strains post-shuffle cannot decode either UAA/UAG or UGA, respectively. When expressed together, the eRF1 Ssa andeRF1_Bam_Bja constructs supported viability of a sup45Δ mutant on 5-FOA media, consistent with the stop codon specificity of the two eRF1 constructs and simultaneously demonstrating that both could function in yeast (Table 3).

The eRF1_Lst construct was UGA-specific and could function in yeast. This construct was derived by swapping the TASNIKS/YLCDNKF motifs (SEQ ID NOs: 1 and 2, respectively, in order of appearance) in yeast eRF1 to RAQNIKS/FLCENTF (SEQ ID NOS: 38 and 39, respectively, in order of appearance; as found in the eRF1 protein sequence of the organism Loxodes striatus). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1Bam_Bja) was derived by swapping the TASNIKS/YLCDNKF motifs (SEQ ID NOs: 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the eRF1_Lst and eRF1_Bam_Bja eRF1 constructs did not support viability of a sup45Δ mutant on 5-FOA media, suggesting that these strains cannot decode either UAA/UAG or UGA, respectively. When expressed together, the eRF1_Lst and eRF1_Bam_Bja constructs supported viability of a sup45Δ mutant on 5-FOA media, consistent with the stop codon specificity of the two eRF1 constructs and simultaneously demonstrating that both could function in yeast (Table 3).

### Whole Gene Swaps

Table 5 highlights all the UGA-specific whole-gene ciliate eRF1 constructs that were tested in conjunction with ciliate eRF3 in yeast. Ciliate eRF1 and eRF3 constructs, under the transcriptional control of the yeast bi-directional GAL1/10 promoter, were tested against the motif-swap constructs. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1Bam_Bja) (LEU2) and the indicated spHIS5-marked UGA-specific whole-gene eRF1/eRF3 constructs, or with the endogenously-regulated (SUP45pro) motif-swap UGA-specific construct (eRF1_Pte1 (m1)) (HIS3) and the indicated LEU2-marked UAA/UAG-specific whole-gene eRF1/eRF3 constructs. Yeast strains were maintained on SC-URA-LEU-HIS+Dex media. Ciliate ORFs were induced on the same selective media containing galactose for 3 days, before re-streaking on media supplemented with 5-FOA, while selecting for only two of the plasmid constructs (LEU2- and spHIS5/HIS3-marked).

The Tth_eRF1_XP 001018735.1 construct coded for a UGA-specific eRF1 protein that could function in yeast when combined with the corresponding

Tth_eRF3_XP_001011280.3 eRF3 construct. The whole gene eRF1/eRF3 constructs were derived from the organism Tetrahymena thermophila. The episomal-based shuffle system, which utilized 5-FOA to counter-select against the URA3-marked omnipotent wild-type yeast eRF1, was employed to test the stop codon specificity and functionality of the ciliate eRF1 construct upon expression in yeast. To provide UAA/UAG-specificity, another construct (eRF1_Bam_Bja) was derived by swapping the TASNIKS/YLCDNKF motifs (SEQ ID NOs: s 1 and 2, respectively, in order of appearance) in yeast eRF1 to KSSNIKS/YICDNKF (SEQ ID NOs: 3 and 4, respectively, in order of appearance; as found in the eRF1 protein sequences of the organisms Blepharisma americanum and Blepharisma japonicum). When expressed separately, the Tth_eRF1_XP_001018735.1 and eRF1_Bam_Bja eRF1 constructs did not support viability of a sup45Δ mutant on 5-FOA media, suggesting that these strains post-shuffle could not decode either UAA/UAG or UGA, respectively (Table 4). When expressed separately, the UGA-specific Tth_eRF1_XP_001018735.1/Tth_eRF3_XP_001011280.3 eRF1/eRF3 construct did not support viability of a sup454 mutant on 5-FOA media, suggesting that this strain could not decode UAA/UAG (Table 5). When expressed together, the Tth_eRF1_XP_001018735.1 and eRF1_Bam_Bja eRF1 constructs did not support viability of a sup45Δ mutant on 5-FOA media (Table 4). However, concurrent expression of the Tth_eRF3_XP_001011280.3 eRF3 construct with the Tth_eRF1_XP_001018735.1 and eRF1_Bam_Bja eRF1 constructs supported viability of a sup45Δ mutant on 5-FOA media (Table 5). These results are consistent with the stop codon specificity of the two eRF1 constructs and simultaneously demonstrated that both can function in yeast. In the case of the UGA-specific Tth_eRF1_XP_001018735.1 eRF1 construct, its function required the corresponding Tth_eRF3_XP_001011280.3 eRF3 construct.

TABLE 7
Constructs used in examples

| | | | | | Modifications/ | | | | | |
|---|---|---|---|---|---|---|---|---|---|---|
| | | | | | Underlined | | | | | |
| | | | | | Sequences | | | | | Nucleic |
| | | | Ciliate source | Ciliate | modified from | Stop codon | ciliate eRF1 | eRF3 | Protein | acid |
| No. | Construct ID | category | organism(s) | nickname(s) | original | specificity | accession # | accession # | sequence | sequence |
| 1 | eRF1_Yeast | S. | n/a | n/a | n/a | UAA/UAG/ | NP_009701. | | SEQ ID | SEQ ID |
| | | cerevisiae | | | | UGA | 3 | | NO: 40 | NO: 101 |
| | | wild- | | | | | | | | |
| | | type | | | | | | | | |
| 2 | eRF1_Bam_Bja | Motif- | Blepharisma | Bam | KSSNIKS / | UAA/UAG | AAK12089. | | SEQ ID | SEQ ID |
| | | swap | americanum | Bja | YICDNKF | | 1 | | NO: 41 | NO: 102 |
| | | | Blepharisma | | (SEQ ID NOS: | | CAC16186. | | | |
| | | | japonicum | | 3 and 4, | | 2 | | | |
| | | | | | respectively, in | | | | | |
| | | | | | order of | | | | | |
| | | | | | appearance) | | | | | |
| 3 | eRF1_Eae1_Eoc1 | Motif- | Euplotes | Eae | TAVNIKS / | UAA/UAG | AAK07830. | | SEQ ID | SEQ ID |
| | | swap | aediculatus | Eoc | YICDNKF | | 1 | | NO: 42 | NO: 103 |
| | | | Euplotes | | (SEQ ID NOs: | | AAG25924. | | | |
| | | | octocarinatus | | 5 and 4, | | 1 | | | |
| | | | | | respectively, in | | | | | |
| | | | | | order of | | | | | |
| | | | | | appearance) | | | | | |
| 4 | eRF1_Sco | Motif- | Stentor | Sco | KAANIKS | UAA/UAG | OMJ89313. | | SEQ ID | SEQ ID |
| | | swap | coeruleus | | (SEQ ID NO: | | 1 | | NO: 43 | NO: 104 |
| | | | | | 6) | | OMJ91237. | | | |
| | | | | | | | 1 | | | |
| | | | | | | | OMJ79310. | | | |
| | | | | | | | 1 | | | |
| 5 | eRF1_Nov | Motif- | Nyctotherus | Nov | KASNIKS / | UAA/UAG | AAX19092. | | SEQ ID | SEQ ID |
| | | swap | ovalis | | YYCGERF | | 1 | | NO: 44 | NO: 105 |
| | | | | | (SEQ ID NOs: | | AAX19093. | | | |
| | | | | | 7 and 8, | | 1 | | | |
| | | | | | respectively, in | | | | | |
| | | | | | order of | | | | | |
| | | | | | appearance) | | | | | |
| 6 | eRF1_Eae2_Eoc2 | Motif- | Euplotes | Eae | TAESIKS / | UAA/UAG | AAK07829. | | SEQ ID | SEQ ID |
| | | swap | aediculatus | Eoc | YICDNKF | | 1 | | NO: 45 | NO: 106 |
| | | | Euplotes | | (SEQ ID NOS: | | CAC14170. | | | |
| | | | octocarinatus | | 9 and 4, | | 1 | | | |
| | | | | | respectively, in | | | | | |
| | | | | | order of | | | | | |
| | | | | | appearance) | | | | | |
| 7 | eRF1_Pte1_(m1) | Motif- | Paramecium | Pte | YFCDPQF | UGA | AAK66860. | | SEQ ID | SEQ ID |
| | | swap | tetraurelia | | (SEQ ID NO: | | 1 | | NO: 46 | NO: 107 |
| | | | | | 10) | | AAK66861. | | | |
| | | | | | | | 1 | | | |
| 8 | eRF1_Pte1_(m2) | Motif- | Paramecium | Pte | EAASIKD / | UGA | AAK66860. | | SEQ ID | SEQ ID |
| | | swap | tetraurelia | | YFCDPQF | | 1 | | NO: 47 | NO: 108 |
| | | | | | (SEQ ID NOS: | | AAK66861. | | | |
| | | | | | 11 and 10, | | 1 | | | |
| | | | | | respectively, in | | | | | |
| | | | | | order of | | | | | |
| | | | | | appearance) | | | | | |
| 9 | eRF1_Tth1 | Motif- | Tetrahymena | Tth | KATNIKD / | UGA | XP_ | | SEQ ID | SEQ ID |
| | | swap | thermophila | | YFCDSKF | | 001018735.1 | | NO: 48 | NO: 109 |
| | | | | | (SEQ ID NOs: | | | | | |
| | | | | | 12 and 13, | | | | | |
| | | | | | respectively, in | | | | | |
| | | | | | order of | | | | | |
| | | | | | appearance) | | | | | |
| 10 | eRF1_Sle1 | Motif- | Stylonychia | Sle | FDFDAES / | UGA | CDW74559. | | SEQ ID | SEQ ID |
| | | swap | lemnae | | TLIKPQF | | 1 | | NO: 49 | NO: 110 |
| | | | | | (SEQ ID NOS: | | | | | |
| | | | | | 14 and 15, | | | | | |
| | | | | | respectively, in | | | | | |
| | | | | | order of | | | | | |
| | | | | | appearance) | | | | | |
| 11 | eRF1_Ppe2 | Motif- | Pseudocohnilembus | Ppe | TGDKIKS / | UGA | KRW99069. | | SEQ ID | SEQ ID |
| | | swap | persalinus | | TIIKNDF (SEQ | | 1 | | NO: 50 | NO: 111 |
| | | | | | ID NOs: 16 and | | | | | |
| | | | | | 17, | | | | | |
| | | | | | respectively, in | | | | | |
| | | | | | order of | | | | | |
| | | | | | appearance) | | | | | |
| 12 | eRF1_Pte2 | Motif- | Paramecium | Pte | EAASIQD / | UGA | CAK80746. | | SEQ ID | SEQ ID |
| | | swap | tetraurelia | | FFCDNYF | | 1 | | NO: 51 | NO: 112 |
| | | | | | (SEQ ID NOS: | | | | | |
| | | | | | 18 and 19, | | | | | |
| | | | | | respectively, in | | | | | |
| | | | | | order of | | | | | |
| | | | | | appearance) | | | | | |
| 13 | eRF1_Imu | Motif- | Ichthyophthirius | Imu | KATNIKD / | UGA | XP_ | | SEQ ID | SEQ ID |
| | | swap | multifiliis | | FVIVNKF | | 004032541.1 | | NO: 52 | NO: 113 |
| | | | | | (SEQ ID NOS: | | | | | |
| | | | | | 12 and 20, | | | | | |
| | | | | | respectively, in | | | | | |
| | | | | | order of | | | | | |
| | | | | | appearance) | | | | | |
| 14 | eRF1_Sle2_Otr_ | Motif- | Stylonychia | Sle | AAQNIKS / | UGA | CDW89307. | | SEQ ID | SEQ ID |
| | Spu_Smy | swap | lemnae | Otr | YFCGGKF | | 1 | | NO: 53 | NO: 114 |
| | | | Oxytricha | Spu | (SEQ ID NOS: | | AAK07828. | | | |
| | | | trifallax | Smy | 21 and 22, | | 1 | | | |
| | | | Stylonychia | | respectively, in | | AAN62568. | | | |
| | | | pustulata | | order of | | 1 | | | |
| | | | Stylonychia | | appearance) | | AAK12091. | | | |
| | | | mytilus | | | | 1 | | | |
| 15 | eRF1_Ppe1 | Motif- | Pseudocohnilembus | Ppe | QANSIKD / | UGA | KRX05899. | | SEQ ID | SEQ ID |
| | | swap | persalinus | | YRCDSKF | | 1 | | NO: 54 | NO: 115 |
| | | | | | (SEQ ID NOs: | | | | | |
| | | | | | 23 and 24, | | | | | |
| | | | | | respectively, in | | | | | |
| | | | | | order of | | | | | |
| | | | | | appearance) | | | | | |
| 16 | eRF1_Tth2 | Motif- | Tetrahymena | Tth | GAASIKN / | UGA | XP_ | | SEQ ID | SEQ ID |
| | | swap | thermophila | | YSCNTIF | | 001018211.4 | | NO: 55 | NO: 116 |
| | | | | | (SEQ ID NOS: | | | | | |
| | | | | | 25 and 26, | | | | | |
| | | | | | respectively, in | | | | | |
| | | | | | order of | | | | | |
| | | | | | appearance) | | | | | |
| 17 | eRF1_Eh1 | Motif- | Eschaneustyla | Ehl | SAQNIKS / | UGA | AAT39331. | | SEQ ID | SEQ ID |
| | | swap | sp. HL- | | YYCDNRF | | 1 | | NO: 56 | NO: 117 |
| | | | 2004 | | (SEQ ID NOS: | | | | | |
| | | | | | 27 and 28, | | | | | |
| | | | | | respectively, in | | | | | |
| | | | | | order of | | | | | |
| | | | | | appearance) | | | | | |
| 18 | eRF1_Gh1 | Motif- | Gonostomum | Ghl | SAGNIKS / | UGA | AAT39330. | | SEQ ID | SEQ ID |
| | | swap | sp. HL-2004 | | YFCDNSF | | 1 | | NO: 57 | NO: 118 |
| | | | | | (SEQ ID NOS: | | | | | |
| | | | | | 29 and 30, | | | | | |
| | | | | | respectively, in | | | | | |
| | | | | | order of | | | | | |
| | | | | | appearance) | | | | | |
| 19 | eRF1_Hh1 | Motif- | Holosticha | Hhl | TAQNIKS / | UGA | AAT39329. | | SEQ ID | SEQ ID |
| | | swap | sp. HL-2004 | | YFCGGKF | | 1 | | NO: 58 | NO: 119 |
| | | | | | (SEQ ID NOs: | | | | | |
| | | | | | 31 and 22, | | | | | |
| | | | | | respectively, in | | | | | |
| | | | | | order of | | | | | |
| | | | | | appearance) | | | | | |
| 20 | eRF1_Uh1 | Motif- | Urostyla sp. | Uhl | SAQSIKS / | UGA | AAT39328. | | SEQ ID | SEQ ID |
| | | swap | HL-2004 | | YFCDNSF | | 1 | | NO: 59 | NO: 120 |
| | | | | | (SEQ ID NOS: | | | | | |
| | | | | | 32 and 30, | | | | | |
| | | | | | respectively, in | | | | | |
| | | | | | order of | | | | | |
| | | | | | appearance) | | | | | |
| 21 | eRF1_Uwj_Pwe | Motif- | Uroleptus sp. | Uwj | AANNIKS / | UGA | AAT39327. | | SEQ ID | SEQ ID |
| | | swap | WJC-2003 | Pwe | YFCGGKF | | 1 | | NO: 60 | NO: 121 |
| | | | Paraurostyla | | (SEQ ID NOs: | | AAT39326. | | | |
| | | | weissei | | 33 and 22, | | 1 | | | |
| | | | | | respectively, in | | | | | |
| | | | | | order of | | | | | |
| | | | | | appearance) | | | | | |
| 22 | eRF1_Smi | Motif- | Stichotrichida | Smi | YNCSGKF | UGA | AAN62567. | | SEQ ID | SEQ ID |
| | | swap | sp. misty | | (SEQ ID NO: | | 1 | | NO: 61 | NO: 122 |
| | | | | | 34) | | | | | |
| 23 | eRF1_Sal | Motif- | Stichotrichida | Sal | QAQNIKS / | UGA | AAN62563. | | SEQ ID | SEQ ID |
| | | swap | sp. Alaska | | YFCGGKF | | 1 | | NO: 62 | NO: 123 |
| | | | | | (SEQ ID NOS: | | AAN62564. | | | |
| | | | | | 35 and 22, | | 1 | | | |
| | | | | | respectively, in | | | | | |
| | | | | | order of | | | | | |
| | | | | | appearance) | | | | | |
| 24 | eRF1_Ssa | Motif- | Spironucleus | Ssa | QADCIKS / | UGA | EST45466.1 | | SEQ ID | SEQ ID |
| | | swap | salmonicida | | YSCDGVF | | | | NO: 63 | NO: 124 |
| | | | | | (SEQ ID NOS: | | | | | |
| | | | | | 36 and 37, | | | | | |
| | | | | | respectively, in | | | | | |
| | | | | | order of | | | | | |
| | | | | | appearance) | | | | | |
| 25 | eRF1_Lst | Motif- | Loxodes | Lst | RAQNIKS / | UGA | BAD90946. | | SEQ ID | SEQ ID |
| | | swap | striatus | | FLCENTF | | 1 | | NO: 64 | NO: 125 |
| | | | | | (SEQ ID NOs: | | | | | |
| | | | | | 38 and 39, | | | | | |
| | | | | | respectively, in | | | | | |
| | | | | | order of | | | | | |
| | | | | | appearance) | | | | | |
| 26 | Eoc_eRF1_ | Whole- | Euplotes | Eoc | | UAA/UAG | CAC14170. | | SEQ ID | SEQ ID |
| | CAC14170.1 | gene | octocarinatus | | | | 1 | | NO: 65 | NO: 126 |
| | | eRF1 | | | | | | | | |
| 27 | Eoc_eRF1_ | Whole- | Euplotes | Eoc | | UAA/UAG | AAG25924. | | SEQ ID | SEQ ID |
| | AAG25924.1 | gene | octocarinatus | | | | 1 | | NO: 66 | NO: 127 |
| | | eRF1 | | | | | | | | |
| 28 | Bja_eRF1_ | Whole- | Blepharisma | Bja | | UAA/UAG | CAC16186. | | SEQ ID | SEQ ID |
| | CAC16186.2 | gene | japonicum | | | | 2 | | NO: 67 | NO: 128 |
| | | eRF1 | | | | | | | | |
| 29 | Tth_eRF1_XP_ | Whole- | Tetrahymena | Tth | | UGA | XP_ | | SEQ ID | SEQ ID |
| | 001018735.1 | gene | thermophila | | | | 001018735.1 | | NO: 68 | NO: 129 |
| | | eRF1 | | | | | | | | |
| 30 | Tth_eRF1_XP_ | Whole- | Tetrahymena | Tth | | UGA | XP_ | | SEQ ID | SEQ ID |
| | 001018211.4 | gene | thermophila | | | | 001018211.4 | | NO: 69 | NO: 130 |
| | | eRF1 | | | | | | | | |
| 31 | Tth_eRF1_XP_ | Whole- | Tetrahymena | Tth | | UGA | XP_ | | SEQ ID | SEQ ID |
| | 001008252.2 | gene | thermophila | | | | 001008252.2 | | NO: 70 | NO: 131 |
| | | eRF1 | | | | | | | | |
| 32 | Pte_eRF1_XP_ | Whole- | Paramecium | Pte | | UGA | XP_ | | SEQ ID | SEQ ID |
| | 001425245.1 | gene | tetraurelia | | | | 001425245.1 | | NO: 71 | NO: 132 |
| | | eRF1 | | | | | | | | |
| 33 | Pte_eRF1_XP_ | Whole- | Paramecium | Pte | | UGA | XP_ | | SEQ ID | SEQ ID |
| | 001448143.1 | gene | tetraurelia | | | | 001448143.1 | | NO: 72 | NO: 133 |
| | | eRF1 | | | | | | | | |
| 34 | Smy_eRF1_ | Whole- | Stylonychia | Smy | | UGA | Q9BMM1.1 | | SEQ ID | SEQ ID |
| | Q9BMM1.1 | gene | mytilus | | | | | | NO: 73 | NO: 134 |
| | | eRF1 | | | | | | | | |
| 35 | Ssa_eRF1_ | Whole- | Spironucleus | Ssa | | UGA | EST45466.1 | | SEQ ID | SEQ ID |
| | EST45466.1 | gene | salmonicida | | | | | | NO: 74 | NO: 135 |
| | | eRF1 | | | | | | | | |
| 36 | Yeast_eRF1_ | Whole- | Saccharomyces | Sce | | UAA/UAG/ | NP_009701. | | SEQ ID | SEQ ID |
| | eRF3 | gene | cerevisiae | | | UGA | 3 | | NO: 75 | NO: 136 |
| | | eRF1/ | | | | | | | | |
| | | eRF3 | | | | | | | | |
| 37 | Yeast_eRF1_ | Whole- | Saccharomyces | Sce | | UAA/UAG/ | | NP_010457.3 | SEQ ID | SEQ ID |
| | eRF3 | gene | cerevisiae | | | UGA | | | NO: 76 | NO: 137 |
| | | eRF1/ | | | | | | | | |
| | | eRF3 | | | | | | | | |
| 38 | Eoc_eRF1_ | Whole- | Euplotes | Eoc | | UAA/UAG | CAC14170. | | SEQ ID | SEQ ID |
| | CAC14170.1/ | gene | octocarinatus | | | | 1 | | NO: 77 | NO: 138 |
| | Eoc_eRF3_ | eRF1/ | | | | | | | | |
| | AAL33628.1 | eRF3 | | | | | | | | |
| 39 | Eoc_eRF1_ | Whole- | Euplotes | Eoc | | UAA/UAG | | AAL336281 | SEQ ID | SEQ ID |
| | CAC14170.1/ | gene | octocarinatus | | | | | | NO: 78 | NO: 139 |
| | Eoc_eRF3_ | eRF1/ | | | | | | | | |
| | AAL33628.1 | eRF3 | | | | | | | | |
| 40 | Eoc_eRF1_ | Whole- | Euplotes | Eoc | | UAA/UAG | AAG25924. | | SEQ ID | SEQ ID |
| | AAG25924.1/ | gene | octocarinatus | | | | 1 | | NO: 79 | NO: 140 |
| | Eoc_eRF3_ | eRF1/ | | | | | | | | |
| | AAL33628.1 | eRF3 | | | | | | | | |
| 41 | Eoc_eRF1_ | Whole- | Euplotes | Eoc | | UAA/UAG | | AAL336281 | SEQ ID | SEQ ID |
| | AAG25924.1/ | gene | octocarinatus | | | | | | NO: 80 | NO: 141 |
| | Eoc_eRF3_ | eRF1/ | | | | | | | | |
| | AAL33628.1 | eRF3 | | | | | | | | |
| 42 | Bja_eRF1_ | Whole- | Blepharisma | Bja | | UAA/UAG | CAC16186. | | SEQ ID | SEQ ID |
| | CAC16186.2/ | gene | japonicum | | | | 2 | | NO: 81 | NO: 142 |
| | Bja_eRF3_ | eRF1/ | | | | | | | | |
| | AAD03251.1 | eRF3 | | | | | | | | |
| 43 | Bja_eRF1_ | Whole- | Blepharisma | Bja | | UAA/UAG | | AAD032511 | SEQ ID | SEQ ID |
| | CAC16186.2/ | gene | japonicum | | | | | | NO: 82 | NO: 143 |
| | Bja_eRF3_ | eRF1/ | | | | | | | | |
| | AAD03251.1 | eRF3 | | | | | | | | |
| 44 | Tth_eRF1_XP_ | Whole- | Tetrahymena | Tth | | UGA | XP_ | | SEQ ID | SEQ ID |
| | 001018735.1/ | gene | thermophila | | | | 001018735.1 | | NO: 83 | NO: 144 |
| | Tth_eRF3_XP_ | eRF1/ | | | | | | | | |
| | 001011280.3 | eRF3 | | | | | | | | |
| 45 | Tth_eRF1_XP_ | Whole- | Tetrahymena | Tth | | UGA | | XP_ | SEQ ID | SEQ ID |
| | 001018735.1/ | gene | thermophila | | | | | 001011280.3 | NO: 84 | NO: 145 |
| | Tth_eRF3_XP_ | eRF1/ | | | | | | | | |
| | 001011280.3 | eRF3 | | | | | | | | |
| 46 | Tth_eRF1_XP_ | Whole- | Tetrahymena | Tth | | UGA | XP_ | | SEQ ID | SEQ ID |
| | 001018211.4/ | gene | thermophila | | | | 001018211.4 | | NO: 85 | NO: 146 |
| | Tth_eRF3 XP_ | eRF1/ | | | | | | | | |
| | 001011280.3 | eRF3 | | | | | | | | |
| 47 | Tth_eRF1_XP_ | Whole- | Tetrahymena | Tth | | UGA | | XP_ | SEQ ID | SEQ ID |
| | 001018211.4/ | gene | thermophila | | | | | 001011280.3 | NO: 86 | NO: 147 |
| | Tth_eRF3_XP_ | eRF1/ | | | | | | | | |
| | 001011280.3 | eRF3 | | | | | | | | |
| 48 | Tth_eRF1_XP_ | Whole- | Tetrahymena | Tth | | UGA | XP_ | | SEQ ID | SEQ ID |
| | 001008252.2/ | gene | thermophila | | | | 001008252.2 | | NO: 87 | NO: 148 |
| | Tth_eRF3_XP_ | eRF1/ | | | | | | | | |
| | 001011280.3 | eRF3 | | | | | | | | |
| 49 | Tth_eRF1_XP_ | Whole- | Tetrahymena | Tth | | UGA | | XP_ | SEQ ID | SEQ ID |
| | 001008252.2/ | gene | thermophila | | | | | 001011280.3 | NO: 88 | NO: 149 |
| | Tth_eRF3_XP_ | eRF1/ | | | | | | | | |
| | 001011280.3 | eRF3 | | | | | | | | |
| 50 | Pte_eRF1_XP_ | Whole- | Paramecium | Pte | | UGA | XP_ | | SEQ ID | SEQ ID |
| | 001425245.1/ | gene | tetraurelia | | | | 001425245.1 | | NO: 89 | NO: 150 |
| | Pte_eRF3_XP_ | eRF1/ | | | | | | | | |
| | 001459190.1 | eRF3 | | | | | | | | |
| 51 | Pte_eRF1_XP_ | Whole- | Paramecium | Pte | | UGA | | XP_ | SEQ ID | SEQ ID |
| | 001425245.1/ | gene | tetraurelia | | | | | 001459190.1 | NO: 90 | NO: 151 |
| | Pte_eRF3_XP_ | eRF1/ | | | | | | | | |
| | 001459190.1 | eRF3 | | | | | | | | |
| 52 | Pte_eRF1_XP_ | Whole- | Paramecium | Pte | | UGA | XP_ | | SEQ ID | SEQ ID |
| | 001448143.1/ | gene | tetraurelia | | | | 001448143.1 | | NO: 91 | NO: 152 |
| | Pte_eRF3_XP_ | eRF1/ | | | | | | | | |
| | 001459190.1 | eRF3 | | | | | | | | |
| 53 | Pte_eRF1_XP_ | Whole- | Paramecium | Pte | | UGA | | XP_ | SEQ ID | SEQ ID |
| | 001448143.1/ | gene | tetraurelia | | | | | 001459190.1 | NO: 92 | NO: 153 |
| | Pte_eRF3_XP_ | eRF1/ | | | | | | | | |
| | 001459190.1 | eRF3 | | | | | | | | |
| 54 | Eoc_eRF1_ | Whole- | Euplotes | Eoc | Replace 7-298 | UAA/UAG | CAC14170.1 | | SEQ ID | SEQ ID |
| | CAC14170.1/ | gene | octocarinatus | | a.a. of | | | | NO: 93 | NO: 154 |
| | N_Yeast_eRF3_ | eRF1/ | | | Eoc_eRF3 with | | | | | |
| | Eoc_eRF3_ | eRF3 | | | 6-253 of | | | | | |
| | AAL33628.1 | | | | Sce_eRF3 | | | | | |
| 55 | Eoc_eRF1_ | Whole- | Euplotes | Eoc | Replace 7-298 | UAA/UAG | | AAL336281 | SEQ ID | SEQ ID |
| | CAC14170.1/ | gene | octocarinatus | | a.a. of | | | | NO: 94 | NO: 155 |
| | N_Yeast_eRF3_ | eRF1/ | | | Eoc_eRF3 with | | | | | |
| | Eoc_eRF3_ | eRF3 | | | 6-253 of | | | | | |
| | AAL33628.1 | | | | Sce_eRF3 | | | | | |
| 56 | Eoc_eRF1_ | Whole- | Euplotes | Eoc | Replace 1-298 | UAA/UAG | AAG25924.1 | | SEQ ID | SEQ ID |
| | AAG25924.1/ | gene | octocarinatus | | a.a. of | | | | NO: 95 | NO: 156 |
| | N_Yeast_eRF3_ | eRF1/ | | | Eoc_eRF3 with | | | | | |
| | Eoc_eRF3__ | eRF3 | | | 1-253 of | | | | | |
| | AAL33628.1 | | | | Sce_eRF3 | | | | | |
| 57 | Eoc_eRF1_ | Whole- | Euplotes | Eoc | Replace 1-298 | UAA/UAG | | AAL336281 | SEQ ID | SEQ ID |
| | AAG25924.1/ | gene | octocarinatus | | a.a. of | | | | NO: 96 | NO: 157 |
| | N_Yeast_eRF3_ | eRF1/ | | | Eoc_eRF3 with | | | | | |
| | Eoc_eRF3_ | eRF3 | | | 1-253 of | | | | | |
| | AAL33628.1 | | | | Sce_eRF3 | | | | | |
| 58 | Pte_eRF1_XP_ | Whole- | Paramecium | Pte | Replace 1-321 | UGA | XP_ | | SEQ ID | SEQ ID |
| | 001425245.1/ | gene | tetraurelia | | a.a. of | | 001425245.1 | | NO: 97 | NO: 158 |
| | N_Yeast_eRF3 | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |
| 59 | Pte_eRF1_XP_ | Whole- | Paramecium | Pte | Replace 1-321 | UGA | | XP_ | SEQ ID | SEQ ID |
| | 001425245.1/ | gene | tetraurelia | | a.a. of | | | 001459190.1 | NO: 98 | NO: 159 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |
| 60 | Pte_eRF1_XP_ | Whole- | Paramecium | Pte | Replace 1-321 | UGA | XP_ | | SEQ ID | SEQ ID |
| | 001448143.1/ | gene | tetraurelia | | a.a. of | | 001448143.1 | | NO: 99 | NO: 160 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | | | | | | |
| 61 | Pte_eRF1_XP_ | Whole- | Paramecium | Pte | Replace 1-321 | UGA | | XP_ | SEQ ID | SEQ ID |
| | 001448143.1/ | gene | tetraurelia | | a.a. of | | | 001459190.1 | NO: 100 | NO: 161 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |

TABLE 8
Sequence Listing

| | Construct | SEQ ID NO | Protein Sequence | SEQ ID NO | |
|---|---|---|---|---|---|
| No. | ID | for PS | (PS) | for NAC | Nucleic Acid Sequence (NAC) |
| 1 | eRF1_Yeast | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 40 | QSLEKARGNGTSMISLVI | 101 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TASNIKSRVNRLSVLSAI | | ACAGATGAATATGGTACTGCCTCGAATATTAAATCTAGGGTTAATCGTC |
| | | | TSTQQKLKLYNTLPKNGL | | TTTCCGTTTTATCTGCTATCACTTCCACCCAACAAAAGTTGAAGCTATA |
| | | | VLYCGDIITEDGKEKKVT | | TAATACTTTGCCCAAGAACGGTTTAGTTTTATATTGTGGTGATATCATC |
| | | | FDIEPYKPINTSLYLCDN | | ACTGAAGATGGTAAAGAAAAAAAGGTCACTTTTGACATCGAACCTTACA |
| | | | KFHTEVLSELLQADDKFG | | AACCTATCAACACATCCTTATATTTGTGTGATAACAAATTTCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 2 | eRF1_Bam_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | Bja | 41 | QSLEKARGNGTSMISLVI | 102 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | KSSNIKSRVNRLSVLSAI | | ACAGATGAATATGGTAAGTCTTCTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYICDN | | ACCGAAGACGGTAAGGAAAAGAAGGTTACCTTCGACATCGAACCATACA |
| | | | KFHTEVLSELLQADDKFG | | AGCCAATCAACACCTCTTTGTACATCTGTGACAACAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 3 | eRF1_Eae1_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | Eoc1 | 42 | QSLEKARGNGTSMISLVI | 103 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TAVNIKSRVNRLSVLSAI | | ACAGATGAATATGGTACCGCTGTTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYICDN | | ACCGAAGACGGTAAGGAAAAGAAGGTTACCTTCGACATCGAACCATACA |
| | | | KFHTEVLSELLQADDKFG | | AGCCAATCAACACCTCTTTGTACATCTGTGACAACAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 4 | eRF1_Sco | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 43 | QSLEKARGNGTSMISLVI | 104 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | KAANIKSRVNRLSVLSAI | | ACAGATGAATATGGTAAGGCTGCTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYLCDN | | ACCGAAGACGGTAAGGAAAAGAAGGTTACCTTCGACATCGAACCATACA |
| | | | KFHTEVLSELLQADDKFG | | AGCCAATCAACACCTCTTTGTACTTGTGTGACAACAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 5 | eRF1_Nov | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 44 | QSLEKARGNGTSMISLVI | 105 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | KASNIKSRVNRLSVLSAI | | ACAGATGAATATGGTAAGGCTTCTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYYCGE | | ACCGAAGACGGTAAGGAAAAGAAGGTTACCTTCGACATCGAACCATACA |
| | | | RFHTEVLSELLQADDKFG | | AGCCAATCAACACCTCTTTGTACTACTGTGGTGAAAGATTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 6 | eRF1_Eae2_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | Eoc2 | 45 | QSLEKARGNGTSMISLVI | 106 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TAESIKSRVNRLSVLSAI | | ACAGATGAATATGGTACCGCTGAATCTATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYICDN | | ACCGAAGACGGTAAGGAAAAGAAGGTTACCTTCGACATCGAACCATACA |
| | | | KFHTEVLSELLQADDKFG | | AGCCAATCAACACCTCTTTGTACATCTGTGACAACAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 7 | eRF1_Pte1_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | (m1) | 46 | QSLEKARGNGTSMISLVI | 107 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TASNIKSRVNRLSVLSAI | | ACAGATGAATATGGTACCGCTTCTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCDP | | ACCGAAGACGGTAAGGAAAAGAAGGTTACCTTCGACATCGAACCATACA |
| | | | QFHTEVLSELLQADDKFG | | AGCCAATCAACACCTCTTTGTACTTCTGTGACCCACAATTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 8 | eRF1_Pte1_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | (m2) | 47 | QSLEKARGNGTSMISLVI | 108 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | EAASIKDRVNRLSVLSAI | | ACAGATGAATATGGTGAAGCTGCTTCTATCAAGGACAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCDP | | ACCGAAGACGGTAAGGAAAAGAAGGTTACCTTCGACATCGAACCATACA |
| | | | QFHTEVLSELLQADDKFG | | AGCCAATCAACACCTCTTTGTACTTCTGTGACCCACAATTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NEGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 9 | eRF1_Tth1 | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 48 | QSLEKARGNGTSMISLVI | 109 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | KATNIKDRVNRLSVLSAI | | ACAGATGAATATGGTAAGGCTACCAACATCAAGGACAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCDS | | ACCGAAGACGGTAAGGAAAAGAAGGTTACCTTCGACATCGAACCATACA |
| | | | KFHTEVLSELLQADDKFG | | AGCCAATCAACACCTCTTTGTACTTCTGTGACTCTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 10 | eRF1_Sle1 | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 49 | QSLEKARGNGTSMISLVI | 110 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | FDFDAESRVNRLSVLSAI | | ACAGATGAATATGGTTTCGACTTCGACGCTGAATCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLTLIKP | | ACCGAAGACGGTAAGGAAAAGAAGGTTACCTTCGACATCGAACCATACA |
| | | | QFHTEVLSELLQADDKFG | | AGCCAATCAACACCTCTTTGACCTTGATCAAGCCACAATTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 11 | eRF1_Ppe2 | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 50 | QSLEKARGNGTSMISLVI | 111 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TGDKIKSRVNRLSVLSAI | | ACAGATGAATATGGTACCGGTGACAAGATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLTIIKN | | ACCGAAGACGGTAAGGAAAAGAAGGTTACCTTCGACATCGAACCATACA |
| | | | DFHTEVLSELLQADDKFG | | AGCCAATCAACACCTCTTTGACCATCATCAAGAACGACTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 12 | eRF1_Pte2 | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 51 | QSLEKARGNGTSMISLVI | 112 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | EAASIQDRVNRLSVLSAI | | ACAGATGAATATGGTGAAGCTGCTTCTATCCAAGACAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLFFCDN | | ACCGAAGACGGTAAGGAAAAGAAGGTTACCTTCGACATCGAACCATACA |
| | | | YFHTEVLSELLQADDKFG | | AGCCAATCAACACCTCTTTGTTCTTCTGTGACAACTACTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 13 | eRF1_Imu | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 52 | QSLEKARGNGTSMISLVI | 113 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | KATNIKDRVNRLSVLSAI | | ACAGATGAATATGGTAAGGCTACCAACATCAAGGACAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLFVIVN | | ACCGAAGACGGTAAGGAAAAGAAGGTTACCTTCGACATCGAACCATACA |
| | | | KFHTEVLSELLQADDKFG | | AGCCAATCAACACCTCTTTGTTCGTTATCGTTAACAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 14 | eRF1_Sle2_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | Otr_Spu_ | 53 | QSLEKARGNGTSMISLVI | 114 | TGGTCCAATCTTTAGAAAAAGCTAGAGGTAATGGTACTTCTATGATTTC |
| | Smy | | PPKGQIPLYQKMLTDEYG | | CTTAGTTATTCCTCCTAAGGGTCAAATTCCACTGTACCAAAAAATGTTA |
| | | | AAQNIKSRVNRLSVLSAI | | ACAGATGAATATGGTGCTGCTCAAAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCGG | | ACCGAAGACGGTAAGGAAAAGAAGGTTACCTTCGACATCGAACCATACA |
| | | | KFHTEVLSELLQADDKFG | | AGCCAATCAACACCTCTTTGTACTTCTGTGGTGGTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 15 | eRF1_Ppe1 | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 54 | QSLEKARGNGTSMISLVI | 115 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | QANSIKDRVNRLSVLSAI | | ACAGATGAATATGGTCAAGCTAACTCTATCAAGGACAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYRCDS | | ACCGAAGACGGTAAGGAAAAGAAGGTTACCTTCGACATCGAACCATACA |
| | | | KFHTEVLSELLQADDKFG | | AGCCAATCAACACCTCTTTGTACAGATGTGACTCTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 16 | eRF1_Tth2 | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 55 | QSLEKARGNGTSMISLVI | 116 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | GAASIKNRVNRLSVLSAI | | ACAGATGAATATGGTGGTGCTGCTTCTATCAAGAACAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYSCNT | | ACCGAAGACGGTAAGGAAAAGAAGGTTACCTTCGACATCGAACCATACA |
| | | | IFHTEVLSELLQADDKFG | | AGCCAATCAACACCTCTTTGTACTCTTGTAACACCATCTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 17 | eRF1_Ehl | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 56 | QSLEKARGNGTSMISLVI | 117 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | SAQNIKSRVNRLSVLSAI | | ACAGATGAATATGGTTCTGCTCAAAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYYCDN | | ACCGAAGACGGTAAGGAAAAGAAGGTTACCTTCGACATCGAACCATACA |
| | | | RFHTEVLSELLQADDKFG | | AGCCAATCAACACCTCTTTGTACTACTGTGACAACAGATTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 18 | eRF1_Ghl | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 57 | QSLEKARGNGTSMISLVI | 118 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | SAGNIKSRVNRLSVLSAI | | ACAGATGAATATGGTTCTGCTGGTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCDN | | ACCGAAGACGGTAAGGAAAAGAAGGTTACCTTCGACATCGAACCATACA |
| | | | SFHTEVLSELLQADDKFG | | AGCCAATCAACACCTCTTTGTACTTCTGTGACAACTCTTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 19 | eRF1_Hhl | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 58 | QSLEKARGNGTSMISLVI | 119 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TAQNIKSRVNRLSVLSAI | | ACAGATGAATATGGTACCGCTCAAAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCGG | | ACCGAAGACGGTAAGGAAAAGAAGGTTACCTTCGACATCGAACCATACA |
| | | | KFHTEVLSELLQADDKFG | | AGCCAATCAACACCTCTTTGTACTTCTGTGGTGGTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 20 | eRF1_Uhl | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 59 | QSLEKARGNGTSMISLVI | 120 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | SAQSIKSRVNRLSVLSAI | | ACAGATGAATATGGTTCTGCTCAATCTATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCDN | | ACCGAAGACGGTAAGGAAAAGAAGGTTACCTTCGACATCGAACCATACA |
| | | | SFHTEVLSELLQADDKFG | | AGCCAATCAACACCTCTTTGTACTTCTGTGACAACTCTTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NEGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 21 | eRF1_Uwj_ | SEQ ID NO: | MDNEVEKNIEIWKVKKLV | SEQ ID NO: | ATGGATAACGAGGTTGAAAAAAATATTGAGATCTGGAAGGTCAAGAAGT |
| | Pwe | 60 | QSLEKARGNGTSMISLVI | 121 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | AANNIKSRVNRLSVLSAI | | ACAGATGAATATGGTGCTGCTAACAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCGG | | ACCGAAGACGGTAAGGAAAAGAAGGTTACCTTCGACATCGAACCATACA |
| | | | KFHTEVLSELLQADDKFG | | AGCCAATCAACACCTCTTTGTACTTCTGTGGTGGTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 22 | eRF1_Smi | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 61 | QSLEKARGNGTSMISLVI | 122 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | TASNIKSRVNRLSVLSAI | | ACAGATGAATATGGTACCGCTTCTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYNCSG | | ACCGAAGACGGTAAGGAAAAGAAGGTTACCTTCGACATCGAACCATACA |
| | | | KFHTEVLSELLQADDKFG | | AGCCAATCAACACCTCTTTGTACAACTGTTCTGGTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NEGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 23 | eRF1_Sal | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 62 | QSLEKARGNGTSMISLVI | 123 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | QAQNIKSRVNRLSVLSAI | | ACAGATGAATATGGTCAAGCTCAAAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYFCGG | | ACCGAAGACGGTAAGGAAAAGAAGGTTACCTTCGACATCGAACCATACA |
| | | | KFHTEVLSELLQADDKFG | | AGCCAATCAACACCTCTTTGTACTTCTGTGGTGGTAAGTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 24 | eRF1_Ssa | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 63 | QSLEKARGNGTSMISLVI | 124 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | QADCIKSRVNRLSVLSAI | | ACAGATGAATATGGTCAAGCTGACTGTATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYSCDG | | ACCGAAGACGGTAAGGAAAAGAAGGTTACCTTCGACATCGAACCATACA |
| | | | VFHTEVLSELLQADDKFG | | AGCCAATCAACACCTCTTTGTACTCTTGTGACGGTGTTTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 25 | eRF1_Lst | SEQ ID NO: | MDNEVEKNIEIWKVKKLV | SEQ ID NO: | ATGGATAACGAGGTTGAAAAAAATATTGAGATCTGGAAGGTCAAGAAGT |
| | | 64 | QSLEKARGNGTSMISLVI | 125 | TGGTCCAATCTTTAGAAAAAGCTAGAGGTAATGGTACTTCTATGATTTC |
| | | | PPKGQIPLYQKMLTDEYG | | CTTAGTTATTCCTCCTAAGGGTCAAATTCCACTGTACCAAAAAATGTTA |
| | | | RAQNIKSRVNRLSVLSAI | | ACAGATGAATATGGTAGAGCTCAAAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLFLCEN | | ACCGAAGACGGTAAGGAAAAGAAGGTTACCTTCGACATCGAACCATACA |
| | | | TFHTEVLSELLQADDKFG | | AGCCAATCAACACCTCTTTGTTCTTGTGTGAAAACACCTTCCATACAGA |
| | | | FIVMDGQGTLFGSVSGNT | | AGTTCTTTCGGAATTGCTTCAAGCTGACGACAAGTTCGGTTTTATAGTC |
| | | | RTVLHKFTVDLPKKHGRG | | ATGGACGGTCAAGGTACTTTGTTTGGTTCTGTGTCCGGTAATACGAGAA |
| | | | GQSALRFARLREEKRHNY | | CTGTTTTACATAAATTTACTGTCGATCTGCCAAAAAAGCATGGTAGAGG |
| | | | VRKVAEVAVQNFITNDKV | | TGGTCAATCTGCGCTTCGTTTTGCTCGTTTAAGAGAAGAAAAAAGACAT |
| | | | NVKGLILAGSADFKTDLA | | AATTATGTGAGAAAGGTCGCCGAAGTTGCTGTTCAAAATTTTATTACTA |
| | | | KSELFDPRLACKVISIVD | | ATGACAAAGTCAATGTTAAGGGTTTAATTTTAGCTGGTTCTGCTGACTT |
| | | | VSYGGENGFNQAIELSAE | | TAAGACCGATTTGGCTAAATCTGAATTATTCGATCCAAGACTAGCATGT |
| | | | ALANVKYVQEKKLLEAYF | | AAGGTTATTTCCATCGTGGATGTTTCTTATGGTGGTGAAAACGGTTTCA |
| | | | DEISQDTGKFCYGIDDTL | | ACCAGGCTATCGAACTTTCTGCCGAAGCGTTGGCCAATGTCAAGTATGT |
| | | | KALDLGAVEKLIVFENLE | | TCAAGAAAAGAAATTATTGGAGGCATATTTTGACGAAATTTCCCAGGAC |
| | | | TIRYTFKDAEDNEVIKFA | | ACTGGTAAATTCTGTTATGGTATAGATGATACTTTAAAGGCATTGGATT |
| | | | EPEAKDKSFAIDKATGQE | | TAGGTGCAGTCGAAAAATTAATTGTTTTCGAAAATTTGGAAACTATCAG |
| | | | MDVVSEEPLIEWLAANYK | | ATATACATTTAAAGATGCCGAGGATAATGAGGTTATAAAATTCGCTGAA |
| | | | NFGATLEFITDKSSEGAQ | | CCAGAAGCCAAGGACAAGTCGTTTGCTATTGACAAAGCTACCGGCCAAG |
| | | | FVTGFGGIGAMLRYKVNF | | AAATGGACGTTGTCTCCGAAGAACCTTTAATTGAATGGCTAGCAGCTAA |
| | | | EQLVDESEDEYYDEDEGS | | CTACAAAAACTTCGGTGCTACCTTGGAATTCATCACAGACAAATCTTCA |
| | | | DYDFI* | | GAAGGTGCCCAATTTGTCACAGGTTTTGGTGGTATTGGTGCCATGCTGC |
| | | | | | GTTACAAAGTTAATTTTGAACAACTAGTTGATGAATCTGAGGATGAATA |
| | | | | | TTATGACGAAGATGAAGGATCCGACTATGATTTCATTTAA |
| 26 | Eoc_eRF1_ | SEQ ID NO: | MSIIDSNVETWKIKRIIK | SEQ ID NO: | ATGTCTATCATCGACTCTAACGTTGAAACCTGGAAGATCAAGAGAATCA |
| | CAC14170.1 | 65 | NLERLRGNGTSMISLLLS | 126 | TCAAGAACTTGGAAAGATTGAGAGGTAACGGTACCTCTATGATCTCTTT |
| | | | PRDAIPKVQGMLAGEYGT | | GTTGTTGTCTCCACGCGACGCTATCCCAAAGGTTCAAGGTATGTTGGCT |
| | | | AESIKSKINRLAVQGAIT | | GGTGAATACGGTACCGCTGAATCTATCAAGTCTAAGATCAACAGATTGG |
| | | | SAKERLKLYNRTPPNGLV | | CTGTTCAAGGTGCTATCACCTCTGCTAAGGAAAGATTGAAGTTGTACAA |
| | | | IYCGIVIGEDKSEKKYCI | | CAGAACCCCACCAAACGGTTTGGTTATCTACTGTGGTATCGTTATCGGT |
| | | | DFEPFRPLNTFKYICDNK | | GAAGACAAGTCTGAAAAGAAGTACTGTATCGACTTCGAACCATTCAGAC |
| | | | FYTKPLFELLENDDVFGF | | CATTGAACACCTTCAAGTACATCTGTGACAACAAGTTCTACACCAAGCC |
| | | | VIVDGSGCLFGTLQGNTK | | ATTGTTCGAATTGTTGGAAAACGACGACGTTTTCGGTTTCGTTATCGTT |
| | | | KIIQNITVSLPKKHGRGG | | GACGGTTCTGGTTGTTTGTTCGGTACCTTGCAAGGTAACACCAAGAAGA |
| | | | QSAPRFGRIREEKRHNYV | | TCATCCAAAACATCACCGTTTCTTTGCCAAAGAAGCACGGTAGAGGTGG |
| | | | RKVAEFATQHFITEDKPN | | TCAATCTGCTCCAAGATTCGGTAGAATCAGAGAAGAAAAGAGACACAAC |
| | | | VKGIILAGSANFKNDLSE | | TACGTTAGAAAGGTTGCTGAATTCGCTACCCAACACTTCATCACCGAAG |
| | | | SDLFDKRLSEIVLKIVDV | | ACAAGCCAAACGTTAAGGGTATCATCTTGGCTGGTTCTGCTAACTTCAA |
| | | | SYGGENGFSQAITLAEDT | | GAACGACTTGTCTGAATCTGACTTGTTCGACAAGAGATTGTCTGAAATC |
| | | | LSNVKFVEEKNLISKYFE | | GTTTTGAAGATCGTTGACGTTTCTTACGGTGGTGAAAACGGTTTCTCTC |
| | | | EIAQDTGMVVFGIEDTLN | | AAGCTATCACCTTGGCTGAAGACACCTTGTCTAACGTTAAGTTCGTTGA |
| | | | SLELGAVGTIICFENLEI | | AGAAAAGAACTTGATCTCTAAGTACTTCGAAGAAATCGCTCAAGACACC |
| | | | NRYEIRNPSTEEIKVIHL | | GGTATGGTTGTTTTCGGTATCGAAGACACCTTGAACTCTTTGGAATTGG |
| | | | CKDQQNDTRYKMIDNNYS | | GTGCTGTTGGTACCATCATCTGTTTCGAAAACTTGGAAATCAACAGATA |
| | | | YFIDONTGLDLEILSCVP | | CGAAATCAGAAACCCATCTACCGAAGAAATCAAGGTTATCCACTTGTGT |
| | | | LTEWLCENYSKYGVRLEF | | AAGGACCAACAAAACGACACCAGATACAAGATGATCGACAACAACTACT |
| | | | ITDKSQEGFQFVNGFGGI | | CTTACTTCATCGACCAAAACACCGGTTTGGACTTGGAAATCTTGTCTTG |
| | | | GGFLRFKLEIENIDYEGE | | TGTTCCATTGACCGAATGGTTGTGTGAAAACTACTCTAAGTACGGTGTT |
| | | | DVGGEEFDADEDFI** | | AGATTGGAATTCATCACCGACAAGTCTCAAGAAGGTTTCCAATTCGTTA |
| | | | | | ACGGTTTCGGTGGTATCGGTGGTTTCTTGAGATTCAAGTTGGAAATCGA |
| | | | | | AAACATCGACTACGAAGGTGAAGACGTTGGTGGTGAAGAATTCGACGCT |
| | | | | | GACGAAGACTTCATCtaatag |
| 27 | Eoc_eRF1_ | SEQ ID NO: | MAKLDDNVETWRIKRLIK | SEQ ID NO: | ATGGCTAAGTTGGACGACAACGTTGAAACCTGGAGAATCAAGAGATTGA |
| | AAG25924.1 | 66 | NLEKLRGDGTSMISLLLS | 127 | TCAAGAACTTGGAAAAGTTGAGAGGTGACGGTACCTCTATGATCTCTTT |
| | | | PRDQISKVQAMLAGEAGT | | GTTGTTGTCTCCACGCGACCAAATCTCTAAGGTTCAAGCTATGTTGGCT |
| | | | AVNIKSRVNRQAVLSAIT | | GGTGAAGCTGGTACCGCTGTTAACATCAAGTCTAGAGTTAACAGACAAG |
| | | | SAKERLKLYSKTPTNGLV | | CTGTTTTGTCTGCTATCACCTCTGCTAAGGAAAGATTGAAGTTGTACTC |
| | | | VYCGTVIGEDDSEKKYTI | | TAAGACCCCAACCAACGGTTTGGTTGTTTACTGTGGTACCGTTATCGGT |
| | | | DFEPFRPLNTFKYICDNK | | GAAGACGACTCTGAAAAGAAGTACACCATCGACTTCGAACCATTCAGAC |
| | | | FCTEPLFELLENDDVFGF | | CATTGAACACCTTCAAGTACATCTGTGACAACAAGTTCTGTACCGAACC |
| | | | VIVDGNGCLFGTLQGNTK | | ATTGTTCGAATTGTTGGAAAACGACGACGTTTTCGGTTTCGTTATCGTT |
| | | | KILQQITVSLPKKHGRGG | | GACGGTAACGGTTGTTTGTTCGGTACCTTGCAAGGTAACACCAAGAAGA |
| | | | QSAPRFGRIREEKRHNYV | | TCTTGCAACAAATCACCGTTTCTTTGCCAAAGAAGCACGGTAGAGGTGG |
| | | | RKVAELATQHFITDDRPN | | TCAATCTGCTCCAAGATTCGGTAGAATCAGAGAAGAAAAGAGACACAAC |
| | | | VKGLVLAGSANFKNDLSE | | TACGTTAGAAAGGTTGCTGAATTGGCTACCCAACACTTCATCACCGACG |
| | | | SDLFDKRLSEVVIKIVDV | | ACAGACCAAACGTTAAGGGTTTGGTTTTGGCTGGTTCTGCTAACTTCAA |
| | | | SYGGENGFSQAISLAEDA | | GAACGACTTGTCTGAATCTGACTTGTTCGACAAGAGATTGTCTGAAGTT |
| | | | LSNVKFVEEKNLISKYFE | | GTTATCAAGATCGTTGACGTTTCTTACGGTGGTGAAAACGGTTTCTCTC |
| | | | EIALDSGMIVFGVEDTLH | | AAGCTATCTCTTTGGCTGAAGACGCTTTGTCTAACGTTAAGTTCGTTGA |
| | | | SLEVGALDLLMCFENLEI | | AGAAAAGAACTTGATCTCTAAGTACTTCGAAGAAATCGCTTTGGACTCT |
| | | | NRYEIRDPANDEIKIYNL | | GGTATGATCGTTTTCGGTGTTGAAGACACCTTGCACTCTTTGGAAGTTG |
| | | | NKEQEKDSKYFKNEKTGT | | GTGCTTTGGACTTGTTGATGTGTTTCGAAAACTTGGAAATCAACAGATA |
| | | | DLEIVKCVALSEWLCENY | | CGAAATCCGCGACCCAGCTAACGACGAAATCAAGATCTACAACTTGAAC |
| | | | SKYGVKLEFITDKSQEGF | | AAGGAACAAGAAAAGGACTCTAAGTACTTCAAGAACGAAAAGACCGGTA |
| | | | QFVNGFGGIGGFLRYKLE | | CCGACTTGGAAATCGTTAAGTGTGTTGCTTTGTCTGAATGGTTGTGTGA |
| | | | MENHDYDKEDVGGEEFNP | | AAACTACTCTAAGTACGGTGTTAAGTTGGAATTCATCACCGACAAGTCT |
| | | | DEDFI** | | CAAGAAGGTTTCCAATTCGTTAACGGTTTCGGTGGTATCGGTGGTTTCT |
| | | | | | TGAGATACAAGTTGGAAATGGAAAACCACGACTACGACAAGGAAGACGT |
| | | | | | TGGTGGTGAAGAATTCAACCCAGACGAAGACTTCATCtaatag |
| 28 | Bja_eRF1_ | SEQ ID NO: | MEGDELTQNIEQWKIKRL | SEQ ID NO: | ATGGAAGGTGACGAATTGACCCAAAACATCGAACAATGGAAGATCAAGA |
| | CAC16186.2 | 67 | IDNLDKARGNGTSLISLI | 128 | GATTGATCGACAACTTGGACAAGGCTAGAGGTAACGGTACCTCTTTGAT |
| | | | IPPREQLPIINKMITEEY | | CTCTTTGATCATCCCACCAAGAGAACAATTGCCAATCATCAACAAGATG |
| | | | GKSSNIKSRIVRQAVQSA | | ATCACCGAAGAATACGGTAAGTCTTCTAACATCAAGTCTAGAATCGTTA |
| | | | LTSTKERLKLYNNRLPAN | | GACAAGCTGTTCAATCTGCTTTGACCTCTACCAAGGAAAGATTGAAGTT |
| | | | GLILYCGEVINEEGVCEK | | GTACAACAACAGATTGCCAGCTAACGGTTTGATCTTGTACTGTGGTGAA |
| | | | KYTIDFQPYRAINTTLYI | | GTTATCAACGAAGAAGGTGTTTGTGAAAAGAAGTACACCATCGACTTCC |
| | | | CDNKFHTQPLKDLLVMDD | | AACCATACAGAGCTATCAACACCACCTTGTACATCTGTGACAACAAGTT |
| | | | KFGFIIIDGNGALFGTLQ | | CCACACCCAACCATTGAAGGACTTGTTGGTTATGGACGACAAGTTCGGT |
| | | | GNTREVLHKFSVDLPKKH | | TTCATCATCATCGACGGTAACGGTGCTTTGTTCGGTACCTTGCAAGGTA |
| | | | RRGGQSALRFARLRMESR | | ACACCAGAGAAGTTTTGCACAAGTTCTCTGTTGACTTGCCAAAGAAGCA |
| | | | NNYLRKVAEQAVVQFISN | | CAGAAGAGGTGGTCAATCTGCTTTGAGATTCGCTAGATTGAGAATGGAA |
| | | | DKVNVAGLIVAGSAEFKN | | TCTAGAAACAACTACTTGAGAAAGGTTGCTGAACAAGCTGTTGTTCAAT |
| | | | VLVQSDLFDQRLAAKVLK | | TCATCTCTAACGACAAGGTTAACGTTGCTGGTTTGATCGTTGCTGGTTC |
| | | | IVDVAYGGENGFTQAIEL | | TGCTGAATTCAAGAACGTTTTGGTTCAATCTGACTTGTTCGACCAAAGA |
| | | | SADTLSNIKFIREKKVMS | | TTGGCTGCTAAGGTTTTGAAGATCGTTGACGTTGCTTACGGTGGTGAAA |
| | | | KFFEEVAQDTKKYCYGVE | | ACGGTTTCACCCAAGCTATCGAATTGTCTGCTGACACCTTGTCTAACAT |
| | | | DTMKTLIMGAVEVILLFE | | CAAGTTCATCAGAGAAAAGAAGGTTATGTCTAAGTTCTTCGAAGAAGTT |
| | | | NLNFTRYVLKNPTTGVEK | | GCTCAAGACACCAAGAAGTACTGTTACGGTGTTGAAGACACCATGAAGA |
| | | | TLYLTPEQEENHDNFMEN | | CCTTGATCATGGGTGCTGTTGAAGTTATCTTGTTGTTCGAAAACTTGAA |
| | | | GEELEALEKGPLPEWIVD | | CTTCACCAGATACGTTTTGAAGAACCCAACCACCGGTGTTGAAAAGACC |
| | | | NYMKFGAGLEFITDRSQE | | TTGTACTTGACCCCAGAACAAGAAGAAAACCACGACAACTTCATGGAAA |
| | | | GAQFVRGFGGLGAFLRYQ | | ACGGTGAAGAATTGGAAGCTTTGGAAAAGGGTCCATTGCCAGAATGGAT |
| | | | VDMAHLNAGEEELDEEWD | | CGTTGACAACTACATGAAGTTCGGTGCTGGTTTGGAATTCATCACCGAC |
| | | | DDFM** | | AGATCTCAAGAAGGTGCTCAATTCGTTAGAGGTTTCGGTGGTTTGGGTG |
| | | | | | CTTTCTTGAGATACCAAGTTGACATGGCTCACTTGAACGCTGGTGAAGA |
| | | | | | AGAATTGGACGAAGAATGGGACGACGACTTCATGtaatag |
| 29 | Tth_eRF1_ | SEQ ID NO: | MEEKDQRQRNIEHFKIKK | SEQ ID NO: | ATGGAAGAAAAGGACCAAAGACAAAGAAACATCGAACACTTCAAGATCA |
| | XP_ | 68 | LMTRLRNTRGSGTSMVSL | 129 | AGAAGTTGATGACCAGATTGAGAAACACCAGAGGTTCTGGTACCTCTAT |
| | 001018735.1 | | IIPPKKQINDSTKLISDE | | GGTTTCTTTGATCATCCCACCAAAGAAGCAAATCAACGACTCTACCAAG |
| | | | FSKATNIKDRVNRQSVQD | | TTGATCTCTGACGAATTCTCTAAGGCTACCAACATCAAGGACAGAGTTA |
| | | | AMVSALQRLKLYQRTPNN | | ACAGACAATCTGTTCAAGACGCTATGGTTTCTGCTTTGCAAAGATTGAA |
| | | | GLILYCGKVLNEEGKEIK | | GTTGTACCAAAGAACCCCAAACAACGGTTTGATCTTGTACTGTGGTAAG |
| | | | LLIDFEPYKPINTSLYFC | | GTTTTGAACGAAGAAGGTAAGGAAATCAAGTTGTTGATCGACTTCGAAC |
| | | | DSKFHVDELGSLLETDPP | | CATACAAGCCAATCAACACCTCTTTGTACTTCTGTGACTCTAAGTTCCA |
| | | | FGFIVMDGQGALYANLQG | | CGTTGACGAATTGGGTTCTTTGTTGGAAACCGACCCACCATTCGGTTTC |
| | | | NTKTVLNKFSVELPKKHG | | ATCGTTATGGACGGTCAAGGTGCTTTGTACGCTAACTTGCAAGGTAACA |
| | | | RGGQSSVRFARLRVEKRH | | CCAAGACCGTTTTGAACAAGTTCTCTGTTGAATTGCCAAAGAAGCACGG |
| | | | NYLRKVCEVATQTFISQD | | TAGAGGTGGTCAATCTTCTGTTAGATTCGCTAGATTGAGAGTTGAAAAG |
| | | | KINVQGLVLAGSGDFKNE | | AGACACAACTACTTGAGAAAGGTTTGTGAAGTTGCTACCCAAACCTTCA |
| | | | LSTTQMEDPRLACKIIKI | | TCTCTCAAGACAAGATCAACGTTCAAGGTTTGGTTTTGGCTGGTTCTGG |
| | | | VDVSYGGENGLNQAIELA | | TGACTTCAAGAACGAATTGTCTACCACCCAAATGTTCGACCCAAGATTG |
| | | | QESLTNVKFVQEKNVISK | | GCTTGTAAGATCATCAAGATCGTTGACGTTTCTTACGGTGGTGAAAACG |
| | | | FFDCIAIDSGTVVYGVQD | | GTTTGAACCAAGCTATCGAATTGGCTCAAGAATCTTTGACCAACGTTAA |
| | | | TMQLLLDGVIENILCFEE | | GTTCGTTCAAGAAAAGAACGTTATCTCTAAGTTCTTCGACTGTATCGCT |
| | | | LTTLRVTRKNKVTEQITH | | ATCGACTCTGGTACCGTTGTTTACGGTGTTCAAGACACCATGCAATTGT |
| | | | IFIPPNELNNPKHFKDGE | | TGTTGGACGGTGTTATCGAAAACATCTTGTGTTTCGAAGAATTGACCAC |
| | | | HELEKIEVENLTEWLAEH | | CTTGAGAGTTACCAGAAAGAACAAGGTTACCGAACAAATCACCCACATC |
| | | | YSEFGAELYFITDKSAEG | | TTCATCCCACCAAACGAATTGAACAACCCAAAGCACTTCAAGGACGGTG |
| | | | CQFVKGFSGIGGFLRYKV | | AACACGAATTGGAAAAGATCGAAGTTGAAAACTTGACCGAATGGTTGGC |
| | | | DLEHIVNPNDEYNYEEEE | | TGAACACTACTCTGAATTCGGTGCTGAATTGTACTTCATCACCGACAAG |
| | | | GFI** | | TCTGCTGAAGGTTGTCAATTCGTTAAGGGTTTCTCTGGTATCGGTGGTT |
| | | | | | TCTTGAGATACAAGGTTGACTTGGAACACATCGTTAACCCAAACGACGA |
| | | | | | ATACAACTACGAAGAAGAAGAAGGTTTCATCtgatag |
| 30 | Tth_eRF1_ | SEQ ID NO: | MEQKPPFQNPLQKLQDRG | SEQ ID NO: | ATGGAACAAAAGCCACCATTCCAAAACCCATTGCAAAAGTTGCAAGACA |
| | XP_ | 69 | TKMDQSSGSCMSKQAEEQ | 130 | GAGGTACCAAGATGGACCAATCTTCTGGTTCTTGTATGTCTAAGCAAGC |
| | 001018211.4 | | KRLQISQYQLRKQLQMLR | | TGAAGAACAAAAGAGATTGCAAATCTCTCAATACCAATTGAGAAAGCAA |
| | | | NMRGEQTSCVSLYIPERK | | TTGCAAATGTTGAGAAACATGAGAGGTGAACAAACCTCTTGTGTTTCTT |
| | | | KLYEVVNYLQQEESGAAS | | TGTACATCCCAGAAAGAAAGAAGTTGTACGAAGTTGTTAACTACTTGCA |
| | | | IKNTQNRKSVQSALSMLR | | ACAAGAAGAATCTGGTGCTGCTTCTATCAAGAACACCCAAAACAGAAAG |
| | | | ERLKNFNLHKKYPKGMIF | | TCTGTTCAATCTGCTTTGTCTATGTTGAGAGAAAGATTGAAGAACTTCA |
| | | | FCADSLDSKRLLIEILDP | | ACTTGCACAAGAAGTACCCAAAGGGTATGATCTTCTTCTGTGCTGACTC |
| | | | PKAVQSFRYSCNTIFYLD | | TTTGGACTCTAAGAGATTGTTGATCGAAATCTTGGACCCACCAAAGGCT |
| | | | DLEYMLKDQPTYGFVVAD | | GTTCAATCTTTCAGATACTCTTGTAACACCATCTTCTACTTGGACGACT |
| | | | GHGYLIATVCGFDIQILQ | | TGGAATACATGTTGAAGGACCAACCAACCTACGGTTTCGTTGTTGCTGA |
| | | | SKQEDLPNKHNKGGQSSL | | CGGTCACGGTTACTTGATCGCTACCGTTTGTGGTTTCGACATCCAAATC |
| | | | RFSRLCDAARERLVKNIA | | TTGCAATCTAAGCAAGAAGACTTGCCAAACAAGCACAACAAGGGTGGTC |
| | | | DAMRRCYANENGTQTNLS | | AATCTTCTTTGAGATTCTCTAGATTGTGTGACGCTGCTAGAGAAAGATT |
| | | | GIVLCGMSDIKDKVQKEL | | GGTTAAGAACATCGCTGACGCTATGAGAAGATGTTACGCTAACGAAAAC |
| | | | QQLCPCIENKIVASYDVS | | GGTACCCAAACCAACTTGTCTGGTATCGTTTTGTGTGGTATGTCTGACA |
| | | | YSGQAGLKQALQMSTEML | | TCAAGGACAAGGTTCAAAAGGAATTGCAACAATTGTGTCCATGTATCGA |
| | | | KLDQLFQEMNLLSDFFAN | | AAACAAGATCGTTGCTTCTTACGACGTTTCTTACTCTGGTCAAGCTGGT |
| | | | FSLETSKVVYGGELTVRA | | TTGAAGCAAGCTTTGCAAATGTCTACCGAAATGTTGAAGTTGGACCAAT |
| | | | LEEGNVKKLILCQDSELQ | | TGTTCCAAGAAATGAACTTGTTGTCTGACTTCTTCGCTAACTTCTCTTT |
| | | | RVTVYNSKTQEETIQYLM | | GGAAACCTCTAAGGTTGTTTACGGTGGTGAATTGACCGTTAGAGCTTTG |
| | | | PSQVKALQDSISKTSDQE | | GAAGAAGGTAACGTTAAGAAGTTGATCTTGTGTCAAGACTCTGAATTGC |
| | | | ANNKKNQLQVYSQQNINE | | AAAGAGTTACCGTTTACAACTCTAAGACCCAAGAAGAAACCATCCAATA |
| | | | WIVENISSFSQDLEIVFV | | CTTGATGCCATCTCAAGTTAAGGCTTTGCAAGACTCTATCTCTAAGACC |
| | | | SDKTQQGVQFSKSFQGVG | | TCTGACCAAGAAGCTAACAACAAGAAGAACCAATTGCAAGTTTACTCTC |
| | | | AYLKYSLDYSSLHAQEKE | | AACAAAACATCAACGAATGGATCGTTGAAAACATCTCTTCTTTCTCTCA |
| | | | NDQLEQEYCYDDEEGFI* | | AGACTTGGAAATCGTTTTCGTTTCTGACAAGACCCAACAAGGTGTTCAA |
| | | | * | | TTCTCTAAGTCTTTCCAAGGTGTTGGTGCTTACTTGAAGTACTCTTTGG |
| | | | | | ACTACTCTTCTTTGCACGCTCAAGAAAAGGAAAACGACCAATTGGAACA |
| | | | | | AGAATACTGTTACGACGACGAAGAAGGTTTCATCtgatag |
| 31 | Tth_eRF1_ | SEQ ID NO: | MIKNIFKLLPISLRAIPL | SEQ ID NO: | ATGATCAAGAACATCTTCAAGTTGTTGCCAATCTCTTTGAGAGCTATCC |
| | XP_ | 70 | KQQQNSFSQICSLYNTKL | 131 | CATTGAAGCAACAACAAAACTCTTTCTCTCAAATCTGTTCTTTGTACAA |
| | 001008252.2 | | FKVINLIQTNNKCFFSFR | | CACCAAGTTGTTCAAGGTTATCAACTTGATCCAAACCAACAACAAGTGT |
| | | | AKETFKKKTSSLEIETHE | | TTCTTCTCTTTCAGAGCTAAGGAAACCTTCAAGAAGAAGACCTCTTCTT |
| | | | QVSDLTRCIYRRMKQFHN | | TGGAAATCGAAACCCACTTCCAAGTTTCTGACTTGACCAGATGTATCTA |
| | | | EYTDIQKILSQEQQQADI | | CAGAAGAATGAAGCAATTCCACAACGAATACACCGACATCCAAAAGATC |
| | | | NLEQLRKKINVLQPLNDV | | TTGTCTCAAGAACAACAACAAGCTGACATCAACTTGGAACAATTGAGAA |
| | | | FEKLEQNIKTLQELQKQK | | AGAAGATCAACGTTTTGCAACCATTGAACGACGTTTTCGAAAAGTTGGA |
| | | | EESASDPEMLALIEEEME | | ACAAAACATCAAGACCTTGCAAGAATTGCAAAAGCAAAAGGAAGAATCT |
| | | | NSKQLIDELQDECLEQLL | | GCTTCTGACCCAGAAATGTTGGCTTTGATCGAAGAAGAAATGGAAAACT |
| | | | PKGKHDDCSEITLEVRGG | | CTAAGCAATTGATCGACGAATTGCAAGACGAATGTTTGGAACAATTGTT |
| | | | AGGSESSLFAEEVFKMYQ | | GCCAAAGGGTAAGCACGACGACTGTTCTGAAATCACCTTGGAAGTTAGA |
| | | | AFFAQQGYQFSIDSFQVD | | GGTGGTGCTGGTGGTTCTGAATCTTCTTTGTTCGCTGAAGAAGTTTTCA |
| | | | MAINKGCKLGVLKVSGTN | | AGATGTACCAAGCTTTCTTCGCTCAACAAGGTTACCAATTCTCTATCGA |
| | | | IYKKMMNESGVHKVIRVP | | CTCTTTCCAAGTTGACATGGCTATCAACAAGGGTTGTAAGTTGGGTGTT |
| | | | ETESKGRLHSSTISVVVM | | TTGAAGGTTTCTGGTACCAACATCTACAAGAAGATGATGAACGAATCTG |
| | | | PVVPMDFKVDEKDLKFEF | | GTGTTCACAAGGTTATCAGAGTTCCAGAAACCGAATCTAAGGGTAGATT |
| | | | MRSQGAGGQHVNKVESAC | | GCACTCTTCTACCATCTCTGTTGTTGTTATGCCAGTTGTTCCAATGGAC |
| | | | RVTHLPTGISVLCQDDRQ | | TTCAAGGTTGACGAAAAGGACTTGAAGTTCGAATTCATGAGATCTCAAG |
| | | | QERNKQRALKLLTEKLFQ | | GTGCTGGTGGTCAACACGTTAACAAGGTTGAATCTGCTTGTAGAGTTAC |
| | | | VEVEKSNQQQSDQRKSQI | | CCACTTGCCAACCGGTATCTCTGTTTTGTGTCAAGACGACAGACAACAA |
| | | | GGGDRSDKIRTYNFPQGR | | GAAAGAAACAAGCAAAGAGCTTTGAAGTTGTTGACCGAAAAGTTGTTCC |
| | | | ITDHRTNLTLFGIEKMMK | | AAGTTGAAGTTGAAAAGTCTAACCAACAACAATCTGACCAAAGAAAGTC |
| | | | GEFLEEFIDEYEEKVNNE | | TCAAATCGGTGGTGGTGACAGATCTGACAAGATCAGAACCTACAACTTC |
| | | | LIESVLKQLEEDENQSQP | | CCACAAGGTAGAATCACCGACCACAGAACCAACTTGACCTTGTTCGGTA |
| | | | KN ** | | TCGAAAAGATGATGAAGGGTGAATTCTTGGAAGAATTCATCGACGAATA |
| | | | | | CGAAGAAAAGGTTAACAACGAATTGATCGAATCTGTTTTGAAGCAATTG |
| | | | | | GAAGAAGACGAAAACCAATCTCAACCAAAGAACtgatag |
| 32 | Pte_eRF1_ | SEQ ID NO: | MDQKLNDAEIALEQFRLK | SEQ ID NO: | ATGGACCAAAAGTTGAACGACGCTGAAATCGCTTTGGAACAATTCAGAT |
| | XP_ | 71 | KLIKTLSQERTAGTSVVS | 132 | TGAAGAAGTTGATCAAGACCTTGTCTCAAGAAAGAACCGCTGGTACCTC |
| | 001425245.1 | | VYIPPKRIISDITNRLNT | | TGTTGTTTCTGTTTACATCCCACCAAAGAGAATCATCTCTGACATCACC |
| | | | QYAEAASIKDKGNRISVQ | | AACAGATTGAACACCCAATACGCTGAAGCTGCTTCTATCAAGGACAAGG |
| | | | EAIQAAILRLRPYNKAPN | | GTAACAGAATCTCTGTTCAAGAAGCTATCCAAGCTGCTATCTTGAGACT |
| | | | NGLVVFCGIVQQADGKGE | | CAGACCATACAACAAGGCTCCAAACAACGGTTTGGTTGTTTTCTGTGGT |
| | | | KKISVVIEPYRPLDLSLY | | ATCGTTCAACAAGCTGACGGTAAGGGTGAAAAGAAGATCTCTGTTGTTA |
| | | | FCDPQFHVEELRALLNID | | TCGAACCATACAGACCATTGGACTTGTCTTTGTACTTCTGTGACCCACA |
| | | | PPFGFIIMDGNGSLFATI | | ATTCCACGTTGAAGAATTGAGAGCTTTGTTGAACATCGACCCACCATTC |
| | | | QGNSKQIIKSFDVDLPKK | | GGTTTCATCATCATGGACGGTAACGGTTCTTTGTTCGCTACCATCCAAG |
| | | | HNKGGQSSVRFARLRMEK | | GTAACTCTAAGCAAATCATCAAGTCTTTCGACGTTGACTTGCCAAAGAA |
| | | | RHNYLRKVCETATTCFIA | | GCACAACAAGGGTGGTCAATCTTCTGTTAGATTCGCTAGATTGAGAATG |
| | | | EDRPNVKGLVLAGSADFK | | GAAAAGAGACACAACTACTTGAGAAAGGTTTGTGAAACCGCTACCACCT |
| | | | NDLAGSQFFDKRLQPLII | | GTTTCATCGCTGAAGACAGACCAAACGTTAAGGGTTTGGTTTTGGCTGG |
| | | | SVVDINYGGEQGLNQAVQ | | TTCTGCTGACTTCAAGAACGACTTGGCTGGTTCTCAATTCTTCGACAAG |
| | | | LSQESLLEVKYIREKNLV | | AGATTGCAACCATTGATCATCTCTGTTGTTGACATCAACTACGGTGGTG |
| | | | GQFFENIDKDTGLVVYGV | | AACAAGGTTTGAACCAAGCTGTTCAATTGTCTCAAGAATCTTTGTTGGA |
| | | | QDTMRAVESQTIKTLVCV | | AGTTAAGTACATCAGAGAAAAGAACTTGGTTGGTCAATTCTTCGAAAAC |
| | | | DTLQYLRLECQSKQTEQK | | ATCGACAAGGACACCGGTTTGGTTGTTTACGGTGTTCAAGACACCATGA |
| | | | AIKYIKGNEGYEAGSLIE | | GAGCTGTTGAATCTCAAACCATCAAGACCTTGGTTTGTGTTGACACCTT |
| | | | EKNGEQFVILVKEDLVEH | | GCAATACTTGAGATTGGAATGTCAATCTAAGCAAACCGAACAAAAGGCT |
| | | | LSEKFKDYGLDFQLITDH | | ATCAAGTACATCAAGGGTAACGAAGGTTACGAAGCTGGTTCTTTGATCG |
| | | | SVEGNQFMKGFSGLGGFL | | AAGAAAAGAACGGTGAACAATTCGTTATCTTGGTTAAGGAAGACTTGGT |
| | | | RFKMDMDYLVQQEDWKDE | | TGAACACTTGTCTGAAAAGTTCAAGGACTACGGTTTGGACTTCCAATTG |
| | | | DEDFI** | | ATCACCGACCACTCTGTTGAAGGTAACCAATTCATGAAGGGTTTCTCTG |
| | | | | | GTTTGGGTGGTTTCTTGAGATTCAAGATGGACATGGACTACTTGGTTCA |
| | | | | | ACAAGAAGACTGGAAGGACGAAGACGAAGACTTCATCtgatag |
| 33 | Pte_eRF1_ | SEQ ID NO: | MNQNQIQEQELEIEQFRL | SEQ ID NO: | ATGAACCAAAACCAAATCCAAGAACAAGAATTGGAAATCGAACAATTCA |
| | XP_ | 72 | SKIIKTLSKTKVIGTSAV | 133 | GATTGTCTAAGATCATCAAGACCTTGTCTAAGACCAAGGTTATCGGTAC |
| | 001448143.1 | | SLYIPPKKIISDITNRLN | | CTCTGCTGTTTCTTTGTACATCCCACCAAAGAAGATCATCTCTGACATC |
| | | | TQFSEAASIQDKVNRTSV | | ACCAACAGATTGAACACCCAATTCTCTGAAGCTGCTTCTATCCAAGACA |
| | | | QDSIQGAVLKLKKYTKAP | | AGGTTAACAGAACCTCTGTTCAAGACTCTATCCAAGGTGCTGTTTTGAA |
| | | | ASGLVLFSGLVEFEKGQK | | GTTGAAGAAGTACACCAAGGCTCCAGCTTCTGGTTTGGTTTTGTTCTCT |
| | | | KISYVIEPFRPLQLSLFF | | GGTTTGGTTGAATTCGAAAAGGGTCAAAAGAAGATCTCTTACGTTATCG |
| | | | CDNYFHIEQLEPLLKLEP | | AACCATTCAGACCATTGCAATTGTCTTTGTTCTTCTGTGACAACTACTT |
| | | | SYGFIIMDGNGALFGKVQ | | CCACATCGAACAATTGGAACCATTGTTGAAGTTGGAACCATCTTACGGT |
| | | | GISKETLKSFNVDLPKKH | | TTCATCATCATGGACGGTAACGGTGCTTTGTTCGGTAAGGTTCAAGGTA |
| | | | NKGGQSSLRFSRIRYWAR | | TCTCTAAGGAAACCTTGAAGTCTTTCAACGTTGACTTGCCAAAGAAGCA |
| | | | HNYLIKVSEQAKNCFISD | | CAACAAGGGTGGTCAATCTTCTTTGAGATTCTCTAGAATCAGATACTGG |
| | | | DKPTIKGLVLAGIADFKN | | GCTAGACACAACTACTTGATCAAGGTTTCTGAACAAGCTAAGAACTGTT |
| | | | KLAESPALDKRLQPLILS | | TCATCTCTGACGACAAGCCAACCATCAAGGGTTTGGTTTTGGCTGGTAT |
| | | | IVDVNYGGENGFNQAIQY | | CGCTGACTTCAAGAACAAGTTGGCTGAATCTCCAGCTTTGGACAAGAGA |
| | | | SQEVLQNQKLQREKDLVA | | TTGCAACCATTGATCTTGTCTATCGTTGACGTTAACTACGGTGGTGAAA |
| | | | KFFLSLDLDNGKSVYGVV | | ACGGTTTCAACCAAGCTATCCAATACTCTCAAGAAGTTTTGCAAAACCA |
| | | | DTMKAIEQELVKQVICIQ | | AAAGTTGCAAAGAGAAAAGGACTTGGTTGCTAAGTTCTTCTTGTCTTTG |
| | | | TLEYSRVECISKQTGVKS | | GACTTGGACAACGGTAAGTCTGTTTACGGTGTTGTTGACACCATGAAGG |
| | | | IKYLKGLDLYEQGSLFED | | CTATCGAACAAGAATTGGTTAAGCAAGTTATCTGTATCCAAACCTTGGA |
| | | | NKGEQFQVTSCQDLVEYL | | ATACTCTAGAGTTGAATGTATCTCTAAGCAAACCGGTGTTAAGTCTATC |
| | | | AENYREKGIDFQLISDNS | | AAGTACTTGAAGGGTTTGGACTTGTACGAACAAGGTTCTTTGTTCGAAG |
| | | | AEGHQFYKGFGGMAGFFR | | ACAACAAGGGTGAACAATTCCAAGTTACCTCTTGTCAAGACTTGGTTGA |
| | | | FSMKMQYNMDSEEEWKSE | | ATACTTGGCTGAAAACTACAGAGAAAAGGGTATCGACTTCCAATTGATC |
| | | | DDEFI** | | TCTGACAACTCTGCTGAAGGTCACCAATTCTACAAGGGTTTCGGTGGTA |
| | | | | | TGGCTGGTTTCTTCAGATTCTCTATGAAGATGCAATACAACATGGACTC |
| | | | | | TGAAGAAGAATGGAAGTCTGAAGACGACGAATTCATCtgatag |
| 34 | Smy_eRF1_ | SEQ ID NO: | MVESIAAGQVGDNKHIEM | SEQ ID NO: | ATGGTTGAATCTATCGCTGCTGGTCAAGTTGGTGACAACAAGCACATCG |
| | Q9BMM1.1 | 73 | WKIKRLINKLENCKGNGT | 134 | AAATGTGGAAGATCAAGAGATTGATCAACAAGTTGGAAAACTGTAAGGG |
| | | | SMVSLIIPPKEDINKSGK | | TAACGGTACCTCTATGGTTTCTTTGATCATCCCACCAAAGGAAGACATC |
| | | | LLVGELSAAQNIKSRITR | | AACAAGTCTGGTAAGTTGTTGGTTGGTGAATTGTCTGCTGCTCAAAACA |
| | | | QSVITAITSTKEKLKLYR | | TCAAGTCTAGAATCACCAGACAATCTGTTATCACCGCTATCACCTCTAC |
| | | | QTPTNGLCIYCGVILMED | | CAAGGAAAAGTTGAAGTTGTACAGACAAACCCCAACCAACGGTTTGTGT |
| | | | GKTEKKINFDFEPFRPIN | | ATCTACTGTGGTGTTATCTTGATGGAAGACGGTAAGACCGAAAAGAAGA |
| | | | QFMYFCGGKFQTEPLTTL | | TCAACTTCGACTTCGAACCATTCAGACCAATCAACCAATTCATGTACTT |
| | | | LADDDKFGFIIVDGNGAL | | CTGTGGTGGTAAGTTCCAAACCGAACCATTGACCACCTTGTTGGCTGAC |
| | | | YATLQGNSREILQKITVE | | GACGACAAGTTCGGTTTCATCATCGTTGACGGTAACGGTGCTTTGTACG |
| | | | LPKKHRKGGQSSVRFARL | | CTACCTTGCAAGGTAACTCTAGAGAAATCTTGCAAAAGATCACCGTTGA |
| | | | REEKRHNYLRKVAELAGS | | ATTGCCAAAGAAGCACAGAAAGGGTGGTCAATCTTCTGTTAGATTCGCT |
| | | | NFITNDKPNVTGLVLAGN | | AGATTGAGAGAAGAAAAGAGACACAACTACTTGAGAAAGGTTGCTGAAT |
| | | | AGFKNELSETDMLDKRLL | | TGGCTGGTTCTAACTTCATCACCAACGACAAGCCAAACGTTACCGGTTT |
| | | | PIIVSIVDVSYGGENGLN | | GGTTTTGGCTGGTAACGCTGGTTTCAAGAACGAATTGTCTGAAACCGAC |
| | | | EAITLSADALTNVKFVAE | | ATGTTGGACAAGAGATTGTTGCCAATCATCGTTTCTATCGTTGACGTTT |
| | | | KKLVSKFFEEISLDTGMI | | CTTACGGTGGTGAAAACGGTTTGAACGAAGCTATCACCTTGTCTGCTGA |
| | | | VFGVQDTMKALELGAVET | | CGCTTTGACCAACGTTAAGTTCGTTGCTGAAAAGAAGTTGGTTTCTAAG |
| | | | ILLFEELEITRYVIKNPV | | TTCTTCGAAGAAATCTCTTTGGACACCGGTATGATCGTTTTCGGTGTTC |
| | | | KGDTRTLFLNPTQQKDSK | | AAGACACCATGAAGGCTTTGGAATTGGGTGCTGTTGAAACCATCTTGTT |
| | | | YFKDQASGLDMDVISEDQ | | GTTCGAAGAATTGGAAATCACCAGATACGTTATCAAGAACCCAGTTAAG |
| | | | LAEWLCHNYQNYAQVEFI | | GGTGACACCAGAACCTTGTTCTTGAACCCAACCCAACAAAAGGACTCTA |
| | | | TDKSQEGYQFVKGFGGIG | | AGTACTTCAAGGACCAAGCTTCTGGTTTGGACATGGACGTTATCTCTGA |
| | | | GFLRYKVDMEEALGDVGD | | AGACCAATTGGCTGAATGGTTGTGTCACAACTACCAAAACTACGCTCAA |
| | | | GGDDFDPDTDFI** | | GTTGAATTCATCACCGACAAGTCTCAAGAAGGTTACCAATTCGTTAAGG |
| | | | | | GTTTCGGTGGTATCGGTGGTTTCTTGAGATACAAGGTTGACATGGAAGA |
| | | | | | AGCTTTGGGTGACGTTGGTGACGGTGGTGACGACTTCGACCCAGACACC |
| | | | | | GACTTCATCtgatag |
| 35 | Ssa_eRF1_ | SEQ ID NO: | MDEAKLLQLEMWRLRKQL | SEQ ID NO: | ATGGACGAAGCTAAGTTGTTGCAATTGGAAATGTGGAGATTGAGAAAGC |
| | EST45466.1 | 74 | QKLDNTNTNSTSVVSLVM | 135 | AATTGCAAAAGTTGGACAACACCAACACCAACTCTACCTCTGTTGTTTC |
| | | | PPGEDINKMVQMLNQEAT | | TTTGGTTATGCCACCAGGTGAAGACATCAACAAGATGGTTCAAATGTTG |
| | | | QADCIKSRQNRQAVQTAI | | AACCAAGAAGCTACCCAAGCTGACTGTATCAAGTCTAGACAAAACAGAC |
| | | | ILAANRCKLYPKMPKNGL | | AAGCTGTTCAAACCGCTATCATCTTGGCTGCTAACAGATGTAAGTTGTA |
| | | | AVFAGEVYVDGKIKKIAV | | CCCAAAGATGCCAAAGAACGGTTTGGCTGTTTTCGCTGGTGAAGTTTAC |
| | | | HFSPCKPIGNFMYSCDGV | | GTTGACGGTAAGATCAAGAAGATCGCTGTTCACTTCTCTCCATGTAAGC |
| | | | FHTQEVKDLLTVEEVYGF | | CAATCGGTAACTTCATGTACTCTTGTGACGGTGTTTTCCACACCCAAGA |
| | | | IIMDGHGTLIATLCGSHR | | AGTTAAGGACTTGTTGACCGTTGAAGAAGTTTACGGTTTCATCATCATG |
| | | | EIKHRMLVDLPKKHGRGG | | GACGGTCACGGTACCTTGATCGCTACCTTGTGTGGTTCTCACAGAGAAA |
| | | | QSSVRFARLRMEARGNYV | | TCAAGCACAGAATGTTGGTTGACTTGCCAAAGAAGCACGGTAGAGGTGG |
| | | | KIITELCTKYFITGDRLN | | TCAATCTTCTGTTAGATTCGCTAGATTGAGAATGGAAGCTAGAGGTAAC |
| | | | VSGVILAGSADFKDVLAG | | TACGTTAAGATCATCACCGAATTGTGTACCAAGTACTTCATCACCGGTG |
| | | | SDFMDPRIKEGIIKQVDI | | ACAGATTGAACGTTTCTGGTGTTATCTTGGCTGGTTCTGCTGACTTCAA |
| | | | GYGMEQGLSQAIEQAGDI | | GGACGTTTTGGCTGGTTCTGACTTCATGGACCCAAGAATCAAGGAAGGT |
| | | | LKDVRLFKEVKIINEFLD | | ATCATCAAGCAAGTTGACATCGGTTACGGTATGGAACAAGGTTTGTCTC |
| | | | HISRDTRKYCFGIIDTLR | | AAGCTATCGAACAAGCTGGTGACATCTTGAAGGACGTTAGATTGTTCAA |
| | | | CLEMGSVEHLVVWEDFPW | | GGAAGTTAAGATCATCAACGAATTCTTGGACCACATCTCTAGAGACACC |
| | | | DRVQCRNSEGTEYYKLVN | | AGAAAGTACTGTTTCGGTATCATCGACACCTTGAGATGTTTGGAAATGG |
| | | | TVTGAINLVDNDESIRQG | | GTTCTGTTGAACACTTGGTTGTTTGGGAAGACTTCCCATGGGACAGAGT |
| | | | EEEIEAELFVEWLAQNIE | | TCAATGTAGAAACTCTGAAGGTACCGAATACTACAAGTTGGTTAACACC |
| | | | KFGAEIHLVTENSNEGTQ | | GTTACCGGTGCTATCAACTTGGTTGACAACGACGAATCTATCAGACAAG |
| | | | FCSGFGGLGGILRWQMDL | | GTGAAGAAGAAATCGAAGCTGAATTGTTCGTTGAATGGTTGGCTCAAAA |
| | | | NELGKYMDIGKENNDLDD | | CATCGAAAAGTTCGGTGCTGAAATCCACTTGGTTACCGAAAACTCTAAC |
| | | | LEDFM** | | GAAGGTACCCAATTCTGTTCTGGTTTCGGTGGTTTGGGTGGTATCTTGA |
| | | | | | GATGGCAAATGGACTTGAACGAATTGGGTAAGTACATGGACATCGGTAA |
| | | | | | GGAAAACAACGACTTGGACGACTTGGAAGACTTCATGtgatag |
| 36 | Yeast_eRF1_ | SEQ ID NO: | MDNEVEKNIEIWKVKKLV | SEQ ID NO: | ATGGACAACGAAGTTGAAAAGAACATCGAAATCTGGAAGGTTAAGAAGT |
| | eRF3 | 75 | QSLEKARGNGTSMISLVI | 136 | TGGTTCAATCTTTGGAAAAGGCTAGAGGTAACGGTACCTCTATGATCTC |
| | | | PPKGQIPLYQKMLTDEYG | | TTTGGTTATCCCACCAAAGGGTCAAATCCCATTGTACCAAAAGATGTTG |
| | | | TASNIKSRVNRLSVLSAI | | ACCGACGAATACGGTACCGCTTCTAACATCAAGTCTAGAGTTAACAGAT |
| | | | TSTQQKLKLYNTLPKNGL | | TGTCTGTTTTGTCTGCTATCACCTCTACCCAACAAAAGTTGAAGTTGTA |
| | | | VLYCGDIITEDGKEKKVT | | CAACACCTTGCCAAAGAACGGTTTGGTTTTGTACTGTGGTGACATCATC |
| | | | FDIEPYKPINTSLYLCDN | | ACCGAAGACGGTAAGGAAAAGAAGGTTACCTTCGACATCGAACCATACA |
| | | | KFHTEVLSELLQADDKFG | | AGCCAATCAACACCTCTTTGTACTTGTGTGACAACAAGTTCCACACCGA |
| | | | FIVMDGQGTLFGSVSGNT | | AGTTTTGTCTGAATTGTTGCAAGCTGACGACAAGTTCGGTTTCATCGTT |
| | | | RTVLHKFTVDLPKKHGRG | | ATGGACGGTCAAGGTACCTTGTTCGGTTCTGTTTCTGGTAACACCAGAA |
| | | | GQSALRFARLREEKRHNY | | CCGTTTTGCACAAGTTCACCGTTGACTTGCCAAAGAAGCACGGTAGAGG |
| | | | VRKVAEVAVQNFITNDKV | | TGGTCAATCTGCTTTGAGATTCGCTAGATTGAGAGAAGAAAAGAGACAC |
| | | | NVKGLILAGSADFKTDLA | | AACTACGTTAGAAAGGTTGCTGAAGTTGCTGTTCAAAACTTCATCACCA |
| | | | KSELFDPRLACKVISIVD | | ACGACAAGGTTAACGTTAAGGGTTTGATCTTGGCTGGTTCTGCTGACTT |
| | | | VSYGGENGFNQAIELSAE | | CAAGACCGACTTGGCTAAGTCTGAATTGTTCGACCCAAGATTGGCTTGT |
| | | | ALANVKYVQEKKLLEAYF | | AAGGTTATCTCTATCGTTGACGTTTCTTACGGTGGTGAAAACGGTTTCA |
| | | | DEISQDTGKFCYGIDDTL | | ACCAAGCTATCGAATTGTCTGCTGAAGCTTTGGCTAACGTTAAGTACGT |
| | | | KALDLGAVEKLIVFENLE | | TCAAGAAAAGAAGTTGTTGGAAGCTTACTTCGACGAAATCTCTCAAGAC |
| | | | TIRYTFKDAEDNEVIKFA | | ACCGGTAAGTTCTGTTACGGTATCGACGACACCTTGAAGGCTTTGGACT |
| | | | EPEAKDKSFAIDKATGQE | | TGGGTGCTGTTGAAAAGTTGATCGTTTTCGAAAACTTGGAAACCATCAG |
| | | | MDVVSEEPLIEWLAANYK | | ATACACCTTCAAGGACGCTGAAGACAACGAAGTTATCAAGTTCGCTGAA |
| | | | NEGATLEFITDKSSEGAQ | | CCAGAAGCTAAGGACAAGTCTTTCGCTATCGACAAGGCTACCGGTCAAG |
| | | | FVTGFGGIGAMLRYKVNF | | AAATGGACGTTGTTTCTGAAGAACCATTGATCGAATGGTTGGCTGCTAA |
| | | | EQLVDESEDEYYDEDEGS | | CTACAAGAACTTCGGTGCTACCTTGGAATTCATCACCGACAAGTCTTCT |
| | | | DYDFI | | GAAGGTGCTCAATTCGTTACCGGTTTCGGTGGTATCGGTGCTATGTTGA |
| | | | | | GATACAAGGTTAACTTCGAACAATTGGTTGACGAATCTGAAGACGAATA |
| | | | | | CTACGACGAAGACGAAGGTTCTGACTACGACTTCATCtgatag |
| 37 | Yeast_eRF1_ | SEQ ID NO: | MSDSNQGNNQQNYQQYSQ | SEQ ID NO: | ATGTCTGACTCTAACCAAGGTAACAACCAACAAAACTACCAACAATACT |
| | eRF3 | 76 | NGNQQQGNNRYQGYQAYN | 137 | CTCAAAACGGTAACCAACAACAAGGTAACAACAGATACCAAGGTTACCA |
| | | | AQAQPAGGYYQNYQGYSG | | AGCTTACAACGCTCAAGCTCAACCAGCTGGTGGTTACTACCAAAACTAC |
| | | | YQQGGYQQYNPDAGYQQQ | | CAAGGTTACTCTGGTTACCAGCAGGGGGGGTATCAGCAGTATAATCCTG |
| | | | YNPQGGYQQYNPQGGYQQ | | ACGCTGGTTACCAACAACAATACAACCCACAAGGTGGTTACCAGCAGTA |
| | | | QFNPQGGRGNYKNENYNN | | TAATCCTCAGGGCGGCTATCAGCAGCAATTCAACCCACAAGGTGGTAGA |
| | | | NLQGYQAGFQPQSQGMSL | | GGTAACTACAAGAACTTCAACTACAACAACAACTTGCAAGGTTACCAAG |
| | | | NDFQKQQKQAAPKPKKTL | | CTGGTTTCCAACCACAATCTCAAGGTATGTCTTTGAACGACTTCCAAAA |
| | | | KLVSSSGIKLANATKKVG | | GCAACAAAAGCAAGCTGCTCCAAAGCCAAAGAAGACCTTGAAGTTGGTT |
| | | | TKPAESDKKEEEKSAETK | | TCTTCTTCTGGTATCAAGTTGGCTAACGCTACCAAGAAGGTTGACACCA |
| | | | EPTKEPTKVEEPVKKEEK | | AGCCAGCTGAATCTGACAAGAAGGAAGAAGAAAAGTCTGCTGAAACCAA |
| | | | PVQTEEKTEEKSELPKVE | | GGAACCAACGAAAGAGCCTACGAAAGTTGAAGAACCAGTTAAGAAGGAA |
| | | | DLKISESTHNTNNANVTS | | GAAAAGCCAGTTCAAACCGAAGAAAAGACCGAAGAAAAGTCTGAATTGC |
| | | | ADALIKEQEEEVDDEVVN | | CAAAGGTTGAAGACTTGAAGATCTCTGAATCTACCCACAACACCAACAA |
| | | | DMFGGKDHVSLIFMGHVD | | CGCTAACGTTACCTCTGCTGACGCTTTGATCAAGGAACAAGAAGAAGAA |
| | | | AGKSTMGGNLLYLTGSVD | | GTTGACGACGAAGTTGTTAACGACATGTTCGGTGGTAAGGACCACGTTT |
| | | | KRTIEKYEREAKDAGRQG | | CTTTGATCTTCATGGGTCACGTTGACGCTGGTAAGTCTACCATGGGTGG |
| | | | WYLSWVMDTNKEERNDGK | | TAACTTGTTGTACTTGACCGGTTCTGTTGACAAGAGAACCATCGAAAAG |
| | | | TIEVGKAYFETEKRRYTI | | TACGAAAGAGAAGCTAAGGACGCTGGTAGACAAGGTTGGTACTTGTCTT |
| | | | LDAPGHKMYVSEMIGGAS | | GGGTTATGGACACCAACAAGGAAGAAAGAAACGACGGTAAGACCATCGA |
| | | | QADVGVLVISARKGEYET | | AGTTGGTAAGGCTTACTTCGAAACCGAAAAGAGAAGATACACCATCTTG |
| | | | GFERGGQTREHALLAKTQ | | GACGCTCCAGGTCACAAGATGTACGTTTCTGAAATGATCGGTGGTGCTT |
| | | | GVNKMVVVVNKMDDPTVN | | CTCAAGCTGACGTTGGTGTTTTGGTTATCTCTGCTAGAAAGGGTGAATA |
| | | | WSKERYDQCVSNVSNFLR | | CGAAACCGGTTTCGAAAGAGGTGGTCAAACCAGAGAACACGCTTTGTTG |
| | | | AIGYNIKTDVVFMPVSGY | | GCTAAGACCCAAGGTGTTAACAAGATGGTTGTTGTTGTTAACAAGATGG |
| | | | SGANLKDHVDPKECPWYT | | ACGACCCAACCGTTAACTGGTCTAAGGAAAGATACGACCAATGTGTTTC |
| | | | GPTLLEYLDTMNHVDRHI | | TAACGTTTCTAACTTCTTGAGAGCTATCGGTTACAACATCAAGACCGAC |
| | | | NAPFMLPIAAKMKDLGTI | | GTTGTTTTCATGCCAGTTTCTGGTTACTCTGGTGCTAACTTGAAGGACC |
| | | | VEGKIESGHIKKGQSTLL | | ACGTTGACCCAAAGGAATGTCCATGGTACACCGGTCCAACCTTGTTGGA |
| | | | MPNKTAVEIQNIYNETEN | | ATACTTGGACACCATGAACCACGTTGACAGACACATCAACGCTCCATTC |
| | | | EVDMAMCGEQVKLRIKGV | | ATGTTGCCAATCGCTGCTAAGATGAAGGACTTGGGTACCATCGTTGAAG |
| | | | EEEDISPGFVLTSPKNPI | | GTAAGATCGAATCTGGTCACATCAAGAAGGGTCAATCTACCTTGTTGAT |
| | | | KSVTKFVAQIAIVELKSI | | GCCAAACAAGACCGCTGTTGAAATCCAAAACATCTACAACGAAACCGAA |
| | | | IAAGFSCVMHVHTAIEEV | | AACGAAGTTGACATGGCTATGTGTGGTGAACAAGTTAAGTTGAGAATCA |
| | | | HIVKLLHKLEKGTNRKSK | | AGGGTGTTGAAGAAGAAGACATCTCTCCAGGTTTCGTTTTGACCTCTCC |
| | | | KPPAFAKKGMKVIAVLET | | AAAGAACCCAATCAAGTCTGTTACCAAGTTCGTTGCTCAAATCGCTATC |
| | | | EAPVCVETYQDYPQLGRF | | GTTGAATTGAAGTCTATCATCGCTGCTGGTTTCTCTTGTGTTATGCACG |
| | | | TLRDQGTTIAIGKIVKIA | | TTCACACCGCTATCGAAGAAGTTCACATCGTTAAGTTGTTGCACAAGTT |
| | | | E* | | GGAAAAGGGTACCAACAGAAAGTCTAAGAAGCCACCAGCTTTCGCTAAG |
| | | | | | AAGGGTATGAAGGTTATCGCTGTTTTGGAAACCGAAGCTCCAGTTTGTG |
| | | | | | TTGAAACCTACCAAGACTACCCACAATTGGGTAGATTCACCTTGCGCGA |
| | | | | | CCAAGGTACCACCATCGCTATCGGTAAGATCGTTAAGATCGCTGAATGA |
| 38 | Eoc_eRF1_ | SEQ ID NO: | MSIIDSNVETWKIKRIIK | SEQ ID NO: | ATGTCTATCATCGACTCTAACGTTGAAACCTGGAAGATCAAGAGAATCA |
| | CAC14170.1/ | 77 | NLERLRGNGTSMISLLLS | 138 | TCAAGAACTTGGAAAGATTGAGAGGTAACGGTACCTCTATGATCTCTTT |
| | Eoc_eRF3_ | | PRDAIPKVQGMLAGEYGT | | GTTGTTGTCTCCACGCGACGCTATCCCAAAGGTTCAAGGTATGTTGGCT |
| | AAL33628.1 | | AESIKSKINRLAVQGAIT | | GGTGAATACGGTACCGCTGAATCTATCAAGTCTAAGATCAACAGATTGG |
| | | | SAKERLKLYNRTPPNGLV | | CTGTTCAAGGTGCTATCACCTCTGCTAAGGAAAGATTGAAGTTGTACAA |
| | | | IYCGIVIGEDKSEKKYCI | | CAGAACCCCACCAAACGGTTTGGTTATCTACTGTGGTATCGTTATCGGT |
| | | | DFEPFRPLNTFKYICDNK | | GAAGACAAGTCTGAAAAGAAGTACTGTATCGACTTCGAACCATTCAGAC |
| | | | FYTKPLFELLENDDVFGF | | CATTGAACACCTTCAAGTACATCTGTGACAACAAGTTCTACACCAAGCC |
| | | | VIVDGSGCLFGTLQGNTK | | ATTGTTCGAATTGTTGGAAAACGACGACGTTTTCGGTTTCGTTATCGTT |
| | | | KIIQNITVSLPKKHGRGG | | GACGGTTCTGGTTGTTTGTTCGGTACCTTGCAAGGTAACACCAAGAAGA |
| | | | QSAPRFGRIREEKRHNYV | | TCATCCAAAACATCACCGTTTCTTTGCCAAAGAAGCACGGTAGAGGTGG |
| | | | RKVAEFATQHFITEDKPN | | TCAATCTGCTCCAAGATTCGGTAGAATCAGAGAAGAAAAGAGACACAAC |
| | | | VKGIILAGSANFKNDLSE | | TACGTTAGAAAGGTTGCTGAATTCGCTACCCAACACTTCATCACCGAAG |
| | | | SDLFDKRLSEIVLKIVDV | | ACAAGCCAAACGTTAAGGGTATCATCTTGGCTGGTTCTGCTAACTTCAA |
| | | | SYGGENGFSQAITLAEDT | | GAACGACTTGTCTGAATCTGACTTGTTCGACAAGAGATTGTCTGAAATC |
| | | | LSNVKFVEEKNLISKYFE | | GTTTTGAAGATCGTTGACGTTTCTTACGGTGGTGAAAACGGTTTCTCTC |
| | | | EIAQDTGMVVFGIEDTLN | | AAGCTATCACCTTGGCTGAAGACACCTTGTCTAACGTTAAGTTCGTTGA |
| | | | SLELGAVGTIICFENLEI | | AGAAAAGAACTTGATCTCTAAGTACTTCGAAGAAATCGCTCAAGACACC |
| | | | NRYEIRNPSTEEIKVIHL | | GGTATGGTTGTTTTCGGTATCGAAGACACCTTGAACTCTTTGGAATTGG |
| | | | CKDQQNDTRYKMIDNNYS | | GTGCTGTTGGTACCATCATCTGTTTCGAAAACTTGGAAATCAACAGATA |
| | | | YFIDQNTGLDLEILSCVP | | CGAAATCAGAAACCCATCTACCGAAGAAATCAAGGTTATCCACTTGTGT |
| | | | LTEWLCENYSKYGVRLEF | | AAGGACCAACAAAACGACACCAGATACAAGATGATCGACAACAACTACT |
| | | | ITDKSQEGFQFVNGFGGI | | CTTACTTCATCGACCAAAACACCGGTTTGGACTTGGAAATCTTGTCTTG |
| | | | GGFLRFKLEIENIDYEGE | | TGTTCCATTGACCGAATGGTTGTGTGAAAACTACTCTAAGTACGGTGTT |
| | | | DVGGEEFDADEDFI** | | AGATTGGAATTCATCACCGACAAGTCTCAAGAAGGTTTCCAATTCGTTA |
| | | | | | ACGGTTTCGGTGGTATCGGTGGTTTCTTGAGATTCAAGTTGGAAATCGA |
| | | | | | AAACATCGACTACGAAGGTGAAGACGTTGGTGGTGAAGAATTCGACGCT |
| | | | | | GACGAAGACTTCATCtaatag |
| 39 | Eoc_eRF1_ | SEQ ID NO: | MDKNSDFPALGSLKVQNQ | SEQ ID NO: | ATGGACAAGAACTCTGACTTCCCAGCTTTGGGTTCTTTGAAGGTTCAAA |
| | CAC14170.1/ | 78 | SSKDKNKKKEKEVKKEDE | 139 | ACCAATCTTCTAAGGACAAGAACAAGAAGAAGGAAAAGGAAGTTAAGAA |
| | Eoc_eRF3_ | | KKDEVKDEPQNNEEAKKT | | GGAAGACGAAAAGAAGGACGAAGTTAAGGACGAACCACAAAACAACGAA |
| | AAL33628.1 | | KLEATGRVFAAKSFTPKE | | GAAGCTAAGAAGACCAAGTTGGAAGCTACCGGTAGAGTTTTCGCTGCTA |
| | | | PVKINYYIPHEEPTLSEM | | AGTCTTTCACCCCAAAGGAACCAGTTAAGATCAACTACTACATCCCACA |
| | | | DFFPTLGGPASTAPVVSE | | CGAAGAACCAACCTTGTCTGAAATGGACTTCTTCCCAACCTTGGGTGGT |
| | | | AQKRHAEFLERHELFKPY | | CCAGCTTCTACCGCTCCAGTTGTTTCTGAAGCTCAAAAGAGACACGCTG |
| | | | ICLIPKELWYVPDPYFDM | | AATTCTTGGAAAGACACGAATTGTTCAAGCCATACATCTGTTTGATCCC |
| | | | FGYPVMALLPGLYAYLWN | | AAAGGAATTGTGGTACGTTCCAGACCCATACTTCGACATGTTCGGTTAC |
| | | | TYNIFFTPEGAWNTNKET | | CCAGTTATGGCTTTGTTGCCAGGTTTGTACGCTTACTTGTGGAACACCT |
| | | | VMSTLGELWTHAEWRDRI | | ACAACATCTTCTTCACCCCAGAAGGTGCTTGGAACACCAACAAGGAAAC |
| | | | IAKEQKETEEWERQMREW | | CGTTATGTCTACCTTGGGTGAATTGTGGACCCACGCTGAATGGAGAGAC |
| | | | EEENAEDDEGLSIDDMEN | | AGAATCATCGCTAAGGAACAAAAGGAAACCGAAGAATGGGAAAGACAAA |
| | | | YGKGKKKNKNKKKKDKAK | | TGAGAGAATGGGAAGAAGAAAACGCTGAAGACGACGAAGGTTTGTCTAT |
| | | | RPPPPPPKSLSSYKRFEK | | CGACGACATGGAAAACTACGGTAAGGGTAAGAAGAAGAACAAGAACAAG |
| | | | KKEDVVVPKKNIGFKEVS | | AAGAAGAAGGACAAGGCTAAAAGACCACCACCACCACCACCAAAGTCTT |
| | | | EITFEEEVVEVDETRQPS | | TGTCTTCTTACAAGAGATTCGAAAAGAAGAAGGAAGACGTTGTTGTTCC |
| | | | SLVFIGPVDAVKSTICGN | | AAAGAAGAACATCGGTTTCAAGGAAGTTTCTGAAATCACCTTCGAAGAA |
| | | | LMFMTGMVDERTIEKFKQ | | GAAGTTGTTGAAGTTGACGAAACCAGACAACCATCTTCTTTGGTTTTCA |
| | | | EAKEKNRDSWWLAYVMDI | | TCGGTCCAGTTGACGCTGTTAAGTCTACCATCTGTGGTAACTTGATGTT |
| | | | NDDEKSKGKTVEVGRATM | | CATGACCGGTATGGTTGACGAAAGAACCATCGAAAAGTTCAAGCAAGAA |
| | | | ETPTKRYTIFDAPGHKNY | | GCTAAGGAAAAGAACAGAGACTCTTGGTGGTTGGCTTACGTTATGGACA |
| | | | VPDMIMGAAMADVAALVI | | TCAACGACGACGAAAAGTCTAAGGGTAAGACCGTTGAAGTTGGTAGAGC |
| | | | SARKGEFEAGFERDGQTR | | TACCATGGAAACCCCAACCAAGAGATACACCATCTTCGACGCTCCAGGT |
| | | | EHAQLARSLGVNKLVVVV | | CACAAGAACTACGTTCCAGACATGATCATGGGTGCTGCTATGGCTGACG |
| | | | NEMDEETVQWSEERYNDI | | TTGCTGCTTTGGTTATCTCTGCTAGAAAGGGTGAATTCGAAGCTGGTTT |
| | | | LSGVTPFLIDQCGYKRED | | CGAACGCGACGGTCAAACCAGAGAACACGCTCAATTGGCTAGATCTTTG |
| | | | LIFVPISGLNGHNIDKLA | | GGTGTTAACAAGTTGGTTGTTGTTGTTAACGAAATGGACGAAGAAACCG |
| | | | SCCPWYTGPTLLEILDCI | | TTCAATGGTCTGAAGAAAGATACAACGACATCTTGTCTGGTGTTACCCC |
| | | | EPPKRNIDGPLRVPVLDK | | ATTCTTGATCGACCAATGTGGTTACAAGAGAGAAGACTTGATCTTCGTT |
| | | | MKDRGVVAFGKVESGVIR | | CCAATCTCTGGTTTGAACGGTCACAACATCGACAAGTTGGCTTCTTGTT |
| | | | IGPKLAVMPNNTKCQVVG | | GTCCATGGTACACCGGTCCAACCTTGTTGGAAATCTTGGACTGTATCGA |
| | | | IYNCKLELVRYANPGENI | | ACCACCAAAGAGAAACATCGACGGTCCATTGAGAGTTCCAGTTTTGGAC |
| | | | QIKVRMIEDENQINKGDV | | AAGATGAAGGACAGAGGTGTTGTTGCTTTCGGTAAGGTTGAATCTGGTG |
| | | | LCPYDNLAPITDLFEAEL | | TTATCAGAATCGGTCCAAAGTTGGCTGTTATGCCAAACAACACCAAGTG |
| | | | TILELLPHRPIITPGYKS | | TCAAGTTGTTGGTATCTACAACTGTAAGTTGGAATTGGTTAGATACGCT |
| | | | MMHLHTISDEIVIQTLTG | | AACCCAGGTGAAAACATCCAAATCAAGGTTAGAATGATCGAAGACGAAA |
| | | | IYELDGSGKEYVKKNPKY | | ACCAAATCAACAAGGGTGACGTTTTGTGTCCATACGACAACTTGGCTCC |
| | | | CKSGSKVIVKISTRVPVC | | AATCACCGACTTGTTCGAAGCTGAATTGACCATCTTGGAATTGTTGCCA |
| | | | LEKYEFIVHMGRFTLRDE | | CACAGACCAATCATCACCCCAGGTTACAAGTCTATGATGCACTTGCACA |
| | | | GKTIALGKVLRYKPAVIK | | CCATCTCTGACGAAATCGTTATCCAAACCTTGACCGGTATCTACGAATT |
| | | | KVEEIPPGVGDEGQAKLE | | GGACGGTTCTGGTAAGGAATACGTTAAGAAGAACCCAAAGTACTGTAAG |
| | | | ESEEFSGSRGDSPSKDDN | | TCTGGTTCTAAGGTTATCGTTAAGATCTCTACCAGAGTTCCAGTTTGTT |
| | | | KYEVITYDPEEDTIIASS | | TGGAAAAGTACGAATTCATCGTTCACATGGGTAGATTCACCTTGCGCGA |
| | | | TGSENAE*** | | CGAAGGTAAGACCATCGCTTTGGGTAAGGTTTTGAGATACAAGCCAGCT |
| | | | | | GTTATCAAGAAGGTTGAAGAAATCCCACCAGGTGTTGGTGACGAAGGTC |
| | | | | | AAGCTAAGTTGGAAGAATCTGAAGAATTCTCTGGTTCTAGAGGTGACTC |
| | | | | | TCCATCTAAGGACGACAACAAGTACGAAGTTATCACCTACGACCCAGAA |
| | | | | | GAAGACACCATCATCGCTTCTTCTACCGGTTCTGAAAACGCTGAAtaat |
| | | | | | ag |
| 40 | Eoc_eRF1_ | SEQ ID NO: | MAKLDDNVETWRIKRLIK | SEQ ID NO: | ATGGCTAAGTTGGACGACAACGTTGAAACCTGGAGAATCAAGAGATTGA |
| | AAG25924.1/ | 79 | NLEKLRGDGTSMISLLLS | 140 | TCAAGAACTTGGAAAAGTTGAGAGGTGACGGTACCTCTATGATCTCTTT |
| | Eoc_eRF3_ | | PRDQISKVQAMLAGEAGT | | GTTGTTGTCTCCACGCGACCAAATCTCTAAGGTTCAAGCTATGTTGGCT |
| | AAL33628.1 | | AVNIKSRVNRQAVLSAIT | | GGTGAAGCTGGTACCGCTGTTAACATCAAGTCTAGAGTTAACAGACAAG |
| | | | SAKERLKLYSKTPTNGLV | | CTGTTTTGTCTGCTATCACCTCTGCTAAGGAAAGATTGAAGTTGTACTC |
| | | | VYCGTVIGEDDSEKKYTI | | TAAGACCCCAACCAACGGTTTGGTTGTTTACTGTGGTACCGTTATCGGT |
| | | | DFEPFRPLNTFKYICDNK | | GAAGACGACTCTGAAAAGAAGTACACCATCGACTTCGAACCATTCAGAC |
| | | | FCTEPLFELLENDDVFGF | | CATTGAACACCTTCAAGTACATCTGTGACAACAAGTTCTGTACCGAACC |
| | | | VIVDGNGCLFGTLQGNTK | | ATTGTTCGAATTGTTGGAAAACGACGACGTTTTCGGTTTCGTTATCGTT |
| | | | KILQQITVSLPKKHGRGG | | GACGGTAACGGTTGTTTGTTCGGTACCTTGCAAGGTAACACCAAGAAGA |
| | | | QSAPRFGRIREEKRHNYV | | TCTTGCAACAAATCACCGTTTCTTTGCCAAAGAAGCACGGTAGAGGTGG |
| | | | RKVAELATQHFITDDRPN | | TCAATCTGCTCCAAGATTCGGTAGAATCAGAGAAGAAAAGAGACACAAC |
| | | | VKGLVLAGSANFKNDLSE | | TACGTTAGAAAGGTTGCTGAATTGGCTACCCAACACTTCATCACCGACG |
| | | | SDLFDKRLSEVVIKIVDV | | ACAGACCAAACGTTAAGGGTTTGGTTTTGGCTGGTTCTGCTAACTTCAA |
| | | | SYGGENGFSQAISLAEDA | | GAACGACTTGTCTGAATCTGACTTGTTCGACAAGAGATTGTCTGAAGTT |
| | | | LSNVKFVEEKNLISKYFE | | GTTATCAAGATCGTTGACGTTTCTTACGGTGGTGAAAACGGTTTCTCTC |
| | | | EIALDSGMIVFGVEDTLH | | AAGCTATCTCTTTGGCTGAAGACGCTTTGTCTAACGTTAAGTTCGTTGA |
| | | | SLEVGALDLLMCFENLEI | | AGAAAAGAACTTGATCTCTAAGTACTTCGAAGAAATCGCTTTGGACTCT |
| | | | NRYEIRDPANDEIKIYNL | | GGTATGATCGTTTTCGGTGTTGAAGACACCTTGCACTCTTTGGAAGTTG |
| | | | NKEQEKDSKYFKNEKTGT | | GTGCTTTGGACTTGTTGATGTGTTTCGAAAACTTGGAAATCAACAGATA |
| | | | DLEIVKCVALSEWLCENY | | CGAAATCCGCGACCCAGCTAACGACGAAATCAAGATCTACAACTTGAAC |
| | | | SKYGVKLEFITDKSQEGF | | AAGGAACAAGAAAAGGACTCTAAGTACTTCAAGAACGAAAAGACCGGTA |
| | | | QFVNGFGGIGGFLRYKLE | | CCGACTTGGAAATCGTTAAGTGTGTTGCTTTGTCTGAATGGTTGTGTGA |
| | | | MENHDYDKEDVGGEEFNP | | AAACTACTCTAAGTACGGTGTTAAGTTGGAATTCATCACCGACAAGTCT |
| | | | DEDFI** | | CAAGAAGGTTTCCAATTCGTTAACGGTTTCGGTGGTATCGGTGGTTTCT |
| | | | | | TGAGATACAAGTTGGAAATGGAAAACCACGACTACGACAAGGAAGACGT |
| | | | | | TGGTGGTGAAGAATTCAACCCAGACGAAGACTTCATCtaatag |
| 41 | Eoc_eRF1_ | SEQ ID NO: | MDKNSDFPALGSLKVQNQ | SEQ ID NO: | ATGGACAAGAACTCTGACTTCCCAGCTTTGGGTTCTTTGAAGGTTCAAA |
| | AAG25924.1/ | 80 | SSKDKNKKKEKEVKKEDE | 141 | ACCAATCTTCTAAGGACAAGAACAAGAAGAAGGAAAAGGAAGTTAAGAA |
| | Eoc_eRF3_ | | KKDEVKDEPQNNEEAKKT | | GGAAGACGAAAAGAAGGACGAAGTTAAGGACGAACCACAAAACAACGAA |
| | AAL33628.1 | | KLEATGRVFAAKSFTPKE | | GAAGCTAAGAAGACCAAGTTGGAAGCTACCGGTAGAGTTTTCGCTGCTA |
| | | | PVKINYYIPHEEPTLSEM | | AGTCTTTCACCCCAAAGGAACCAGTTAAGATCAACTACTACATCCCACA |
| | | | DFFPTLGGPASTAPVVSE | | CGAAGAACCAACCTTGTCTGAAATGGACTTCTTCCCAACCTTGGGTGGT |
| | | | AQKRHAEFLERHELFKPY | | CCAGCTTCTACCGCTCCAGTTGTTTCTGAAGCTCAAAAGAGACACGCTG |
| | | | ICLIPKELWYVPDPYFDM | | AATTCTTGGAAAGACACGAATTGTTCAAGCCATACATCTGTTTGATCCC |
| | | | FGYPVMALLPGLYAYLWN | | AAAGGAATTGTGGTACGTTCCAGACCCATACTTCGACATGTTCGGTTAC |
| | | | TYNIFFTPEGAWNTNKET | | CCAGTTATGGCTTTGTTGCCAGGTTTGTACGCTTACTTGTGGAACACCT |
| | | | VMSTLGELWTHAEWRDRI | | ACAACATCTTCTTCACCCCAGAAGGTGCTTGGAACACCAACAAGGAAAC |
| | | | IAKEQKETEEWERQMREW | | CGTTATGTCTACCTTGGGTGAATTGTGGACCCACGCTGAATGGAGAGAC |
| | | | EEENAEDDEGLSIDDMEN | | AGAATCATCGCTAAGGAACAAAAGGAAACCGAAGAATGGGAAAGACAAA |
| | | | YGKGKKKNKNKKKKDKAK | | TGAGAGAATGGGAAGAAGAAAACGCTGAAGACGACGAAGGTTTGTCTAT |
| | | | RPPPPPPKSLSSYKRFEK | | CGACGACATGGAAAACTACGGTAAGGGTAAGAAGAAGAACAAGAACAAG |
| | | | KKEDVVVPKKNIGFKEVS | | AAGAAGAAGGACAAGGCTAAAAGACCACCACCACCACCACCAAAGTCTT |
| | | | EITFEEEVVEVDETRQPS | | TGTCTTCTTACAAGAGATTCGAAAAGAAGAAGGAAGACGTTGTTGTTCC |
| | | | SLVFIGPVDAVKSTICGN | | AAAGAAGAACATCGGTTTCAAGGAAGTTTCTGAAATCACCTTCGAAGAA |
| | | | LMFMTGMVDERTIEKFKQ | | GAAGTTGTTGAAGTTGACGAAACCAGACAACCATCTTCTTTGGTTTTCA |
| | | | EAKEKNRDSWWLAYVMDI | | TCGGTCCAGTTGACGCTGTTAAGTCTACCATCTGTGGTAACTTGATGTT |
| | | | NDDEKSKGKTVEVGRATM | | CATGACCGGTATGGTTGACGAAAGAACCATCGAAAAGTTCAAGCAAGAA |
| | | | ETPTKRYTIFDAPGHKNY | | GCTAAGGAAAAGAACAGAGACTCTTGGTGGTTGGCTTACGTTATGGACA |
| | | | VPDMIMGAAMADVAALVI | | TCAACGACGACGAAAAGTCTAAGGGTAAGACCGTTGAAGTTGGTAGAGC |
| | | | SARKGEFEAGFERDGQTR | | TACCATGGAAACCCCAACCAAGAGATACACCATCTTCGACGCTCCAGGT |
| | | | EHAQLARSLGVNKLVVVV | | CACAAGAACTACGTTCCAGACATGATCATGGGTGCTGCTATGGCTGACG |
| | | | NEMDEETVQWSEERYNDI | | TTGCTGCTTTGGTTATCTCTGCTAGAAAGGGTGAATTCGAAGCTGGTTT |
| | | | LSGVTPFLIDQCGYKRED | | CGAACGCGACGGTCAAACCAGAGAACACGCTCAATTGGCTAGATCTTTG |
| | | | LIFVPISGLNGHNIDKLA | | GGTGTTAACAAGTTGGTTGTTGTTGTTAACGAAATGGACGAAGAAACCG |
| | | | SCCPWYTGPTLLEILDCI | | TTCAATGGTCTGAAGAAAGATACAACGACATCTTGTCTGGTGTTACCCC |
| | | | EPPKRNIDGPLRVPVLDK | | ATTCTTGATCGACCAATGTGGTTACAAGAGAGAAGACTTGATCTTCGTT |
| | | | MKDRGVVAFGKVESGVIR | | CCAATCTCTGGTTTGAACGGTCACAACATCGACAAGTTGGCTTCTTGTT |
| | | | IGPKLAVMPNNTKCQVVG | | GTCCATGGTACACCGGTCCAACCTTGTTGGAAATCTTGGACTGTATCGA |
| | | | IYNCKLELVRYANPGENI | | ACCACCAAAGAGAAACATCGACGGTCCATTGAGAGTTCCAGTTTTGGAC |
| | | | QIKVRMIEDENQINKGDV | | AAGATGAAGGACAGAGGTGTTGTTGCTTTCGGTAAGGTTGAATCTGGTG |
| | | | LCPYDNLAPITDLFEAEL | | TTATCAGAATCGGTCCAAAGTTGGCTGTTATGCCAAACAACACCAAGTG |
| | | | TILELLPHRPIITPGYKS | | TCAAGTTGTTGGTATCTACAACTGTAAGTTGGAATTGGTTAGATACGCT |
| | | | MMHLHTISDEIVIQTLTG | | AACCCAGGTGAAAACATCCAAATCAAGGTTAGAATGATCGAAGACGAAA |
| | | | IYELDGSGKEYVKKNPKY | | ACCAAATCAACAAGGGTGACGTTTTGTGTCCATACGACAACTTGGCTCC |
| | | | CKSGSKVIVKISTRVPVC | | AATCACCGACTTGTTCGAAGCTGAATTGACCATCTTGGAATTGTTGCCA |
| | | | LEKYEFIVHMGRFTLRDE | | CACAGACCAATCATCACCCCAGGTTACAAGTCTATGATGCACTTGCACA |
| | | | GKTIALGKVLRYKPAVIK | | CCATCTCTGACGAAATCGTTATCCAAACCTTGACCGGTATCTACGAATT |
| | | | KVEEIPPGVGDEGQAKLE | | GGACGGTTCTGGTAAGGAATACGTTAAGAAGAACCCAAAGTACTGTAAG |
| | | | ESEEFSGSRGDSPSKDDN | | TCTGGTTCTAAGGTTATCGTTAAGATCTCTACCAGAGTTCCAGTTTGTT |
| | | | KYEVITYDPEEDTIIASS | | TGGAAAAGTACGAATTCATCGTTCACATGGGTAGATTCACCTTGCGCGA |
| | | | TGSENAE*** | | CGAAGGTAAGACCATCGCTTTGGGTAAGGTTTTGAGATACAAGCCAGCT |
| | | | | | GTTATCAAGAAGGTTGAAGAAATCCCACCAGGTGTTGGTGACGAAGGTC |
| | | | | | AAGCTAAGTTGGAAGAATCTGAAGAATTCTCTGGTTCTAGAGGTGACTC |
| | | | | | TCCATCTAAGGACGACAACAAGTACGAAGTTATCACCTACGACCCAGAA |
| | | | | | GAAGACACCATCATCGCTTCTTCTACCGGTTCTGAAAACGCTGAAtaat |
| | | | | | ag |
| 42 | Bja_eRF1_ | SEQ ID NO: | MEGDELTQNIEQWKIKRL | SEQ ID NO: | ATGGAAGGTGACGAATTGACCCAAAACATCGAACAATGGAAGATCAAGA |
| | CAC16186.2/ | 81 | IDNLDKARGNGTSLISLI | 142 | GATTGATCGACAACTTGGACAAGGCTAGAGGTAACGGTACCTCTTTGAT |
| | Bja_eRF3_ | | IPPREQLPIINKMITEEY | | CTCTTTGATCATCCCACCAAGAGAACAATTGCCAATCATCAACAAGATG |
| | AAD03251.1 | | GKSSNIKSRIVRQAVQSA | | ATCACCGAAGAATACGGTAAGTCTTCTAACATCAAGTCTAGAATCGTTA |
| | | | LTSTKERLKLYNNRLPAN | | GACAAGCTGTTCAATCTGCTTTGACCTCTACCAAGGAAAGATTGAAGTT |
| | | | GLILYCGEVINEEGVCEK | | GTACAACAACAGATTGCCAGCTAACGGTTTGATCTTGTACTGTGGTGAA |
| | | | KYTIDFQPYRAINTTLYI | | GTTATCAACGAAGAAGGTGTTTGTGAAAAGAAGTACACCATCGACTTCC |
| | | | CDNKFHTQPLKDLLVMDD | | AACCATACAGAGCTATCAACACCACCTTGTACATCTGTGACAACAAGTT |
| | | | KFGFIIIDGNGALFGTLQ | | CCACACCCAACCATTGAAGGACTTGTTGGTTATGGACGACAAGTTCGGT |
| | | | GNTREVLHKFSVDLPKKH | | TTCATCATCATCGACGGTAACGGTGCTTTGTTCGGTACCTTGCAAGGTA |
| | | | RRGGQSALRFARLRMESR | | ACACCAGAGAAGTTTTGCACAAGTTCTCTGTTGACTTGCCAAAGAAGCA |
| | | | NNYLRKVAEQAVVQFISN | | CAGAAGAGGTGGTCAATCTGCTTTGAGATTCGCTAGATTGAGAATGGAA |
| | | | DKVNVAGLIVAGSAEFKN | | TCTAGAAACAACTACTTGAGAAAGGTTGCTGAACAAGCTGTTGTTCAAT |
| | | | VLVQSDLFDQRLAAKVLK | | TCATCTCTAACGACAAGGTTAACGTTGCTGGTTTGATCGTTGCTGGTTC |
| | | | IVDVAYGGENGFTQAIEL | | TGCTGAATTCAAGAACGTTTTGGTTCAATCTGACTTGTTCGACCAAAGA |
| | | | SADTLSNIKFIREKKVMS | | TTGGCTGCTAAGGTTTTGAAGATCGTTGACGTTGCTTACGGTGGTGAAA |
| | | | KFFEEVAQDTKKYCYGVE | | ACGGTTTCACCCAAGCTATCGAATTGTCTGCTGACACCTTGTCTAACAT |
| | | | DTMKTLIMGAVEVILLFE | | CAAGTTCATCAGAGAAAAGAAGGTTATGTCTAAGTTCTTCGAAGAAGTT |
| | | | NLNFTRYVLKNPTTGVEK | | GCTCAAGACACCAAGAAGTACTGTTACGGTGTTGAAGACACCATGAAGA |
| | | | TLYLTPEQEENHDNFMEN | | CCTTGATCATGGGTGCTGTTGAAGTTATCTTGTTGTTCGAAAACTTGAA |
| | | | GEELEALEKGPLPEWIVD | | CTTCACCAGATACGTTTTGAAGAACCCAACCACCGGTGTTGAAAAGACC |
| | | | NYMKFGAGLEFITDRSQE | | TTGTACTTGACCCCAGAACAAGAAGAAAACCACGACAACTTCATGGAAA |
| | | | GAQFVRGFGGLGAFLRYQ | | ACGGTGAAGAATTGGAAGCTTTGGAAAAGGGTCCATTGCCAGAATGGAT |
| | | | VDMAHLNAGEEELDEEWD | | CGTTGACAACTACATGAAGTTCGGTGCTGGTTTGGAATTCATCACCGAC |
| | | | DDFM** | | AGATCTCAAGAAGGTGCTCAATTCGTTAGAGGTTTCGGTGGTTTGGGTG |
| | | | | | CTTTCTTGAGATACCAAGTTGACATGGCTCACTTGAACGCTGGTGAAGA |
| | | | | | AGAATTGGACGAAGAATGGGACGACGACTTCATGtaatag |
| 43 | Bja_eRF1_ | SEQ ID NO: | MVDSGKSTSCGHLIYKCG | SEQ ID NO: | ATGGTTGACTCTGGTAAGTCTACCTCTTGTGGTCACTTGATCTACAAGT |
| | CAC16186.2/ | 82 | GIDKRTIEKYEKEAKEMG | 143 | GTGGTGGTATCGACAAGAGAACCATCGAAAAGTACGAAAAGGAAGCTAA |
| | Bja_eRF3_ | | KSSFKYAWVLDKLKAERE | | GGAAATGGGTAAGTCTTCTTTCAAGTACGCTTGGGTTTTGGACAAGTTG |
| | AAD03251.1 | | RGITIDISLFKFQTDKFY | | AAGGCTGAAAGAGAAAGAGGTATCACCATCGACATCTCTTTGTTCAAGT |
| | | | FTIIDAPGHRDFIKNMIT | | TCCAAACCGACAAGTTCTACTTCACCATCATCGACGCTCCAGGTCACAG |
| | | | GTSQADVAILIIAAGKGE | | AGACTTCATCAAGAACATGATCACCGGTACCTCTCAAGCTGACGTTGCT |
| | | | FEAGYSKNGQTREHALLA | | ATCTTGATCATCGCTGCTGGTAAGGGTGAATTCGAAGCTGGTTACTCTA |
| | | | FTLGVKQMVVGVNKMDDK | | AGAACGGTCAAACCAGAGAACACGCTTTGTTGGCTTTCACCTTGGGTGT |
| | | | SAEWKQDRYLEIKQEVSE | | TAAGCAAATGGTTGTTGGTGTTAACAAGATGGACGACAAGTCTGCTGAA |
| | | | YLKKVGYNPAKVPFIPIS | | TGGAAGCAAGACAGATACTTGGAAATCAAGCAAGAAGTTTCTGAATACT |
| | | | GWLGDNMVEKSTNMPWYD | | TGAAGAAGGTTGGTTACAACCCAGCTAAGGTTCCATTCATCCCAATCTC |
| | | | GPTLLGALDNVQPPKRHV | | TGGTTGGTTGGGTGACAACATGGTTGAAAAGTCTACCAACATGCCATGG |
| | | | DKPLRLPVQDVYKISGIG | | TACGACGGTCCAACCTTGTTGGGTGCTTTGGACAACGTTCAACCACCAA |
| | | | TVPVGRVETGVLRPGTVV | | AGAGACACGTTGACAAGCCATTGAGATTGCCAGTTCAAGACGTTTACAA |
| | | | VFAPSGISTEVKSVEMHH | | GATCTCTGGTATCGGTACCGTTCCAGTTGGTAGAGTTGAAACCGGTGTT |
| | | | ESLEEALPGDNVGFNIKN | | CTCAGACCAGGTACCGTTGTTGTTTTCGCTCCATCTGGTATCTCTACCG |
| | | | IAVNQIKRGYVASDSRSD | | AAGTTAAGTCTGTTGAAATGCACCACGAATCTTTGGAAGAAGCTTTGCC |
| | | | PARESIDFTAQVIILHHP | | AGGTGACAACGTTGGTTTCAACATCAAGAACATCGCTGTTAACCAAATC |
| | | | GQISVGYTPVLDCHTAHI | | AAGAGAGGTTACGTTGCTTCTGACTCTAGATCTGACCCAGCTAGAGAAT |
| | | | ACRFNDLKQKVDRRSGAV | | CTATCGACTTCACCGCTCAAGTTATCATCTTGCACCACCCAGGTCAAAT |
| | | | LEQEPKFVKSGDAAIVTL | | CTCTGTTGGTTACACCCCAGTTTTGGACTGTCACACCGCTCACATCGCT |
| | | | IPTKSMCVEPFTEYPPLG | | TGTAGATTCAACGACTTGAAGCAAAAGGTTGACAGAAGATCTGGTGCTG |
| | | | RFAVRDMRQTVAV*** | | TTTTGGAACAAGAACCAAAGTTCGTTAAGTCTGGTGACGCTGCTATCGT |
| | | | | | TACCTTGATCCCAACCAAGTCTATGTGTGTTGAACCATTCACCGAATAC |
| | | | | | CCACCATTGGGTAGATTCGCTGTTAGAGACATGAGACAAACCGTTGCTG |
| | | | | | TTtaatag |
| 44 | Tth_eRF1_ | SEQ ID NO: | MEEKDQRQRNIEHFKIKK | SEQ ID NO: | ATGGAAGAAAAGGACCAAAGACAAAGAAACATCGAACACTTCAAGATCA |
| | XP_ | 83 | LMTRLRNTRGSGTSMVSL | 144 | AGAAGTTGATGACCAGATTGAGAAACACCAGAGGTTCTGGTACCTCTAT |
| | 001018735. | | IIPPKKQINDSTKLISDE | | GGTTTCTTTGATCATCCCACCAAAGAAGCAAATCAACGACTCTACCAAG |
| | 1/ | | FSKATNIKDRVNRQSVQD | | TTGATCTCTGACGAATTCTCTAAGGCTACCAACATCAAGGACAGAGTTA |
| | Tth_eRF3_ | | AMVSALQRLKLYQRTPNN | | ACAGACAATCTGTTCAAGACGCTATGGTTTCTGCTTTGCAAAGATTGAA |
| | XP_ | | GLILYCGKVLNEEGKEIK | | GTTGTACCAAAGAACCCCAAACAACGGTTTGATCTTGTACTGTGGTAAG |
| | 001011280.3 | | LLIDFEPYKPINTSLYFC | | GTTTTGAACGAAGAAGGTAAGGAAATCAAGTIGTTGATCGACTTCGAAC |
| | | | DSKFHVDELGSLLETDPP | | CATACAAGCCAATCAACACCTCTTTGTACTTCTGTGACTCTAAGTTCCA |
| | | | FGFIVMDGQGALYANLQG | | CGTTGACGAATTGGGTTCTTTGTTGGAAACCGACCCACCATTCGGTTTC |
| | | | NTKTVLNKFSVELPKKHG | | ATCGTTATGGACGGTCAAGGTGCTTTGTACGCTAACTTGCAAGGTAACA |
| | | | RGGQSSVRFARLRVEKRH | | CCAAGACCGTTTTGAACAAGTTCTCTGTTGAATTGCCAAAGAAGCACGG |
| | | | NYLRKVCEVATQTFISQD | | TAGAGGTGGTCAATCTTCTGTTAGATTCGCTAGATTGAGAGTTGAAAAG |
| | | | KINVQGLVLAGSGDFKNE | | AGACACAACTACTTGAGAAAGGTTTGTGAAGTTGCTACCCAAACCTTCA |
| | | | LSTTQMFDPRLACKIIKI | | TCTCTCAAGACAAGATCAACGTTCAAGGTTTGGTTTTGGCTGGTTCTGG |
| | | | VDVSYGGENGLNQAIELA | | TGACTTCAAGAACGAATTGTCTACCACCCAAATGTTCGACCCAAGATTG |
| | | | QESLTNVKFVQEKNVISK | | GCTTGTAAGATCATCAAGATCGTTGACGTTTCTTACGGTGGTGAAAACG |
| | | | FFDCIAIDSGTVVYGVQD | | GTTTGAACCAAGCTATCGAATTGGCTCAAGAATCTTTGACCAACGTTAA |
| | | | TMQLLLDGVIENILCFEE | | GTTCGTTCAAGAAAAGAACGTTATCTCTAAGTTCTTCGACTGTATCGCT |
| | | | LTTLRVTRKNKVTEQITH | | ATCGACTCTGGTACCGTTGTTTACGGTGTTCAAGACACCATGCAATTGT |
| | | | IFIPPNELNNPKHFKDGE | | TGTTGGACGGTGTTATCGAAAACATCTTGTGTTTCGAAGAATTGACCAC |
| | | | HELEKIEVENLTEWLAEH | | CTTGAGAGTTACCAGAAAGAACAAGGTTACCGAACAAATCACCCACATC |
| | | | YSEFGAELYFITDKSAEG | | TTCATCCCACCAAACGAATTGAACAACCCAAAGCACTTCAAGGACGGTG |
| | | | CQFVKGFSGIGGFLRYKV | | AACACGAATTGGAAAAGATCGAAGTTGAAAACTTGACCGAATGGTTGGC |
| | | | DLEHIVNPNDEYNYEEEE | | TGAACACTACTCTGAATTCGGTGCTGAATTGTACTTCATCACCGACAAG |
| | | | GFI** | | TCTGCTGAAGGTTGTCAATTCGTTAAGGGTTTCTCTGGTATCGGTGGTT |
| | | | | | TCTTGAGATACAAGGTTGACTTGGAACACATCGTTAACCCAAACGACGA |
| | | | | | ATACAACTACGAAGAAGAAGAAGGTTTCATCtgatag |
| 45 | Tth_eRF1_ | SEQ ID NO: | MDYQEAKRLAKEEKLRKL | SEQ ID NO: | ATGGACTACCAAGAAGCTAAGAGATTGGCTAAGGAAGAAAAGTTGAGAA |
| | XP_ | 84 | KAKIQENKVEDFQVTEQQ | 145 | AGTTGAAGGCTAAGATCCAAGAAAACAAGGTTGAAGACTTCCAAGTTAC |
| | 001018735. | | GPLPPYDDQGNQQGKWLT | | CGAACAACAAGGTCCATTGCCACCATACGACGACCAAGGTAACCAACAA |
| | 1/ | | TKYKFGEWYYDMNGNPVF | | GGTAAGTGGTTGACCACCAAGTACAAGTTCGGTGAATGGTACTACGACA |
| | Tth_eRF3_ | | ISPDDEYDONGSLYDAES | | TGAACGGTAACCCAGTTTTCATCTCTCCAGACGACGAATACGACCAAAA |
| | XP_ | | NPYLDYAYDKFGNPCPVI | | CGGTTCTTTGTACGACGCTGAATCTAACCCATACTTGGACTACGCTTAC |
| | 001011280.3 | | LMTQDNEYFLFTPIFEIQ | | GACAAGTTCGGTAACCCATGTCCAGTTATCTTGATGACCCAAGACAACG |
| | | | IPQSIIKDINQKQEKPAT | | AATACTTCTTGTTCACCCCAATCTTCGAAATCCAAATCCCACAATCTAT |
| | | | QKVETVAKEKKAPIQKAP | | CATCAAGGACATCAACCAAAAGCAAGAAAAGCCAGCTACCCAAAAGGTT |
| | | | APPKIPKRLLKEQEAANS | | GAAACCGTTGCTAAGGAAAAGAAGGCTCCAATCCAAAAGGCTCCAGCTC |
| | | | SAVVADYKHEKLVTVQEA | | CACCAAAGATCCCAAAGAGATTGTTGAAGGAACAAGAAGCTGCTAACTC |
| | | | IDFESKNFKEGQEMVKVD | | TTCTGCTGTTGTTGCTGACTACAAGCACGAAAAGTTGGTTACCGTTCAA |
| | | | RERDSVNIVFIGHVDAGK | | GAAGCTATCGACTTCGAATCTAAGAACTTCAAGGAAGGTCAAGAAATGG |
| | | | STLSGRILKNCGEVDETE | | TTAAGGTTGACAGAGAAAGAGACTCTGTTAACATCGTTTTCATCGGTCA |
| | | | IRKFELEAKEKNRESWVL | | CGTTGACGCTGGTAAGTCTACCTTGTCTGGTAGAATCTTGAAGAACTGT |
| | | | AYIMDINEEERSKGITVE | | GGTGAAGTTGACGAAACCGAAATCAGAAAGTTCGAATTGGAAGCTAAGG |
| | | | CGKAHFQLANKRFVLLDA | | AAAAGAACAGAGAATCTTGGGTTTTGGCTTACATCATGGACATCAACGA |
| | | | PGHKNYVPNMIAGACQAD | | AGAAGAAAGATCTAAGGGTATCACCGTTGAATGTGGTAAGGCTCACTTC |
| | | | VAALIISARQGEFEAGFE | | CAATTGGCTAACAAGAGATTCGTTTTGTTGGACGCTCCAGGTCACAAGA |
| | | | GGQTQEHAHLAKALGVQH | | ACTACGTTCCAAACATGATCGCTGGTGCTTGTCAAGCTGACGTTGCTGC |
| | | | MICVVSKMDEVNWDKKRY | | TTTGATCATCTCTGCTAGACAAGGTGAATTCGAAGCTGGTTTCGAAGGT |
| | | | DHIHDSVEPFLRNQVGIQ | | GGTCAAACCCAAGAACACGCTCACTTGGCTAAGGCTTTGGGTGTTCAAC |
| | | | SIEWVPINGFLNENIDTP | | ACATGATCTGTGTTGTTTCTAAGATGGACGAAGTTAACTGGGACAAGAA |
| | | | IPTERCEWYKGDTLFDKF | | GAGATACGACCACATCCACGACTCTGTTGAACCATTCTTGAGAAACCAA |
| | | | NKVPVPLRDPNGPVRIPV | | GTTGGTATCCAATCTATCGAATGGGTTCCAATCAACGGTTTCTTGAACG |
| | | | LDKLKDQGQFLFGKIESG | | AAAACATCGACACCCCAATCCCAACCGAAAGATGTGAATGGTACAAGGG |
| | | | TIRDDLWVTLMPYRKQFQ | | TGACACCTTGTTCGACAAGTTCAACAAGGTTCCAGTTCCATTGCGCGAC |
| | | | ILSIYNTKDQRVLYASAG | | CCAAACGGTCCAGTTAGAATCCCAGTTTTGGACAAGTTGAAGGACCAAG |
| | | | ENVKIKLKGLEDKDIERG | | GTCAATTCTTGTTCGGTAAGATCGAATCTGGTACCATCCGCGACGACTT |
| | | | YMVCSTEDLCPITQLFIA | | GTGGGTTACCTTGATGCCATACAGAAAGCAATTCCAAATCTTGTCTATC |
| | | | EITILQLPEHKPIMSQGY | | TACAACACCAAGGACCAAAGAGTTTTGTACGCTTCTGCTGGTGAAAACG |
| | | | SCVLHMHTSVAEIEIEEV | | TTAAGATCAAGTTGAAGGGTTTGGAAGACAAGGACATCGAAAGAGGTTA |
| | | | EAVQNPENKKLTKNTFLK | | CATGGTTTGTTCTACCGAAGACTTGTGTCCAATCACCCAATTGTTCATC |
| | | | SNQTGVVKIGIKGGLMCL | | GCTGAAATCACCATCTTGCAATTGCCAGAACACAAGCCAATCATGTCTC |
| | | | EKFETISQLGRFTLRDEE | | AAGGTTACTCTTGTGTTTTGCACATGCACACCTCTGTTGCTGAAATCGA |
| | | | KTIGFGRVMKIKPYKV* | | AATCGAAGAAGTTGAAGCTGTTCAAAACCCAGAAAACAAGAAGTTGACC |
| | | | | | AAGAACACCTTCTTGAAGTCTAACCAAACCGGTGTTGTTAAGATCGGTA |
| | | | | | TCAAGGGTGGTTTGATGTGTTTGGAAAAGTTCGAAACCATCTCTCAATT |
| | | | | | GGGTAGATTCACCTTGCGCGACGAAGAAAAGACCATCGGTTTCGGTAGA |
| | | | | | GTTATGAAGATCAAGCCATACAAGGTTtga |
| 46 | Tth_eRF1_XP_ | SEQ ID NO: | MEQKPPFQNPLQKLQDRG | SEQ ID NO: | ATGGAACAAAAGCCACCATTCCAAAACCCATTGCAAAAGTTGCAAGACA |
| | 001018211. | 85 | TKMDQSSGSCMSKQAEEQ | 146 | GAGGTACCAAGATGGACCAATCTTCTGGTTCTTGTATGTCTAAGCAAGC |
| | 4/ | | KRLQISQYQLRKQLQMLR | | TGAAGAACAAAAGAGATTGCAAATCTCTCAATACCAATTGAGAAAGCAA |
| | Tth_eRF3_XP_ | | NMRGEQTSCVSLYIPERK | | TTGCAAATGTTGAGAAACATGAGAGGTGAACAAACCTCTTGTGTTTCTT |
| | 001011280. | | KLYEVVNYLQQEESGAAS | | TGTACATCCCAGAAAGAAAGAAGTTGTACGAAGTTGTTAACTACTTGCA |
| | 3 | | IKNTQNRKSVQSALSMLR | | ACAAGAAGAATCTGGTGCTGCTTCTATCAAGAACACCCAAAACAGAAAG |
| | | | ERLKNFNLHKKYPKGMIF | | TCTGTTCAATCTGCTTTGTCTATGTTGAGAGAAAGATTGAAGAACTTCA |
| | | | FCADSLDSKRLLIEILDP | | ACTTGCACAAGAAGTACCCAAAGGGTATGATCTTCTTCTGTGCTGACTC |
| | | | PKAVQSFRYSCNTIFYLD | | TTTGGACTCTAAGAGATTGTTGATCGAAATCTTGGACCCACCAAAGGCT |
| | | | DLEYMLKDQPTYGFVVAD | | GTTCAATCTTTCAGATACTCTTGTAACACCATCTTCTACTTGGACGACT |
| | | | GHGYLIATVCGFDIQILQ | | TGGAATACATGTTGAAGGACCAACCAACCTACGGTTTCGTTGTTGCTGA |
| | | | SKQEDLPNKHNKGGQSSL | | CGGTCACGGTTACTTGATCGCTACCGTTTGTGGTTTCGACATCCAAATC |
| | | | RFSRLCDAARERLVKNIA | | TTGCAATCTAAGCAAGAAGACTTGCCAAACAAGCACAACAAGGGTGGTC |
| | | | DAMRRCYANENGTQTNLS | | AATCTTCTTTGAGATTCTCTAGATTGTGTGACGCTGCTAGAGAAAGATT |
| | | | GIVLCGMSDIKDKVQKEL | | GGTTAAGAACATCGCTGACGCTATGAGAAGATGTTACGCTAACGAAAAC |
| | | | QQLCPCIENKIVASYDVS | | GGTACCCAAACCAACTTGTCTGGTATCGTTTTGTGTGGTATGTCTGACA |
| | | | YSGQAGLKQALQMSTEML | | TCAAGGACAAGGTTCAAAAGGAATTGCAACAATTGTGTCCATGTATCGA |
| | | | KLDQLFQEMNLLSDFFAN | | AAACAAGATCGTTGCTTCTTACGACGTTTCTTACTCTGGTCAAGCTGGT |
| | | | FSLETSKVVYGGELTVRA | | TTGAAGCAAGCTTTGCAAATGTCTACCGAAATGTTGAAGTTGGACCAAT |
| | | | LEEGNVKKLILCQDSELQ | | TGTTCCAAGAAATGAACTTGTTGTCTGACTTCTTCGCTAACTTCTCTTT |
| | | | RVTVYNSKTQEETIQYLM | | GGAAACCTCTAAGGTTGTTTACGGTGGTGAATTGACCGTTAGAGCTTTG |
| | | | PSQVKALQDSISKTSDQE | | GAAGAAGGTAACGTTAAGAAGTTGATCTTGTGTCAAGACTCTGAATTGC |
| | | | ANNKKNQLQVYSQQNINE | | AAAGAGTTACCGTTTACAACTCTAAGACCCAAGAAGAAACCATCCAATA |
| | | | WIVENISSFSQDLEIVFV | | CTTGATGCCATCTCAAGTTAAGGCTTTGCAAGACTCTATCTCTAAGACC |
| | | | SDKTQQGVQFSKSFQGVG | | TCTGACCAAGAAGCTAACAACAAGAAGAACCAATTGCAAGTTTACTCTC |
| | | | AYLKYSLDYSSLHAQEKE | | AACAAAACATCAACGAATGGATCGTTGAAAACATCTCTTCTTTCTCTCA |
| | | | NDQLEQEYCYDDEEGFI* | | AGACTTGGAAATCGTTTTCGTTTCTGACAAGACCCAACAAGGTGTTCAA |
| | | | * | | TTCTCTAAGTCTTTCCAAGGTGTTGGTGCTTACTTGAAGTACTCTTTGG |
| | | | | | ACTACTCTTCTTTGCACGCTCAAGAAAAGGAAAACGACCAATTGGAACA |
| | | | | | AGAATACTGTTACGACGACGAAGAAGGTTTCATCtgatag |
| 47 | Tth_eRF1_XP_ | SEQ ID NO: | MDYQEAKRLAKEEKLRKL | SEQ ID NO: | ATGGACTACCAAGAAGCTAAGAGATTGGCTAAGGAAGAAAAGTTGAGAA |
| | 001018211. | 86 | KAKIQENKVEDFQVTEQQ | 147 | AGTTGAAGGCTAAGATCCAAGAAAACAAGGTTGAAGACTTCCAAGTTAC |
| | 4/ | | GPLPPYDDQGNQQGKWLT | | CGAACAACAAGGTCCATTGCCACCATACGACGACCAAGGTAACCAACAA |
| | Tth_eRF3_XP_ | | TKYKFGEWYYDMNGNPVF | | GGTAAGTGGTTGACCACCAAGTACAAGTTCGGTGAATGGTACTACGACA |
| | 001011280. | | ISPDDEYDQNGSLYDAES | | TGAACGGTAACCCAGTTTTCATCTCTCCAGACGACGAATACGACCAAAA |
| | 3 | | NPYLDYAYDKFGNPCPVI | | CGGTTCTTTGTACGACGCTGAATCTAACCCATACTTGGACTACGCTTAC |
| | | | LMTQDNEYFLFTPIFEIQ | | GACAAGTTCGGTAACCCATGTCCAGTTATCTTGATGACCCAAGACAACG |
| | | | IPQSIIKDINQKQEKPAT | | AATACTTCTTGTTCACCCCAATCTTCGAAATCCAAATCCCACAATCTAT |
| | | | QKVETVAKEKKAPIQKAP | | CATCAAGGACATCAACCAAAAGCAAGAAAAGCCAGCTACCCAAAAGGTT |
| | | | APPKIPKRLLKEQEAANS | | GAAACCGTTGCTAAGGAAAAGAAGGCTCCAATCCAAAAGGCTCCAGCTC |
| | | | SAVVADYKHEKLVTVQEA | | CACCAAAGATCCCAAAGAGATTGTTGAAGGAACAAGAAGCTGCTAACTC |
| | | | IDFESKNFKEGQEMVKVD | | TTCTGCTGTTGTTGCTGACTACAAGCACGAAAAGTTGGTTACCGTTCAA |
| | | | RERDSVNIVFIGHVDAGK | | GAAGCTATCGACTTCGAATCTAAGAACTTCAAGGAAGGTCAAGAAATGG |
| | | | STLSGRILKNCGEVDETE | | TTAAGGTTGACAGAGAAAGAGACTCTGTTAACATCGTTTTCATCGGTCA |
| | | | IRKFELEAKEKNRESWVL | | CGTTGACGCTGGTAAGTCTACCTTGTCTGGTAGAATCTTGAAGAACTGT |
| | | | AYIMDINEEERSKGITVE | | GGTGAAGTTGACGAAACCGAAATCAGAAAGTTCGAATTGGAAGCTAAGG |
| | | | CGKAHFQLANKRFVLLDA | | AAAAGAACAGAGAATCTTGGGTTTTGGCTTACATCATGGACATCAACGA |
| | | | PGHKNYVPNMIAGACQAD | | AGAAGAAAGATCTAAGGGTATCACCGTTGAATGTGGTAAGGCTCACTTC |
| | | | VAALIISARQGEFEAGFE | | CAATTGGCTAACAAGAGATTCGTTTTGTTGGACGCTCCAGGTCACAAGA |
| | | | GGQTQEHAHLAKALGVQH | | ACTACGTTCCAAACATGATCGCTGGTGCTTGTCAAGCTGACGTTGCTGC |
| | | | MICVVSKMDEVNWDKKRY | | TTTGATCATCTCTGCTAGACAAGGTGAATTCGAAGCTGGTTTCGAAGGT |
| | | | DHIHDSVEPFLRNQVGIQ | | GGTCAAACCCAAGAACACGCTCACTTGGCTAAGGCTTTGGGTGTTCAAC |
| | | | SIEWVPINGFLNENIDTP | | ACATGATCTGTGTTGTTTCTAAGATGGACGAAGTTAACTGGGACAAGAA |
| | | | IPTERCEWYKGDTLFDKF | | GAGATACGACCACATCCACGACTCTGTTGAACCATTCTTGAGAAACCAA |
| | | | NKVPVPLRDPNGPVRIPV | | GTTGGTATCCAATCTATCGAATGGGTTCCAATCAACGGTTTCTTGAACG |
| | | | LDKLKDQGQFLFGKIESG | | AAAACATCGACACCCCAATCCCAACCGAAAGATGTGAATGGTACAAGGG |
| | | | TIRDDLWVTLMPYRKQFQ | | TGACACCTTGTTCGACAAGTTCAACAAGGTTCCAGTTCCATTGCGCGAC |
| | | | ILSIYNTKDQRVLYASAG | | CCAAACGGTCCAGTTAGAATCCCAGTTTTGGACAAGTTGAAGGACCAAG |
| | | | ENVKIKLKGLEDKDIERG | | GTCAATTCTTGTTCGGTAAGATCGAATCTGGTACCATCCGCGACGACTT |
| | | | YMVCSTEDLCPITQLFIA | | GTGGGTTACCTTGATGCCATACAGAAAGCAATTCCAAATCTTGTCTATC |
| | | | EITILQLPEHKPIMSQGY | | TACAACACCAAGGACCAAAGAGTTTTGTACGCTTCTGCTGGTGAAAACG |
| | | | SCVLHMHTSVAEIEIEEV | | TTAAGATCAAGTTGAAGGGTTTGGAAGACAAGGACATCGAAAGAGGTTA |
| | | | EAVQNPENKKLTKNTFLK | | CATGGTTTGTTCTACCGAAGACTTGTGTCCAATCACCCAATTGTTCATC |
| | | | SNQTGVVKIGIKGGLMCL | | GCTGAAATCACCATCTTGCAATTGCCAGAACACAAGCCAATCATGTCTC |
| | | | EKFETISQLGRFTLRDEE | | AAGGTTACTCTTGTGTTTTGCACATGCACACCTCTGTTGCTGAAATCGA |
| | | | KTIGFGRVMKIKPYKV* | | AATCGAAGAAGTTGAAGCTGTTCAAAACCCAGAAAACAAGAAGTTGACC |
| | | | | | AAGAACACCTTCTTGAAGTCTAACCAAACCGGTGTTGTTAAGATCGGTA |
| | | | | | TCAAGGGTGGTTTGATGTGTTTGGAAAAGTTCGAAACCATCTCTCAATT |
| | | | | | GGGTAGATTCACCTTGCGCGACGAAGAAAAGACCATCGGTTTCGGTAGA |
| | | | | | GTTATGAAGATCAAGCCATACAAGGTTtga |
| 48 | Tth_eRF1_XP_ | SEQ ID NO: | MIKNIFKLLPISLRAIPL | SEQ ID NO: | ATGATCAAGAACATCTTCAAGTTGTTGCCAATCTCTTTGAGAGCTATCC |
| | 001008252. | 87 | KQQQNSFSQICSLYNTKL | 148 | CATTGAAGCAACAACAAAACTCTTTCTCTCAAATCTGTTCTTTGTACAA |
| | 2/ | | FKVINLIQTNNKCFFSFR | | CACCAAGTTGTTCAAGGTTATCAACTTGATCCAAACCAACAACAAGTGT |
| | Tth_eRF3_XP_ | | AKETFKKKTSSLEIETHE | | TTCTTCTCTTTCAGAGCTAAGGAAACCTTCAAGAAGAAGACCTCTTCTT |
| | 001011280. | | QVSDLTRCIYRRMKQFHN | | TGGAAATCGAAACCCACTTCCAAGTTTCTGACTTGACCAGATGTATCTA |
| | 3 | | EYTDIQKILSQEQQQADI | | CAGAAGAATGAAGCAATTCCACAACGAATACACCGACATCCAAAAGATC |
| | | | NLEQLRKKINVLQPLNDV | | TTGTCTCAAGAACAACAACAAGCTGACATCAACTTGGAACAATTGAGAA |
| | | | FEKLEQNIKTLQELQKQK | | AGAAGATCAACGTTTTGCAACCATTGAACGACGTTTTCGAAAAGTTGGA |
| | | | EESASDPEMLALIEEEME | | ACAAAACATCAAGACCTTGCAAGAATTGCAAAAGCAAAAGGAAGAATCT |
| | | | NSKQLIDELQDECLEQLL | | GCTTCTGACCCAGAAATGTTGGCTTTGATCGAAGAAGAAATGGAAAACT |
| | | | PKGKHDDCSEITLEVRGG | | CTAAGCAATTGATCGACGAATTGCAAGACGAATGTTTGGAACAATTGTT |
| | | | AGGSESSLFAEEVFKMYQ | | GCCAAAGGGTAAGCACGACGACTGTTCTGAAATCACCTTGGAAGTTAGA |
| | | | AFFAQQGYQFSIDSFQVD | | GGTGGTGCTGGTGGTTCTGAATCTTCTTTGTTCGCTGAAGAAGTTTTCA |
| | | | MAINKGCKLGVLKVSGTN | | AGATGTACCAAGCTTTCTTCGCTCAACAAGGTTACCAATTCTCTATCGA |
| | | | IYKKMMNESGVHKVIRVP | | CTCTTTCCAAGTTGACATGGCTATCAACAAGGGTTGTAAGTTGGGTGTT |
| | | | ETESKGRLHSSTISVVVM | | TTGAAGGTTTCTGGTACCAACATCTACAAGAAGATGATGAACGAATCTG |
| | | | PVVPMDFKVDEKDLKFEF | | GTGTTCACAAGGTTATCAGAGTTCCAGAAACCGAATCTAAGGGTAGATT |
| | | | MRSQGAGGQHVNKVESAC | | GCACTCTTCTACCATCTCTGTTGTTGTTATGCCAGTTGTTCCAATGGAC |
| | | | RVTHLPTGISVLCQDDRQ | | TTCAAGGTTGACGAAAAGGACTTGAAGTTCGAATTCATGAGATCTCAAG |
| | | | QERNKQRALKLLTEKLFQ | | GTGCTGGTGGTCAACACGTTAACAAGGTTGAATCTGCTTGTAGAGTTAC |
| | | | VEVEKSNQQQSDQRKSQI | | CCACTTGCCAACCGGTATCTCTGTTTTGTGTCAAGACGACAGACAACAA |
| | | | GGGDRSDKIRTYNFPQGR | | GAAAGAAACAAGCAAAGAGCTTTGAAGTTGTTGACCGAAAAGTTGTTCC |
| | | | ITDHRTNLTLFGIEKMMK | | AAGTTGAAGTTGAAAAGTCTAACCAACAACAATCTGACCAAAGAAAGTC |
| | | | GEFLEEFIDEYEEKVNNE | | TCAAATCGGTGGTGGTGACAGATCTGACAAGATCAGAACCTACAACTTC |
| | | | LIESVLKQLEEDENQSQP | | CCACAAGGTAGAATCACCGACCACAGAACCAACTTGACCTTGTTCGGTA |
| | | | KN** | | TCGAAAAGATGATGAAGGGTGAATTCTTGGAAGAATTCATCGACGAATA |
| | | | | | CGAAGAAAAGGTTAACAACGAATTGATCGAATCTGTTTTGAAGCAATTG |
| | | | | | GAAGAAGACGAAAACCAATCTCAACCAAAGAACtgatag |
| 49 | Tth_eRF1_XP_ | SEQ ID NO: | MDYQEAKRLAKEEKLRKL | SEQ ID NO: | ATGGACTACCAAGAAGCTAAGAGATTGGCTAAGGAAGAAAAGTTGAGAA |
| | 001008252. | 88 | KAKIQENKVEDFQVTEQQ | 149 | AGTTGAAGGCTAAGATCCAAGAAAACAAGGTTGAAGACTTCCAAGTTAC |
| | 2/ | | GPLPPYDDQGNQQGKWLT | | CGAACAACAAGGTCCATTGCCACCATACGACGACCAAGGTAACCAACAA |
| | Tth_eRF3_XP_ | | TKYKFGEWYYDMNGNPVF | | GGTAAGTGGTTGACCACCAAGTACAAGTTCGGTGAATGGTACTACGACA |
| | 001011280. | | ISPDDEYDQNGSLYDAES | | TGAACGGTAACCCAGTTTTCATCTCTCCAGACGACGAATACGACCAAAA |
| | 3 | | NPYLDYAYDKFGNPCPVI | | CGGTTCTTTGTACGACGCTGAATCTAACCCATACTTGGACTACGCTTAC |
| | | | LMTQDNEYFLFTPIFEIQ | | GACAAGTTCGGTAACCCATGTCCAGTTATCTTGATGACCCAAGACAACG |
| | | | IPQSIIKDINQKQEKPAT | | AATACTTCTTGTTCACCCCAATCTTCGAAATCCAAATCCCACAATCTAT |
| | | | QKVETVAKEKKAPIQKAP | | CATCAAGGACATCAACCAAAAGCAAGAAAAGCCAGCTACCCAAAAGGTT |
| | | | APPKIPKRLLKEQEAANS | | GAAACCGTTGCTAAGGAAAAGAAGGCTCCAATCCAAAAGGCTCCAGCTC |
| | | | SAVVADYKHEKLVTVQEA | | CACCAAAGATCCCAAAGAGATTGTTGAAGGAACAAGAAGCTGCTAACTC |
| | | | IDFESKNFKEGQEMVKVD | | TTCTGCTGTTGTTGCTGACTACAAGCACGAAAAGTTGGTTACCGTTCAA |
| | | | RERDSVNIVFIGHVDAGK | | GAAGCTATCGACTTCGAATCTAAGAACTTCAAGGAAGGTCAAGAAATGG |
| | | | STLSGRILKNCGEVDETE | | TTAAGGTTGACAGAGAAAGAGACTCTGTTAACATCGTTTTCATCGGTCA |
| | | | IRKFELEAKEKNRESWVL | | CGTTGACGCTGGTAAGTCTACCTTGTCTGGTAGAATCTTGAAGAACTGT |
| | | | AYIMDINEEERSKGITVE | | GGTGAAGTTGACGAAACCGAAATCAGAAAGTTCGAATTGGAAGCTAAGG |
| | | | CGKAHFQLANKRFVLLDA | | AAAAGAACAGAGAATCTTGGGTTTTGGCTTACATCATGGACATCAACGA |
| | | | PGHKNYVPNMIAGACQAD | | AGAAGAAAGATCTAAGGGTATCACCGTTGAATGTGGTAAGGCTCACTTC |
| | | | VAALIISARQGEFEAGFE | | CAATTGGCTAACAAGAGATTCGTTTTGTTGGACGCTCCAGGTCACAAGA |
| | | | GGQTQEHAHLAKALGVQH | | ACTACGTTCCAAACATGATCGCTGGTGCTTGTCAAGCTGACGTTGCTGC |
| | | | MICVVSKMDEVNWDKKRY | | TTTGATCATCTCTGCTAGACAAGGTGAATTCGAAGCTGGTTTCGAAGGT |
| | | | DHIHDSVEPFLRNQVGIQ | | GGTCAAACCCAAGAACACGCTCACTTGGCTAAGGCTTTGGGTGTTCAAC |
| | | | SIEWVPINGFLNENIDTP | | ACATGATCTGTGTTGTTTCTAAGATGGACGAAGTTAACTGGGACAAGAA |
| | | | IPTERCEWYKGDTLFDKF | | GAGATACGACCACATCCACGACTCTGTTGAACCATTCTTGAGAAACCAA |
| | | | NKVPVPLRDPNGPVRIPV | | GTTGGTATCCAATCTATCGAATGGGTTCCAATCAACGGTTTCTTGAACG |
| | | | LDKLKDQGQFLFGKIESG | | AAAACATCGACACCCCAATCCCAACCGAAAGATGTGAATGGTACAAGGG |
| | | | TIRDDLWVTLMPYRKQFQ | | TGACACCTTGTTCGACAAGTTCAACAAGGTTCCAGTTCCATTGCGCGAC |
| | | | ILSIYNTKDQRVLYASAG | | CCAAACGGTCCAGTTAGAATCCCAGTTTTGGACAAGTTGAAGGACCAAG |
| | | | ENVKIKLKGLEDKDIERG | | GTCAATTCTTGTTCGGTAAGATCGAATCTGGTACCATCCGCGACGACTT |
| | | | YMVCSTEDLCPITQLFIA | | GTGGGTTACCTTGATGCCATACAGAAAGCAATTCCAAATCTTGTCTATC |
| | | | EITILQLPEHKPIMSQGY | | TACAACACCAAGGACCAAAGAGTTTTGTACGCTTCTGCTGGTGAAAACG |
| | | | SCVLHMHTSVAEIEIEEV | | TTAAGATCAAGTTGAAGGGTTTGGAAGACAAGGACATCGAAAGAGGTTA |
| | | | EAVQNPENKKLTKNTFLK | | CATGGTTTGTTCTACCGAAGACTTGTGTCCAATCACCCAATTGTTCATC |
| | | | SNQTGVVKIGIKGGLMCL | | GCTGAAATCACCATCTTGCAATTGCCAGAACACAAGCCAATCATGTCTC |
| | | | EKFETISQLGRFTLRDEE | | AAGGTTACTCTTGTGTTTTGCACATGCACACCTCTGTTGCTGAAATCGA |
| | | | KTIGFGRVMKIKPYKV* | | AATCGAAGAAGTTGAAGCTGTTCAAAACCCAGAAAACAAGAAGTTGACC |
| | | | | | AAGAACACCTTCTTGAAGTCTAACCAAACCGGTGTTGTTAAGATCGGTA |
| | | | | | TCAAGGGTGGTTTGATGTGTTTGGAAAAGTTCGAAACCATCTCTCAATT |
| | | | | | GGGTAGATTCACCTTGCGCGACGAAGAAAAGACCATCGGTTTCGGTAGA |
| | | | | | GTTATGAAGATCAAGCCATACAAGGTTtga |
| 50 | Pte_eRF1_XP_ | SEQ ID NO: | MDQKLNDAEIALEQFRLK | SEQ ID NO: | ATGGACCAAAAGTTGAACGACGCTGAAATCGCTTTGGAACAATTCAGAT |
| | 001425245. | 89 | KLIKTLSQERTAGTSVVS | 150 | TGAAGAAGTTGATCAAGACCTTGTCTCAAGAAAGAACCGCTGGTACCTC |
| | 1/ | | VYIPPKRIISDITNRLNT | | TGTTGTTTCTGTTTACATCCCACCAAAGAGAATCATCTCTGACATCACC |
| | Pte_eRF3_XP_ | | QYAEAASIKDKGNRISVQ | | AACAGATTGAACACCCAATACGCTGAAGCTGCTTCTATCAAGGACAAGG |
| | 001459190. | | EAIQAAILRLRPYNKAPN | | GTAACAGAATCTCTGTTCAAGAAGCTATCCAAGCTGCTATCTTGAGACT |
| | 1 | | NGLVVFCGIVQQADGKGE | | CAGACCATACAACAAGGCTCCAAACAACGGTTTGGTTGTTTTCTGTGGT |
| | | | KKISVVIEPYRPLDLSLY | | ATCGTTCAACAAGCTGACGGTAAGGGTGAAAAGAAGATCTCTGTTGTTA |
| | | | FCDPQFHVEELRALLNID | | TCGAACCATACAGACCATTGGACTTGTCTTTGTACTTCTGTGACCCACA |
| | | | PPFGFIIMDGNGSLFATI | | ATTCCACGTTGAAGAATTGAGAGCTTTGTTGAACATCGACCCACCATTC |
| | | | QGNSKQIIKSFDVDLPKK | | GGTTTCATCATCATGGACGGTAACGGTTCTTTGTTCGCTACCATCCAAG |
| | | | HNKGGQSSVRFARLRMEK | | GTAACTCTAAGCAAATCATCAAGTCTTTCGACGTTGACTTGCCAAAGAA |
| | | | RHNYLRKVCETATTCFIA | | GCACAACAAGGGTGGTCAATCTTCTGTTAGATTCGCTAGATTGAGAATG |
| | | | EDRPNVKGLVLAGSADFK | | GAAAAGAGACACAACTACTTGAGAAAGGTTTGTGAAACCGCTACCACCT |
| | | | NDLAGSQFFDKRLQPLII | | GTTTCATCGCTGAAGACAGACCAAACGTTAAGGGTTTGGTTTTGGCTGG |
| | | | SVVDINYGGEQGLNQAVQ | | TTCTGCTGACTTCAAGAACGACTTGGCTGGTTCTCAATTCTTCGACAAG |
| | | | LSQESLLEVKYIREKNLV | | AGATTGCAACCATTGATCATCTCTGTTGTTGACATCAACTACGGTGGTG |
| | | | GQFFENIDKDTGLVVYGV | | AACAAGGTTTGAACCAAGCTGTTCAATTGTCTCAAGAATCTTTGTTGGA |
| | | | QDTMRAVESQTIKTLVCV | | AGTTAAGTACATCAGAGAAAAGAACTTGGTTGGTCAATTCTTCGAAAAC |
| | | | DTLQYLRLECQSKQTEQK | | ATCGACAAGGACACCGGTTTGGTTGTTTACGGTGTTCAAGACACCATGA |
| | | | AIKYIKGNEGYEAGSLIE | | GAGCTGTTGAATCTCAAACCATCAAGACCTTGGTTTGTGTTGACACCTT |
| | | | EKNGEQFVILVKEDLVEH | | GCAATACTTGAGATTGGAATGTCAATCTAAGCAAACCGAACAAAAGGCT |
| | | | LSEKFKDYGLDFQLITDH | | ATCAAGTACATCAAGGGTAACGAAGGTTACGAAGCTGGTTCTTTGATCG |
| | | | SVEGNQFMKGFSGLGGFL | | AAGAAAAGAACGGTGAACAATTCGTTATCTTGGTTAAGGAAGACTTGGT |
| | | | RFKMDMDYLVQQEDWKDE | | TGAACACTTGTCTGAAAAGTTCAAGGACTACGGTTTGGACTTCCAATTG |
| | | | DEDFI** | | ATCACCGACCACTCTGTTGAAGGTAACCAATTCATGAAGGGTTTCTCTG |
| | | | | | GTTTGGGTGGTTTCTTGAGATTCAAGATGGACATGGACTACTTGGTTCA |
| | | | | | ACAAGAAGACTGGAAGGACGAAGACGAAGACTTCATCtgatag |
| 51 | Pte_eRF1_XP_ | SEQ ID NO: | MSYQYGQQMGQYPYDPNM | SEQ ID NO: | ATGTCTTACCAATACGGTCAACAAATGGGTCAATACCCATACGACCCAA |
| | 001425245. | 90 | NMMGFDPQMYQEYAYYYL | 151 | ACATGAACATGATGGGTTTCGACCCACAAATGTACCAAGAATACGCTTA |
| | 1/ | | GGPPTPPPPKGPYPGITH | | CTACTACTTGGGTGGTCCACCAACCCCACCACCACCAAAGGGTCCATAC |
| | Pte_eRF3_XP_ | | EDYESFDINKQILFQRFL | | CCAGGTATCACCCACGAAGACTACGAATCTTTCGACATCAACAAGCAAA |
| | 001459190. | | GETAAYYAKHLPKYQKEM | | TCTTGTTCCAAAGATTCTTGGGTGAAACCGCTGCTTACTACGCTAAGCA |
| | 1 | | EEFLNTNTAYQMNESEKQ | | CTTGCCAAAGTACCAAAAGGAAATGGAAGAATTCTTGAACACCAACACC |
| | | | LMQSYLDFKKKEKEYESF | | GCTTACCAAATGAACGAATCTGAAAAGCAATTGATGCAATCTTACTTGG |
| | | | LKQLEQQALNPELEQQKK | | ACTTCAAGAAGAAGGAAAAGGAATACGAATCTTTCTTGAAGCAATTGGA |
| | | | LEQQKLEQQKIEQQKLEE | | ACAACAAGCTTTGAACCCAGAATTGGAACAACAAAAGAAGTTGGAACAA |
| | | | QKKQQQLEQQKQQQQQQQ | | CAAAAGTTGGAACAACAAAAGATCGAACAACAAAAGTTGGAAGAACAAA |
| | | | QQPQQEQPKEGATTAVRP | | AGAAGCAACAACAATTGGAACAACAAAAGCAACAACAGCAACAGCAGCA |
| | | | KKKLNLNAKPLEIALNPP | | ACAGCAACCACAACAAGAACAACCAAAGGAAGGTGCTACCACCGCTGTT |
| | | | KMPNFPKHPDFLDFDKFW | | AGACCAAAGAAGAAGTTGAACTTGAACGCTAAGCCATTGGAAATCGCTT |
| | | | NNYSTYISLYNPSCEDEY | | TGAACCCACCAAAGATGCCAAACTTCCCAAAGCACCCAGACTTCTTGGA |
| | | | KNYPKPEQLKKKEADEEA | | CTTCGACAAGTTCTGGAACAACTACTCTACCTACATCTCTTTGTACAAC |
| | | | KRKKKQEEIERAIKKRQD | | CCATCTTGTGAAGACGAATACAAGAACTACCCAAAGCCAGAACAATTGA |
| | | | AQERAKDKPAQSVNLVEQ | | AGAAGAAGGAAGCTGACGAAGAAGCTAAGAGAAAGAAGAAGCAAGAAGA |
| | | | VVKLEGEVDLQKYVDPDE | | AATCGAAAGAGCTATCAAGAAGAGACAAGACGCTCAAGAAAGAGCTAAG |
| | | | TRQPVNLVFIGHVDAGKS | | GACAAGCCAGCTCAATCTGTTAACTTGGTTGAACAAGTTGTTAAGTTGG |
| | | | TLCGRLLLELGEVSEADI | | AAGGTGAAGTTGACTTGCAAAAGTACGTTGACCCAGACGAAACCAGACA |
| | | | KKYEQEAVQNNRDSWWLA | | ACCAGTTAACTTGGTTTTCATCGGTCACGTTGACGCTGGTAAGTCTACC |
| | | | YVMDQNEEEKQKGKTVEC | | TTGTGTGGTAGATTGTTGTTGGAATTGGGTGAAGTTTCTGAAGCTGACA |
| | | | GKAQFVTKQKRFILADAP | | TCAAGAAGTACGAACAAGAAGCTGTTCAAAACAACAGAGACTCTTGGTG |
| | | | GHKNYVPNMIMGACQADL | | GTTGGCTTACGTTATGGACCAAAACGAAGAAGAAAAGCAAAAGGGTAAG |
| | | | AGLIVSAKTGEFESGFEK | | ACCGTTGAATGTGGTAAGGCTCAATTCGTTACCAAGCAAAAGAGATTCA |
| | | | GGQTQEHALLAKSLGVDH | | TCTTGGCTGACGCTCCAGGTCACAAGAACTACGTTCCAAACATGATCAT |
| | | | IIIIVTKMDTIDWNQDRE | | GGGTGCTTGTCAAGCTGACTTGGCTGGTTTGATCGTTTCTGCTAAGACC |
| | | | NLISQNIQEFVLKQCKED | | GGTGAATTCGAATCTGGTTTCGAAAAGGGTGGTCAAACCCAAGAACACG |
| | | | NIYVIPIDALSGSNIKSR | | CTTTGTTGGCTAAGTCTTTGGGTGTTGACCACATCATCATCATCGTTAC |
| | | | VDESKCNWYKGPSLIDLI | | CAAGATGGACACCATCGACTGGAACCAAGACAGATTCAACTTGATCTCT |
| | | | DTVSIPKRNEEGPIRMPI | | CAAAACATCCAAGAATTCGTTTTGAAGCAATGTAAGTTCGACAACATCT |
| | | | LDKFKDMGSLYIYGKLES | | ACGTTATCCCAATCGACGCTTTGTCTGGTTCTAACATCAAGTCTAGAGT |
| | | | GKIIEGLDVSIYPKKQPF | | TGACGAATCTAAGTGTAACTGGTACAAGGGTCCATCTTTGATCGACTTG |
| | | | QITELYNMKDQKMKYAKA | | ATCGACACCGTTTCTATCCCAAAGAGAAACGAAGAAGGTCCAATCAGAA |
| | | | GENIKIKVKNIEEEEIKR | | TGCCAATCTTGGACAAGTTCAAGGACATGGGTTCTTTGTACATCTACGG |
| | | | GYMMCNLTSNPCLVSQEF | | TAAGTTGGAATCTGGTAAGATCATCGAAGGTTTGGACGTTTCTATCTAC |
| | | | QAKIRLLDLPESRRIFSE | | CCAAAGAAGCAACCATTCCAAATCACCGAATTGTACAACATGAAGGACC |
| | | | GYQCIMHLHSAVEEIEIS | | AAAAGATGAAGTACGCTAAGGCTGGTGAAAACATCAAGATCAAGGTTAA |
| | | | CVEAVIDAETKKSIKQNF | | GAACATCGAAGAAGAAGAAATCAAGAGAGGTTACATGATGTGTAACTTG |
| | | | LKSFNEGIAKISIKNPVC | | ACCTCTAACCCATGTTTGGTTTCTCAAGAATTCCAAGCTAAGATCAGAT |
| | | | MEKYETLAQLGRFALRDD | | TGTTGGACTTGCCAGAATCTAGAAGAATCTTCTCTGAAGGTTACCAATG |
| | | | GKTIGFGEILKVKPVKQG | | TATCATGCACTTGCACTCTGCTGTTGAAGAAATCGAAATCTCTTGTGTT |
| | | | * | | GAAGCTGTTATCGACGCTGAAACCAAGAAGTCTATCAAGCAAAACTTCT |
| | | | | | TGAAGTCTTTCAACGAAGGTATCGCTAAGATCTCTATCAAGAACCCAGT |
| | | | | | TTGTATGGAAAAGTACGAAACCTTGGCTCAATTGGGTAGATTCGCTTTG |
| | | | | | CGCGACGACGGTAAGACCATCGGTTTCGGTGAAATCTTGAAGGTTAAGC |
| | | | | | CAGTTAAGCAAGGTTGA |
| 52 | Pte_eRF1_XP_ | SEQ ID NO: | MNQNQIQEQELEIEQFRL | SEQ ID NO: | ATGAACCAAAACCAAATCCAAGAACAAGAATTGGAAATCGAACAATTCA |
| | 001448143. | 91 | SKIIKTLSKTKVIGTSAV | 152 | GATTGTCTAAGATCATCAAGACCTTGTCTAAGACCAAGGTTATCGGTAC |
| | 1/ | | SLYIPPKKIISDITNRLN | | CTCTGCTGTTTCTTTGTACATCCCACCAAAGAAGATCATCTCTGACATC |
| | Pte_eRF3_XP_ | | TQFSEAASIQDKVNRTSV | | ACCAACAGATTGAACACCCAATTCTCTGAAGCTGCTTCTATCCAAGACA |
| | 001459190. | | QDSIQGAVLKLKKYTKAP | | AGGTTAACAGAACCTCTGTTCAAGACTCTATCCAAGGTGCTGTTTTGAA |
| | 1 | | ASGLVLFSGLVEFEKGQK | | GTTGAAGAAGTACACCAAGGCTCCAGCTTCTGGTTTGGTTTTGTTCTCT |
| | | | KISYVIEPFRPLQLSLFF | | GGTTTGGTTGAATTCGAAAAGGGTCAAAAGAAGATCTCTTACGTTATCG |
| | | | CDNYFHIEQLEPLLKLEP | | AACCATTCAGACCATTGCAATTGTCTTTGTTCTTCTGTGACAACTACTT |
| | | | SYGFIIMDGNGALFGKVQ | | CCACATCGAACAATTGGAACCATTGTTGAAGTTGGAACCATCTTACGGT |
| | | | GISKETLKSFNVDLPKKH | | TTCATCATCATGGACGGTAACGGTGCTTTGTTCGGTAAGGTTCAAGGTA |
| | | | NKGGQSSLRFSRIRYWAR | | TCTCTAAGGAAACCTTGAAGTCTTTCAACGTTGACTTGCCAAAGAAGCA |
| | | | HNYLIKVSEQAKNCFISD | | CAACAAGGGTGGTCAATCTTCTTTGAGATTCTCTAGAATCAGATACTGG |
| | | | DKPTIKGLVLAGIADEKN | | GCTAGACACAACTACTTGATCAAGGTTTCTGAACAAGCTAAGAACTGTT |
| | | | KLAESPALDKRLQPLILS | | TCATCTCTGACGACAAGCCAACCATCAAGGGTTTGGTTTTGGCTGGTAT |
| | | | IVDVNYGGENGFNQAIQY | | CGCTGACTTCAAGAACAAGTTGGCTGAATCTCCAGCTTTGGACAAGAGA |
| | | | SQEVLQNQKLQREKDLVA | | TTGCAACCATTGATCTTGTCTATCGTTGACGTTAACTACGGTGGTGAAA |
| | | | KFFLSLDLDNGKSVYGVV | | ACGGTTTCAACCAAGCTATCCAATACTCTCAAGAAGTTTTGCAAAACCA |
| | | | DTMKAIEQELVKQVICIQ | | AAAGTTGCAAAGAGAAAAGGACTTGGTTGCTAAGTTCTTCTTGTCTTTG |
| | | | TLEYSRVECISKQTGVKS | | GACTTGGACAACGGTAAGTCTGTTTACGGTGTTGTTGACACCATGAAGG |
| | | | IKYLKGLDLYEQGSLFED | | CTATCGAACAAGAATTGGTTAAGCAAGTTATCTGTATCCAAACCTTGGA |
| | | | NKGEQFQVTSCQDLVEYL | | ATACTCTAGAGTTGAATGTATCTCTAAGCAAACCGGTGTTAAGTCTATC |
| | | | AENYREKGIDFQLISDNS | | AAGTACTTGAAGGGTTTGGACTTGTACGAACAAGGTTCTTTGTTCGAAG |
| | | | AEGHQFYKGFGGMAGFFR | | ACAACAAGGGTGAACAATTCCAAGTTACCTCTTGTCAAGACTTGGTTGA |
| | | | FSMKMQYNMDSEEEWKSE | | ATACTTGGCTGAAAACTACAGAGAAAAGGGTATCGACTTCCAATTGATC |
| | | | DDEFI** | | TCTGACAACTCTGCTGAAGGTCACCAATTCTACAAGGGTTTCGGTGGTA |
| | | | | | TGGCTGGTTTCTTCAGATTCTCTATGAAGATGCAATACAACATGGACTC |
| | | | | | TGAAGAAGAATGGAAGTCTGAAGACGACGAATTCATCtgatag |
| 53 | Pte_eRF1_XP_ | SEQ ID NO: | MSYQYGQQMGQYPYDPNM | SEQ ID NO: | ATGTCTTACCAATACGGTCAACAAATGGGTCAATACCCATACGACCCAA |
| | 001448143. | 92 | NMMGFDPQMYQEYAYYYL | 153 | ACATGAACATGATGGGTTTCGACCCACAAATGTACCAAGAATACGCTTA |
| | 1/ | | GGPPTPPPPKGPYPGITH | | CTACTACTTGGGTGGTCCACCAACCCCACCACCACCAAAGGGTCCATAC |
| | Pte_eRF3_XP_ | | EDYESFDINKQILFQRFL | | CCAGGTATCACCCACGAAGACTACGAATCTTTCGACATCAACAAGCAAA |
| | 001459190. | | GETAAYYAKHLPKYQKEM | | TCTTGTTCCAAAGATTCTTGGGTGAAACCGCTGCTTACTACGCTAAGCA |
| | 1 | | EEFLNTNTAYQMNESEKQ | | CTTGCCAAAGTACCAAAAGGAAATGGAAGAATTCTTGAACACCAACACC |
| | | | LMQSYLDFKKKEKEYESF | | GCTTACCAAATGAACGAATCTGAAAAGCAATTGATGCAATCTTACTTGG |
| | | | LKQLEQQALNPELEQQKK | | ACTTCAAGAAGAAGGAAAAGGAATACGAATCTTTCTTGAAGCAATTGGA |
| | | | LEQQKLEQQKIEQQKLEE | | ACAACAAGCTTTGAACCCAGAATTGGAACAACAAAAGAAGTTGGAACAA |
| | | | QKKQQQLEQQKQQQQQQQ | | CAAAAGTTGGAACAACAAAAGATCGAACAACAAAAGTTGGAAGAACAAA |
| | | | QQPQQEQPKEGATTAVRP | | AGAAGCAACAACAATTGGAACAACAAAAGCAACAACAGCAACAGCAGCA |
| | | | KKKLNLNAKPLEIALNPP | | ACAGCAACCACAACAAGAACAACCAAAGGAAGGTGCTACCACCGCTGTT |
| | | | KMPNFPKHPDFLDFDKFW | | AGACCAAAGAAGAAGTTGAACTTGAACGCTAAGCCATTGGAAATCGCTT |
| | | | NNYSTYISLYNPSCEDEY | | TGAACCCACCAAAGATGCCAAACTTCCCAAAGCACCCAGACTTCTTGGA |
| | | | KNYPKPEQLKKKEADEEA | | CTTCGACAAGTTCTGGAACAACTACTCTACCTACATCTCTTTGTACAAC |
| | | | KRKKKQEEIERAIKKRQD | | CCATCTTGTGAAGACGAATACAAGAACTACCCAAAGCCAGAACAATTGA |
| | | | AQERAKDKPAQSVNLVEQ | | AGAAGAAGGAAGCTGACGAAGAAGCTAAGAGAAAGAAGAAGCAAGAAGA |
| | | | VVKLEGEVDLQKYVDPDE | | AATCGAAAGAGCTATCAAGAAGAGACAAGACGCTCAAGAAAGAGCTAAG |
| | | | TRQPVNLVFIGHVDAGKS | | GACAAGCCAGCTCAATCTGTTAACTTGGTTGAACAAGTTGTTAAGTTGG |
| | | | TLCGRLLLELGEVSEADI | | AAGGTGAAGTTGACTTGCAAAAGTACGTTGACCCAGACGAAACCAGACA |
| | | | KKYEQEAVQNNRDSWWLA | | ACCAGTTAACTTGGTTTTCATCGGTCACGTTGACGCTGGTAAGTCTACC |
| | | | YVMDQNEEEKQKGKTVEC | | TTGTGTGGTAGATTGTTGTTGGAATTGGGTGAAGTTTCTGAAGCTGACA |
| | | | GKAQFVTKQKRFILADAP | | TCAAGAAGTACGAACAAGAAGCTGTTCAAAACAACAGAGACTCTTGGTG |
| | | | GHKNYVPNMIMGACQADL | | GTTGGCTTACGTTATGGACCAAAACGAAGAAGAAAAGCAAAAGGGTAAG |
| | | | AGLIVSAKTGEFESGFEK | | ACCGTTGAATGTGGTAAGGCTCAATTCGTTACCAAGCAAAAGAGATTCA |
| | | | GGQTQEHALLAKSLGVDH | | TCTTGGCTGACGCTCCAGGTCACAAGAACTACGTTCCAAACATGATCAT |
| | | | IIIIVTKMDTIDWNQDRF | | GGGTGCTTGTCAAGCTGACTTGGCTGGTTTGATCGTTTCTGCTAAGACC |
| | | | NLISQNIQEFVLKQCKED | | GGTGAATTCGAATCTGGTTTCGAAAAGGGTGGTCAAACCCAAGAACACG |
| | | | NIYVIPIDALSGSNIKSR | | CTTTGTTGGCTAAGTCTTTGGGTGTTGACCACATCATCATCATCGTTAC |
| | | | VDESKCNWYKGPSLIDLI | | CAAGATGGACACCATCGACTGGAACCAAGACAGATTCAACTTGATCTCT |
| | | | DTVSIPKRNEEGPIRMPI | | CAAAACATCCAAGAATTCGTTTTGAAGCAATGTAAGTTCGACAACATCT |
| | | | LDKFKDMGSLYIYGKLES | | ACGTTATCCCAATCGACGCTTTGTCTGGTTCTAACATCAAGTCTAGAGT |
| | | | GKIIEGLDVSIYPKKQPF | | TGACGAATCTAAGTGTAACTGGTACAAGGGTCCATCTTTGATCGACTTG |
| | | | QITELYNMKDQKMKYAKA | | ATCGACACCGTTTCTATCCCAAAGAGAAACGAAGAAGGTCCAATCAGAA |
| | | | GENIKIKVKNIEEEEIKR | | TGCCAATCTTGGACAAGTTCAAGGACATGGGTTCTTTGTACATCTACGG |
| | | | GYMMCNLTSNPCLVSQEF | | TAAGTTGGAATCTGGTAAGATCATCGAAGGTTTGGACGTTTCTATCTAC |
| | | | QAKIRLLDLPESRRIFSE | | CCAAAGAAGCAACCATTCCAAATCACCGAATTGTACAACATGAAGGACC |
| | | | GYQCIMHLHSAVEEIEIS | | AAAAGATGAAGTACGCTAAGGCTGGTGAAAACATCAAGATCAAGGTTAA |
| | | | CVEAVIDAETKKSIKQNF | | GAACATCGAAGAAGAAGAAATCAAGAGAGGTTACATGATGTGTAACTTG |
| | | | LKSFNEGIAKISIKNPVC | | ACCTCTAACCCATGTTTGGTTTCTCAAGAATTCCAAGCTAAGATCAGAT |
| | | | MEKYETLAQLGRFALRDD | | TGTTGGACTTGCCAGAATCTAGAAGAATCTTCTCTGAAGGTTACCAATG |
| | | | GKTIGFGEILKVKPVKQG | | TATCATGCACTTGCACTCTGCTGTTGAAGAAATCGAAATCTCTTGTGTT |
| | | | * | | GAAGCTGTTATCGACGCTGAAACCAAGAAGTCTATCAAGCAAAACTTCT |
| | | | | | TGAAGTCTTTCAACGAAGGTATCGCTAAGATCTCTATCAAGAACCCAGT |
| | | | | | TTGTATGGAAAAGTACGAAACCTTGGCTCAATTGGGTAGATTCGCTTTG |
| | | | | | CGCGACGACGGTAAGACCATCGGTTTCGGTGAAATCTTGAAGGTTAAGC |
| | | | | | CAGTTAAGCAAGGTTGA |
| 54 | Eoc_eRF1_ | SEQ ID NO: | MSIIDSNVETWKIKRIIK | SEQ ID NO: | ATGTCTATCATCGACTCTAACGTTGAAACCTGGAAGATCAAGAGAATCA |
| | CAC14170.1/ | 93 | NLERLRGNGTSMISLLLS | 154 | TCAAGAACTTGGAAAGATTGAGAGGTAACGGTACCTCTATGATCTCTTT |
| | N_Yeast_ | | PRDAIPKVQGMLAGEYGT | | GTTGTTGTCTCCACGCGACGCTATCCCAAAGGTTCAAGGTATGTTGGCT |
| | eRF3_ | | AESIKSKINRLAVQGAIT | | GGTGAATACGGTACCGCTGAATCTATCAAGTCTAAGATCAACAGATTGG |
| | Eoc_eRF3_ | | SAKERLKLYNRTPPNGLV | | CTGTTCAAGGTGCTATCACCTCTGCTAAGGAAAGATTGAAGTTGTACAA |
| | AAL33628. | | IYCGIVIGEDKSEKKYCI | | CAGAACCCCACCAAACGGTTTGGTTATCTACTGTGGTATCGTTATCGGT |
| | 1 | | DFEPFRPLNTFKYICDNK | | GAAGACAAGTCTGAAAAGAAGTACTGTATCGACTTCGAACCATTCAGAC |
| | | | FYTKPLFELLENDDVFGF | | CATTGAACACCTTCAAGTACATCTGTGACAACAAGTTCTACACCAAGCC |
| | | | VIVDGSGCLFGTLQGNTK | | ATTGTTCGAATTGTTGGAAAACGACGACGTTTTCGGTTTCGTTATCGTT |
| | | | KIIQNITVSLPKKHGRGG | | GACGGTTCTGGTTGTTTGTTCGGTACCTTGCAAGGTAACACCAAGAAGA |
| | | | QSAPRFGRIREEKRHNYV | | TCATCCAAAACATCACCGTTTCTTTGCCAAAGAAGCACGGTAGAGGTGG |
| | | | RKVAEFATQHFITEDKPN | | TCAATCTGCTCCAAGATTCGGTAGAATCAGAGAAGAAAAGAGACACAAC |
| | | | VKGIILAGSANFKNDLSE | | TACGTTAGAAAGGTTGCTGAATTCGCTACCCAACACTTCATCACCGAAG |
| | | | SDLFDKRLSEIVLKIVDV | | ACAAGCCAAACGTTAAGGGTATCATCTTGGCTGGTTCTGCTAACTTCAA |
| | | | SYGGENGFSQAITLAEDT | | GAACGACTTGTCTGAATCTGACTTGTTCGACAAGAGATTGTCTGAAATC |
| | | | LSNVKFVEEKNLISKYFE | | GTTTTGAAGATCGTTGACGTTTCTTACGGTGGTGAAAACGGTTTCTCTC |
| | | | EIAQDTGMVVFGIEDTLN | | AAGCTATCACCTTGGCTGAAGACACCTTGTCTAACGTTAAGTTCGTTGA |
| | | | SLELGAVGTIICFENLEI | | AGAAAAGAACTTGATCTCTAAGTACTTCGAAGAAATCGCTCAAGACACC |
| | | | NRYEIRNPSTEEIKVIHL | | GGTATGGTTGTTTTCGGTATCGAAGACACCTTGAACTCTTTGGAATTGG |
| | | | CKDQQNDTRYKMIDNNYS | | GTGCTGTTGGTACCATCATCTGTTTCGAAAACTTGGAAATCAACAGATA |
| | | | YFIDQNTGLDLEILSCVP | | CGAAATCAGAAACCCATCTACCGAAGAAATCAAGGTTATCCACTTGTGT |
| | | | LTEWLCENYSKYGVRLEF | | AAGGACCAACAAAACGACACCAGATACAAGATGATCGACAACAACTACT |
| | | | ITDKSQEGFQFVNGFGGI | | CTTACTTCATCGACCAAAACACCGGTTTGGACTTGGAAATCTTGTCTTG |
| | | | GGFLRFKLEIENIDYEGE | | TGTTCCATTGACCGAATGGTTGTGTGAAAACTACTCTAAGTACGGTGTT |
| | | | DVGGEEFDADEDFI** | | AGATTGGAATTCATCACCGACAAGTCTCAAGAAGGTTTCCAATTCGTTA |
| | | | | | ACGGTTTCGGTGGTATCGGTGGTTTCTTGAGATTCAAGTTGGAAATCGA |
| | | | | | AAACATCGACTACGAAGGTGAAGACGTTGGTGGTGAAGAATTCGACGCT |
| | | | | | GACGAAGACTTCATCtaaTAG |
| 55 | Eoc_eRF1_ | SEQ ID NO: | MDKNSDQGNNQQNYQQYS | SEQ ID NO: | ATGGACAAGAACTCTgACCAAGGTAACAACCAACAAAACTACCAACAAT |
| | CAC14170.1/ | 94 | QNGNQQQGNNRYQGYQAY | 155 | ACTCTCAAAACGGTAACCAACAACAAGGTAACAACAGATACCAAGGTTA |
| | N_Yeast_ | | NAQAQPAGGYYQNYQGYS | | CCAAGCTTACAACGCTCAAGCTCAACCAGCTGGTGGTTACTACCAAAAC |
| | eRF3_ | | GYQQGGYQQYNPDAGYQQ | | TACCAAGGTTACTCTGGTTACCAGCAGGGGGGGTATCAGCAGTATAATC |
| | Eoc_eRF3_ | | QYNPQGGYQQYNPQGGYQ | | CTGACGCTGGTTACCAACAACAATACAACCCACAAGGTGGTTACCAGCA |
| | AAL33628. | | QQFNPQGGRGNYKNFNYN | | GTATAATCCTCAGGGCGGCTATCAGCAGCAATTCAACCCACAAGGTGGT |
| | 1 | | NNLQGYQAGFQPQSOGMS | | AGAGGTAACTACAAGAACTTCAACTACAACAACAACTTGCAAGGTTACC |
| | | | LNDFQKQQKQAAPKPKKT | | AAGCTGGTTTCCAACCACAATCTCAAGGTATGTCTTTGAACGACTTCCA |
| | | | LKLVSSSGIKLANATKKV | | AAAGCAACAAAAGCAAGCTGCTCCAAAGCCAAAGAAGACCTTGAAGTTG |
| | | | DTKPAESDKKEEEKSAET | | GTTTCTTCTTCTGGTATCAAGTTGGCTAACGCTACCAAGAAGGTTGACA |
| | | | KEPTKEPTKVEEPVKKEE | | CCAAGCCAGCTGAATCTGACAAGAAGGAAGAAGAAAAGTCTGCTGAAAC |
| | | | KPVQTEEKTEEKSELPKV | | CAAGGAACCAACGAAAGAGCCTACGAAAGTTGAAGAACCAGTTAAGAAG |
| | | | EDLKISESTHNTNNANVT | | GAAGAAAAGCCAGTTCAAACCGAAGAAAAGACCGAAGAAAAGTCTGAAT |
| | | | SADALIKEQEEEVDDEVV | | TGCCAAAGGTTGAAGACTTGAAGATCTCTGAATCTACCCACAACACCAA |
| | | | NDVDETRQPSSLVFIGPV | | CAACGCTAACGTTACCTCTGCTGACGCTTTGATCAAGGAACAAGAAGAA |
| | | | DAVKSTICGNLMFMTGMV | | GAAGTTGACGACGAAGTTGTTAACGACGTTGACGAAACCAGACAACCAT |
| | | | DERTIEKFKQEAKEKNRD | | CTTCTTTGGTTTTCATCGGTCCAGTTGACGCTGTTAAGTCTACCATCTG |
| | | | SWWLAYVMDINDDEKSKG | | TGGTAACTTGATGTTCATGACCGGTATGGTTGACGAAAGAACCATCGAA |
| | | | KTVEVGRATMETPTKRYT | | AAGTTCAAGCAAGAAGCTAAGGAAAAGAACAGAGACTCTTGGTGGTTGG |
| | | | IFDAPGHKNYVPDMIMGA | | CTTACGTTATGGACATCAACGACGACGAAAAGTCTAAGGGTAAGACCGT |
| | | | AMADVAALVISARKGEFE | | TGAAGTTGGTAGAGCTACCATGGAAACCCCAACCAAGAGATACACCATC |
| | | | AGFERDGOTREHAQLARS | | TTCGACGCTCCAGGTCACAAGAACTACGTTCCAGACATGATCATGGGTG |
| | | | LGVNKLVVVVNEMDEETV | | CTGCTATGGCTGACGTTGCTGCTTTGGTTATCTCTGCTAGAAAGGGTGA |
| | | | QWSEERYNDILSGVTPFL | | ATTCGAAGCTGGTTTCGAACGCGACGGTCAAACCAGAGAACACGCTCAA |
| | | | IDQCGYKREDLIFVPISG | | TTGGCTAGATCTTTGGGTGTTAACAAGTTGGTTGTTGTTGTTAACGAAA |
| | | | LNGHNIDKLASCCPWYTG | | TGGACGAAGAAACCGTTCAATGGTCTGAAGAAAGATACAACGACATCTT |
| | | | PTLLEILDCIEPPKRNID | | GTCTGGTGTTACCCCATTCTTGATCGACCAATGTGGTTACAAGAGAGAA |
| | | | GPLRVPVLDKMKDRGVVA | | GACTTGATCTTCGTTCCAATCTCTGGTTTGAACGGTCACAACATCGACA |
| | | | FGKVESGVIRIGPKLAVM | | AGTTGGCTTCTTGTTGTCCATGGTACACCGGTCCAACCTTGTTGGAAAT |
| | | | PNNTKCQVVGIYNCKLEL | | CTTGGACTGTATCGAACCACCAAAGAGAAACATCGACGGTCCATTGAGA |
| | | | VRYANPGENIQIKVRMIE | | GTTCCAGTTTTGGACAAGATGAAGGACAGAGGTGTTGTTGCTTTCGGTA |
| | | | DENQINKGDVLCPYDNLA | | AGGTTGAATCTGGTGTTATCAGAATCGGTCCAAAGTTGGCTGTTATGCC |
| | | | PITDLFEAELTILELLPH | | AAACAACACCAAGTGTCAAGTTGTTGGTATCTACAACTGTAAGTTGGAA |
| | | | RPIITPGYKSMMHLHTIS | | TTGGTTAGATACGCTAACCCAGGTGAAAACATCCAAATCAAGGTTAGAA |
| | | | DEIVIQTLTGIYELDGSG | | TGATCGAAGACGAAAACCAAATCAACAAGGGTGACGTTTTGTGTCCATA |
| | | | KEYVKKNPKYCKSGSKVI | | CGACAACTTGGCTCCAATCACCGACTTGTTCGAAGCTGAATTGACCATC |
| | | | VKISTRVPVCLEKYEFIV | | TTGGAATTGTTGCCACACAGACCAATCATCACCCCAGGTTACAAGTCTA |
| | | | HMGRFTLRDEGKTIALGK | | TGATGCACTTGCACACCATCTCTGACGAAATCGTTATCCAAACCTTGAC |
| | | | VLRYKPAVIKKVEEIPPG | | CGGTATCTACGAATTGGACGGTTCTGGTAAGGAATACGTTAAGAAGAAC |
| | | | VGDEGQAKLEESEEFSGS | | CCAAAGTACTGTAAGTCTGGTTCTAAGGTTATCGTTAAGATCTCTACCA |
| | | | RGDSPSKDDNKYEVITYD | | GAGTTCCAGTTTGTTTGGAAAAGTACGAATTCATCGTTCACATGGGTAG |
| | | | PEEDTIIASSTGSENAE* | | ATTCACCTTGCGCGACGAAGGTAAGACCATCGCTTTGGGTAAGGTTTTG |
| | | | | | AGATACAAGCCAGCTGTTATCAAGAAGGTTGAAGAAATCCCACCAGGTG |
| | | | | | TTGGTGACGAAGGTCAAGCTAAGTTGGAAGAATCTGAAGAATTCTCTGG |
| | | | | | TTCTAGAGGTGACTCTCCATCTAAGGACGACAACAAGTACGAAGTTATC |
| | | | | | ACCTACGACCCAGAAGAAGACACCATCATCGCTTCTTCTACCGGTTCTG |
| | | | | | AAAACGCTGAAtaatag |
| 56 | Eoc_eRF1_ | SEQ ID NO: | MAKLDDNVETWRIKRLIK | SEQ ID NO: | ATGGCTAAGTTGGACGACAACGTTGAAACCTGGAGAATCAAGAGATTGA |
| | AAG25924.1/ | 95 | NLEKLRGDGTSMISLLLS | 156 | TCAAGAACTTGGAAAAGTTGAGAGGTGACGGTACCTCTATGATCTCTTT |
| | N_Yeast_ | | PRDQISKVQAMLAGEAGT | | GTTGTTGTCTCCACGCGACCAAATCTCTAAGGTTCAAGCTATGTTGGCT |
| | eRF3_ | | AVNIKSRVNRQAVLSAIT | | GGTGAAGCTGGTACCGCTGTTAACATCAAGTCTAGAGTTAACAGACAAG |
| | Eoc_eRF3_ | | SAKERLKLYSKTPTNGLV | | CTGTTTTGTCTGCTATCACCTCTGCTAAGGAAAGATTGAAGTTGTACTC |
| | AAL33628. | | VYCGTVIGEDDSEKKYTI | | TAAGACCCCAACCAACGGTTTGGTTGTTTACTGTGGTACCGTTATCGGT |
| | 1 | | DFEPFRPLNTFKYICDNK | | GAAGACGACTCTGAAAAGAAGTACACCATCGACTTCGAACCATTCAGAC |
| | | | FCTEPLFELLENDDVFGF | | CATTGAACACCTTCAAGTACATCTGTGACAACAAGTTCTGTACCGAACC |
| | | | VIVDGNGCLFGTLQGNTK | | ATTGTTCGAATTGTTGGAAAACGACGACGTTTTCGGTTTCGTTATCGTT |
| | | | KILQQITVSLPKKHGRGG | | GACGGTAACGGTTGTTTGTTCGGTACCTTGCAAGGTAACACCAAGAAGA |
| | | | QSAPREGRIREEKRHNYV | | TCTTGCAACAAATCACCGTTTCTTTGCCAAAGAAGCACGGTAGAGGTGG |
| | | | RKVAELATQHFITDDRPN | | TCAATCTGCTCCAAGATTCGGTAGAATCAGAGAAGAAAAGAGACACAAC |
| | | | VKGLVLAGSANFKNDLSE | | TACGTTAGAAAGGTTGCTGAATTGGCTACCCAACACTTCATCACCGACG |
| | | | SDLFDKRLSEVVIKIVDV | | ACAGACCAAACGTTAAGGGTTTGGTTTTGGCTGGTTCTGCTAACTTCAA |
| | | | SYGGENGFSQAISLAEDA | | GAACGACTTGTCTGAATCTGACTTGTTCGACAAGAGATTGTCTGAAGTT |
| | | | LSNVKFVEEKNLISKYFE | | GTTATCAAGATCGTTGACGTTTCTTACGGTGGTGAAAACGGTTTCTCTC |
| | | | EIALDSGMIVFGVEDTLH | | AAGCTATCTCTTTGGCTGAAGACGCTTTGTCTAACGTTAAGTTCGTTGA |
| | | | SLEVGALDLLMCFENLEI | | AGAAAAGAACTTGATCTCTAAGTACTTCGAAGAAATCGCTTTGGACTCT |
| | | | NRYEIRDPANDEIKIYNL | | GGTATGATCGTTTTCGGTGTTGAAGACACCTTGCACTCTTTGGAAGTTG |
| | | | NKEQEKDSKYFKNEKTGT | | GTGCTTTGGACTTGTTGATGTGTTTCGAAAACTTGGAAATCAACAGATA |
| | | | DLEIVKCVALSEWLCENY | | CGAAATCCGCGACCCAGCTAACGACGAAATCAAGATCTACAACTTGAAC |
| | | | SKYGVKLEFITDKSQEGF | | AAGGAACAAGAAAAGGACTCTAAGTACTTCAAGAACGAAAAGACCGGTA |
| | | | QFVNGFGGIGGFLRYKLE | | CCGACTTGGAAATCGTTAAGTGTGTTGCTTTGTCTGAATGGTTGTGTGA |
| | | | MENHDYDKEDVGGEEFNP | | AAACTACTCTAAGTACGGTGTTAAGTTGGAATTCATCACCGACAAGTCT |
| | | | DEDFI** | | CAAGAAGGTTTCCAATTCGTTAACGGTTTCGGTGGTATCGGTGGTTTCT |
| | | | | | TGAGATACAAGTTGGAAATGGAAAACCACGACTACGACAAGGAAGACGT |
| | | | | | TGGTGGTGAAGAATTCAACCCAGACGAAGACTTCATCtaaTAG |
| 57 | Eoc_eRF1_ | SEQ ID NO: | MSDSNOGNNQQNYQQYSQ | SEQ ID NO: | ATGTCTGACTCTAACCAAGGTAACAACCAACAAAACTACCAACAATACT |
| | AAG25924.1/ | 96 | NGNQQQGNNRYQGYQAYN | 157 | CTCAAAACGGTAACCAACAACAAGGTAACAACAGATACCAAGGTTACCA |
| | N_Yeast_ | | AQAQPAGGYYQNYQGYSG | | AGCTTACAACGCTCAAGCTCAACCAGCTGGTGGTTACTACCAAAACTAC |
| | eRF3_ | | YQQGGYQQYNPDAGYQQQ | | CAAGGTTACTCTGGTTACCAGCAGGGGGGGTATCAGCAGTATAATCCTG |
| | Eoc_eRF3_ | | YNPQGGYQQYNPQGGYQQ | | ACGCTGGTTACCAACAACAATACAACCCACAAGGTGGTTACCAGCAGTA |
| | AAL33628. | | QFNPQGGRGNYKNFNYNN | | TAATCCTCAGGGCGGCTATCAGCAGCAATTCAACCCACAAGGTGGTAGA |
| | 1 | | NLQGYQAGFQPQSOGMSL | | GGTAACTACAAGAACTTCAACTACAACAACAACTTGCAAGGTTACCAAG |
| | | | NDFQKQQKQAAPKPKKTL | | CTGGTTTCCAACCACAATCTCAAGGTATGTCTTTGAACGACTTCCAAAA |
| | | | KLVSSSGIKLANATKKVD | | GCAACAAAAGCAAGCTGCTCCAAAGCCAAAGAAGACCTTGAAGTTGGTT |
| | | | TKPAESDKKEEEKSAETK | | TCTTCTTCTGGTATCAAGTTGGCTAACGCTACCAAGAAGGTTGACACCA |
| | | | EPTKEPTKVEEPVKKEEK | | AGCCAGCTGAATCTGACAAGAAGGAAGAAGAAAAGTCTGCTGAAACCAA |
| | | | PVQTEEKTEEKSELPKVE | | GGAACCAACGAAAGAGCCTACGAAAGTTGAAGAACCAGTTAAGAAGGAA |
| | | | DLKISESTHNTNNANVTS | | GAAAAGCCAGTTCAAACCGAAGAAAAGACCGAAGAAAAGTCTGAATTGC |
| | | | ADALIKEQEEEVDDEVVN | | CAAAGGTTGAAGACTTGAAGATCTCTGAATCTACCCACAACACCAACAA |
| | | | DVDETRQPSSLVFIGPVD | | CGCTAACGTTACCTCTGCTGACGCTTTGATCAAGGAACAAGAAGAAGAA |
| | | | AVKSTICGNLMFMTGMVD | | GTTGACGACGAAGTTGTTAACGACGTTGACGAAACCAGACAACCATCTT |
| | | | ERTIEKFKQEAKEKNRDS | | CTTTGGTTTTCATCGGTCCAGTTGACGCTGTTAAGTCTACCATCTGTGG |
| | | | WWLAYVMDINDDEKSKGK | | TAACTTGATGTTCATGACCGGTATGGTTGACGAAAGAACCATCGAAAAG |
| | | | TVEVGRATMETPTKRYTI | | TTCAAGCAAGAAGCTAAGGAAAAGAACAGAGACTCTTGGTGGTTGGCTT |
| | | | FDAPGHKNYVPDMIMGAA | | ACGTTATGGACATCAACGACGACGAAAAGTCTAAGGGTAAGACCGTTGA |
| | | | MADVAALVISARKGEFEA | | AGTTGGTAGAGCTACCATGGAAACCCCAACCAAGAGATACACCATCTTC |
| | | | GFERDGQTREHAQLARSL | | GACGCTCCAGGTCACAAGAACTACGTTCCAGACATGATCATGGGTGCTG |
| | | | GVNKLVVVVNEMDEETVQ | | CTATGGCTGACGTTGCTGCTTTGGTTATCTCTGCTAGAAAGGGTGAATT |
| | | | WSEERYNDILSGVTPFLI | | CGAAGCTGGTTTCGAACGCGACGGTCAAACCAGAGAACACGCTCAATTG |
| | | | DQCGYKREDLIFVPISGL | | GCTAGATCTTTGGGTGTTAACAAGTTGGTTGTTGTTGTTAACGAAATGG |
| | | | NGHNIDKLASCCPWYTGP | | ACGAAGAAACCGTTCAATGGTCTGAAGAAAGATACAACGACATCTTGTC |
| | | | TLLEILDCIEPPKRNIDG | | TGGTGTTACCCCATTCTTGATCGACCAATGTGGTTACAAGAGAGAAGAC |
| | | | PLRVPVLDKMKDRGVVAF | | TTGATCTTCGTTCCAATCTCTGGTTTGAACGGTCACAACATCGACAAGT |
| | | | GKVESGVIRIGPKLAVMP | | TGGCTTCTTGTTGTCCATGGTACACCGGTCCAACCTTGTTGGAAATCTT |
| | | | NNTKCQVVGIYNCKLELV | | GGACTGTATCGAACCACCAAAGAGAAACATCGACGGTCCATTGAGAGTT |
| | | | RYANPGENIQIKVRMIED | | CCAGTTTTGGACAAGATGAAGGACAGAGGTGTTGTTGCTTTCGGTAAGG |
| | | | ENQINKGDVLCPYDNLAP | | TTGAATCTGGTGTTATCAGAATCGGTCCAAAGTTGGCTGTTATGCCAAA |
| | | | ITDLFEAELTILELLPHR | | CAACACCAAGTGTCAAGTTGTTGGTATCTACAACTGTAAGTTGGAATTG |
| | | | PIITPGYKSMMHLHTISD | | GTTAGATACGCTAACCCAGGTGAAAACATCCAAATCAAGGTTAGAATGA |
| | | | EIVIQTLTGIYELDGSGK | | TCGAAGACGAAAACCAAATCAACAAGGGTGACGTTTTGTGTCCATACGA |
| | | | EYVKKNPKYCKSGSKVIV | | CAACTTGGCTCCAATCACCGACTTGTTCGAAGCTGAATTGACCATCTTG |
| | | | KISTRVPVCLEKYEFIVH | | GAATTGTTGCCACACAGACCAATCATCACCCCAGGTTACAAGTCTATGA |
| | | | MGRFTLRDEGKTIALGKV | | TGCACTTGCACACCATCTCTGACGAAATCGTTATCCAAACCTTGACCGG |
| | | | LRYKPAVIKKVEEIPPGV | | TATCTACGAATTGGACGGTTCTGGTAAGGAATACGTTAAGAAGAACCCA |
| | | | GDEGQAKLEESEEFSGSR | | AAGTACTGTAAGTCTGGTTCTAAGGTTATCGTTAAGATCTCTACCAGAG |
| | | | GDSPSKDDNKYEVITYDP | | TTCCAGTTTGTTTGGAAAAGTACGAATTCATCGTTCACATGGGTAGATT |
| | | | EEDTIIASSTGSENAE** | | CACCTTGCGCGACGAAGGTAAGACCATCGCTTTGGGTAAGGTTTTGAGA |
| | | | * | | TACAAGCCAGCTGTTATCAAGAAGGTTGAAGAAATCCCACCAGGTGTTG |
| | | | | | GTGACGAAGGTCAAGCTAAGTTGGAAGAATCTGAAGAATTCTCTGGTTC |
| | | | | | TAGAGGTGACTCTCCATCTAAGGACGACAACAAGTACGAAGTTATCACC |
| | | | | | TACGACCCAGAAGAAGACACCATCATCGCTTCTTCTACCGGTTCTGAAA |
| | | | | | ACGCTGAAtaatag |
| 58 | Pte_eRF1_XP_ | SEQ ID NO: | MDQKLNDAEIALEQFRLK | SEQ ID NO: | ATGGACCAAAAGTTGAACGACGCTGAAATCGCTTTGGAACAATTCAGAT |
| | 001425245. | 97 | KLIKTLSQERTAGTSVVS | 158 | TGAAGAAGTTGATCAAGACCTTGTCTCAAGAAAGAACCGCTGGTACCTC |
| | 1/ | | VYIPPKRIISDITNRLNT | | TGTTGTTTCTGTTTACATCCCACCAAAGAGAATCATCTCTGACATCACC |
| | N_Yeast_ | | QYAEAASIKDKGNRISVQ | | AACAGATTGAACACCCAATACGCTGAAGCTGCTTCTATCAAGGACAAGG |
| | eRF3_ | | EAIQAAILRLRPYNKAPN | | GTAACAGAATCTCTGTTCAAGAAGCTATCCAAGCTGCTATCTTGAGACT |
| | Pte_eRF3_ | | NGLVVFCGIVQQADGKGE | | CAGACCATACAACAAGGCTCCAAACAACGGTTTGGTTGTTTTCTGTGGT |
| | XP_ | | KKISVVIEPYRPLDLSLY | | ATCGTTCAACAAGCTGACGGTAAGGGTGAAAAGAAGATCTCTGTTGTTA |
| | 001459190.1 | | FCDPQFHVEELRALLNID | | TCGAACCATACAGACCATTGGACTTGTCTTTGTACTTCTGTGACCCACA |
| | | | PPFGFIIMDGNGSLFATI | | ATTCCACGTTGAAGAATTGAGAGCTTTGTTGAACATCGACCCACCATTC |
| | | | QGNSKQIIKSFDVDLPKK | | GGTTTCATCATCATGGACGGTAACGGTTCTTTGTTCGCTACCATCCAAG |
| | | | HNKGGQSSVRFARLRMEK | | GTAACTCTAAGCAAATCATCAAGTCTTTCGACGTTGACTTGCCAAAGAA |
| | | | RHNYLRKVCETATTCFIA | | GCACAACAAGGGTGGTCAATCTTCTGTTAGATTCGCTAGATTGAGAATG |
| | | | EDRPNVKGLVLAGSADFK | | GAAAAGAGACACAACTACTTGAGAAAGGTTTGTGAAACCGCTACCACCT |
| | | | NDLAGSQFFDKRLQPLII | | GTTTCATCGCTGAAGACAGACCAAACGTTAAGGGTTTGGTTTTGGCTGG |
| | | | SVVDINYGGEQGLNQAVQ | | TTCTGCTGACTTCAAGAACGACTTGGCTGGTTCTCAATTCTTCGACAAG |
| | | | LSQESLLEVKYIREKNLV | | AGATTGCAACCATTGATCATCTCTGTTGTTGACATCAACTACGGTGGTG |
| | | | GQFFENIDKDTGLVVYGV | | AACAAGGTTTGAACCAAGCTGTTCAATTGTCTCAAGAATCTTTGTTGGA |
| | | | QDTMRAVESQTIKTLVCV | | AGTTAAGTACATCAGAGAAAAGAACTTGGTTGGTCAATTCTTCGAAAAC |
| | | | DTLQYLRLECQSKQTEQK | | ATCGACAAGGACACCGGTTTGGTTGTTTACGGTGTTCAAGACACCATGA |
| | | | AIKYIKGNEGYEAGSLIE | | GAGCTGTTGAATCTCAAACCATCAAGACCTTGGTTTGTGTTGACACCTT |
| | | | EKNGEQFVILVKEDLVEH | | GCAATACTTGAGATTGGAATGTCAATCTAAGCAAACCGAACAAAAGGCT |
| | | | LSEKFKDYGLDFQLITDH | | ATCAAGTACATCAAGGGTAACGAAGGTTACGAAGCTGGTTCTTTGATCG |
| | | | SVEGNQFMKGFSGLGGFL | | AAGAAAAGAACGGTGAACAATTCGTTATCTTGGTTAAGGAAGACTTGGT |
| | | | RFKMDMDYLVQQEDWKDE | | TGAACACTTGTCTGAAAAGTTCAAGGACTACGGTTTGGACTTCCAATTG |
| | | | DEDFI** | | ATCACCGACCACTCTGTTGAAGGTAACCAATTCATGAAGGGTTTCTCTG |
| | | | | | GTTTGGGTGGTTTCTTGAGATTCAAGATGGACATGGACTACTTGGTTCA |
| | | | | | ACAAGAAGACTGGAAGGACGAAGACGAAGACTTCATCtgaTAG |
| 59 | Pte_eRF1_XP_ | SEQ ID NO: | MSDSNQGNNQQNYQQYSQ | SEQ ID NO: | ATGTCTGACTCTAACCAAGGTAACAACCAACAAAACTACCAACAATACT |
| | 001425245. | 98 | NGNQQQGNNRYQGYQAYN | 159 | CTCAAAACGGTAACCAACAACAAGGTAACAACAGATACCAAGGTTACCA |
| | 1/ | | AQAQPAGGYYQNYQGYSG | | AGCTTACAACGCTCAAGCTCAACCAGCTGGTGGTTACTACCAAAACTAC |
| | N_Yeast_ | | YQQGGYQQYNPDAGYQQQ | | CAAGGTTACTCTGGTTACCAGCAGGGGGGGTATCAGCAGTATAATCCTG |
| | eRF3_ | | YNPQGGYQQYNPQGGYQQ | | ACGCTGGTTACCAACAACAATACAACCCACAAGGTGGTTACCAGCAGTA |
| | Pte_eRF3_ | | QFNPQGGRGNYKNENYNN | | TAATCCTCAGGGCGGCTATCAGCAGCAATTCAACCCACAAGGTGGTAGA |
| | XP_ | | NLQGYQAGFQPQSQGMSL | | GGTAACTACAAGAACTTCAACTACAACAACAACTTGCAAGGTTACCAAG |
| | 001459190.1 | | NDFQKQQKQAAPKPKKTL | | CTGGTTTCCAACCACAATCTCAAGGTATGTCTTTGAACGACTTCCAAAA |
| | | | KLVSSSGIKLANATKKVD | | GCAACAAAAGCAAGCTGCTCCAAAGCCAAAGAAGACCTTGAAGTTGGTT |
| | | | TKPAESDKKEEEKSAETK | | TCTTCTTCTGGTATCAAGTTGGCTAACGCTACCAAGAAGGTTGACACCA |
| | | | EPTKEPTKVEEPVKKEEK | | AGCCAGCTGAATCTGACAAGAAGGAAGAAGAAAAGTCTGCTGAAACCAA |
| | | | PVQTEEKTEEKSELPKVE | | GGAACCAACGAAAGAGCCTACGAAAGTTGAAGAACCAGTTAAGAAGGAA |
| | | | DLKISESTHNTNNANVTS | | GAAAAGCCAGTTCAAACCGAAGAAAAGACCGAAGAAAAGTCTGAATTGC |
| | | | ADALIKEQEEEVDDEVVN | | CAAAGGTTGAAGACTTGAAGATCTCTGAATCTACCCACAACACCAACAA |
| | | | DPDETRQPVNLVFIGHVD | | CGCTAACGTTACCTCTGCTGACGCTTTGATCAAGGAACAAGAAGAAGAA |
| | | | AGKSTLCGRLLLELGEVS | | GTTGACGACGAAGTTGTTAACGACCCAGACGAAACCAGACAACCAGTTA |
| | | | EADIKKYEQEAVQNNRDS | | ACTTGGTTTTCATCGGTCACGTTGACGCTGGTAAGTCTACCTTGTGTGG |
| | | | WWLAYVMDQNEEEKQKGK | | TAGATTGTTGTTGGAATTGGGTGAAGTTTCTGAAGCTGACATCAAGAAG |
| | | | TVECGKAQFVTKQKRFIL | | TACGAACAAGAAGCTGTTCAAAACAACAGAGACTCTTGGTGGTTGGCTT |
| | | | ADAPGHKNYVPNMIMGAC | | ACGTTATGGACCAAAACGAAGAAGAAAAGCAAAAGGGTAAGACCGTTGA |
| | | | QADLAGLIVSAKTGEFES | | ATGTGGTAAGGCTCAATTCGTTACCAAGCAAAAGAGATTCATCTTGGCT |
| | | | GFEKGGQTQEHALLAKSL | | GACGCTCCAGGTCACAAGAACTACGTTCCAAACATGATCATGGGTGCTT |
| | | | GVDHIIIIVTKMDTIDWN | | GTCAAGCTGACTTGGCTGGTTTGATCGTTTCTGCTAAGACCGGTGAATT |
| | | | QDRFNLISQNIQEFVLKQ | | CGAATCTGGTTTCGAAAAGGGTGGTCAAACCCAAGAACACGCTTTGTTG |
| | | | CKEDNIYVIPIDALSGSN | | GCTAAGTCTTTGGGTGTTGACCACATCATCATCATCGTTACCAAGATGG |
| | | | IKSRVDESKCNWYKGPSL | | ACACCATCGACTGGAACCAAGACAGATTCAACTTGATCTCTCAAAACAT |
| | | | IDLIDTVSIPKRNEEGPI | | CCAAGAATTCGTTTTGAAGCAATGTAAGTTCGACAACATCTACGTTATC |
| | | | RMPILDKFKDMGSLYIYG | | CCAATCGACGCTTTGTCTGGTTCTAACATCAAGTCTAGAGTTGACGAAT |
| | | | KLESGKIIEGLDVSIYPK | | CTAAGTGTAACTGGTACAAGGGTCCATCTTTGATCGACTTGATCGACAC |
| | | | KQPFQITELYNMKDQKMK | | CGTTTCTATCCCAAAGAGAAACGAAGAAGGTCCAATCAGAATGCCAATC |
| | | | YAKAGENIKIKVKNIEEE | | TTGGACAAGTTCAAGGACATGGGTTCTTTGTACATCTACGGTAAGTTGG |
| | | | EIKRGYMMCNLTSNPCLV | | AATCTGGTAAGATCATCGAAGGTTTGGACGTTTCTATCTACCCAAAGAA |
| | | | SQEFQAKIRLLDLPESRR | | GCAACCATTCCAAATCACCGAATTGTACAACATGAAGGACCAAAAGATG |
| | | | IFSEGYQCIMHLHSAVEE | | AAGTACGCTAAGGCTGGTGAAAACATCAAGATCAAGGTTAAGAACATCG |
| | | | IEISCVEAVIDAETKKSI | | AAGAAGAAGAAATCAAGAGAGGTTACATGATGTGTAACTTGACCTCTAA |
| | | | KQNFLKSFNEGIAKISIK | | CCCATGTTTGGTTTCTCAAGAATTCCAAGCTAAGATCAGATTGTTGGAC |
| | | | NPVCMEKYETLAQLGRFA | | TTGCCAGAATCTAGAAGAATCTTCTCTGAAGGTTACCAATGTATCATGC |
| | | | LRDDGKTIGFGEILKVKP | | ACTTGCACTCTGCTGTTGAAGAAATCGAAATCTCTTGTGTTGAAGCTGT |
| | | | VKQG* | | TATCGACGCTGAAACCAAGAAGTCTATCAAGCAAAACTTCTTGAAGTCT |
| | | | | | TTCAACGAAGGTATCGCTAAGATCTCTATCAAGAACCCAGTTTGTATGG |
| | | | | | AAAAGTACGAAACCTTGGCTCAATTGGGTAGATTCGCTTTGCGCGACGA |
| | | | | | CGGTAAGACCATCGGTTTCGGTGAAATCTTGAAGGTTAAGCCAGTTAAG |
| | | | | | CAAGGTTGA |
| 60 | Pte_eRF1_XP_ | SEQ ID NO: | MNONQIQEQELEIEQFRL | SEQ ID NO: | ATGAACCAAAACCAAATCCAAGAACAAGAATTGGAAATCGAACAATTCA |
| | 001448143. | 99 | SKIIKTLSKTKVIGTSAV | 160 | GATTGTCTAAGATCATCAAGACCTTGTCTAAGACCAAGGTTATCGGTAC |
| | 1/ | | SLYIPPKKIISDITNRLN | | CTCTGCTGTTTCTTTGTACATCCCACCAAAGAAGATCATCTCTGACATC |
| | N_Yeast_ | | TQFSEAASIQDKVNRTSV | | ACCAACAGATTGAACACCCAATTCTCTGAAGCTGCTTCTATCCAAGACA |
| | eRF3_ | | QDSIQGAVLKLKKYTKAP | | AGGTTAACAGAACCTCTGTTCAAGACTCTATCCAAGGTGCTGTTTTGAA |
| | Pte_eRF3_ | | ASGLVLFSGLVEFEKGQK | | GTTGAAGAAGTACACCAAGGCTCCAGCTTCTGGTTTGGTTTTGTTCTCT |
| | XP_ | | KISYVIEPFRPLQLSLFF | | GGTTTGGTTGAATTCGAAAAGGGTCAAAAGAAGATCTCTTACGTTATCG |
| | 001459190.1 | | CDNYFHIEQLEPLLKLEP | | AACCATTCAGACCATTGCAATTGTCTTTGTTCTTCTGTGACAACTACTT |
| | | | SYGFIIMDGNGALFGKVQ | | CCACATCGAACAATTGGAACCATTGTTGAAGTTGGAACCATCTTACGGT |
| | | | GISKETLKSFNVDLPKKH | | TTCATCATCATGGACGGTAACGGTGCTTTGTTCGGTAAGGTTCAAGGTA |
| | | | NKGGQSSLRFSRIRYWAR | | TCTCTAAGGAAACCTTGAAGTCTTTCAACGTTGACTTGCCAAAGAAGCA |
| | | | HNYLIKVSEQAKNCFISD | | CAACAAGGGTGGTCAATCTTCTTTGAGATTCTCTAGAATCAGATACTGG |
| | | | DKPTIKGLVLAGIADEKN | | GCTAGACACAACTACTTGATCAAGGTTTCTGAACAAGCTAAGAACTGTT |
| | | | KLAESPALDKRLQPLILS | | TCATCTCTGACGACAAGCCAACCATCAAGGGTTTGGTTTTGGCTGGTAT |
| | | | IVDVNYGGENGFNQAIQY | | CGCTGACTTCAAGAACAAGTTGGCTGAATCTCCAGCTTTGGACAAGAGA |
| | | | SQEVLQNQKLQREKDLVA | | TTGCAACCATTGATCTTGTCTATCGTTGACGTTAACTACGGTGGTGAAA |
| | | | KFFLSLDLDNGKSVYGVV | | ACGGTTTCAACCAAGCTATCCAATACTCTCAAGAAGTTTTGCAAAACCA |
| | | | DTMKAIEQELVKQVICIQ | | AAAGTTGCAAAGAGAAAAGGACTTGGTTGCTAAGTTCTTCTTGTCTTTG |
| | | | TLEYSRVECISKQTGVKS | | GACTTGGACAACGGTAAGTCTGTTTACGGTGTTGTTGACACCATGAAGG |
| | | | IKYLKGLDLYEQGSLFED | | CTATCGAACAAGAATTGGTTAAGCAAGTTATCTGTATCCAAACCTTGGA |
| | | | NKGEQFQVTSCQDLVEYL | | ATACTCTAGAGTTGAATGTATCTCTAAGCAAACCGGTGTTAAGTCTATC |
| | | | AENYREKGIDFQLISDNS | | AAGTACTTGAAGGGTTTGGACTTGTACGAACAAGGTTCTTTGTTCGAAG |
| | | | AEGHQFYKGFGGMAGFER | | ACAACAAGGGTGAACAATTCCAAGTTACCTCTTGTCAAGACTTGGTTGA |
| | | | FSMKMQYNMDSEEEWKSE | | ATACTTGGCTGAAAACTACAGAGAAAAGGGTATCGACTTCCAATTGATC |
| | | | DDEFI ** | | TCTGACAACTCTGCTGAAGGTCACCAATTCTACAAGGGTTTCGGTGGTA |
| | | | | | TGGCTGGTTTCTTCAGATTCTCTATGAAGATGCAATACAACATGGACTC |
| | | | | | TGAAGAAGAATGGAAGTCTGAAGACGACGAATTCATCtgaTAG |
| 61 | Pte_eRF1_XP_ | SEQ ID NO: | MSDSNQGNNQQNYQQYSQ | SEQ ID NO: | ATGTCTGACTCTAACCAAGGTAACAACCAACAAAACTACCAACAATACT |
| | 001448143. | 100 | NGNQQQGNNRYQGYQAYN | 161 | CTCAAAACGGTAACCAACAACAAGGTAACAACAGATACCAAGGTTACCA |
| | 1/ | | AQAQPAGGYYQNYQGYSG | | AGCTTACAACGCTCAAGCTCAACCAGCTGGTGGTTACTACCAAAACTAC |
| | N_Yeast_ | | YQQGGYQQYNPDAGYQQQ | | CAAGGTTACTCTGGTTACCAGCAGGGGGGGTATCAGCAGTATAATCCTG |
| | eRF3_ | | YNPQGGYQQYNPQGGYQQ | | ACGCTGGTTACCAACAACAATACAACCCACAAGGTGGTTACCAGCAGTA |
| | Pte_eRF3_ | | QFNPQGGRGNYKNFNYNN | | TAATCCTCAGGGCGGCTATCAGCAGCAATTCAACCCACAAGGTGGTAGA |
| | XP_ | | NLQGYQAGFQPQSQGMSL | | GGTAACTACAAGAACTTCAACTACAACAACAACTTGCAAGGTTACCAAG |
| | 001459190.1 | | NDFQKQQKQAAPKPKKTL | | CTGGTTTCCAACCACAATCTCAAGGTATGTCTTTGAACGACTTCCAAAA |
| | | | KLVSSSGIKLANATKKVD | | GCAACAAAAGCAAGCTGCTCCAAAGCCAAAGAAGACCTTGAAGTTGGTT |
| | | | TKPAESDKKEEEKSAETK | | TCTTCTTCTGGTATCAAGTTGGCTAACGCTACCAAGAAGGTTGACACCA |
| | | | EPTKEPTKVEEPVKKEEK | | AGCCAGCTGAATCTGACAAGAAGGAAGAAGAAAAGTCTGCTGAAACCAA |
| | | | PVQTEEKTEEKSELPKVE | | GGAACCAACGAAAGAGCCTACGAAAGTTGAAGAACCAGTTAAGAAGGAA |
| | | | DLKISESTHNTNNANVTS | | GAAAAGCCAGTTCAAACCGAAGAAAAGACCGAAGAAAAGTCTGAATTGC |
| | | | ADALIKEQEEEVDDEVVN | | CAAAGGTTGAAGACTTGAAGATCTCTGAATCTACCCACAACACCAACAA |
| | | | DPDETRQPVNLVFIGHVD | | CGCTAACGTTACCTCTGCTGACGCTTTGATCAAGGAACAAGAAGAAGAA |
| | | | AGKSTLCGRLLLELGEVS | | GTTGACGACGAAGTTGTTAACGACCCAGACGAAACCAGACAACCAGTTA |
| | | | EADIKKYEQEAVQNNRDS | | ACTTGGTTTTCATCGGTCACGTTGACGCTGGTAAGTCTACCTTGTGTGG |
| | | | WWLAYVMDQNEEEKQKGK | | TAGATTGTTGTTGGAATTGGGTGAAGTTTCTGAAGCTGACATCAAGAAG |
| | | | TVECGKAQFVTKQKRFIL | | TACGAACAAGAAGCTGTTCAAAACAACAGAGACTCTTGGTGGTTGGCTT |
| | | | ADAPGHKNYVPNMIMGAC | | ACGTTATGGACCAAAACGAAGAAGAAAAGCAAAAGGGTAAGACCGTTGA |
| | | | QADLAGLIVSAKTGEFES | | ATGTGGTAAGGCTCAATTCGTTACCAAGCAAAAGAGATTCATCTTGGCT |
| | | | GFEKGGQTQEHALLAKSL | | GACGCTCCAGGTCACAAGAACTACGTTCCAAACATGATCATGGGTGCTT |
| | | | GVDHIIIIVTKMDTIDWN | | GTCAAGCTGACTTGGCTGGTTTGATCGTTTCTGCTAAGACCGGTGAATT |
| | | | QDRENLISQNIQEFVLKQ | | CGAATCTGGTTTCGAAAAGGGTGGTCAAACCCAAGAACACGCTTTGTTG |
| | | | CKFDNIYVIPIDALSGSN | | GCTAAGTCTTTGGGTGTTGACCACATCATCATCATCGTTACCAAGATGG |
| | | | IKSRVDESKCNWYKGPSL | | ACACCATCGACTGGAACCAAGACAGATTCAACTTGATCTCTCAAAACAT |
| | | | IDLIDTVSIPKRNEEGPI | | CCAAGAATTCGTTTTGAAGCAATGTAAGTTCGACAACATCTACGTTATC |
| | | | RMPILDKFKDMGSLYIYG | | CCAATCGACGCTTTGTCTGGTTCTAACATCAAGTCTAGAGTTGACGAAT |
| | | | KLESGKIIEGLDVSIYPK | | CTAAGTGTAACTGGTACAAGGGTCCATCTTTGATCGACTTGATCGACAC |
| | | | KQPFQITELYNMKDQKMK | | CGTTTCTATCCCAAAGAGAAACGAAGAAGGTCCAATCAGAATGCCAATC |
| | | | YAKAGENIKIKVKNIEEE | | TTGGACAAGTTCAAGGACATGGGTTCTTTGTACATCTACGGTAAGTTGG |
| | | | EIKRGYMMCNLTSNPCLV | | AATCTGGTAAGATCATCGAAGGTTTGGACGTTTCTATCTACCCAAAGAA |
| | | | SQEFQAKIRLLDLPESRR | | GCAACCATTCCAAATCACCGAATTGTACAACATGAAGGACCAAAAGATG |
| | | | IFSEGYQCIMHLHSAVEE | | AAGTACGCTAAGGCTGGTGAAAACATCAAGATCAAGGTTAAGAACATCG |
| | | | IEISCVEAVIDAETKKSI | | AAGAAGAAGAAATCAAGAGAGGTTACATGATGTGTAACTTGACCTCTAA |
| | | | KQNFLKSFNEGIAKISIK | | CCCATGTTTGGTTTCTCAAGAATTCCAAGCTAAGATCAGATTGTTGGAC |
| | | | NPVCMEKYETLAQLGRFA | | TTGCCAGAATCTAGAAGAATCTTCTCTGAAGGTTACCAATGTATCATGC |
| | | | LRDDGKTIGFGEILKVKP | | ACTTGCACTCTGCTGTTGAAGAAATCGAAATCTCTTGTGTTGAAGCTGT |
| | | | VKQG* | | TATCGACGCTGAAACCAAGAAGTCTATCAAGCAAAACTTCTTGAAGTCT |
| | | | | | TTCAACGAAGGTATCGCTAAGATCTCTATCAAGAACCCAGTTTGTATGG |
| | | | | | AAAAGTACGAAACCTTGGCTCAATTGGGTAGATTCGCTTTGCGCGACGA |
| | | | | | CGGTAAGACCATCGGTTTCGGTGAAATCTTGAAGGTTAAGCCAGTTAAG |
| | | | | | CAAGGTTGA |

The examples and embodiments described herein are for illustrative purposes only and various modifications or changes suggested to persons skilled in the art are to be included within the spirit and purview of this application and scope of the appended claims.

### REFERENCES

Inagaki, et al. Convergence and constraint in eukaryotic release factor (eRF1) domain 1: the evolution of stop codon specificity. Nucleic Acids Research. 2002. Jan. 15; 30 (2): 532-44.
Seit-Nebi, et al. Conversion of omnipotent translation termination factor eRF1 into ciliate-like UGA-only unipotent eRF1. EMBO Rep. 2002 Sep.; 3 (9): 881-6.
Ito, et al. Omnipotent decoding potential resides in eukaryotic translation termination factor eRF1 of variant-code organisms and is modulated by the interactions of amino acid sequences within domain 1. Proc Natl Acad Sci USA. 2002 Jun. 25; 99 (13): 8494-8499.
Kisselev. Polypeptide Release Factors in Prokaryotes and Eukaryotes: Same Function, Different Structure. Structure. 2002 January; 10 (1): 8-9.
Haase, et al. Superloser: A Plasmid Shuffling Vector for Saccharomyces cerevisiae with Exceedingly Low Background. G3 (Bethesda). 2019 Aug. 8; 9 (8): 2699-2707.
Boeke, et al. 5-Fluoroorotic acid as a selective agent in yeast molecular genetics. Methods Enzymol. 1987; 154:164-75.
Hirsh, D. Tryptophan transfer tRNA as the UGA suppressor. J. Mol. Biol. 1971; 58, 439-458.
Hofstetter, et al. The readthrough protein A1 is essential for the formation of viable Qβ particles. Biochim. Biophys. Acta 1974; 374, 238-251.
Beier and Grimm. Misreading of termination codons in eukaryotes by natural nonsense suppressor tRNAs. Nucleic Acids Res. 2001 Dec. 1; 29 (23): 4767-82.
Wada and Ito. A genetic approach for analyzing the co-operative function of the tRNA mimicry complex, eRF1/eRF3, in translation termination on the ribosome. Nucleic Acids Res. 2014 July; 42 (12): 7851-7866.
Lacoux, et al. The catalytic activity of the translation termination factor methyltransferase Mtq2-Trm112 complex is required for large ribosomal subunit biogenesis. Nucleic Acids Res. 2020 Dec. 2; 48 (21): 12310-12325.

1. A method comprising:
a. rewriting a first stop codon to a second stop codon in a genome of a first organism;
b. rewriting a third stop codon to the second stop codon in the genome of the first organism; and
c. introducing a release factor into the first organism, wherein the release factor is configured to recognize only the second stop codon as a stop codon, and wherein the release factor does not recognize the first stop codon or the third stop codon as a stop codon.

2. (canceled)

3. The method of claim 1, wherein the release factor does not recognize the first stop codon and the third stop codon as stop codons.

4. (canceled)

5. The method of claim 1, wherein the first stop codon and/or the third stop codon is UAA or UAG; the second stop codon is UGA; and wherein the third stop codon is different from the first stop codon.

6. (canceled)

7. The method of claim 1, wherein
(a) the release factor comprises a class 1 release factor or a class 2 release factor, wherein the class 1 release factor comprises a release factor 1 (RF1) or a release factor 2 (RF2), and wherein the class 2 release factor comprises a release factor 3 (RF3), optionally wherein the RF1 is a eukaryotic RF1 (eRF1) and the RF3 is a eukaryotic RF3 (eRF3); or
(b) the release factor is a release factor 1/release factor 3 (RF1/RF3) complex, optionally wherein the RF1/RF3 complex is a eukaryotic RF1/RF3 (eRF1/eRF3) complex.

8. (canceled)

9. (canceled)

10. (canceled)

11. (canceled)

12. (canceled)

13. (canceled)

14. The method of claim 7, wherein the release factor modulates protein translation upon recognizing the second stop codon as a stop codon, wherein the modulating protein translation comprises terminating protein translation.

15. (canceled)

16. The method of claim 7, wherein:
(i) the release factor comprises a recognition domain comprising one or more mutations that allow the release factor to recognize only the second stop codon as a stop codon;
(ii) the release factor comprises a first recognition domain swapped with a second recognition domain, wherein the second recognition domain is from a release factor of a second organism or the second recognition domain is identified using a phylogenetic screening, directed evolution, library screening, machine learning, or a combination thereof; or
(iii) the release factor is from the second organism.

17. (canceled)

18. (canceled)

19. (canceled)

20. (canceled)

21. The method of claim 16, wherein the second organism comprises a ciliate comprising Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Urostyla sp. HL-2004, Uroleptus sp. WJC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

22. (canceled)

23. The method of claim 16, wherein the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12) YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof.

24. The method of claim 16, wherein the release factor comprises an amino acid sequence selected from the group consisting of SEQ ID NOs: 40-64.

25. The method of claim 16, wherein the release factor from the second organism comprises an eRF1, wherein the eRF1 from the second organism comprises an amino acid sequence that has at least 20% sequence identity to an eRF1 of the first organism.

26. (canceled)

27. The method of claim 25, wherein the release factor comprises an amino acid sequence selected from the group consisting of SEQ ID NOs: 65-74.

28. The method of claim 16, wherein the release factor from the second organism comprises an eRF1/eRF3 complex, wherein the eRF1 of the eRF1/eRF3 complex from the second organism comprises an amino acid sequence that has at least 20% sequence identity to an eRF1 of the first organism, and wherein the eRF3 of the eRF1/eRF3 complex from the second organism comprises an amino acid sequence that has at least 25% sequence identity to an eRF3 of the first organism.

29. (canceled)

30. The method of claim 28, wherein the eRF1 of the eRF1/eRF3 complex comprises an amino acid sequence selected from the group consisting of SEQ ID NOs: 75, 77, 79, 81, 83, 85, 87, 89, and 91, and wherein the eRF3 of the eRF1/eRF3 complex comprises an amino acid sequence selected from the group consisting of SEQ ID NOs: 76, 78, 80, 82, 84, 86, 88, 90, and 92.

31. (canceled)

32. (canceled)

33. The method of claim 16, wherein the release factor from the second organism comprises an eRF1 and forms a complex with a chimeric eRF3, wherein the eRF1 of the second organism comprises an amino acid sequence that has at least 40% sequence identity to an eRF1 of the first organism, and wherein the chimeric eRF3 comprises (i) an eRF3 from the first organism or a fragment thereof and (ii) an eRF3 from a second organism or a fragment thereof.

34. (canceled)

35. (canceled)

36. The method of claim 33, wherein the second organism comprises Euplotes octocarinatus, wherein the chimeric eRF3 comprises an eRF3 of Euplotes octocarinatus, and wherein:
(i) amino acids 7-298 of the eRF3 of Euplotes octocarinatus are replaced with amino acids 6-253 of the eRF3 from the first organism; or
(ii) amino acids 1-298 of the eRF3 of Euplotes octocarinatus are replaced with amino acids 1-253 of the eRF3 from the first organism.

37. (canceled)

38. The method of claim 36, wherein the chimeric eRF3 comprises an amino acid sequence comprising SEQ ID NO: 93, SEQ ID NO: 94, SEQ ID NO: 95, or SEQ ID NO: 96.

39. (canceled)

40. (canceled)

41. The method of claim 33, wherein the second organism comprises Paramecium tetraurelia, and wherein the chimeric eRF3 comprises an eRF3 of Paramecium tetraurelia, wherein amino acids 1-321 of the eRF3 of Paramecium tetraurelia is replaced with amino acids 1-253 of the eRF3 from the first organism.

42. The method of claim 41, wherein the chimeric eRF3 comprises an amino acid sequence comprising SEQ ID NO: 97, SEQ ID NO: 98, SEQ ID NO: 99, or SEQ ID NO: 100.

43. The method of claim 1, wherein the first organism comprises a eukaryotic cell comprising a yeast cell, a fungal cell, a plant cell, an animal cell, an insect cell, a mammalian cell, or a combination thereof, or a prokaryotic cell comprising an archaebacteria cell, a bacterial cell, or a combination thereof.

44. (canceled)

45. (canceled)

46. The method of claim 43, wherein the yeast cell comprises Saccharomyces cerevisiae.

47. The method of claim 1, further comprising inserting an additional stop codon next to the second stop codon, wherein the additional stop codon is UGA, and wherein the inserting the additional stop codon enhances translation termination.

48. (canceled)

49. (canceled)

50. The method of claim 1, wherein the first organism does not comprise a gene encoding an endogenous RF1, RF2, or a combination thereof in the genome, wherein the gene comprises SUP35, SUP45, or a combination thereof.

51. (canceled)

52. The method of claim 1, further comprising:
(a) reassigning the first stop codon and/or the third stop codon to encode a natural amino acid comprising alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; or a non-canonical amino acid (ncAA) comprising an azide-containing ncAA, an alkene-containing ncAA, an alkyne-containing ncAA, p-azidophenylalanine, 2-aminoisobutyric acid (Aib), N6-[(propargyloxy) carbonyl]-L-lysine, O-4-allyl-L-tyrosine, or a combination thereof, and
(b) providing (i) one or more tRNA molecules that recognize the first stop codon and/or the third stop codon and one or more aminoacyl-tRNA synthetases (aaRSs) for charging the one or more tRNA molecules with the natural amino acid or the ncAA; (ii) a tRNA pre-charged with the natural amino acid or the ncAA; or (iii) both (i) and (ii).

53. (canceled)

54. (canceled)

55. (canceled)

56. (canceled)

57. (canceled)

58. The method of claim 1, wherein the release factor is expressed from a gene integrated into the genome or an episomal element.

59.-262. (canceled)


SEQUENCE LISTING (162 sequences)

SEQ ID NO: 1 - protein, 7 aa, Saccharomyces cerevisiae
TASNIKS

SEQ ID NO: 2 - protein, 7 aa, Saccharomyces cerevisiae
YLCDNKF

SEQ ID NO: 3 - protein, 7 aa, Artificial Sequence
  Description of Artificial Sequence Synthetic peptide
KSSNIKS

SEQ ID NO: 4 - protein, 7 aa, Artificial Sequence
  Description of Artificial Sequence Synthetic peptide
YICDNKF

SEQ ID NO: 5 - protein, 7 aa, Artificial Sequence
  Description of Artificial Sequence Synthetic peptide
TAVNIKS

SEQ ID NO: 6 - protein, 7 aa, Artificial Sequence
  Description of Artificial Sequence Synthetic peptide
KAANIKS

SEQ ID NO: 7 - protein, 7 aa, Artificial Sequence
  Description of Artificial Sequence Synthetic peptide
KASNIKS

SEQ ID NO: 8 - protein, 7 aa, Artificial Sequence
  Description of Artificial Sequence Synthetic peptide
YYCGERF

SEQ ID NO: 9 - protein, 7 aa, Artificial Sequence
  Description of Artificial Sequence Synthetic peptide
TAESIKS

SEQ ID NO: 10 - protein, 7 aa, Artificial Sequence
  Description of Artificial Sequence Synthetic peptide
YFCDPQF

SEQ ID NO: 11 - protein, 7 aa, Artificial Sequence
  Description of Artificial Sequence Synthetic peptide
EAASIKD

SEQ ID NO: 12 - protein, 7 aa, Artificial Sequence
  Description of Artificial Sequence Synthetic peptide
KATNIKD

SEQ ID NO: 13 - protein, 7 aa, Artificial Sequence
  Description of Artificial Sequence Synthetic peptide
YFCDSKF

SEQ ID NO: 14 - protein, 7 aa, Artificial Sequence
  Description of Artificial Sequence Synthetic peptide
FDFDAES

SEQ ID NO: 15 - protein, 7 aa, Artificial Sequence
  Description of Artificial Sequence Synthetic peptide
TLIKPQF

SEQ ID NO: 16 - protein, 7 aa, Artificial Sequence
  Description of Artificial Sequence Synthetic peptide
TGDKIKS

SEQ ID NO: 17 - protein, 7 aa, Artificial Sequence
  Description of Artificial Sequence Synthetic peptide
TIIKNDF

SEQ ID NO: 18 - protein, 7 aa, Artificial Sequence
  Description of Artificial Sequence Synthetic peptide
EAASIQD

SEQ ID NO: 19 - protein, 7 aa, Artificial Sequence
  Description of Artificial Sequence Synthetic peptide
FFCDNYF

SEQ ID NO: 20 - protein, 7 aa, Artificial Sequence
  Description of Artificial Sequence Synthetic peptide
FVIVNKF

SEQ ID NO: 21 - protein, 7 aa, Artificial Sequence
  Description of Artificial Sequence Synthetic peptide
AAQNIKS

SEQ ID NO: 22 - protein, 7 aa, Artificial Sequence
  Description of Artificial Sequence Synthetic peptide
YFCGGKF

SEQ ID NO: 23 - protein, 7 aa, Artificial Sequence
  Description of Artificial Sequence Synthetic peptide
QANSIKD

SEQ ID NO: 24 - protein, 7 aa, Artificial Sequence
  Description of Artificial Sequence Synthetic peptide
YRCDSKF

SEQ ID NO: 25 - protein, 7 aa, Artificial Sequence
  Description of Artificial Sequence Synthetic peptide
GAASIKN

SEQ ID NO: 26 - protein, 7 aa, Artificial Sequence
  Description of Artificial Sequence Synthetic peptide
YSCNTIF

SEQ ID NO: 27 - protein, 7 aa, Artificial Sequence
  Description of Artificial Sequence Synthetic peptide
SAQNIKS

SEQ ID NO: 28 - protein, 7 aa, Artificial Sequence
  Description of Artificial Sequence Synthetic peptide
YYCDNRF

SEQ ID NO: 29 - protein, 7 aa, Artificial Sequence
  Description of Artificial Sequence Synthetic peptide
SAGNIKS

SEQ ID NO: 30 - protein, 7 aa, Artificial Sequence
  Description of Artificial Sequence Synthetic peptide
YFCDNSF

SEQ ID NO: 31 - protein, 7 aa, Artificial Sequence
  Description of Artificial Sequence Synthetic peptide
TAQNIKS

SEQ ID NO: 32 - protein, 7 aa, Artificial Sequence
  Description of Artificial Sequence Synthetic peptide
SAQSIKS

SEQ ID NO: 33 - protein, 7 aa, Artificial Sequence
  Description of Artificial Sequence Synthetic peptide
AANNIKS

SEQ ID NO: 34 - protein, 7 aa, Artificial Sequence
  Description of Artificial Sequence Synthetic peptide
YNCSGKF

SEQ ID NO: 35 - protein, 7 aa, Artificial Sequence
  Description of Artificial Sequence Synthetic peptide
QAQNIKS

SEQ ID NO: 36 - protein, 7 aa, Artificial Sequence
  Description of Artificial Sequence Synthetic peptide
QADCIKS

SEQ ID NO: 37 - protein, 7 aa, Artificial Sequence
  Description of Artificial Sequence Synthetic peptide
YSCDGVF

SEQ ID NO: 38 - protein, 7 aa, Artificial Sequence
  Description of Artificial Sequence Synthetic peptide
RAQNIKS

SEQ ID NO: 39 - protein, 7 aa, Artificial Sequence
  Description of Artificial Sequence Synthetic peptide
FLCENTF

SEQ ID NO: 40 - protein, 437 aa, Saccharomyces cerevisiae
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 41 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGKSSNIKSRVNRLSVLSAITSTQQKLKLYNTLPKNGLVLYCGDIITEDGKEKKVTFDIEPYKPINTSLYICDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 42 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGTAVNIKSRVNRLSVLSAITSTQQKLKLYNTLPKNGLVLYCGDIITEDGKEKKVTFDIEPYKPINTSLYICDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 43 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGKAANIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 44 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGKASNIKSRVNRLSVLSAITSTQQKLKLYNTLPKNGLVLYCGDIITEDGKEKKVTFDIEPYKPINTSLYYCGERFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 45 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGTAESIKSRVNRLSVLSAITSTQQKLKLYNTLPKNGLVLYCGDIITEDGKEKKVTFDIEPYKPINTSLYICDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 46 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYFCDPQFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 47 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGEAASIKDRVNRLSVLSAITSTQQKLKLYNTLPKNGLVLYCGDIITEDGKEKKVTFDIEPYKPINTSLYFCDPQFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 48 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGKATNIKDRVNRLSVLSAITSTQQKLKLYNTLPKNGLVLYCGDIITEDGKEKKVTFDIEPYKPINTSLYFCDSKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 49 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGFDFDAESRVNRLSVLSAITSTQQKLKLYNTLPKNGLVLYCGDIITEDGKEKKVTFDIEPYKPINTSLTLIKPQFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 50 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGTGDKIKSRVNRLSVLSAITSTQQKLKLYNTLPKNGLVLYCGDIITEDGKEKKVTFDIEPYKPINTSLTIIKNDFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 51 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGEAASIQDRVNRLSVLSAITSTQQKLKLYNTLPKNGLVLYCGDIITEDGKEKKVTFDIEPYKPINTSLFFCDNYFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 52 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGKATNIKDRVNRLSVLSAITSTQQKLKLYNTLPKNGLVLYCGDIITEDGKEKKVTFDIEPYKPINTSLFVIVNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 53 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGAAQNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 54 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGQANSIKDRVNRLSVLSAITSTQQKLKLYNTLPKNGLVLYCGDIITEDGKEKKVTFDIEPYKPINTSLYRCDSKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 55 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGGAASIKNRVNRLSVLSAITSTQQKLKLYNTLPKNGLVLYCGDIITEDGKEKKVTFDIEPYKPINTSLYSCNTIFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 56 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGSAQNIKSRVNRLSVLSAITSTQQKLKLYNTLPKNGLVLYCGDIITEDGKEKKVTFDIEPYKPINTSLYYCDNRFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 57 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGSAGNIKSRVNRLSVLSAITSTQQKLKLYNTLPKNGLVLYCGDIITEDGKEKKVTFDIEPYKPINTSLYFCDNSFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 58 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGTAQNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 59 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGSAQSIKSRVNRLSVLSAITSTQQKLKLYNTLPKNGLVLYCGDIITEDGKEKKVTFDIEPYKPINTSLYFCDNSFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 60 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGAANNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 61 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYNCSGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 62 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGQAQNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 63 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGQADCIKSRVNRLSVLSAITSTQQKLKLYNTLPKNGLVLYCGDIITEDGKEKKVTFDIEPYKPINTSLYSCDGVFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 64 - protein, 437 aa, Artificial Sequence
  Description of Artificial Sequence Synthetic polypeptide
MDNEVEKNIEIWKVKKLVQSLEKARGNGTSMISLVIPPKGQIPLYQKMLTDEYGRAQNIKSRVNRLSVLSAITSTQQKLKLYNTLPKNGLVLYCGDIITEDGKEKKVTFDIEPYKPINTSLFLCENTFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 65 - protein, 446 aa, Artificial Sequence
  Description of Artificial Sequence Synthetic polypeptide
MSIIDSNVETWKIKRIIKNLERLRGNGTSMISLLLSPRDAIPKVQGMLAGEYGTAESIKSKINRLAVQGAITSAKERLKLYNRTPPNGLVIYCGIVIGEDKSEKKYCIDFEPFRPLNTFKYICDNKFYTKPLFELLENDDVFGFVIVDGSGCLFGTLQGNTKKIIQNITVSLPKKHGRGGQSAPRFGRIREEKRHNYVRKVAEFATQHFITEDKPNVKGIILAGSANFKNDLSESDLFDKRLSEIVLKIVDVSYGGENGFSQAITLAEDTLSNVKFVEEKNLISKYFEEIAQDTGMVVFGIEDTLNSLELGAVGTIICFENLEINRYEIRNPSTEEIKVIHLCKDQQNDTRYKMIDNNYSYFIDQNTGLDLEILSCVPLTEWLCENYSKYGVRLEFITDKSQEGFQFVNGFGGIGGFLRFKLEIENIDYEGEDVGGEEFDADEDFI

SEQ ID NO: 66 - protein, 437 aa, Artificial Sequence
  Description of Artificial Sequence Synthetic polypeptide
MAKLDDNVETWRIKRLIKNLEKLRGDGTSMISLLLSPRDQISKVQAMLAGEAGTAVNIKSRVNRQAVLSAITSAKERLKLYSKTPTNGLVVYCGTVIGEDDSEKKYTIDFEPFRPLNTFKYICDNKFCTEPLFELLENDDVFGFVIVDGNGCLFGTLQGNTKKILQQITVSLPKKHGRGGQSAPRFGRIREEKRHNYVRKVAELATQHFITDDRPNVKGLVLAGSANFKNDLSESDLFDKRLSEVVIKIVDVSYGGENGFSQAISLAEDALSNVKFVEEKNLISKYFEEIALDSGMIVFGVEDTLHSLEVGALDLLMCFENLEINRYEIRDPANDEIKIYNLNKEQEKDSKYFKNEKTGTDLEIVKCVALSEWLCENYSKYGVKLEFITDKSQEGFQFVNGFGGIGGFLRYKLEMENHDYDKEDVGGEEFNPDEDFI

SEQ ID NO: 67 - protein, 436 aa, Artificial Sequence
  Description of Artificial Sequence Synthetic polypeptide
MEGDELTQNIEQWKIKRLIDNLDKARGNGTSLISLIIPPREQLPIINKMITEEYGKSSNIKSRIVRQAVQSALTSTKERLKLYNNRLPANGLILYCGEVINEEGVCEKKYTIDFQPYRAINTTLYICDNKFHTQPLKDLLVMDDKFGFIIIDGNGALFGTLQGNTREVLHKFSVDLPKKHRRGGQSALRFARLRMESRNNYLRKVAEQAVVQFISNDKVNVAGLIVAGSAEFKNVLVQSDLFDQRLAAKVLKIVDVAYGGENGFTQAIELSADTLSNIKFIREKKVMSKFFEEVAQDTKKYCYGVEDTMKTLIMGAVEVILLFENLNFTRYVLKNPTTGVEKTLYLTPEQEENHDNFMENGEELEALEKGPLPEWIVDNYMKFGAGLEFITDRSQEGAQFVRGFGGLGAFLRYQVDMAHLNAGEEELDEEWDDDFM

SEQ ID NO: 68 - protein, 435 aa, Artificial Sequence
  Description of Artificial Sequence Synthetic polypeptide
MEEKDQRQRNIEHFKIKKLMTRLRNTRGSGTSMVSLIIPPKKQINDSTKLISDEFSKATNIKDRVNRQSVQDAMVSALQRLKLYQRTPNNGLILYCGKVLNEEGKEIKLLIDFEPYKPINTSLYFCDSKFHVDELGSLLETDPPFGFIVMDGQGALYANLQGNTKTVLNKFSVELPKKHGRGGQSSVRFARLRVEKRHNYLRKVCEVATQTFISQDKINVQGLVLAGSGDFKNELSTTQMFDPRLACKIIKIVDVSYGGENGLNQAIELAQESLTNVKFVQEKNVISKFFDCIAIDSGTVVYGVQDTMQLLLDGVIENILCFEELTTLRVTRKNKVTEQITHIFIPPNELNNPKHFKDGEHELEKIEVENLTEWLAEHYSEFGAELYFITDKSAEGCQFVKGFSGIGGFLRYKVDLEHIVNPNDEYNYEEEEGFI

SEQ ID NO: 69 - protein, 485 aa, Artificial Sequence
  Description of Artificial Sequence Synthetic polypeptide
MEQKPPFQNPLQKLQDRGTKMDQSSGSCMSKQAEEQKRLQISQYQLRKQLQMLRNMRGEQTSCVSLYIPERKKLYEVVNYLQQEESGAASIKNTQNRKSVQSALSMLRERLKNFNLHKKYPKGMIFFCADSLDSKRLLIEILDPPKAVQSFRYSCNTIFYLDDLEYMLKDQPTYGFVVADGHGYLIATVCGFDIQILQSKQEDLPNKHNKGGQSSLRFSRLCDAARERLVKNIADAMRRCYANENGTQTNLSGIVLCGMSDIKDKVQKELQQLCPCIENKIVASYDVSYSGQAGLKQALQMSTEMLKLDQLFQEMNLLSDFFANFSLETSKVVYGGELTVRALEEGNVKKLILCQDSELQRVTVYNSKTQEETIQYLMPSQVKALQDSISKTSDQEANNKKNQLQVYSQQNINEWIVENISSFSQDLEIVFVSDKTQQGVQFSKSFQGVGAYLKYSLDYSSLHAQEKENDQLEQEYCYDDEEGFI

SEQ ID NO: 70 - protein, 452 aa, Artificial Sequence
  Description of Artificial Sequence Synthetic polypeptide
MIKNIFKLLPISLRAIPLKQQQNSFSQICSLYNTKLFKVINLIQTNNKCFFSFRAKETFKKKTSSLEIETHFQVSDLTRCIYRRMKQFHNEYTDIQKILSQEQQQADINLEQLRKKINVLQPLNDVFEKLEQNIKTLQELQKQKEESASDPEMLALIEEEMENSKQLIDELQDECLEQLLPKGKHDDCSEITLEVRGGAGGSESSLFAEEVFKMYQAFFAQQGYQFSIDSFQVDMAINKGCKLGVLKVSGTNIYKKMMNESGVHKVIRVPETESKGRLHSSTISVVVMPVVPMDFKVDEKDLKFEFMRSQGAGGQHVNKVESACRVTHLPTGISVLCQDDRQQERNKQRALKLLTEKLFQVEVEKSNQQQSDQRKSQIGGGDRSDKIRTYNFPQGRITDHRTNLTLFGIEKMMKGEFLEEFIDEYEEKVNNELIESVLKQLEEDENQSQPKN

SEQ ID NO: 71 - protein, 437 aa, Artificial Sequence
  Description of Artificial Sequence Synthetic polypeptide
MDQKLNDAEIALEQFRLKKLIKTLSQERTAGTSVVSVYIPPKRIISDITNRLNTQYAEAASIKDKGNRISVQEAIQAAILRLRPYNKAPNNGLVVFCGIVQQADGKGEKKISVVIEPYRPLDLSLYFCDPQFHVEELRALLNIDPPFGFIIMDGNGSLFATIQGNSKQIIKSFDVDLPKKHNKGGQSSVRFARLRMEKRHNYLRKVCETATTCFIAEDRPNVKGLVLAGSADFKNDLAGSQFFDKRLQPLIISVVDINYGGEQGLNQAVQLSQESLLEVKYIREKNLVGQFFENIDKDTGLVVYGVQDTMRAVESQTIKTLVCVDTLQYLRLECQSKQTEQKAIKYIKGNEGYEAGSLIEEKNGEQFVILVKEDLVEHLSEKFKDYGLDFQLITDHSVEGNQFMKGFSGLGGFLRFKMDMDYLVQQEDWKDEDEDFI

SEQ ID NO: 72 - protein, 437 aa, Artificial Sequence
  Description of Artificial Sequence Synthetic polypeptide
MNQNQIQEQELEIEQFRLSKIIKTLSKTKVIGTSAVSLYIPPKKIISDITNRLNTQFSEAASIQDKVNRTSVQDSIQGAVLKLKKYTKAPASGLVLFSGLVEFEKGQKKISYVIEPFRPLQLSLFFCDNYFHIEQLEPLLKLEPSYGFIIMDGNGALFGKVQGISKETLKSFNVDLPKKHNKGGQSSLRFSRIRYWARHNYLIKVSEQAKNCFISDDKPTIKGLVLAGIADFKNKLAESPALDKRLQPLILSIVDVNYGGENGFNQAIQYSQEVLQNQKLQREKDLVAKFFLSLDLDNGKSVYGVVDTMKAIEQELVKQVICIQTLEYSRVECISKQTGVKSIKYLKGLDLYEQGSLFEDNKGEQFQVTSCQDLVEYLAENYREKGIDFQLISDNSAEGHQFYKGFGGMAGFFRFSMKMQYNMDSEEEWKSEDDEFI

SEQ ID NO: 73 - protein, 444 aa, Artificial Sequence
  Description of Artificial Sequence Synthetic polypeptide
MVESIAAGQVGDNKHIEMWKIKRLINKLENCKGNGTSMVSLIIPPKEDINKSGKLLVGELSAAQNIKSRITRQSVITAITSTKEKLKLYRQTPTNGLCIYCGVILMEDGKTEKKINFDFEPFRPINQFMYFCGGKFQTEPLTTLLADDDKFGFIIVDGNGALYATLQGNSREILQKITVELPKKHRKGGQSSVRFARLREEKRHNYLRKVAELAGSNFITNDKPNVTGLVLAGNAGFKNELSETDMLDKRLLPIIVSIVDVSYGGENGLNEAITLSADALTNVKFVAEKKLVSKFFEEISLDTGMIVFGVQDTMKALELGAVETILLFEELEITRYVIKNPVKGDTRTLFLNPTQQKDSKYFKDQASGLDMDVISEDQLAEWLCHNYQNYAQVEFITDKSQEGYQFVKGFGGIGGFLRYKVDMEEALGDVGDGGDDFDPDTDFI

SEQ ID NO: 74 - protein, 437 aa, Artificial Sequence
  Description of Artificial Sequence Synthetic polypeptide
MDEAKLLQLEMWRLRKQLQKLDNTNTNSTSVVSLVMPPGEDINKMVQMLNQEATQADCIKSRQNRQAVQTAIILAANRCKLYPKMPKNGLAVFAGEVYVDGKIKKIAVHFSPCKPIGNFMYSCDGVFHTQEVKDLLTVEEVYGFIIMDGHGTLIATLCGSHREIKHRMLVDLPKKHGRGGQSSVRFARLRMEARGNYVKIITELCTKYFITGDRLNVSGVILAGSADFKDVLAGSDFMDPRIKEGIIKQVDIGYGMEQGLSQAIEQAGDILKDVRLFKEVKIINEFLDHISRDTRKYCFGIIDTLRCLEMGSVEHLVVWEDFPWDRVQCRNSEGTEYYKLVNTVTGAINLVDNDESIRQGEEEIEAELFVEWLAQNIEKFGAEIHLVTENSNEGTQFCSGFGGLGGILRWQMDLNELGKYMDIGKENNDLDDLEDFM

SEQ ID NO: 75 - protein, 437 aa, Artificial Sequence; identical to SEQ ID NO: 40
  Description of Artificial Sequence Synthetic polypeptide

SEQ ID NO: 76 - protein, 685 aa, Artificial Sequence
  Description of Artificial Sequence Synthetic polypeptide
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVNDMFGGKDHVSLIFMGHVDAGKSTMGGNLLYLTGSVDKRTIEKYEREAKDAGRQGWYLSWVMDTNKEERNDGKTIEVGKAYFETEKRRYTILDAPGHKMYVSEMIGGASQADVGVLVISARKGEYETGFERGGQTREHALLAKTQGVNKMVVVVNKMDDPTVNWSKERYDQCVSNVSNFLRAIGYNIKTDVVFMPVSGYSGANLKDHVDPKECPWYTGPTLLEYLDTMNHVDRHINAPFMLPIAAKMKDLGTIVEGKIESGHIKKGQSTLLMPNKTAVEIQNIYNETENEVDMAMCGEQVKLRIKGVEEEDISPGFVLTSPKNPIKSVTKFVAQIAIVELKSIIAAGFSCVMHVHTAIEEVHIVKLLHKLEKGTNRKSKKPPAFAKKGMKVIAVLETEAPVCVETYQDYPQLGRFTLRDQGTTIAIGKIVKIAE

SEQ ID NO: 77 - protein, 446 aa, Artificial Sequence; identical to SEQ ID NO: 65
  Description of Artificial Sequence Synthetic polypeptide

SEQ ID NO: 78 - protein, 799 aa, Artificial Sequence
  Description of Artificial Sequence Synthetic polypeptide
MDKNSDFPALGSLKVQNQSSKDKNKKKEKEVKKEDEKKDEVKDEPQNNEEAKKTKLEATGRVFAAKSFTPKEPVKINYYIPHEEPTLSEMDFFPTLGGPASTAPVVSEAQKRHAEFLERHELFKPYICLIPKELWYVPDPYFDMFGYPVMALLPGLYAYLWNTYNIFFTPEGAWNTNKETVMSTLGELWTHAEWRDRIIAKEQKETEEWERQMREWEEENAEDDEGLSIDDMENYGKGKKKNKNKKKKDKAKRPPPPPPKSLSSYKRFEKKKEDVVVPKKNIGFKEVSEITFEEEVVEVDETRQPSSLVFIGPVDAVKSTICGNLMFMTGMVDERTIEKFKQEAKEKNRDSWWLAYVMDINDDEKSKGKTVEVGRATMETPTKRYTIFDAPGHKNYVPDMIMGAAMADVAALVISARKGEFEAGFERDGQTREHAQLARSLGVNKLVVVVNEMDEETVQWSEERYNDILSGVTPFLIDQCGYKREDLIFVPISGLNGHNIDKLASCCPWYTGPTLLEILDCIEPPKRNIDGPLRVPVLDKMKDRGVVAFGKVESGVIRIGPKLAVMPNNTKCQVVGIYNCKLELVRYANPGENIQIKVRMIEDENQINKGDVLCPYDNLAPITDLFEAELTILELLPHRPIITPGYKSMMHLHTISDEIVIQTLTGIYELDGSGKEYVKKNPKYCKSGSKVIVKISTRVPVCLEKYEFIVHMGRFTLRDEGKTIALGKVLRYKPAVIKKVEEIPPGVGDEGQAKLEESEEFSGSRGDSPSKDDNKYEVITYDPEEDTIIASSTGSENAE

SEQ ID NO: 79 - protein, 437 aa, Artificial Sequence; identical to SEQ ID NO: 66
  Description of Artificial Sequence Synthetic polypeptide

SEQ ID NO: 80 - protein, 799 aa, Artificial Sequence; identical to SEQ ID NO: 78
  Description of Artificial Sequence Synthetic polypeptide

SEQ ID NO: 81 - protein, 436 aa, Artificial Sequence; identical to SEQ ID NO: 67
  Description of Artificial Sequence Synthetic polypeptide

SEQ ID NO: 82 - protein, 409 aa, Artificial Sequence
  Description of Artificial Sequence Synthetic polypeptide
MVDSGKSTSCGHLIYKCGGIDKRTIEKYEKEAKEMGKSSFKYAWVLDKLKAERERGITIDISLFKFQTDKFYFTIIDAPGHRDFIKNMITGTSQADVAILIIAAGKGEFEAGYSKNGQTREHALLAFTLGVKQMVVGVNKMDDKSAEWKQDRYLEIKQEVSEYLKKVGYNPAKVPFIPISGWLGDNMVEKSTNMPWYDGPTLLGALDNVQPPKRHVDKPLRLPVQDVYKISGIGTVPVGRVETGVLRPGTVVVFAPSGISTEVKSVEMHHESLEEALPGDNVGFNIKNIAVNQIKRGYVASDSRSDPARESIDFTAQVIILHHPGQISVGYTPVLDCHTAHIACRFNDLKQKVDRRSGAVLEQEPKFVKSGDAAIVTLIPTKSMCVEPFTEYPPLGRFAVRDMRQTVAV

SEQ ID NO: 83 - protein, 435 aa, Artificial Sequence; identical to SEQ ID NO: 68
  Description of Artificial Sequence Synthetic polypeptide

SEQ ID NO: 84 - protein, 646 aa, Artificial Sequence
  Description of Artificial Sequence Synthetic polypeptide
MDYQEAKRLAKEEKLRKLKAKIQENKVEDFQVTEQQGPLPPYDDQGNQQGKWLTTKYKFGEWYYDMNGNPVFISPDDEYDQNGSLYDAESNPYLDYAYDKFGNPCPVILMTQDNEYFLFTPIFEIQIPQSIIKDINQKQEKPATQKVETVAKEKKAPIQKAPAPPKIPKRLLKEQEAANSSAVVADYKHEKLVTVQEAIDFESKNFKEGQEMVKVDRERDSVNIVFIGHVDAGKSTLSGRILKNCGEVDETEIRKFELEAKEKNRESWVLAYIMDINEEERSKGITVECGKAHFQLANKRFVLLDAPGHKNYVPNMIAGACQADVAALIISARQGEFEAGFEGGQTQEHAHLAKALGVQHMICVVSKMDEVNWDKKRYDHIHDSVEPFLRNQVGIQSIEWVPINGFLNENIDTPIPTERCEWYKGDTLFDKFNKVPVPLRDPNGPVRIPVLDKLKDQGQFLFGKIESGTIRDDLWVTLMPYRKQFQILSIYNTKDQRVLYASAGENVKIKLKGLEDKDIERGYMVCSTEDLCPITQLFIAEITILQLPEHKPIMSQGYSCVLHMHTSVAEIEIEEVEAVQNPENKKLTKNTFLKSNQTGVVKIGIKGGLMCLEKFETISQLGRFTLRDEEKTIGFGRVMKIKPYKV

SEQ ID NO: 85 - protein, 485 aa, Artificial Sequence; identical to SEQ ID NO: 69
  Description of Artificial Sequence Synthetic polypeptide

SEQ ID NO: 86 - protein, 646 aa, Artificial Sequence; identical to SEQ ID NO: 84
  Description of Artificial Sequence Synthetic polypeptide

SEQ ID NO: 87 - protein, 452 aa, Artificial Sequence; identical to SEQ ID NO: 70
  Description of Artificial Sequence Synthetic polypeptide

SEQ ID NO: 88 - protein, 646 aa, Artificial Sequence; identical to SEQ ID NO: 84
  Description of Artificial Sequence Synthetic polypeptide

SEQ ID NO: 89 - protein, 437 aa, Artificial Sequence; identical to SEQ ID NO: 71
  Description of Artificial Sequence Synthetic polypeptide

SEQ ID NO: 90 - protein, 756 aa, Artificial Sequence
  Description of Artificial Sequence Synthetic polypeptide
MSYQYGQQMGQYPYDPNMNMMGFDPQMYQEYAYYYLGGPPTPPPPKGPYPGITHEDYESFDINKQILFQRFLGETAAYYAKHLPKYQKEMEEFLNTNTAYQMNESEKQLMQSYLDFKKKEKEYESFLKQLEQQALNPELEQQKKLEQQKLEQQKIEQQKLEEQKKQQQLEQQKQQQQQQQQQPQQEQPKEGATTAVRPKKKLNLNAKPLEIALNPPKMPNFPKHPDFLDFDKFWNNYSTYISLYNPSCEDEYKNYPKPEQLKKKEADEEAKRKKKQEEIERAIKKRQDAQERAKDKPAQSVNLVEQVVKLEGEVDLQKYVDPDETRQPVNLVFIGHVDAGKSTLCGRLLLELGEVSEADIKKYEQEAVQNNRDSWWLAYVMDQNEEEKQKGKTVECGKAQFVTKQKRFILADAPGHKNYVPNMIMGACQADLAGLIVSAKTGEFESGFEKGGQTQEHALLAKSLGVDHIIIIVTKMDTIDWNQDRFNLISQNIQEFVLKQCKFDNIYVIPIDALSGSNIKSRVDESKCNWYKGPSLIDLIDTVSIPKRNEEGPIRMPILDKFKDMGSLYIYGKLESGKIIEGLDVSIYPKKQPFQITELYNMKDQKMKYAKAGENIKIKVKNIEEEEIKRGYMMCNLTSNPCLVSQEFQAKIRLLDLPESRRIFSEGYQCIMHLHSAVEEIEISCVEAVIDAETKKSIKQNFLKSFNEGIAKISIKNPVCMEKYETLAQLGRFALRDDGKTIGFGEILKVKPVKQG

SEQ ID NO: 91 - protein, 437 aa, Artificial Sequence; identical to SEQ ID NO: 72
  Description of Artificial Sequence Synthetic polypeptide

SEQ ID NO: 92 - protein, 756 aa, Artificial Sequence; identical to SEQ ID NO: 90
  Description of Artificial Sequence Synthetic polypeptide

SEQ ID NO: 93 - protein, 446 aa, Artificial Sequence; identical to SEQ ID NO: 65
  Description of Artificial Sequence Synthetic polypeptide

SEQ ID NO: 94 - protein, 755 aa, Artificial Sequence
  Description of Artificial Sequence Synthetic polypeptide
MDKNSDQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADALIKEQEEEVDDEVVNDVDETRQPSSLVFIGPVDAVKSTICGNLMFMTGMVDERTIEKFKQEAKEKNRDSWWLAYVMDINDDEKSKGKTVEVGRATMETPTKRYTIFDAPGHKNYVPDMIMGAAMADVAALVISARKGEFEAGFERDGQTREHAQLARSLGVNKLVVVVNEMDEETVQWSEERYNDILSGVTPFLIDQCGYKREDLIFVPISGLNGHNIDKLASCCPWYTGPTLLEILDCIEPPKRNIDGPLRVPVLDKMKDRGVVAFGKVESGVIRIGPKLAVMPNNTKCQVVGIYNCKLELVRYANPGENIQIKVRMIEDENQINKGDVLCPYDNLAPITDLFEAELTILELLPHRPIITPGYKSMMHLHTISDEIVIQTLTGIYELDGSGKEYVKKNPKYCKSGSKVIVKISTRVPVCLEKYEFIVHMGRFTLRDEGKTIALGKVLRYKPAVIKKVEEIPPGVGDEGQAKLEESEEFSGSRGDSPSKDDNKYEVITYDPEEDTIIASSTGSENAE

SEQ ID NO: 95 - protein, 437 aa, Artificial Sequence; identical to SEQ ID NO: 66
  Description of Artificial Sequence Synthetic polypeptide

SEQ ID NO: 96 - protein, 754 aa, Artificial Sequence
  Description of Artificial Sequence Synthetic polypeptide
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADALIKEQEEEVDDEVVNDVDETRQPSSLVFIGPVDAVKSTICGNLMFMTGMVDERTIEKFKQEAKEKNRDSWWLAYVMDINDDEKSKGKTVEVGRATMETPTKRYTIFDAPGHKNYVPDMIMGAAMADVAALVISARKGEFEAGFERDGQTREHAQLARSLGVNKLVVVVNEMDEETVQWSEERYNDILSGVTPFLIDQCGYKREDLIFVPISGLNGHNIDKLASCCPWYTGPTLLEILDCIEPPKRNIDGPLRVPVLDKMKDRGVVAFGKVESGVIRIGPKLAVMPNNTKCQVVGIYNCKLELVRYANPGENIQIKVRMIEDENQINKGDVLCPYDNLAPITDLFEAELTILELLPHRPIITPGYKSMMHLHTISDEIVIQTLTGIYELDGSGKEYVKKNPKYCKSGSKVIVKISTRVPVCLEKYEFIVHMGRFTLRDEGKTIALGKVLRYKPAVIKKVEEIPPGVGDEGQAKLEESEEFSGSRGDSPSKDDNKYEVITYDPEEDTIIASSTGSENAE

SEQ ID NO: 97 - protein, 437 aa, Artificial Sequence; identical to SEQ ID NO: 71
  Description of Artificial Sequence Synthetic polypeptide

SEQ ID NO: 98 - protein, 688 aa, Artificial Sequence
  Description of Artificial Sequence Synthetic polypeptide
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADALIKEQEEEVDDEVVNDPDETRQPVNLVFIGHVDAGKSTLCGRLLLELGEVSEADIKKYEQEAVQNNRDSWWLAYVMDQNEEEKQKGKTVECGKAQFVTKQKRFILADAPGHKNYVPNMIMGACQADLAGLIVSAKTGEFESGFEKGGQTQEHALLAKSLGVDHIIIIVTKMDTIDWNQDRFNLISQNIQEFVLKQCKFDNIYVIPIDALSGSNIKSRVDESKCNWYKGPSLIDLIDTVSIPKRNEEGPIRMPILDKFKDMGSLYIYGKLESGKIIEGLDVSIYPKKQPFQITELYNMKDQKMKYAKAGENIKIKVKNIEEEEIKRGYMMCNLTSNPCLVSQEFQAKIRLLDLPESRRIFSEGYQCIMHLHSAVEEIEISCVEAVIDAETKKSIKQNFLKSFNEGIAKISIKNPVCMEKYETLAQLGRFALRDDGKTIGFGEILKVKPVKQG

SEQ ID NO: 99 - protein, 437 aa, Artificial Sequence; identical to SEQ ID NO: 72
  Description of Artificial Sequence Synthetic polypeptide

SEQ ID NO: 100 - protein, 688 aa, Artificial Sequence; identical to SEQ ID NO: 98
  Description of Artificial Sequence Synthetic polypeptide

SEQ ID NO: 101 - DNA, 1314 bp, Saccharomyces cerevisiae
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtactgcctcgaatattaaatctagggttaatcgtctttccgttttatctgctatcacttccacccaacaaaagttgaagctatataatactttgcccaagaacggtttagttttatattgtggtgatatcatcactgaagatggtaaagaaaaaaaggtcacttttgacatcgaaccttacaaacctatcaacacatccttatatttgtgtgataacaaatttcatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 102 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaagtcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacatctgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 103 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgctgttaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacatctgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 104 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggctgctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttgtgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 105 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactactgtggtgaaagattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 106 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgctgaatctatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacatctgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 107 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacccacaattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 108 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgaagctgcttctatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacccacaattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 109 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggctaccaacatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgactctaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 110 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtttcgacttcgacgctgaatctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgaccttgatcaagccacaattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 111 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccggtgacaagatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgaccatcatcaagaacgacttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 112 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgaagctgcttctatccaagacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgttcttctgtgacaactacttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 113 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggctaccaacatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgttcgttatcgttaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 114 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgctgctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 115 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtcaagctaactctatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacagatgtgactctaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 116 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtggtgctgcttctatcaagaacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactcttgtaacaccatcttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 117 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggttctgctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactactgtgacaacagattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 118 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggttctgctggtaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacaactctttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 119 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 120 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggttctgctcaatctatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacaactctttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 121 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgctgctaacaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 122 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacaactgttctggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 123 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtcaagctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 124 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtcaagctgactgtatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactcttgtgacggtgttttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 125 - DNA, 1314 bp, Artificial Sequence
  Description of Artificial Sequence Synthetic polynucleotide
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtagagctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgttcttgtgtgaaaacaccttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 126 - DNA, 1344 bp, Artificial Sequence
  Description of Artificial Sequence Synthetic polynucleotide
atgtctatcatcgactctaacgttgaaacctggaagatcaagagaatcatcaagaacttggaaagattgagaggtaacggtacctctatgatctctttgttgttgtctccacgcgacgctatcccaaaggttcaaggtatgttggctggtgaatacggtaccgctgaatctatcaagtctaagatcaacagattggctgttcaaggtgctatcacctctgctaaggaaagattgaagttgtacaacagaaccccaccaaacggtttggttatctactgtggtatcgttatcggtgaagacaagtctgaaaagaagtactgtatcgacttcgaaccattcagaccattgaacaccttcaagtacatctgtgacaacaagttctacaccaagccattgttcgaattgttggaaaacgacgacgttttcggtttcgttatcgttgacggttctggttgtttgttcggtaccttgcaaggtaacaccaagaagatcatccaaaacatcaccgtttctttgccaaagaagcacggtagaggtggtcaatctgctccaagattcggtagaatcagagaagaaaagagacacaactacgttagaaaggttgctgaattcgctacccaacacttcatcaccgaagacaagccaaacgttaagggtatcatcttggctggttctgctaacttcaagaacgacttgtctgaatctgacttgttcgacaagagattgtctgaaatcgttttgaagatcgttgacgtttcttacggtggtgaaaacggtttctctcaagctatcaccttggctgaagacaccttgtctaacgttaagttcgttgaagaaaagaacttgatctctaagtacttcgaagaaatcgctcaagacaccggtatggttgttttcggtatcgaagacaccttgaactctttggaattgggtgctgttggtaccatcatctgtttcgaaaacttggaaatcaacagatacgaaatcagaaacccatctaccgaagaaatcaaggttatccacttgtgtaaggaccaacaaaacgacaccagatacaagatgatcgacaacaactactcttacttcatcgaccaaaacaccggtttggacttggaaatcttgtcttgtgttccattgaccgaatggttgtgtgaaaactactctaagtacggtgttagattggaattcatcaccgacaagtctcaagaaggtttccaattcgttaacggtttcggtggtatcggtggtttcttgagattcaagttggaaatcgaaaacatcgactacgaaggtgaagacgttggtggtgaagaattcgacgctgacgaagacttcatctaatag

SEQ ID NO: 127 - DNA, 1317 bp, Artificial Sequence
  Description of Artificial Sequence Synthetic polynucleotide
atggctaagttggacgacaacgttgaaacctggagaatcaagagattgatcaagaacttggaaaagttgagaggtgacggtacctctatgatctctttgttgttgtctccacgcgaccaaatctctaaggttcaagctatgttggctggtgaagctggtaccgctgttaacatcaagtctagagttaacagacaagctgttttgtctgctatcacctctgctaaggaaagattgaagttgtactctaagaccccaaccaacggtttggttgtttactgtggtaccgttatcggtgaagacgactctgaaaagaagtacaccatcgacttcgaaccattcagaccattgaacaccttcaagtacatctgtgacaacaagttctgtaccgaaccattgttcgaattgttggaaaacgacgacgttttcggtttcgttatcgttgacggtaacggttgtttgttcggtaccttgcaaggtaacaccaagaagatcttgcaacaaatcaccgtttctttgccaaagaagcacggtagaggtggtcaatctgctccaagattcggtagaatcagagaagaaaagagacacaactacgttagaaaggttgctgaattggctacccaacacttcatcaccgacgacagaccaaacgttaagggtttggttttggctggttctgctaacttcaagaacgacttgtctgaatctgacttgttcgacaagagattgtctgaagttgttatcaagatcgttgacgtttcttacggtggtgaaaacggtttctctcaagctatctctttggctgaagacgctttgtctaacgttaagttcgttgaagaaaagaacttgatctctaagtacttcgaagaaatcgctttggactctggtatgatcgttttcggtgttgaagacaccttgcactctttggaagttggtgctttggacttgttgatgtgtttcgaaaacttggaaatcaacagatacgaaatccgcgacccagctaacgacgaaatcaagatctacaacttgaacaaggaacaagaaaaggactctaagtacttcaagaacgaaaagaccggtaccgacttggaaatcgttaagtgtgttgctttgtctgaatggttgtgtgaaaactactctaagtacggtgttaagttggaattcatcaccgacaagtctcaagaaggtttccaattcgttaacggtttcggtggtatcggtggtttcttgagatacaagttggaaatggaaaaccacgactacgacaaggaagacgttggtggtgaagaattcaacccagacgaagacttcatctaatag

SEQ ID NO: 128 - DNA, 1314 bp, Artificial Sequence
  Description of Artificial Sequence Synthetic polynucleotide
atggaaggtgacgaattgacccaaaacatcgaacaatggaagatcaagagattgatcgacaacttggacaaggctagaggtaacggtacctctttgatctctttgatcatcccaccaagagaacaattgccaatcatcaacaagatgatcaccgaagaatacggtaagtcttctaacatcaagtctagaatcgttagacaagctgttcaatctgctttgacctctaccaaggaaagattgaagttgtacaacaacagattgccagctaacggtttgatcttgtactgtggtgaagttatcaacgaagaaggtgtttgtgaaaagaagtacaccatcgacttccaaccatacagagctatcaacaccaccttgtacatctgtgacaacaagttccacacccaaccattgaaggacttgttggttatggacgacaagttcggtttcatcatcatcgacggtaacggtgctttgttcggtaccttgcaaggtaacaccagagaagttttgcacaagttctctgttgacttgccaaagaagcacagaagaggtggtcaatctgctttgagattcgctagattgagaatggaatctagaaacaactacttgagaaaggttgctgaacaagctgttgttcaattcatctctaacgacaaggttaacgttgctggtttgatcgttgctggttctgctgaattcaagaacgttttggttcaatctgacttgttcgaccaaagattggctgctaaggttttgaagatcgttgacgttgcttacggtggtgaaaacggtttcacccaagctatcgaattgtctgctgacaccttgtctaacatcaagttcatcagagaaaagaaggttatgtctaagttcttcgaagaagttgctcaagacaccaagaagtactgttacggtgttgaagacaccatgaagaccttgatcatgggtgctgttgaagttatcttgttgttcgaaaacttgaacttcaccagatacgttttgaagaacccaaccaccggtgttgaaaagaccttgtacttgaccccagaacaagaagaaaaccacgacaacttcatggaaaacggtgaagaattggaagctttggaaaagggtccattgccagaatggatcgttgacaactacatgaagttcggtgctggtttggaattcatcaccgacagatctcaagaaggtgctcaattcgttagaggtttcggtggtttgggtgctttcttgagataccaagttgacatggctcacttgaacgctggtgaagaagaattggacgaagaatgggacgacgacttcatgtaatag

SEQ ID NO: 129 - DNA, 1311 bp, Artificial Sequence
  Description of Artificial Sequence Synthetic polynucleotide
atggaagaaaaggaccaaagacaaagaaacatcgaacacttcaagatcaagaagttgatgaccagattgagaaacaccagaggttctggtacctctatggtttctttgatcatcccaccaaagaagcaaatcaacgactctaccaagttgatctctgacgaattctctaaggctaccaacatcaaggacagagttaacagacaatctgttcaagacgctatggtttctgctttgcaaagattgaagttgtaccaaagaaccccaaacaacggtttgatcttgtactgtggtaaggttttgaacgaagaaggtaaggaaatcaagttgttgatcgacttcgaaccatacaagccaatcaacacctctttgtacttctgtgactctaagttccacgttgacgaattgggttctttgttggaaaccgacccaccattcggtttcatcgttatggacggtcaaggtgctttgtacgctaacttgcaaggtaacaccaagaccgttttgaacaagttctctgttgaattgccaaagaagcacggtagaggtggtcaatcttctgttagattcgctagattgagagttgaaaagagacacaactacttgagaaaggtttgtgaagttgctacccaaaccttcatctctcaagacaagatcaacgttcaaggtttggttttggctggttctggtgacttcaagaacgaattgtctaccacccaaatgttcgacccaagattggcttgtaagatcatcaagatcgttgacgtttcttacggtggtgaaaacggtttgaaccaagctatcgaattggctcaagaatctttgaccaacgttaagttcgttcaagaaaagaacgttatctctaagttcttcgactgtatcgctatcgactctggtaccgttgtttacggtgttcaagacaccatgcaattgttgttggacggtgttatcgaaaacatcttgtgtttcgaagaattgaccaccttgagagttaccagaaagaacaaggttaccgaacaaatcacccacatcttcatcccaccaaacgaattgaacaacccaaagcacttcaaggacggtgaacacgaattggaaaagatcgaagttgaaaacttgaccgaatggttggctgaacactactctgaattcggtgctgaattgtacttcatcaccgacaagtctgctgaaggttgtcaattcgttaagggtttctctggtatcggtggtttcttgagatacaaggttgacttggaacacatcgttaacccaaacgacgaatacaactacgaagaagaagaaggtttcatctgatag

SEQ ID NO: 130 - DNA, 1461 bp, Artificial Sequence
  Description of Artificial Sequence Synthetic polynucleotide
atggaacaaaagccaccattccaaaacccattgcaaaagttgcaagacagaggtaccaagatggaccaatcttctggttcttgtatgtctaagcaagctgaagaacaaaagagattgcaaatctctcaataccaattgagaaagcaattgcaaatgttgagaaacatgagaggtgaacaaacctcttgtgtttctttgtacatcccagaaagaaagaagttgtacgaagttgttaactacttgcaacaagaagaatctggtgctgcttctatcaagaacacccaaaacagaaagtctgttcaatctgctttgtctatgttgagagaaagattgaagaacttcaacttgcacaagaagtacccaaagggtatgatcttcttctgtgctgactctttggactctaagagattgttgatcgaaatcttggacccaccaaaggctgttcaatctttcagatactcttgtaacaccatcttctacttggacgacttggaatacatgttgaaggaccaaccaacctacggtttcgttgttgctgacggtcacggttacttgatcgctaccgtttgtggtttcgacatccaaatcttgcaatctaagcaagaagacttgccaaacaagcacaacaagggtggtcaatcttctttgagattctctagattgtgtgacgctgctagagaaagattggttaagaacatcgctgacgctatgagaagatgttacgctaacgaaaacggtacccaaaccaacttgtctggtatcgttttgtgtggtatgtctgacatcaaggacaaggttcaaaaggaattgcaacaattgtgtccatgtatcgaaaacaagatcgttgcttcttacgacgtttcttactctggtcaagctggtttgaagcaagctttgcaaatgtctaccgaaatgttgaagttggaccaattgttccaagaaatgaacttgttgtctgacttcttcgctaacttctctttggaaacctctaaggttgtttacggtggtgaattgaccgttagagctttggaagaaggtaacgttaagaagttgatcttgtgtcaagactctgaattgcaaagagttaccgtttacaactctaagacccaagaagaaaccatccaatacttgatgccatctcaagttaaggctttgcaagactctatctctaagacctctgaccaagaagctaacaacaagaagaaccaattgcaagtttactctcaacaaaacatcaacgaatggatcgttgaaaacatctcttctttctctcaagacttggaaatcgttttcgtttctgacaagacccaacaaggtgttcaattctctaagtctttccaaggtgttggtgcttacttgaagtactctttggactactcttctttgcacgctcaagaaaaggaaaacgaccaattggaacaagaatactgttacgacgacgaagaaggtttcatctgatag

SEQ ID NO: 131 - DNA, 1362 bp, Artificial Sequence
  Description of Artificial Sequence Synthetic polynucleotide
atgatcaagaacatcttcaagttgttgccaatctctttgagagctatcccattgaagcaacaacaaaactctttctctcaaatctgttctttgtacaacaccaagttgttcaaggttatcaacttgatccaaaccaacaacaagtgtttcttctctttcagagctaaggaaaccttcaagaagaagacctcttctttggaaatcgaaacccacttccaagtttctgacttgaccagatgtatctacagaagaatgaagcaattccacaacgaatacaccgacatccaaaagatcttgtctcaagaacaacaacaagctgacatcaacttggaacaattgagaaagaagatcaacgttttgcaaccattgaacgacgttttcgaaaagttggaacaaaacatcaagaccttgcaagaattgcaaaagcaaaaggaagaatctgcttctgacccagaaatgttggctttgatcgaagaagaaatggaaaactctaagcaattgatcgacgaattgcaagacgaatgtttggaacaattgttgccaaagggtaagcacgacgactgttctgaaatcaccttggaagttagaggtggtgctggtggttctgaatcttctttgttcgctgaagaagttttcaagatgtaccaagctttcttcgctcaacaaggttaccaattctctatcgactctttccaagttgacatggctatcaacaagggttgtaagttgggtgttttgaaggtttctggtaccaacatctacaagaagatgatgaacgaatctggtgttcacaaggttatcagagttccagaaaccgaatctaagggtagattgcactcttctaccatctctgttgttgttatgccagttgttccaatggacttcaaggttgacgaaaaggacttgaagttcgaattcatgagatctcaaggtgctggtggtcaacacgttaacaaggttgaatctgcttgtagagttacccacttgccaaccggtatctctgttttgtgtcaagacgacagacaacaagaaagaaacaagcaaagagctttgaagttgttgaccgaaaagttgttccaagttgaagttgaaaagtctaaccaacaacaatctgaccaaagaaagtctcaaatcggtggtggtgacagatctgacaagatcagaacctacaacttcccacaaggtagaatcaccgaccacagaaccaacttgaccttgttcggtatcgaaaagatgatgaagggtgaattcttggaagaattcatcgacgaatacgaagaaaaggttaacaacgaattgatcgaatctgttttgaagcaattggaagaagacgaaaaccaatctcaaccaaagaactgatag

SEQ ID NO: 132 - DNA, 1317 bp, Artificial Sequence
  Description of Artificial Sequence Synthetic polynucleotide
atggaccaaaagttgaacgacgctgaaatcgctttggaacaattcagattgaagaagttgatcaagaccttgtctcaagaaagaaccgctggtacctctgttgtttctgtttacatcccaccaaagagaatcatctctgacatcaccaacagattgaacacccaatacgctgaagctgcttctatcaaggacaagggtaacagaatctctgttcaagaagctatccaagctgctatcttgagactcagaccatacaacaaggctccaaacaacggtttggttgttttctgtggtatcgttcaacaagctgacggtaagggtgaaaagaagatctctgttgttatcgaaccatacagaccattggacttgtctttgtacttctgtgacccacaattccacgttgaagaattgagagctttgttgaacatcgacccaccattcggtttcatcatcatggacggtaacggttctttgttcgctaccatccaaggtaactctaagcaaatcatcaagtctttcgacgttgacttgccaaagaagcacaacaagggtggtcaatcttctgttagattcgctagattgagaatggaaaagagacacaactacttgagaaaggtttgtgaaaccgctaccacctgtttcatcgctgaagacagaccaaacgttaagggtttggttttggctggttctgctgacttcaagaacgacttggctggttctcaattcttcgacaagagattgcaaccattgatcatctctgttgttgacatcaactacggtggtgaacaaggtttgaaccaagctgttcaattgtctcaagaatctttgttggaagttaagtacatcagagaaaagaacttggttggtcaattcttcgaaaacatcgacaaggacaccggtttggttgtttacggtgttcaagacaccatgagagctgttgaatctcaaaccatcaagaccttggtttgtgttgacaccttgcaatacttgagattggaatgtcaatctaagcaaaccgaacaaaaggctatcaagtacatcaagggtaacgaaggttacgaagctggttctttgatcgaagaaaagaacggtgaacaattcgttatcttggttaaggaagacttggttgaacacttgtctgaaaagttcaaggactacggtttggacttccaattgatcaccgaccactctgttgaaggtaaccaattcatgaagggtttctctggtttgggtggtttcttgagattcaagatggacatggactacttggttcaacaagaagactggaaggacgaagacgaagacttcatctgatag

SEQ ID NO: 133 - DNA, 1317 bp, Artificial Sequence
  Description of Artificial Sequence Synthetic polynucleotide
atgaaccaaaaccaaatccaagaacaagaattggaaatcgaacaattcagattgtctaagatcatcaagaccttgtctaagaccaaggttatcggtacctctgctgtttctttgtacatcccaccaaagaagatcatctctgacatcaccaacagattgaacacccaattctctgaagctgcttctatccaagacaaggttaacagaacctctgttcaagactctatccaaggtgctgttttgaagttgaagaagtacaccaaggctccagcttctggtttggttttgttctctggtttggttgaattcgaaaagggtcaaaagaagatctcttacgttatcgaaccattcagaccattgcaattgtctttgttcttctgtgacaactacttccacatcgaacaattggaaccattgttgaagttggaaccatcttacggtttcatcatcatggacggtaacggtgctttgttcggtaaggttcaaggtatctctaaggaaaccttgaagtctttcaacgttgacttgccaaagaagcacaacaagggtggtcaatcttctttgagattctctagaatcagatactgggctagacacaactacttgatcaaggtttctgaacaagctaagaactgtttcatctctgacgacaagccaaccatcaagggtttggttttggctggtatcgctgacttcaagaacaagttggctgaatctccagctttggacaagagattgcaaccattgatcttgtctatcgttgacgttaactacggtggtgaaaacggtttcaaccaagctatccaatactctcaagaagttttgcaaaaccaaaagttgcaaagagaaaaggacttggttgctaagttcttcttgtctttggacttggacaacggtaagtctgtttacggtgttgttgacaccatgaaggctatcgaacaagaattggttaagcaagttatctgtatccaaaccttggaatactctagagttgaatgtatctctaagcaaaccggtgttaagtctatcaagtacttgaagggtttggacttgtacgaacaaggttctttgttcgaagacaacaagggtgaacaattccaagttacctcttgtcaagacttggttgaatacttggctgaaaactacagagaaaagggtatcgacttccaattgatctctgacaactctgctgaaggtcaccaattctacaagggtttcggtggtatggctggtttcttcagattctctatgaagatgcaatacaacatggactctgaagaagaatggaagtctgaagacgacgaattcatctgatag

SEQ ID NO: 134 - DNA, 1338 bp, Artificial Sequence
  Description of Artificial Sequence Synthetic polynucleotide
atggttgaatctatcgctgctggtcaagttggtgacaacaagcacatcgaaatgtggaagatcaagagattgatcaacaagttggaaaactgtaagggtaacggtacctctatggtttctttgatcatcccaccaaaggaagacatcaacaagtctggtaagttgttggttggtgaattgtctgctgctcaaaacatcaagtctagaatcaccagacaatctgttatcaccgctatcacctctaccaaggaaaagttgaagttgtacagacaaaccccaaccaacggtttgtgtatctactgtggtgttatcttgatggaagacggtaagaccgaaaagaagatcaacttcgacttcgaaccattcagaccaatcaaccaattcatgtacttctgtggtggtaagttccaaaccgaaccattgaccaccttgttggctgacgacgacaagttcggtttcatcatcgttgacggtaacggtgctttgtacgctaccttgcaaggtaactctagagaaatcttgcaaaagatcaccgttgaattgccaaagaagcacagaaagggtggtcaatcttctgttagattcgctagattgagagaagaaaagagacacaactacttgagaaaggttgctgaattggctggttctaacttcatcaccaacgacaagccaaacgttaccggtttggttttggctggtaacgctggtttcaagaacgaattgtctgaaaccgacatgttggacaagagattgttgccaatcatcgtttctatcgttgacgtttcttacggtggtgaaaacggtttgaacgaagctatcaccttgtctgctgacgctttgaccaacgttaagttcgttgctgaaaagaagttggtttctaagttcttcgaagaaatctctttggacaccggtatgatcgttttcggtgttcaagacaccatgaaggctttggaattgggtgctgttgaaaccatcttgttgttcgaagaattggaaatcaccagatacgttatcaagaacccagttaagggtgacaccagaaccttgttcttgaacccaacccaacaaaaggactctaagtacttcaaggaccaagcttctggtttggacatggacgttatctctgaagaccaattggctgaatggttgtgtcacaactaccaaaactacgctcaagttgaattcatcaccgacaagtctcaagaaggttaccaattcgttaagggtttcggtggtatcggtggtttcttgagatacaaggttgacatggaagaagctttgggtgacgttggtgacggtggtgacgacttcgacccagacaccgacttcatctgatag

SEQ ID NO: 135 - DNA, 1317 bp, Artificial Sequence
  Description of Artificial Sequence Synthetic polynucleotide
atggacgaagctaagttgttgcaattggaaatgtggagattgagaaagcaattgcaaaagttggacaacaccaacaccaactctacctctgttgtttctttggttatgccaccaggtgaagacatcaacaagatggttcaaatgttgaaccaagaagctacccaagctgactgtatcaagtctagacaaaacagacaagctgttcaaaccgctatcatcttggctgctaacagatgtaagttgtacccaaagatgccaaagaacggtttggctgttttcgctggtgaagtttacgttgacggtaagatcaagaagatcgctgttcacttctctccatgtaagccaatcggtaacttcatgtactcttgtgacggtgttttccacacccaagaagttaaggacttgttgaccgttgaagaagtttacggtttcatcatcatggacggtcacggtaccttgatcgctaccttgtgtggttctcacagagaaatcaagcacagaatgttggttgacttgccaaagaagcacggtagaggtggtcaatcttctgttagattcgctagattgagaatggaagctagaggtaactacgttaagatcatcaccgaattgtgtaccaagtacttcatcaccggtgacagattgaacgtttctggtgttatcttggctggttctgctgacttcaaggacgttttggctggttctgacttcatggacccaagaatcaaggaaggtatcatcaagcaagttgacatcggttacggtatggaacaaggtttgtctcaagctatcgaacaagctggtgacatcttgaaggacgttagattgttcaaggaagttaagatcatcaacgaattcttggaccacatctctagagacaccagaaagtactgtttcggtatcatcgacaccttgagatgtttggaaatgggttctgttgaacacttggttgtttgggaagacttcccatgggacagagttcaatgtagaaactctgaaggtaccgaatactacaagttggttaacaccgttaccggtgctatcaacttggttgacaacgacgaatctatcagacaaggtgaagaagaaatcgaagctgaattgttcgttgaatggttggctcaaaacatcgaaaagttcggtgctgaaatccacttggttaccgaaaactctaacgaaggtacccaattctgttctggtttcggtggtttgggtggtatcttgagatggcaaatggacttgaacgaattgggtaagtacatggacatcggtaaggaaaacaacgacttggacgacttggaagacttcatgtgatag

SEQ ID NO: 136 - DNA, 1317 bp, Artificial Sequence
  Description of Artificial Sequence Synthetic polynucleotide
atggacaacgaagttgaaaagaacatcgaaatctggaaggttaagaagttggttcaatctttggaaaaggctagaggtaacggtacctctatgatctctttggttatcccaccaaagggtcaaatcccattgtaccaaaagatgttgaccgacgaatacggtaccgcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttgtgtgacaacaagttccacaccgaagttttgtctgaattgttgcaagctgacgacaagttcggtttcatcgttatggacggtcaaggtaccttgttcggttctgtttctggtaacaccagaaccgttttgcacaagttcaccgttgacttgccaaagaagcacggtagaggtggtcaatctgctttgagattcgctagattgagagaagaaaagagacacaactacgttagaaaggttgctgaagttgctgttcaaaacttcatcaccaacgacaaggttaacgttaagggtttgatcttggctggttctgctgacttcaagaccgacttggctaagtctgaattgttcgacccaagattggcttgtaaggttatctctatcgttgacgtttcttacggtggtgaaaacggtttcaaccaagctatcgaattgtctgctgaagctttggctaacgttaagtacgttcaagaaaagaagttgttggaagcttacttcgacgaaatctctcaagacaccggtaagttctgttacggtatcgacgacaccttgaaggctttggacttgggtgctgttgaaaagttgatcgttttcgaaaacttggaaaccatcagatacaccttcaaggacgctgaagacaacgaagttatcaagttcgctgaaccagaagctaaggacaagtctttcgctatcgacaaggctaccggtcaagaaatggacgttgtttctgaagaaccattgatcgaatggttggctgctaactacaagaacttcggtgctaccttggaattcatcaccgacaagtcttctgaaggtgctcaattcgttaccggtttcggtggtatcggtgctatgttgagatacaaggttaacttcgaacaattggttgacgaatctgaagacgaatactacgacgaagacgaaggttctgactacgacttcatctgatag

SEQ ID NO: 137 - DNA, 2058 bp, Artificial Sequence
  Description of Artificial Sequence Synthetic polynucleotide
atgtctgactctaaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacatgttcggtggtaaggaccacgtttctttgatcttcatgggtcacgttgacgctggtaagtctaccatgggtggtaacttgttgtacttgaccggttctgttgacaagagaaccatcgaaaagtacgaaagagaagctaaggacgctggtagacaaggttggtacttgtcttgggttatggacaccaacaaggaagaaagaaacgacggtaagaccatcgaagttggtaaggcttacttcgaaaccgaaaagagaagatacaccatcttggacgctccaggtcacaagatgtacgtttctgaaatgatcggtggtgcttctcaagctgacgttggtgttttggttatctctgctagaaagggtgaatacgaaaccggtttcgaaagaggtggtcaaaccagagaacacgctttgttggctaagacccaaggtgttaacaagatggttgttgttgttaacaagatggacgacccaaccgttaactggtctaaggaaagatacgaccaatgtgtttctaacgtttctaacttcttgagagctatcggttacaacatcaagaccgacgttgttttcatgccagtttctggttactctggtgctaacttgaaggaccacgttgacccaaaggaatgtccatggtacaccggtccaaccttgttggaatacttggacaccatgaaccacgttgacagacacatcaacgctccattcatgttgccaatcgctgctaagatgaaggacttgggtaccatcgttgaaggtaagatcgaatctggtcacatcaagaagggtcaatctaccttgttgatgccaaacaagaccgctgttgaaatccaaaacatctacaacgaaaccgaaaacgaagttgacatggctatgtgtggtgaacaagttaagttgagaatcaagggtgttgaagaagaagacatctctccaggtttcgttttgacctctccaaagaacccaatcaagtctgttaccaagttcgttgctcaaatcgctatcgttgaattgaagtctatcatcgctgctggtttctcttgtgttatgcacgttcacaccgctatcgaagaagttcacatcgttaagttgttgcacaagttggaaaagggtaccaacagaaagtctaagaagccaccagctttcgctaagaagggtatgaaggttatcgctgttttggaaaccgaagctccagtttgtgttgaaacctaccaagactacccacaattgggtagattcaccttgcgcgaccaaggtaccaccatcgctatcggtaagatcgttaagatcgctgaatga

SEQ ID NO: 138 - DNA, 1344 bp, Artificial Sequence; identical to SEQ ID NO: 126
  Description of Artificial Sequence Synthetic polynucleotide

SEQ ID NO: 139 - DNA, 2403 bp, Artificial Sequence
  Description of Artificial Sequence Synthetic polynucleotide
atggacaagaactctgacttcccagctttgggttctttgaaggttcaaaaccaatcttctaaggacaagaacaagaagaaggaaaaggaagttaagaaggaagacgaaaagaaggacgaagttaaggacgaaccacaaaacaacgaagaagctaagaagaccaagttggaagctaccggtagagttttcgctgctaagtctttcaccccaaaggaaccagttaagatcaactactacatcccacacgaagaaccaaccttgtctgaaatggacttcttcccaaccttgggtggtccagcttctaccgctccagttgtttctgaagctcaaaagagacacgctgaattcttggaaagacacgaattgttcaagccatacatctgtttgatcccaaaggaattgtggtacgttccagacccatacttcgacatgttcggttacccagttatggctttgttgccaggtttgtacgcttacttgtggaacacctacaacatcttcttcaccccagaaggtgcttggaacaccaacaaggaaaccgttatgtctaccttgggtgaattgtggacccacgctgaatggagagacagaatcatcgctaaggaacaaaaggaaaccgaagaatgggaaagacaaatgagagaatgggaagaagaaaacgctgaagacgacgaaggtttgtctatcgacgacatggaaaactacggtaagggtaagaagaagaacaagaacaagaagaagaaggacaaggctaaaagaccaccaccaccaccaccaaagtctttgtcttcttacaagagattcgaaaagaagaaggaagacgttgttgttccaaagaagaacatcggtttcaaggaagtttctgaaatcaccttcgaagaagaagttgttgaagttgacgaaaccagacaaccatcttctttggttttcatcggtccagttgacgctgttaagtctaccatctgtggtaacttgatgttcatgaccggtatggttgacgaaagaaccatcgaaaagttcaagcaagaagctaaggaaaagaacagagactcttggtggttggcttacgttatggacatcaacgacgacgaaaagtctaagggtaagaccgttgaagttggtagagctaccatggaaaccccaaccaagagatacaccatcttcgacgctccaggtcacaagaactacgttccagacatgatcatgggtgctgctatggctgacgttgctgctttggttatctctgctagaaagggtgaattcgaagctggtttcgaacgcgacggtcaaaccagagaacacgctcaattggctagatctttgggtgttaacaagttggttgttgttgttaacgaaatggacgaagaaaccgttcaatggtctgaagaaagatacaacgacatcttgtctggtgttaccccattcttgatcgaccaatgtggttacaagagagaagacttgatcttcgttccaatctctggtttgaacggtcacaacatcgacaagttggcttcttgttgtccatggtacaccggtccaaccttgttggaaatcttggactgtatcgaaccaccaaagagaaacatcgacggtccattgagagttccagttttggacaagatgaaggacagaggtgttgttgctttcggtaaggttgaatctggtgttatcagaatcggtccaaagttggctgttatgccaaacaacaccaagtgtcaagttgttggtatctacaactgtaagttggaattggttagatacgctaacccaggtgaaaacatccaaatcaaggttagaatgatcgaagacgaaaaccaaatcaacaagggtgacgttttgtgtccatacgacaacttggctccaatcaccgacttgttcgaagctgaattgaccatcttggaattgttgccacacagaccaatcatcaccccaggttacaagtctatgatgcacttgcacaccatctctgacgaaatcgttatccaaaccttgaccggtatctacgaattggacggttctggtaaggaatacgttaagaagaacccaaagtactgtaagtctggttctaaggttatcgttaagatctctaccagagttccagtttgtttggaaaagtacgaattcatcgttcacatgggtagattcaccttgcgcgacgaaggtaagaccatcgctttgggtaaggttttgagatacaagccagctgttatcaagaaggttgaagaaatcccaccaggtgttggtgacgaaggtcaagctaagttggaagaatctgaagaattctctggttctagaggtgactctccatctaaggacgacaacaagtacgaagttatcacctacgacccagaagaagacaccatcatcgcttcttctaccggttctgaaaacgctgaataatag

SEQ ID NO: 140 - DNA, 1317 bp, Artificial Sequence; identical to SEQ ID NO: 127
  Description of Artificial Sequence Synthetic polynucleotide

SEQ ID NO: 141 - DNA, 2403 bp, Artificial Sequence; identical to SEQ ID NO: 139
  Description of Artificial Sequence Synthetic polynucleotide

SEQ ID NO: 142 - DNA, 1314 bp, Artificial Sequence; identical to SEQ ID NO: 128
  Description of Artificial Sequence Synthetic polynucleotide

SEQ ID NO: 143 - DNA, 1233 bp, Artificial Sequence
  Description of Artificial Sequence Synthetic polynucleotide
atggttgactctggtaagtctacctcttgtggtcacttgatctacaagtgtggtggtatcgacaagagaaccatcgaaaagtacgaaaaggaagctaaggaaatgggtaagtcttctttcaagtacgcttgggttttggacaagttgaaggctgaaagagaaagaggtatcaccatcgacatctctttgttcaagttccaaaccgacaagttctacttcaccatcatcgacgctccaggtcacagagacttcatcaagaacatgatcaccggtacctctcaagctgacgttgctatcttgatcatcgctgctggtaagggtgaattcgaagctggttactctaagaacggtcaaaccagagaacacgctttgttggctttcaccttgggtgttaagcaaatggttgttggtgttaacaagatggacgacaagtctgctgaatggaagcaagacagatacttggaaatcaagcaagaagtttctgaatacttgaagaaggttggttacaacccagctaaggttccattcatcccaatctctggttggttgggtgacaacatggttgaaaagtctaccaacatgccatggtacgacggtccaaccttgttgggtgctttggacaacgttcaaccaccaaagagacacgttgacaagccattgagattgccagttcaagacgtttacaagatctctggtatcggtaccgttccagttggtagagttgaaaccggtgttctcagaccaggtaccgttgttgttttcgctccatctggtatctctaccgaagttaagtctgttgaaatgcaccacgaatctttggaagaagctttgccaggtgacaacgttggtttcaacatcaagaacatcgctgttaaccaaatcaagagaggttacgttgcttctgactctagatctgacccagctagagaatctatcgacttcaccgctcaagttatcatcttgcaccacccaggtcaaatctctgttggttacaccccagttttggactgtcacaccgctcacatcgcttgtagattcaacgacttgaagcaaaaggttgacagaagatctggtgctgttttggaacaagaaccaaagttcgttaagtctggtgacgctgctatcgttaccttgatcccaaccaagtctatgtgtgttgaaccattcaccgaatacccaccattgggtagattcgctgttagagacatgagacaaaccgttgctgtttaatag

SEQ ID NO: 144 - DNA, 1311 bp, Artificial Sequence; identical to SEQ ID NO: 129
  Description of Artificial Sequence Synthetic polynucleotide

SEQ ID NO: 145 - DNA, 1941 bp, Artificial Sequence
  Description of Artificial Sequence Synthetic polynucleotide
atggactaccaagaagctaagagattggctaaggaagaaaagttgagaaagttgaaggctaagatccaagaaaacaaggttgaagacttccaagttaccgaacaacaaggtccattgccaccatacgacgaccaaggtaaccaacaaggtaagtggttgaccaccaagtacaagttcggtgaatggtactacgacatgaacggtaacccagttttcatctctccagacgacgaatacgaccaaaacggttctttgtacgacgctgaatctaacccatacttggactacgcttacgacaagttcggtaacccatgtccagttatcttgatgacccaagacaacgaatacttcttgttcaccccaatcttcgaaatccaaatcccacaatctatcatcaaggacatcaaccaaaagcaagaaaagccagctacccaaaaggttgaaaccgttgctaaggaaaagaaggctccaatccaaaaggctccagctccaccaaagatcccaaagagattgttgaaggaacaagaagctgctaactcttctgctgttgttgctgactacaagcacgaaaagttggttaccgttcaagaagctatcgacttcgaatctaagaacttcaaggaaggtcaagaaatggttaaggttgacagagaaagagactctgttaacatcgttttcatcggtcacgttgacgctggtaagtctaccttgtctggtagaatcttgaagaactgtggtgaagttgacgaaaccgaaatcagaaagttcgaattggaagctaaggaaaagaacagagaatcttgggttttggcttacatcatggacatcaacgaagaagaaagatctaagggtatcaccgttgaatgtggtaaggctcacttccaattggctaacaagagattcgttttgttggacgctccaggtcacaagaactacgttccaaacatgatcgctggtgcttgtcaagctgacgttgctgctttgatcatctctgctagacaaggtgaattcgaagctggtttcgaaggtggtcaaacccaagaacacgctcacttggctaaggctttgggtgttcaacacatgatctgtgttgtttctaagatggacgaagttaactgggacaagaagagatacgaccacatccacgactctgttgaaccattcttgagaaaccaagttggtatccaatctatcgaatgggttccaatcaacggtttcttgaacgaaaacatcgacaccccaatcccaaccgaaagatgtgaatggtacaagggtgacaccttgttcgacaagttcaacaaggttccagttccattgcgcgacccaaacggtccagttagaatcccagttttggacaagttgaaggaccaaggtcaattcttgttcggtaagatcgaatctggtaccatccgcgacgacttgtgggttaccttgatgccatacagaaagcaattccaaatcttgtctatctacaacaccaaggaccaaagagttttgtacgcttctgctggtgaaaacgttaagatcaagttgaagggtttggaagacaaggacatcgaaagaggttacatggtttgttctaccgaagacttgtgtccaatcacccaattgttcatcgctgaaatcaccatcttgcaattgccagaacacaagccaatcatgtctcaaggttactcttgtgttttgcacatgcacacctctgttgctgaaatcgaaatcgaagaagttgaagctgttcaaaacccagaaaacaagaagttgaccaagaacaccttcttgaagtctaaccaaaccggtgttgttaagatcggtatcaagggtggtttgatgtgtttggaaaagttcgaaaccatctctcaattgggtagattcaccttgcgcgacgaagaaaagaccatcggtttcggtagagttatgaagatcaagccatacaaggtttga

SEQ ID NO: 146 - DNA, 1461 bp, Artificial Sequence; identical to SEQ ID NO: 130
  Description of Artificial Sequence Synthetic polynucleotide

SEQ ID NO: 147 - DNA, 1941 bp, Artificial Sequence; identical to SEQ ID NO: 145
  Description of Artificial Sequence Synthetic polynucleotide

SEQ ID NO: 148 - DNA, 1362 bp, Artificial Sequence; identical to SEQ ID NO: 131
  Description of Artificial Sequence Synthetic polynucleotide

SEQ ID NO: 149 - DNA, 1941 bp, Artificial Sequence; identical to SEQ ID NO: 145
  Description of Artificial Sequence Synthetic polynucleotide

SEQ ID NO: 150 - DNA, 1317 bp, Artificial Sequence; identical to SEQ ID NO: 132
  Description of Artificial Sequence Synthetic polynucleotide

SEQ ID NO: 151 - DNA, 2271 bp, Artificial Sequence
  Description of Artificial Sequence Synthetic polynucleotide
atgtcttaccaatacggtcaacaaatgggtcaatacccatacgacccaaacatgaacatgatgggtttcgacccacaaatgtaccaagaatacgcttactactacttgggtggtccaccaaccccaccaccaccaaagggtccatacccaggtatcacccacgaagactacgaatctttcgacatcaacaagcaaatcttgttccaaagattcttgggtgaaaccgctgcttactacgctaagcacttgccaaagtaccaaaaggaaatggaagaattcttgaacaccaacaccgcttaccaaatgaacgaatctgaaaagcaattgatgcaatcttacttggacttcaagaagaaggaaaaggaatacgaatctttcttgaagcaattggaacaacaagctttgaacccagaattggaacaacaaaagaagttggaacaacaaaagttggaacaacaaaagatcgaacaacaaaagttggaagaacaaaagaagcaacaacaattggaacaacaaaagcaacaacagcaacagcagcaacagcaaccacaacaagaacaaccaaaggaaggtgctaccaccgctgttagaccaaagaagaagttgaacttgaacgctaagccattggaaatcgctttgaacccaccaaagatgccaaacttcccaaagcacccagacttcttggacttcgacaagttctggaacaactactctacctacatctctttgtacaacccatcttgtgaagacgaatacaagaactacccaaagccagaacaattgaagaagaaggaagctgacgaagaagctaagagaaagaagaagcaagaagaaatcgaaagagctatcaagaagagacaagacgctcaagaaagagctaaggacaagccagctcaatctgttaacttggttgaacaagttgttaagttggaaggtgaagttgacttgcaaaagtacgttgacccagacgaaaccagacaaccagttaacttggttttcatcggtcacgttgacgctggtaagtctaccttgtgtggtagattgttgttggaattgggtgaagtttctgaagctgacatcaagaagtacgaacaagaagctgttcaaaacaacagagactcttggtggttggcttacgttatggaccaaaacgaagaagaaaagcaaaagggtaagaccgttgaatgtggtaaggctcaattcgttaccaagcaaaagagattcatcttggctgacgctccaggtcacaagaactacgttccaaacatgatcatgggtgcttgtcaagctgacttggctggtttgatcgtttctgctaagaccggtgaattcgaatctggtttcgaaaagggtggtcaaacccaagaacacgctttgttggctaagtctttgggtgttgaccacatcatcatcatcgttaccaagatggacaccatcgactggaaccaagacagattcaacttgatctctcaaaacatccaagaattcgttttgaagcaatgtaagttcgacaacatctacgttatcccaatcgacgctttgtctggttctaacatcaagtctagagttgacgaatctaagtgtaactggtacaagggtccatctttgatcgacttgatcgacaccgtttctatcccaaagagaaacgaagaaggtccaatcagaatgccaatcttggacaagttcaaggacatgggttctttgtacatctacggtaagttggaatctggtaagatcatcgaaggtttggacgtttctatctacccaaagaagcaaccattccaaatcaccgaattgtacaacatgaaggaccaaaagatgaagtacgctaaggctggtgaaaacatcaagatcaaggttaagaacatcgaagaagaagaaatcaagagaggttacatgatgtgtaacttgacctctaacccatgtttggtttctcaagaattccaagctaagatcagattgttggacttgccagaatctagaagaatcttctctgaaggttaccaatgtatcatgcacttgcactctgctgttgaagaaatcgaaatctcttgtgttgaagctgttatcgacgctgaaaccaagaagtctatcaagcaaaacttcttgaagtctttcaacgaaggtatcgctaagatctctatcaagaacccagtttgtatggaaaagtacgaaaccttggctcaattgggtagattcgctttgcgcgacgacggtaagaccatcggtttcggtgaaatcttgaaggttaagccagttaagcaaggttga

SEQ ID NO: 152 - DNA, 1317 bp, Artificial Sequence; identical to SEQ ID NO: 133
  Description of Artificial Sequence Synthetic polynucleotide

SEQ ID NO: 153 - DNA, 2271 bp, Artificial Sequence; identical to SEQ ID NO: 151
  Description of Artificial Sequence Synthetic polynucleotide

SEQ ID NO: 154 - DNA, 1344 bp, Artificial Sequence; identical to SEQ ID NO: 126
  Description of Artificial Sequence Synthetic polynucleotide

SEQ ID NO: 155 - DNA, 2271 bp, Artificial Sequence
  Description of Artificial Sequence Synthetic polynucleotide
atggacaagaactctgaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacgttgacgaaaccagacaaccatcttctttggttttcatcggtccagttgacgctgttaagtctaccatctgtggtaacttgatgttcatgaccggtatggttgacgaaagaaccatcgaaaagttcaagcaagaagctaaggaaaagaacagagactcttggtggttggcttacgttatggacatcaacgacgacgaaaagtctaagggtaagaccgttgaagttggtagagctaccatggaaaccccaaccaagagatacaccatcttcgacgctccaggtcacaagaactacgttccagacatgatcatgggtgctgctatggctgacgttgctgctttggttatctctgctagaaagggtgaattcgaagctggtttcgaacgcgacggtcaaaccagagaacacgctcaattggctagatctttgggtgttaacaagttggttgttgttgttaacgaaatggacgaagaaaccgttcaatggtctgaagaaagatacaacgacatcttgtctggtgttaccccattcttgatcgaccaatgtggttacaagagagaagacttgatcttcgttccaatctctggtttgaacggtcacaacatcgacaagttggcttcttgttgtccatggtacaccggtccaaccttgttggaaatcttggactgtatcgaaccaccaaagagaaacatcgacggtccattgagagttccagttttggacaagatgaaggacagaggtgttgttgctttcggtaaggttgaatctggtgttatcagaatcggtccaaagttggctgttatgccaaacaacaccaagtgtcaagttgttggtatctacaactgtaagttggaattggttagatacgctaacccaggtgaaaacatccaaatcaaggttagaatgatcgaagacgaaaaccaaatcaacaagggtgacgttttgtgtccatacgacaacttggctccaatcaccgacttgttcgaagctgaattgaccatcttggaattgttgccacacagaccaatcatcaccccaggttacaagtctatgatgcacttgcacaccatctctgacgaaatcgttatccaaaccttgaccggtatctacgaattggacggttctggtaaggaatacgttaagaagaacccaaagtactgtaagtctggttctaaggttatcgttaagatctctaccagagttccagtttgtttggaaaagtacgaattcatcgttcacatgggtagattcaccttgcgcgacgaaggtaagaccatcgctttgggtaaggttttgagatacaagccagctgttatcaagaaggttgaagaaatcccaccaggtgttggtgacgaaggtcaagctaagttggaagaatctgaagaattctctggttctagaggtgactctccatctaaggacgacaacaagtacgaagttatcacctacgacccagaagaagacaccatcatcgcttcttctaccggttctgaaaacgctgaataatag

SEQ ID NO: 156 - DNA, 1317 bp, Artificial Sequence; identical to SEQ ID NO: 127
  Description of Artificial Sequence Synthetic polynucleotide

SEQ ID NO: 157 - DNA, 2268 bp, Artificial Sequence
  Description of Artificial Sequence Synthetic polynucleotide
atgtctgactctaaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacgttgacgaaaccagacaaccatcttctttggttttcatcggtccagttgacgctgttaagtctaccatctgtggtaacttgatgttcatgaccggtatggttgacgaaagaaccatcgaaaagttcaagcaagaagctaaggaaaagaacagagactcttggtggttggcttacgttatggacatcaacgacgacgaaaagtctaagggtaagaccgttgaagttggtagagctaccatggaaaccccaaccaagagatacaccatcttcgacgctccaggtcacaagaactacgttccagacatgatcatgggtgctgctatggctgacgttgctgctttggttatctctgctagaaagggtgaattcgaagctggtttcgaacgcgacggtcaaaccagagaacacgctcaattggctagatctttgggtgttaacaagttggttgttgttgttaacgaaatggacgaagaaaccgttcaatggtctgaagaaagatacaacgacatcttgtctggtgttaccccattcttgatcgaccaatgtggttacaagagagaagacttgatcttcgttccaatctctggtttgaacggtcacaacatcgacaagttggcttcttgttgtccatggtacaccggtccaaccttgttggaaatcttggactgtatcgaaccaccaaagagaaacatcgacggtccattgagagttccagttttggacaagatgaaggacagaggtgttgttgctttcggtaaggttgaatctggtgttatcagaatcggtccaaagttggctgttatgccaaacaacaccaagtgtcaagttgttggtatctacaactgtaagttggaattggttagatacgctaacccaggtgaaaacatccaaatcaaggttagaatgatcgaagacgaaaaccaaatcaacaagggtgacgttttgtgtccatacgacaacttggctccaatcaccgacttgttcgaagctgaattgaccatcttggaattgttgccacacagaccaatcatcaccccaggttacaagtctatgatgcacttgcacaccatctctgacgaaatcgttatccaaaccttgaccggtatctacgaattggacggttctggtaaggaatacgttaagaagaacccaaagtactgtaagtctggttctaaggttatcgttaagatctctaccagagttccagtttgtttggaaaagtacgaattcatcgttcacatgggtagattcaccttgcgcgacgaaggtaagaccatcgctttgggtaaggttttgagatacaagccagctgttatcaagaaggttgaagaaatcccaccaggtgttggtgacgaaggtcaagctaagttggaagaatctgaagaattctctggttctagaggtgactctccatctaaggacgacaacaagtacgaagttatcacctacgacccagaagaagacaccatcatcgcttcttctaccggttctgaaaacgctgaataatag

SEQ ID NO: 158 - DNA, 1317 bp, Artificial Sequence; identical to SEQ ID NO: 132
  Description of Artificial Sequence Synthetic polynucleotide

SEQ ID NO: 159 - DNA, 2067 bp, Artificial Sequence
  Description of Artificial Sequence Synthetic polynucleotide
atgtctgactctaaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacccagacgaaaccagacaaccagttaacttggttttcatcggtcacgttgacgctggtaagtctaccttgtgtggtagattgttgttggaattgggtgaagtttctgaagctgacatcaagaagtacgaacaagaagctgttcaaaacaacagagactcttggtggttggcttacgttatggaccaaaacgaagaagaaaagcaaaagggtaagaccgttgaatgtggtaaggctcaattcgttaccaagcaaaagagattcatcttggctgacgctccaggtcacaagaactacgttccaaacatgatcatgggtgcttgtcaagctgacttggctggtttgatcgtttctgctaagaccggtgaattcgaatctggtttcgaaaagggtggtcaaacccaagaacacgctttgttggctaagtctttgggtgttgaccacatcatcatcatcgttaccaagatggacaccatcgactggaaccaagacagattcaacttgatctctcaaaacatccaagaattcgttttgaagcaatgtaagttcgacaacatctacgttatcccaatcgacgctttgtctggttctaacatcaagtctagagttgacgaatctaagtgtaactggtacaagggtccatctttgatcgacttgatcgacaccgtttctatcccaaagagaaacgaagaaggtccaatcagaatgccaatcttggacaagttcaaggacatgggttctttgtacatctacggtaagttggaatctggtaagatcatcgaaggtttggacgtttctatctacccaaagaagcaaccattccaaatcaccgaattgtacaacatgaaggaccaaaagatgaagtacgctaaggctggtgaaaacatcaagatcaaggttaagaacatcgaagaagaagaaatcaagagaggttacatgatgtgtaacttgacctctaacccatgtttggtttctcaagaattccaagctaagatcagattgttggacttgccagaatctagaagaatcttctctgaaggttaccaatgtatcatgcacttgcactctgctgttgaagaaatcgaaatctcttgtgttgaagctgttatcgacgctgaaaccaagaagtctatcaagcaaaacttcttgaagtctttcaacgaaggtatcgctaagatctctatcaagaacccagtttgtatggaaaagtacgaaaccttggctcaattgggtagattcgctttgcgcgacgacggtaagaccatcggtttcggtgaaatcttgaaggttaagccagttaagcaaggttga

SEQ ID NO: 160 - DNA, 1317 bp, Artificial Sequence; identical to SEQ ID NO: 133
  Description of Artificial Sequence Synthetic polynucleotide

SEQ ID NO: 161 - DNA, 2067 bp, Artificial Sequence; identical to SEQ ID NO: 159
  Description of Artificial Sequence Synthetic polynucleotide

SEQ ID NO: 162 - protein, 4 aa, Artificial Sequence
  Description of Artificial Sequence Synthetic peptide
NIKS